SEQUENCE LISTING (1387 sequences)

SEQ ID NO: 1 - RNA, 19 nt, Artificial Sequence
  siRNA strand - sequence modified as per summary sequence table at the end of the description
ucgaaguauuccgcguacg

SEQ ID NO: 2 - RNA, 19 nt, Artificial Sequence
  siRNA strand - sequence modified as per summary sequence table at the end of the description
cguacgcggaauacuucga

SEQ ID NO: 3 - RNA, 19 nt, Artificial Sequence
  siRNA strand - sequence modified as per summary sequence table at the end of the description
aaccagaagaagcagguga

SEQ ID NO: 4 - RNA, 19 nt, Artificial Sequence
  siRNA strand - sequence modified as per summary sequence table at the end of the description
ucaccugcuucuucugguu

SEQ ID NO: 5 - RNA, 19 nt, Artificial Sequence
  siRNA strand
ucaccugcuucuucugguu

SEQ ID NO: 6 - RNA, 19 nt, Artificial Sequence
  siRNA strand
aaccagaagaagcagguga

SEQ ID NO: 7 - RNA, 19 nt, Artificial Sequence
  siRNA strand
ucaccugcuucuucugguu

SEQ ID NO: 8 - RNA, 19 nt, Artificial Sequence
  siRNA strand
augucuuucacacuggcuu

SEQ ID NO: 9 - RNA, 19 nt, Artificial Sequence
  siRNA strand
aagccagugugaaagacau

SEQ ID NO: 10 - RNA, 19 nt, Artificial Sequence
  siRNA strand
auugaguacacgcagacug

SEQ ID NO: 11 - RNA, 19 nt, Artificial Sequence
  siRNA strand
cagucugcguguacucaau

SEQ ID NO: 12 - RNA, 19 nt, Artificial Sequence
  siRNA strand
aaguugauggugaucccgg

SEQ ID NO: 13 - RNA, 19 nt, Artificial Sequence
  siRNA strand
ccgggaucaccaucaacuu

SEQ ID NO: 14 - RNA, 19 nt, Artificial Sequence
  siRNA strand
uucuggaucguccacuggc

SEQ ID NO: 15 - RNA, 19 nt, Artificial Sequence
  siRNA strand
gccaguggacgauccagaa

SEQ ID NO: 16 - RNA, 19 nt, Artificial Sequence
  siRNA strand
auucacagaacagaggaac

SEQ ID NO: 17 - RNA, 19 nt, Artificial Sequence
  siRNA strand
guuccucuguucugugaau

SEQ ID NO: 18 - RNA, 19 nt, Artificial Sequence
  siRNA strand
guagucauggcuguccucu

SEQ ID NO: 19 - RNA, 19 nt, Artificial Sequence
  siRNA strand
agaggacagccaugacuac

SEQ ID NO: 20 - RNA, 19 nt, Artificial Sequence
  siRNA strand
aguuguaguaaguucccag

SEQ ID NO: 21 - RNA, 19 nt, Artificial Sequence
  siRNA strand
cugggaacuuacuacaacu

SEQ ID NO: 22 - RNA, 19 nt, Artificial Sequence
  siRNA strand
uuguacccuaggaaauacc

SEQ ID NO: 23 - RNA, 19 nt, Artificial Sequence
  siRNA strand
gguauuuccuaggguacaa

SEQ ID NO: 24 - RNA, 19 nt, Artificial Sequence
  siRNA strand
aaccagaagaagcagguga

SEQ ID NO: 25 - RNA, 19 nt, Artificial Sequence
  siRNA strand
ucaccugcuucuucugguu

SEQ ID NO: 26 - RNA, 19 nt, Artificial Sequence
  siRNA strand
uaacaacccagcguggaau

SEQ ID NO: 27 - RNA, 19 nt, Artificial Sequence
  siRNA strand
auuccacgcuggguuguua

SEQ ID NO: 28 - RNA, 19 nt, Artificial Sequence
  siRNA strand
guuucucucauccaggccg

SEQ ID NO: 29 - RNA, 19 nt, Artificial Sequence
  siRNA strand
cggccuggaugagagaaac

SEQ ID NO: 30 - RNA, 19 nt, Artificial Sequence
  siRNA strand
gcaucuucugggcuuuggc

SEQ ID NO: 31 - RNA, 19 nt, Artificial Sequence
  siRNA strand
gccaaagcccagaagaugc

SEQ ID NO: 32 - RNA, 19 nt, Artificial Sequence
  siRNA strand
ucacacuggaaggugaaug

SEQ ID NO: 33 - RNA, 19 nt, Artificial Sequence
  siRNA strand
cauucaccuuccaguguga

SEQ ID NO: 34 - RNA, 19 nt, Artificial Sequence
  siRNA strand
cacagaugugucgaccccg

SEQ ID NO: 35 - RNA, 19 nt, Artificial Sequence
  siRNA strand
cggggucgacacaucugug

SEQ ID NO: 36 - RNA, 19 nt, Artificial Sequence
  siRNA strand
uguacccuaggaaauacca

SEQ ID NO: 37 - RNA, 19 nt, Artificial Sequence
  siRNA strand
ugguauuuccuaggguaca

SEQ ID NO: 38 - RNA, 19 nt, Artificial Sequence
  siRNA strand
aggucagcucgcaccagag

SEQ ID NO: 39 - RNA, 19 nt, Artificial Sequence
  siRNA strand
cucuggugcgagcugaccu

SEQ ID NO: 40 - RNA, 19 nt, Artificial Sequence
  siRNA strand
agugcaucucaggucagcu

SEQ ID NO: 41 - RNA, 19 nt, Artificial Sequence
  siRNA strand
agcugaccugagaugcacu

SEQ ID NO: 42 - RNA, 19 nt, Artificial Sequence
  siRNA strand
ugccgagacagcucacaga

SEQ ID NO: 43 - RNA, 19 nt, Artificial Sequence
  siRNA strand
ucugugagcugucucggca

SEQ ID NO: 44 - RNA, 19 nt, Artificial Sequence
  siRNA strand
agugggugccgagacagcu

SEQ ID NO: 45 - RNA, 19 nt, Artificial Sequence
  siRNA strand
agcugucucggcacccacu

SEQ ID NO: 46 - RNA, 19 nt, Artificial Sequence
  siRNA strand
aagugggugccgagacagc

SEQ ID NO: 47 - RNA, 19 nt, Artificial Sequence
  siRNA strand
gcugucucggcacccacuu

SEQ ID NO: 48 - RNA, 19 nt, Artificial Sequence
  siRNA strand
acaucaggcggcagugacu

SEQ ID NO: 49 - RNA, 19 nt, Artificial Sequence
  siRNA strand
agucacugccgccugaugu

SEQ ID NO: 50 - RNA, 19 nt, Artificial Sequence
  siRNA strand
aacaacaucaggcggcagu

SEQ ID NO: 51 - RNA, 19 nt, Artificial Sequence
  siRNA strand
acugccgccugauguuguu

SEQ ID NO: 52 - RNA, 19 nt, Artificial Sequence
  siRNA strand
uaacaacaucaggcggcag

SEQ ID NO: 53 - RNA, 19 nt, Artificial Sequence
  siRNA strand
cugccgccugauguuguua

SEQ ID NO: 54 - RNA, 19 nt, Artificial Sequence
  siRNA strand
aguaacaacaucaggcggc

SEQ ID NO: 55 - RNA, 19 nt, Artificial Sequence
  siRNA strand
gccgccugauguuguuacu

SEQ ID NO: 56 - RNA, 19 nt, Artificial Sequence
  siRNA strand
aagaguaacaacaucaggc

SEQ ID NO: 57 - RNA, 19 nt, Artificial Sequence
  siRNA strand
gccugauguuguuacucuu

SEQ ID NO: 58 - RNA, 19 nt, Artificial Sequence
  siRNA strand
uccuuuuggaguggaagag

SEQ ID NO: 59 - RNA, 19 nt, Artificial Sequence
  siRNA strand
cucuuccacuccaaaagga

SEQ ID NO: 60 - RNA, 19 nt, Artificial Sequence
  siRNA strand
acgggcauccuuuuggagu

SEQ ID NO: 61 - RNA, 19 nt, Artificial Sequence
  siRNA strand
acuccaaaaggaugcccgu

SEQ ID NO: 62 - RNA, 19 nt, Artificial Sequence
  siRNA strand
uuucucuuggaguccucac

SEQ ID NO: 63 - RNA, 19 nt, Artificial Sequence
  siRNA strand
gugaggacuccaagagaaa

SEQ ID NO: 64 - RNA, 19 nt, Artificial Sequence
  siRNA strand
aguagcacccccgccgaag

SEQ ID NO: 65 - RNA, 19 nt, Artificial Sequence
  siRNA strand
cuucggcgggggugcuacu

SEQ ID NO: 66 - RNA, 19 nt, Artificial Sequence
  siRNA strand
agaguagcacccccgccga

SEQ ID NO: 67 - RNA, 19 nt, Artificial Sequence
  siRNA strand
ucggcgggggugcuacucu

SEQ ID NO: 68 - RNA, 19 nt, Artificial Sequence
  siRNA strand
accagaguagcacccccgc

SEQ ID NO: 69 - RNA, 19 nt, Artificial Sequence
  siRNA strand
gcgggggugcuacucuggu

SEQ ID NO: 70 - RNA, 19 nt, Artificial Sequence
  siRNA strand
auaccagaguagcaccccc

SEQ ID NO: 71 - RNA, 19 nt, Artificial Sequence
  siRNA strand
gggggugcuacucugguau

SEQ ID NO: 72 - RNA, 19 nt, Artificial Sequence
  siRNA strand
aauaccagaguagcacccc

SEQ ID NO: 73 - RNA, 19 nt, Artificial Sequence
  siRNA strand
ggggugcuacucugguauu

SEQ ID NO: 74 - RNA, 19 nt, Artificial Sequence
  siRNA strand
uacccuaggaaauaccaga

SEQ ID NO: 75 - RNA, 19 nt, Artificial Sequence
  siRNA strand
ucugguauuuccuagggua

SEQ ID NO: 76 - RNA, 19 nt, Artificial Sequence
  siRNA strand
uguacccuaggaaauacca

SEQ ID NO: 77 - RNA, 19 nt, Artificial Sequence
  siRNA strand
ugguauuuccuaggguaca

SEQ ID NO: 78 - RNA, 19 nt, Artificial Sequence
  siRNA strand
uuguacccuaggaaauacc

SEQ ID NO: 79 - RNA, 19 nt, Artificial Sequence
  siRNA strand
gguauuuccuaggguacaa

SEQ ID NO: 80 - RNA, 19 nt, Artificial Sequence
  siRNA strand
ugaguacaccuggcugacc

SEQ ID NO: 81 - RNA, 19 nt, Artificial Sequence
  siRNA strand
ggucagccagguguacuca

SEQ ID NO: 82 - RNA, 19 nt, Artificial Sequence
  siRNA strand
ugccugaguacaccuggcu

SEQ ID NO: 83 - RNA, 19 nt, Artificial Sequence
  siRNA strand
agccagguguacucaggca

SEQ ID NO: 84 - RNA, 19 nt, Artificial Sequence
  siRNA strand
acgcagacugccugaguac

SEQ ID NO: 85 - RNA, 19 nt, Artificial Sequence
  siRNA strand
guacucaggcagucugcgu

SEQ ID NO: 86 - RNA, 19 nt, Artificial Sequence
  siRNA strand
acacgcagacugccugagu

SEQ ID NO: 87 - RNA, 19 nt, Artificial Sequence
  siRNA strand
acucaggcagucugcgugu

SEQ ID NO: 88 - RNA, 19 nt, Artificial Sequence
  siRNA strand
aguacacgcagacugccug

SEQ ID NO: 89 - RNA, 19 nt, Artificial Sequence
  siRNA strand
caggcagucugcguguacu

SEQ ID NO: 90 - RNA, 19 nt, Artificial Sequence
  siRNA strand
ugaguacacgcagacugcc

SEQ ID NO: 91 - RNA, 19 nt, Artificial Sequence
  siRNA strand
ggcagucugcguguacuca

SEQ ID NO: 92 - RNA, 19 nt, Artificial Sequence
  siRNA strand
uugaguacacgcagacugc

SEQ ID NO: 93 - RNA, 19 nt, Artificial Sequence
  siRNA strand
gcagucugcguguacucaa

SEQ ID NO: 94 - RNA, 19 nt, Artificial Sequence
  siRNA strand
auugaguacacgcagacug

SEQ ID NO: 95 - RNA, 19 nt, Artificial Sequence
  siRNA strand
cagucugcguguacucaau

SEQ ID NO: 96 - RNA, 19 nt, Artificial Sequence
  siRNA strand
uggcgauugaguacacgca

SEQ ID NO: 97 - RNA, 19 nt, Artificial Sequence
  siRNA strand
ugcguguacucaaucgcca

SEQ ID NO: 98 - RNA, 19 nt, Artificial Sequence
  siRNA strand
aguggcgauugaguacacg

SEQ ID NO: 99 - RNA, 19 nt, Artificial Sequence
  siRNA strand
cguguacucaaucgccacu

SEQ ID NO: 100 - RNA, 19 nt, Artificial Sequence
  siRNA strand
aaguggcgauugaguacac

SEQ ID NO: 101 - RNA, 19 nt, Artificial Sequence
  siRNA strand
guguacucaaucgccacuu

SEQ ID NO: 102 - RNA, 19 nt, Artificial Sequence
  siRNA strand
agaaguggcgauugaguac

SEQ ID NO: 103 - RNA, 19 nt, Artificial Sequence
  siRNA strand
guacucaaucgccacuucu

SEQ ID NO: 104 - RNA, 19 nt, Artificial Sequence
  siRNA strand
uaagauccugggagaagug

SEQ ID NO: 105 - RNA, 19 nt, Artificial Sequence
  siRNA strand
cacuucucccaggaucuua

SEQ ID NO: 106 - RNA, 19 nt, Artificial Sequence
  siRNA strand
auucccggcggguaagauc

SEQ ID NO: 107 - RNA, 19 nt, Artificial Sequence
  siRNA strand
gaucuuacccgccgggaau

SEQ ID NO: 108 - RNA, 19 nt, Artificial Sequence
  siRNA strand
agauucccggcggguaaga

SEQ ID NO: 109 - RNA, 19 nt, Artificial Sequence
  siRNA strand
ucuuacccgccgggaaucu

SEQ ID NO: 110 - RNA, 19 nt, Artificial Sequence
  siRNA strand
uagauucccggcggguaag

SEQ ID NO: 111 - RNA, 19 nt, Artificial Sequence
  siRNA strand
cuuacccgccgggaaucua

SEQ ID NO: 112 - RNA, 19 nt, Artificial Sequence
  siRNA strand
acuagauucccggcgggua

SEQ ID NO: 113 - RNA, 19 nt, Artificial Sequence
  siRNA strand
uacccgccgggaaucuagu

SEQ ID NO: 114 - RNA, 19 nt, Artificial Sequence
  siRNA strand
aggcacuagauucccggcg

SEQ ID NO: 115 - RNA, 19 nt, Artificial Sequence
  siRNA strand
cgccgggaaucuagugccu

SEQ ID NO: 116 - RNA, 19 nt, Artificial Sequence
  siRNA strand
aaggcacuagauucccggc

SEQ ID NO: 117 - RNA, 19 nt, Artificial Sequence
  siRNA strand
gccgggaaucuagugccuu

SEQ ID NO: 118 - RNA, 19 nt, Artificial Sequence
  siRNA strand
uuggcgguuucacugcgga

SEQ ID NO: 119 - RNA, 19 nt, Artificial Sequence
  siRNA strand
uccgcagugaaaccgccaa

SEQ ID NO: 120 - RNA, 19 nt, Artificial Sequence
  siRNA strand
uuuggcgguuucacugcgg

SEQ ID NO: 121 - RNA, 19 nt, Artificial Sequence
  siRNA strand
ccgcagugaaaccgccaaa

SEQ ID NO: 122 - RNA, 19 nt, Artificial Sequence
  siRNA strand
uguaguaaguucccaggcg

SEQ ID NO: 123 - RNA, 19 nt, Artificial Sequence
  siRNA strand
cgccugggaacuuacuaca

SEQ ID NO: 124 - RNA, 19 nt, Artificial Sequence
  siRNA strand
uuguaguaaguucccaggc

SEQ ID NO: 125 - RNA, 19 nt, Artificial Sequence
  siRNA strand
gccugggaacuuacuacaa

SEQ ID NO: 126 - RNA, 19 nt, Artificial Sequence
  siRNA strand
aguuguaguaaguucccag

SEQ ID NO: 127 - RNA, 19 nt, Artificial Sequence
  siRNA strand
cugggaacuuacuacaacu

SEQ ID NO: 128 - RNA, 19 nt, Artificial Sequence
  siRNA strand
agacggagcuggaguugua

SEQ ID NO: 129 - RNA, 19 nt, Artificial Sequence
  siRNA strand
uacaacuccagcuccgucu

SEQ ID NO: 130 - RNA, 19 nt, Artificial Sequence
  siRNA strand
uagacggagcuggaguugu

SEQ ID NO: 131 - RNA, 19 nt, Artificial Sequence
  siRNA strand
acaacuccagcuccgucua

SEQ ID NO: 132 - RNA, 19 nt, Artificial Sequence
  siRNA strand
aauagacggagcuggaguu

SEQ ID NO: 133 - RNA, 19 nt, Artificial Sequence
  siRNA strand
aacuccagcuccgucuauu

SEQ ID NO: 134 - RNA, 19 nt, Artificial Sequence
  siRNA strand
ugaggggucccuccccaaa

SEQ ID NO: 135 - RNA, 19 nt, Artificial Sequence
  siRNA strand
uuuggggagggaccccuca

SEQ ID NO: 136 - RNA, 19 nt, Artificial Sequence
  siRNA strand
agaagcaggugaggggucc

SEQ ID NO: 137 - RNA, 19 nt, Artificial Sequence
  siRNA strand
ggaccccucaccugcuucu

SEQ ID NO: 138 - RNA, 19 nt, Artificial Sequence
  siRNA strand
agaaugaaccagaagaagc

SEQ ID NO: 139 - RNA, 19 nt, Artificial Sequence
  siRNA strand
gcuucuucugguucauucu

SEQ ID NO: 140 - RNA, 19 nt, Artificial Sequence
  siRNA strand
uggagaaugaaccagaaga

SEQ ID NO: 141 - RNA, 19 nt, Artificial Sequence
  siRNA strand
ucuucugguucauucucca

SEQ ID NO: 142 - RNA, 19 nt, Artificial Sequence
  siRNA strand
agcaucagccggcggugcu

SEQ ID NO: 143 - RNA, 19 nt, Artificial Sequence
  siRNA strand
agcaccgccggcugaugcu

SEQ ID NO: 144 - RNA, 19 nt, Artificial Sequence
  siRNA strand
uacucggcccuguagggga

SEQ ID NO: 145 - RNA, 19 nt, Artificial Sequence
  siRNA strand
uccccuacagggccgagua

SEQ ID NO: 146 - RNA, 19 nt, Artificial Sequence
  siRNA strand
ucguacucggcccuguagg

SEQ ID NO: 147 - RNA, 19 nt, Artificial Sequence
  siRNA strand
ccuacagggccgaguacga

SEQ ID NO: 148 - RNA, 19 nt, Artificial Sequence
  siRNA strand
acuucguacucggcccugu

SEQ ID NO: 149 - RNA, 19 nt, Artificial Sequence
  siRNA strand
acagggccgaguacgaagu

SEQ ID NO: 150 - RNA, 19 nt, Artificial Sequence
  siRNA strand
uccacuucguacucggccc

SEQ ID NO: 151 - RNA, 19 nt, Artificial Sequence
  siRNA strand
gggccgaguacgaagugga

SEQ ID NO: 152 - RNA, 19 nt, Artificial Sequence
  siRNA strand
aggaucacuaggcccucgg

SEQ ID NO: 153 - RNA, 19 nt, Artificial Sequence
  siRNA strand
ccgagggccuagugauccu

SEQ ID NO: 154 - RNA, 19 nt, Artificial Sequence
  siRNA strand
agcuaugucuuucacacug

SEQ ID NO: 155 - RNA, 19 nt, Artificial Sequence
  siRNA strand
cagugugaaagacauagcu

SEQ ID NO: 156 - RNA, 19 nt, Artificial Sequence
  siRNA strand
acaacccagcguggaauuc

SEQ ID NO: 157 - RNA, 19 nt, Artificial Sequence
  siRNA strand
gaauuccacgcuggguugu

SEQ ID NO: 158 - RNA, 19 nt, Artificial Sequence
  siRNA strand
aacaacccagcguggaauu

SEQ ID NO: 159 - RNA, 19 nt, Artificial Sequence
  siRNA strand
aauuccacgcuggguuguu

SEQ ID NO: 160 - RNA, 19 nt, Artificial Sequence
  siRNA strand
agcgguaacaacccagcgu

SEQ ID NO: 161 - RNA, 19 nt, Artificial Sequence
  siRNA strand
acgcuggguuguuaccgcu

SEQ ID NO: 162 - RNA, 19 nt, Artificial Sequence
  siRNA strand
uagcgguaacaacccagcg

SEQ ID NO: 163 - RNA, 19 nt, Artificial Sequence
  siRNA strand
cgcuggguuguuaccgcua

SEQ ID NO: 164 - RNA, 19 nt, Artificial Sequence
  siRNA strand
uguagcgguaacaacccag

SEQ ID NO: 165 - RNA, 19 nt, Artificial Sequence
  siRNA strand
cuggguuguuaccgcuaca

SEQ ID NO: 166 - RNA, 19 nt, Artificial Sequence
  siRNA strand
agcuguagcgguaacaacc

SEQ ID NO: 167 - RNA, 19 nt, Artificial Sequence
  siRNA strand
gguuguuaccgcuacagcu

SEQ ID NO: 168 - RNA, 19 nt, Artificial Sequence
  siRNA strand
acguagcuguagcgguaac

SEQ ID NO: 169 - RNA, 19 nt, Artificial Sequence
  siRNA strand
guuaccgcuacagcuacgu

SEQ ID NO: 170 - RNA, 19 nt, Artificial Sequence
  siRNA strand
aguuugagcaugagguccu

SEQ ID NO: 171 - RNA, 19 nt, Artificial Sequence
  siRNA strand
aggaccucaugcucaaacu

SEQ ID NO: 172 - RNA, 19 nt, Artificial Sequence
  siRNA strand
agccggaguuugagcauga

SEQ ID NO: 173 - RNA, 19 nt, Artificial Sequence
  siRNA strand
ucaugcucaaacuccggcu

SEQ ID NO: 174 - RNA, 19 nt, Artificial Sequence
  siRNA strand
agccguacaccgaggugau

SEQ ID NO: 175 - RNA, 19 nt, Artificial Sequence
  siRNA strand
aucaccucgguguacggcu

SEQ ID NO: 176 - RNA, 19 nt, Artificial Sequence
  siRNA strand
ugcagccguacaccgaggu

SEQ ID NO: 177 - RNA, 19 nt, Artificial Sequence
  siRNA strand
accucgguguacggcugca

SEQ ID NO: 178 - RNA, 19 nt, Artificial Sequence
  siRNA strand
uccagacgaccgccaugau

SEQ ID NO: 179 - RNA, 19 nt, Artificial Sequence
  siRNA strand
aucauggcggucgucugga

SEQ ID NO: 180 - RNA, 19 nt, Artificial Sequence
  siRNA strand
uuccagacgaccgccauga

SEQ ID NO: 181 - RNA, 19 nt, Artificial Sequence
  siRNA strand
ucauggcggucgucuggaa

SEQ ID NO: 182 - RNA, 19 nt, Artificial Sequence
  siRNA strand
acgaaggggucguaguagc

SEQ ID NO: 183 - RNA, 19 nt, Artificial Sequence
  siRNA strand
gcuacuacgaccccuucgu

SEQ ID NO: 184 - RNA, 19 nt, Artificial Sequence
  siRNA strand
aagaccaccggcugcacgg

SEQ ID NO: 185 - RNA, 19 nt, Artificial Sequence
  siRNA strand
ccgugcagccgguggucuu

SEQ ID NO: 186 - RNA, 19 nt, Artificial Sequence
  siRNA strand
acuucacaggccuggaaga

SEQ ID NO: 187 - RNA, 19 nt, Artificial Sequence
  siRNA strand
ucuuccaggccugugaagu

SEQ ID NO: 188 - RNA, 19 nt, Artificial Sequence
  siRNA strand
ucagguucacuucacaggc

SEQ ID NO: 189 - RNA, 19 nt, Artificial Sequence
  siRNA strand
gccugugaagugaaccuga

SEQ ID NO: 190 - RNA, 19 nt, Artificial Sequence
  siRNA strand
agcgucagguucacuucac

SEQ ID NO: 191 - RNA, 19 nt, Artificial Sequence
  siRNA strand
gugaagugaaccugacgcu

SEQ ID NO: 192 - RNA, 19 nt, Artificial Sequence
  siRNA strand
ucgagccuguuguccagcg

SEQ ID NO: 193 - RNA, 19 nt, Artificial Sequence
  siRNA strand
cgcuggacaacaggcucga

SEQ ID NO: 194 - RNA, 19 nt, Artificial Sequence
  siRNA strand
agucgagccuguuguccag

SEQ ID NO: 195 - RNA, 19 nt, Artificial Sequence
  siRNA strand
cuggacaacaggcucgacu

SEQ ID NO: 196 - RNA, 19 nt, Artificial Sequence
  siRNA strand
aguacggggugcugaggac

SEQ ID NO: 197 - RNA, 19 nt, Artificial Sequence
  siRNA strand
guccucagcaccccguacu

SEQ ID NO: 198 - RNA, 19 nt, Artificial Sequence
  siRNA strand
aaguacggggugcugagga

SEQ ID NO: 199 - RNA, 19 nt, Artificial Sequence
  siRNA strand
uccucagcaccccguacuu

SEQ ID NO: 200 - RNA, 19 nt, Artificial Sequence
  siRNA strand
uggggaaguacggggugcu

SEQ ID NO: 201 - RNA, 19 nt, Artificial Sequence
  siRNA strand
agcaccccguacuucccca

SEQ ID NO: 202 - RNA, 19 nt, Artificial Sequence
  siRNA strand
uuggggcgaguaguagcug

SEQ ID NO: 203 - RNA, 19 nt, Artificial Sequence
  siRNA strand
cagcuacuacucgccccaa

SEQ ID NO: 204 - RNA, 19 nt, Artificial Sequence
  siRNA strand
uuuggggcgaguaguagcu

SEQ ID NO: 205 - RNA, 19 nt, Artificial Sequence
  siRNA strand
agcuacuacucgccccaaa

SEQ ID NO: 206 - RNA, 19 nt, Artificial Sequence
  siRNA strand
uggguuuggggcgaguagu

SEQ ID NO: 207 - RNA, 19 nt, Artificial Sequence
  siRNA strand
acuacucgccccaaaccca

SEQ ID NO: 208 - RNA, 19 nt, Artificial Sequence
  siRNA strand
agcaguggguuuggggcga

SEQ ID NO: 209 - RNA, 19 nt, Artificial Sequence
  siRNA strand
ucgccccaaacccacugcu

SEQ ID NO: 210 - RNA, 19 nt, Artificial Sequence
  siRNA strand
accgugaggugccaggagc

SEQ ID NO: 211 - RNA, 19 nt, Artificial Sequence
  siRNA strand
gcuccuggcaccucacggu

SEQ ID NO: 212 - RNA, 19 nt, Artificial Sequence
  siRNA strand
agccguaguccagagaggg

SEQ ID NO: 213 - RNA, 19 nt, Artificial Sequence
  siRNA strand
cccucucuggacuacggcu

SEQ ID NO: 214 - RNA, 19 nt, Artificial Sequence
  siRNA strand
aaccagagggccaagccgu

SEQ ID NO: 215 - RNA, 19 nt, Artificial Sequence
  siRNA strand
acggcuuggcccucugguu

SEQ ID NO: 216 - RNA, 19 nt, Artificial Sequence
  siRNA strand
aaaccagagggccaagccg

SEQ ID NO: 217 - RNA, 19 nt, Artificial Sequence
  siRNA strand
cggcuuggcccucugguuu

SEQ ID NO: 218 - RNA, 19 nt, Artificial Sequence
  siRNA strand
ucaaaccagagggccaagc

SEQ ID NO: 219 - RNA, 19 nt, Artificial Sequence
  siRNA strand
gcuuggcccucugguuuga

SEQ ID NO: 220 - RNA, 19 nt, Artificial Sequence
  siRNA strand
auaggcaucaaaccagagg

SEQ ID NO: 221 - RNA, 19 nt, Artificial Sequence
  siRNA strand
ccucugguuugaugccuau

SEQ ID NO: 222 - RNA, 19 nt, Artificial Sequence
  siRNA strand
acggcaaaucauacuucug

SEQ ID NO: 223 - RNA, 19 nt, Artificial Sequence
  siRNA strand
cagaaguaugauuugccgu

SEQ ID NO: 224 - RNA, 19 nt, Artificial Sequence
  siRNA strand
ugcacggcaaaucauacuu

SEQ ID NO: 225 - RNA, 19 nt, Artificial Sequence
  siRNA strand
aaguaugauuugccgugca

SEQ ID NO: 226 - RNA, 19 nt, Artificial Sequence
  siRNA strand
ugggugcacggcaaaucau

SEQ ID NO: 227 - RNA, 19 nt, Artificial Sequence
  siRNA strand
augauuugccgugcaccca

SEQ ID NO: 228 - RNA, 19 nt, Artificial Sequence
  siRNA strand
ucguccacuggcccugggu

SEQ ID NO: 229 - RNA, 19 nt, Artificial Sequence
  siRNA strand
acccagggccaguggacga

SEQ ID NO: 230 - RNA, 19 nt, Artificial Sequence
  siRNA strand
uggaucguccacuggcccu

SEQ ID NO: 231 - RNA, 19 nt, Artificial Sequence
  siRNA strand
agggccaguggacgaucca

SEQ ID NO: 232 - RNA, 19 nt, Artificial Sequence
  siRNA strand
ucuggaucguccacuggcc

SEQ ID NO: 233 - RNA, 19 nt, Artificial Sequence
  siRNA strand
ggccaguggacgauccaga

SEQ ID NO: 234 - RNA, 19 nt, Artificial Sequence
  siRNA strand
uucuggaucguccacuggc

SEQ ID NO: 235 - RNA, 19 nt, Artificial Sequence
  siRNA strand
gccaguggacgauccagaa

SEQ ID NO: 236 - RNA, 19 nt, Artificial Sequence
  siRNA strand
uccucucggcguagggcug

SEQ ID NO: 237 - RNA, 19 nt, Artificial Sequence
  siRNA strand
cagcccuacgccgagagga

SEQ ID NO: 238 - RNA, 19 nt, Artificial Sequence
  siRNA strand
auagugcacccgcacaccg

SEQ ID NO: 239 - RNA, 19 nt, Artificial Sequence
  siRNA strand
cggugugcgggugcacuau

SEQ ID NO: 240 - RNA, 19 nt, Artificial Sequence
  siRNA strand
agccauagugcacccgcac

SEQ ID NO: 241 - RNA, 19 nt, Artificial Sequence
  siRNA strand
gugcgggugcacuauggcu

SEQ ID NO: 242 - RNA, 19 nt, Artificial Sequence
  siRNA strand
aagccauagugcacccgca

SEQ ID NO: 243 - RNA, 19 nt, Artificial Sequence
  siRNA strand
ugcgggugcacuauggcuu

SEQ ID NO: 244 - RNA, 19 nt, Artificial Sequence
  siRNA strand
uacaagccauagugcaccc

SEQ ID NO: 245 - RNA, 19 nt, Artificial Sequence
  siRNA strand
gggugcacuauggcuugua

SEQ ID NO: 246 - RNA, 19 nt, Artificial Sequence
  siRNA strand
uuguacaagccauagugca

SEQ ID NO: 247 - RNA, 19 nt, Artificial Sequence
  siRNA strand
ugcacuauggcuuguacaa

SEQ ID NO: 248 - RNA, 19 nt, Artificial Sequence
  siRNA strand
agaggaacucuccagggca

SEQ ID NO: 249 - RNA, 19 nt, Artificial Sequence
  siRNA strand
ugcccuggagaguuccucu

SEQ ID NO: 250 - RNA, 19 nt, Artificial Sequence
  siRNA strand
acgcaguuucucucaucca

SEQ ID NO: 251 - RNA, 19 nt, Artificial Sequence
  siRNA strand
uggaugagagaaacugcgu

SEQ ID NO: 252 - RNA, 19 nt, Artificial Sequence
  siRNA strand
ugagaugcaugugcugucc

SEQ ID NO: 253 - RNA, 19 nt, Artificial Sequence
  siRNA strand
ggacagcacaugcaucuca

SEQ ID NO: 254 - RNA, 19 nt, Artificial Sequence
  siRNA strand
agugagaugcaugugcugu

SEQ ID NO: 255 - RNA, 19 nt, Artificial Sequence
  siRNA strand
acagcacaugcaucucacu

SEQ ID NO: 256 - RNA, 19 nt, Artificial Sequence
  siRNA strand
ugcccaucacagaccuugg

SEQ ID NO: 257 - RNA, 19 nt, Artificial Sequence
  siRNA strand
ccaaggucugugaugggca

SEQ ID NO: 258 - RNA, 19 nt, Artificial Sequence
  siRNA strand
agacaaucaggcugcccau

SEQ ID NO: 259 - RNA, 19 nt, Artificial Sequence
  siRNA strand
augggcagccugauugucu

SEQ ID NO: 260 - RNA, 19 nt, Artificial Sequence
  siRNA strand
uugagacaaucaggcugcc

SEQ ID NO: 261 - RNA, 19 nt, Artificial Sequence
  siRNA strand
ggcagccugauugucucaa

SEQ ID NO: 262 - RNA, 19 nt, Artificial Sequence
  siRNA strand
ugccguugagacaaucagg

SEQ ID NO: 263 - RNA, 19 nt, Artificial Sequence
  siRNA strand
ccugauugucucaacggca

SEQ ID NO: 264 - RNA, 19 nt, Artificial Sequence
  siRNA strand
uucgucgcugccguugaga

SEQ ID NO: 265 - RNA, 19 nt, Artificial Sequence
  siRNA strand
ucucaacggcagcgacgaa

SEQ ID NO: 266 - RNA, 19 nt, Artificial Sequence
  siRNA strand
auggcaccccuuccuggca

SEQ ID NO: 267 - RNA, 19 nt, Artificial Sequence
  siRNA strand
ugccaggaaggggugccau

SEQ ID NO: 268 - RNA, 19 nt, Artificial Sequence
  siRNA strand
augucccacauggcacccc

SEQ ID NO: 269 - RNA, 19 nt, Artificial Sequence
  siRNA strand
ggggugccaugugggacau

SEQ ID NO: 270 - RNA, 19 nt, Artificial Sequence
  siRNA strand
ugaaugucccacauggcac

SEQ ID NO: 271 - RNA, 19 nt, Artificial Sequence
  siRNA strand
gugccaugugggacauuca

SEQ ID NO: 272 - RNA, 19 nt, Artificial Sequence
  siRNA strand
aaggugaaugucccacaug

SEQ ID NO: 273 - RNA, 19 nt, Artificial Sequence
  siRNA strand
caugugggacauucaccuu

SEQ ID NO: 274 - RNA, 19 nt, Artificial Sequence
  siRNA strand
agcuccgguccucacacug

SEQ ID NO: 275 - RNA, 19 nt, Artificial Sequence
  siRNA strand
cagugugaggaccggagcu

SEQ ID NO: 276 - RNA, 19 nt, Artificial Sequence
  siRNA strand
ugcggguugggcuucuuca

SEQ ID NO: 277 - RNA, 19 nt, Artificial Sequence
  siRNA strand
ugaagaagcccaacccgca

SEQ ID NO: 278 - RNA, 19 nt, Artificial Sequence
  siRNA strand
acacugcggguugggcuuc

SEQ ID NO: 279 - RNA, 19 nt, Artificial Sequence
  siRNA strand
gaagcccaacccgcagugu

SEQ ID NO: 280 - RNA, 19 nt, Artificial Sequence
  siRNA strand
aacaaugcggcuggagggg

SEQ ID NO: 281 - RNA, 19 nt, Artificial Sequence
  siRNA strand
ccccuccagccgcauuguu

SEQ ID NO: 282 - RNA, 19 nt, Artificial Sequence
  siRNA strand
ugugucgaccccgaaccug

SEQ ID NO: 283 - RNA, 19 nt, Artificial Sequence
  siRNA strand
cagguucggggucgacaca

SEQ ID NO: 284 - RNA, 19 nt, Artificial Sequence
  siRNA strand
augugucgaccccgaaccu

SEQ ID NO: 285 - RNA, 19 nt, Artificial Sequence
  siRNA strand
agguucggggucgacacau

SEQ ID NO: 286 - RNA, 19 nt, Artificial Sequence
  siRNA strand
agaugugucgaccccgaac

SEQ ID NO: 287 - RNA, 19 nt, Artificial Sequence
  siRNA strand
guucggggucgacacaucu

SEQ ID NO: 288 - RNA, 19 nt, Artificial Sequence
  siRNA strand
uaucacccagcggucagcg

SEQ ID NO: 289 - RNA, 19 nt, Artificial Sequence
  siRNA strand
cgcugaccgcugggugaua

SEQ ID NO: 290 - RNA, 19 nt, Artificial Sequence
  siRNA strand
uuaucacccagcggucagc

SEQ ID NO: 291 - RNA, 19 nt, Artificial Sequence
  siRNA strand
gcugaccgcugggugauaa

SEQ ID NO: 292 - RNA, 19 nt, Artificial Sequence
  siRNA strand
uguuaucacccagcgguca

SEQ ID NO: 293 - RNA, 19 nt, Artificial Sequence
  siRNA strand
ugaccgcugggugauaaca

SEQ ID NO: 294 - RNA, 19 nt, Artificial Sequence
  siRNA strand
agcuguuaucacccagcgg

SEQ ID NO: 295 - RNA, 19 nt, Artificial Sequence
  siRNA strand
ccgcugggugauaacagcu

SEQ ID NO: 296 - RNA, 19 nt, Artificial Sequence
  siRNA strand
aggaacacgguccacagca

SEQ ID NO: 297 - RNA, 19 nt, Artificial Sequence
  siRNA strand
ugcuguggaccguguuccu

SEQ ID NO: 298 - RNA, 19 nt, Artificial Sequence
  siRNA strand
agcgcgaguucugccacac

SEQ ID NO: 299 - RNA, 19 nt, Artificial Sequence
  siRNA strand
guguggcagaacucgcgcu

SEQ ID NO: 300 - RNA, 19 nt, Artificial Sequence
  siRNA strand
ucgugguacgggugcagga

SEQ ID NO: 301 - RNA, 19 nt, Artificial Sequence
  siRNA strand
uccugcacccguaccacga

SEQ ID NO: 302 - RNA, 19 nt, Artificial Sequence
  siRNA strand
uucgugguacgggugcagg

SEQ ID NO: 303 - RNA, 19 nt, Artificial Sequence
  siRNA strand
ccugcacccguaccacgaa

SEQ ID NO: 304 - RNA, 19 nt, Artificial Sequence
  siRNA strand
ucuucgugguacgggugca

SEQ ID NO: 305 - RNA, 19 nt, Artificial Sequence
  siRNA strand
ugcacccguaccacgaaga

SEQ ID NO: 306 - RNA, 19 nt, Artificial Sequence
  siRNA strand
ucgaagaagugggagcgcg

SEQ ID NO: 307 - RNA, 19 nt, Artificial Sequence
  siRNA strand
cgcgcucccacuucuucga

SEQ ID NO: 308 - RNA, 19 nt, Artificial Sequence
  siRNA strand
uaauccagcagugcaggcc

SEQ ID NO: 309 - RNA, 19 nt, Artificial Sequence
  siRNA strand
ggccugcacugcuggauua

SEQ ID NO: 310 - RNA, 19 nt, Artificial Sequence
  siRNA strand
agcccguaauccagcagug

SEQ ID NO: 311 - RNA, 19 nt, Artificial Sequence
  siRNA strand
cacugcuggauuacgggcu

SEQ ID NO: 312 - RNA, 19 nt, Artificial Sequence
  siRNA strand
ugggaucaacugcacaucc

SEQ ID NO: 313 - RNA, 19 nt, Artificial Sequence
  siRNA strand
ggaugugcaguugauccca

SEQ ID NO: 314 - RNA, 19 nt, Artificial Sequence
  siRNA strand
agcgauagaccucgcugca

SEQ ID NO: 315 - RNA, 19 nt, Artificial Sequence
  siRNA strand
ugcagcgaggucuaucgcu

SEQ ID NO: 316 - RNA, 19 nt, Artificial Sequence
  siRNA strand
uagcgauagaccucgcugc

SEQ ID NO: 317 - RNA, 19 nt, Artificial Sequence
  siRNA strand
gcagcgaggucuaucgcua

SEQ ID NO: 318 - RNA, 19 nt, Artificial Sequence
  siRNA strand
ugguagcgauagaccucgc

SEQ ID NO: 319 - RNA, 19 nt, Artificial Sequence
  siRNA strand
gcgaggucuaucgcuacca

SEQ ID NO: 320 - RNA, 19 nt, Artificial Sequence
  siRNA strand
ucaccugguagcgauagac

SEQ ID NO: 321 - RNA, 19 nt, Artificial Sequence
  siRNA strand
gucuaucgcuaccagguga

SEQ ID NO: 322 - RNA, 19 nt, Artificial Sequence
  siRNA strand
agcaugcguggcgucaccu

SEQ ID NO: 323 - RNA, 19 nt, Artificial Sequence
  siRNA strand
aggugacgccacgcaugcu

SEQ ID NO: 324 - RNA, 19 nt, Artificial Sequence
  siRNA strand
acagcaugcguggcgucac

SEQ ID NO: 325 - RNA, 19 nt, Artificial Sequence
  siRNA strand
gugacgccacgcaugcugu

SEQ ID NO: 326 - RNA, 19 nt, Artificial Sequence
  siRNA strand
acacagcaugcguggcguc

SEQ ID NO: 327 - RNA, 19 nt, Artificial Sequence
  siRNA strand
gacgccacgcaugcugugu

SEQ ID NO: 328 - RNA, 19 nt, Artificial Sequence
  siRNA strand
aguuaggccggccacagcc

SEQ ID NO: 329 - RNA, 19 nt, Artificial Sequence
  siRNA strand
ggcuguggccggccuaacu

SEQ ID NO: 330 - RNA, 19 nt, Artificial Sequence
  siRNA strand
uaguuaggccggccacagc

SEQ ID NO: 331 - RNA, 19 nt, Artificial Sequence
  siRNA strand
gcuguggccggccuaacua

SEQ ID NO: 332 - RNA, 19 nt, Artificial Sequence
  siRNA strand
aguaguuaggccggccaca

SEQ ID NO: 333 - RNA, 19 nt, Artificial Sequence
  siRNA strand
uguggccggccuaacuacu

SEQ ID NO: 334 - RNA, 19 nt, Artificial Sequence
  siRNA strand
acgccgaaguaguuaggcc

SEQ ID NO: 335 - RNA, 19 nt, Artificial Sequence
  siRNA strand
ggccuaacuacuucggcgu

SEQ ID NO: 336 - RNA, 19 nt, Artificial Sequence
  siRNA strand
agacgccgaaguaguuagg

SEQ ID NO: 337 - RNA, 19 nt, Artificial Sequence
  siRNA strand
ccuaacuacuucggcgucu

SEQ ID NO: 338 - RNA, 19 nt, Artificial Sequence
  siRNA strand
uagacgccgaaguaguuag

SEQ ID NO: 339 - RNA, 19 nt, Artificial Sequence
  siRNA strand
cuaacuacuucggcgucua

SEQ ID NO: 340 - RNA, 19 nt, Artificial Sequence
  siRNA strand
uguagacgccgaaguaguu

SEQ ID NO: 341 - RNA, 19 nt, Artificial Sequence
  siRNA strand
aacuacuucggcgucuaca

SEQ ID NO: 342 - RNA, 19 nt, Artificial Sequence
  siRNA strand
acaccugugaugcgggugu

SEQ ID NO: 343 - RNA, 19 nt, Artificial Sequence
  siRNA strand
acacccgcaucacaggugu

SEQ ID NO: 344 - RNA, 19 nt, Artificial Sequence
  siRNA strand
ugaucacaccugugaugcg

SEQ ID NO: 345 - RNA, 19 nt, Artificial Sequence
  siRNA strand
cgcaucacaggugugauca

SEQ ID NO: 346 - RNA, 19 nt, Artificial Sequence
  siRNA strand
accacuugcuggauccagc

SEQ ID NO: 347 - RNA, 19 nt, Artificial Sequence
  siRNA strand
gcuggauccagcaaguggu

SEQ ID NO: 348 - RNA, 19 nt, Artificial Sequence
  siRNA strand
uuugcaggggggcaguucc

SEQ ID NO: 349 - RNA, 19 nt, Artificial Sequence
  siRNA strand
ggaacugccccccugcaaa

SEQ ID NO: 350 - RNA, 19 nt, Artificial Sequence
  siRNA strand
agacaagaugccaccuccu

SEQ ID NO: 351 - RNA, 19 nt, Artificial Sequence
  siRNA strand
aggagguggcaucuugucu

SEQ ID NO: 352 - RNA, 19 nt, Artificial Sequence
  siRNA strand
acgagacaagaugccaccu

SEQ ID NO: 353 - RNA, 19 nt, Artificial Sequence
  siRNA strand
agguggcaucuugucucgu

SEQ ID NO: 354 - RNA, 19 nt, Artificial Sequence
  siRNA strand
agggacgagacaagaugcc

SEQ ID NO: 355 - RNA, 19 nt, Artificial Sequence
  siRNA strand
ggcaucuugucucgucccu

SEQ ID NO: 356 - RNA, 19 nt, Artificial Sequence
  siRNA strand
ucagggacgagacaagaug

SEQ ID NO: 357 - RNA, 19 nt, Artificial Sequence
  siRNA strand
caucuugucucgucccuga

SEQ ID NO: 358 - RNA, 19 nt, Artificial Sequence
  siRNA strand
aucagggacgagacaagau

SEQ ID NO: 359 - RNA, 19 nt, Artificial Sequence
  siRNA strand
aucuugucucgucccugau

SEQ ID NO: 360 - RNA, 19 nt, Artificial Sequence
  siRNA strand
acgucuugacccccagcug

SEQ ID NO: 361 - RNA, 19 nt, Artificial Sequence
  siRNA strand
cagcugggggucaagacgu

SEQ ID NO: 362 - RNA, 19 nt, Artificial Sequence
  siRNA strand
aggggacgucuugaccccc

SEQ ID NO: 363 - RNA, 19 nt, Artificial Sequence
  siRNA strand
gggggucaagacguccccu

SEQ ID NO: 364 - RNA, 19 nt, Artificial Sequence
  siRNA strand
ucaggggacgucuugaccc

SEQ ID NO: 365 - RNA, 19 nt, Artificial Sequence
  siRNA strand
gggucaagacguccccuga

SEQ ID NO: 366 - RNA, 19 nt, Artificial Sequence
  siRNA strand
uugcauuaggcagcagugg

SEQ ID NO: 367 - RNA, 19 nt, Artificial Sequence
  siRNA strand
ccacugcugccuaaugcaa

SEQ ID NO: 368 - RNA, 19 nt, Artificial Sequence
  siRNA strand
ugccuugcauuaggcagca

SEQ ID NO: 369 - RNA, 19 nt, Artificial Sequence
  siRNA strand
ugcugccuaaugcaaggca

SEQ ID NO: 370 - RNA, 19 nt, Artificial Sequence
  siRNA strand
agccacugccuugcauuag

SEQ ID NO: 371 - RNA, 19 nt, Artificial Sequence
  siRNA strand
cuaaugcaaggcaguggcu

SEQ ID NO: 372 - RNA, 19 nt, Artificial Sequence
  siRNA strand
agaaccagcauucuugcug

SEQ ID NO: 373 - RNA, 19 nt, Artificial Sequence
  siRNA strand
cagcaagaaugcugguucu

SEQ ID NO: 374 - RNA, 19 nt, Artificial Sequence
  siRNA strand
acagaguggggcgcaccuc

SEQ ID NO: 375 - RNA, 19 nt, Artificial Sequence
  siRNA strand
gaggugcgccccacucugu

SEQ ID NO: 376 - RNA, 19 nt, Artificial Sequence
  siRNA strand
agaccaggggcuuccgaag

SEQ ID NO: 377 - RNA, 19 nt, Artificial Sequence
  siRNA strand
cuucggaagccccuggucu

SEQ ID NO: 378 - RNA, 19 nt, Artificial Sequence
  siRNA strand
uuagaccaggggcuuccga

SEQ ID NO: 379 - RNA, 19 nt, Artificial Sequence
  siRNA strand
ucggaagccccuggucuaa

SEQ ID NO: 380 - RNA, 19 nt, Artificial Sequence
  siRNA strand
aguuagaccaggggcuucc

SEQ ID NO: 381 - RNA, 19 nt, Artificial Sequence
  siRNA strand
ggaagccccuggucuaacu

SEQ ID NO: 382 - RNA, 19 nt, Artificial Sequence
  siRNA strand
aaguuagaccaggggcuuc

SEQ ID NO: 383 - RNA, 19 nt, Artificial Sequence
  siRNA strand
gaagccccuggucuaacuu

SEQ ID NO: 384 - RNA, 19 nt, Artificial Sequence
  siRNA strand
aucccaaguuagaccaggg

SEQ ID NO: 385 - RNA, 19 nt, Artificial Sequence
  siRNA strand
cccuggucuaacuugggau

SEQ ID NO: 386 - RNA, 19 nt, Artificial Sequence
  siRNA strand
agaucccaaguuagaccag

SEQ ID NO: 387 - RNA, 19 nt, Artificial Sequence
  siRNA strand
cuggucuaacuugggaucu

SEQ ID NO: 388 - RNA, 19 nt, Artificial Sequence
  siRNA strand
uucccagaucccaaguuag

SEQ ID NO: 389 - RNA, 19 nt, Artificial Sequence
  siRNA strand
cuaacuugggaucugggaa

SEQ ID NO: 390 - RNA, 19 nt, Artificial Sequence
  siRNA strand
ugaggguccccuccgaugg

SEQ ID NO: 391 - RNA, 19 nt, Artificial Sequence
  siRNA strand
ccaucggaggggacccuca

SEQ ID NO: 392 - RNA, 19 nt, Artificial Sequence
  siRNA strand
uacaguggcagcaggccca

SEQ ID NO: 393 - RNA, 19 nt, Artificial Sequence
  siRNA strand
ugggccugcugccacugua

SEQ ID NO: 394 - RNA, 19 nt, Artificial Sequence
  siRNA strand
uuacaguggcagcaggccc

SEQ ID NO: 395 - RNA, 19 nt, Artificial Sequence
  siRNA strand
gggccugcugccacuguaa

SEQ ID NO: 396 - RNA, 19 nt, Artificial Sequence
  siRNA strand
uuuggcuuacaguggcagc

SEQ ID NO: 397 - RNA, 19 nt, Artificial Sequence
  siRNA strand
gcugccacuguaagccaaa

SEQ ID NO: 398 - RNA, 19 nt, Artificial Sequence
  siRNA strand
accuuuuggcuuacagugg

SEQ ID NO: 399 - RNA, 19 nt, Artificial Sequence
  siRNA strand
ccacuguaagccaaaaggu

SEQ ID NO: 400 - RNA, 19 nt, Artificial Sequence
  siRNA strand
agucaggacuuccccaccu

SEQ ID NO: 401 - RNA, 19 nt, Artificial Sequence
  siRNA strand
agguggggaaguccugacu

SEQ ID NO: 402 - RNA, 19 nt, Artificial Sequence
  siRNA strand
ugaucaggcagcuuuauuc

SEQ ID NO: 403 - RNA, 19 nt, Artificial Sequence
  siRNA strand
gaauaaagcugccugauca

SEQ ID NO: 404 - RNA, 19 nt, Artificial Sequence
  siRNA strand
uugaucaggcagcuuuauu

SEQ ID NO: 405 - RNA, 19 nt, Artificial Sequence
  siRNA strand
aauaaagcugccugaucaa

SEQ ID NO: 406 - RNA, 19 nt, Artificial Sequence
  siRNA strand
uuuuuuugaucaggcagcu

SEQ ID NO: 407 - RNA, 19 nt, Artificial Sequence
  siRNA strand
agcugccugaucaaaaaaa

SEQ ID NO: 408 - RNA, 19 nt, Artificial Sequence
  siRNA strand
uucguacucggcccuguag

SEQ ID NO: 409 - RNA, 19 nt, Artificial Sequence
  siRNA strand
cuacagggccgaguacgaa

SEQ ID NO: 410 - RNA, 19 nt, Artificial Sequence
  siRNA strand
aguccuugaccccaucaca

SEQ ID NO: 411 - RNA, 19 nt, Artificial Sequence
  siRNA strand
ugugauggggucaaggacu

SEQ ID NO: 412 - RNA, 19 nt, Artificial Sequence
  siRNA strand
uugaaggacaccucuccag

SEQ ID NO: 413 - RNA, 19 nt, Artificial Sequence
  siRNA strand
cuggagagguguccuucaa

SEQ ID NO: 414 - RNA, 19 nt, Artificial Sequence
  siRNA strand
auacauggccagucggucc

SEQ ID NO: 415 - RNA, 19 nt, Artificial Sequence
  siRNA strand
ggaccgacuggccauguau

SEQ ID NO: 416 - RNA, 19 nt, Artificial Sequence
  siRNA strand
agguccugugggaucaacu

SEQ ID NO: 417 - RNA, 19 nt, Artificial Sequence
  siRNA strand
aguugaucccacaggaccu

SEQ ID NO: 418 - RNA, 19 nt, Artificial Sequence
  siRNA strand
uucugggcuuuggcgguuu

SEQ ID NO: 419 - RNA, 19 nt, Artificial Sequence
  siRNA strand
aaaccgccaaagcccagaa

SEQ ID NO: 420 - RNA, 19 nt, Artificial Sequence
  siRNA strand
uugcgguagccggcacaca

SEQ ID NO: 421 - RNA, 19 nt, Artificial Sequence
  siRNA strand
ugugugccggcuaccgcaa

SEQ ID NO: 422 - RNA, 19 nt, Artificial Sequence
  siRNA strand
uaacaacccagcguggaau

SEQ ID NO: 423 - RNA, 19 nt, Artificial Sequence
  siRNA strand
auuccacgcuggguuguua

SEQ ID NO: 424 - RNA, 19 nt, Artificial Sequence
  siRNA strand
aucacccagcggucagcga

SEQ ID NO: 425 - RNA, 19 nt, Artificial Sequence
  siRNA strand
ucgcugaccgcugggugau

SEQ ID NO: 426 - RNA, 19 nt, Artificial Sequence
  siRNA strand
auacuugucccccugcuug

SEQ ID NO: 427 - RNA, 19 nt, Artificial Sequence
  siRNA strand
caagcagggggacaaguau

SEQ ID NO: 428 - RNA, 19 nt, Artificial Sequence
  siRNA strand
aucacuggagcagacauca

SEQ ID NO: 429 - RNA, 19 nt, Artificial Sequence
  siRNA strand
ugaugucugcuccagugau

SEQ ID NO: 430 - RNA, 19 nt, Artificial Sequence
  siRNA strand
ucaccuugaaggacaccuc

SEQ ID NO: 431 - RNA, 19 nt, Artificial Sequence
  siRNA strand
gagguguccuucaagguga

SEQ ID NO: 432 - RNA, 19 nt, Artificial Sequence
  siRNA strand
aauacuugucccccugcuu

SEQ ID NO: 433 - RNA, 19 nt, Artificial Sequence
  siRNA strand
aagcagggggacaaguauu

SEQ ID NO: 434 - RNA, 19 nt, Artificial Sequence
  siRNA strand
uccauucccagaucccaag

SEQ ID NO: 435 - RNA, 19 nt, Artificial Sequence
  siRNA strand
cuugggaucugggaaugga

SEQ ID NO: 436 - RNA, 19 nt, Artificial Sequence
  siRNA strand
uuguacccuaggaaauacc

SEQ ID NO: 437 - RNA, 19 nt, Artificial Sequence
  siRNA strand
gguauuuccuaggguacaa

SEQ ID NO: 438 - RNA, 19 nt, Artificial Sequence
  siRNA strand
ucuugcccuugcgguagcc

SEQ ID NO: 439 - RNA, 19 nt, Artificial Sequence
  siRNA strand
ggcuaccgcaagggcaaga

SEQ ID NO: 440 - RNA, 19 nt, Artificial Sequence
  siRNA strand
agaauacuugucccccugc

SEQ ID NO: 441 - RNA, 19 nt, Artificial Sequence
  siRNA strand
gcagggggacaaguauucu

SEQ ID NO: 442 - RNA, 19 nt, Artificial Sequence
  siRNA strand
agcauucuugcugcugagc

SEQ ID NO: 443 - RNA, 19 nt, Artificial Sequence
  siRNA strand
gcucagcagcaagaaugcu

SEQ ID NO: 444 - RNA, 19 nt, Artificial Sequence
  siRNA strand
uuccauucccagaucccaa

SEQ ID NO: 445 - RNA, 19 nt, Artificial Sequence
  siRNA strand
uugggaucugggaauggaa

SEQ ID NO: 446 - RNA, 19 nt, Artificial Sequence
  siRNA strand
agcaucuucugggcuuugg

SEQ ID NO: 447 - RNA, 19 nt, Artificial Sequence
  siRNA strand
ccaaagcccagaagaugcu

SEQ ID NO: 448 - RNA, 19 nt, Artificial Sequence
  siRNA strand
uucuugcccuugcgguagc

SEQ ID NO: 449 - RNA, 19 nt, Artificial Sequence
  siRNA strand
gcuaccgcaagggcaagaa

SEQ ID NO: 450 - RNA, 19 nt, Artificial Sequence
  siRNA strand
uuccaaagggcagcugagc

SEQ ID NO: 451 - RNA, 19 nt, Artificial Sequence
  siRNA strand
gcucagcugcccuuuggaa

SEQ ID NO: 452 - RNA, 19 nt, Artificial Sequence
  siRNA strand
augccaccuccugccacca

SEQ ID NO: 453 - RNA, 19 nt, Artificial Sequence
  siRNA strand
ugguggcaggagguggcau

SEQ ID NO: 454 - RNA, 19 nt, Artificial Sequence
  siRNA strand
agccucuguacagaguggg

SEQ ID NO: 455 - RNA, 19 nt, Artificial Sequence
  siRNA strand
cccacucuguacagaggcu

SEQ ID NO: 456 - RNA, 19 nt, Artificial Sequence
  siRNA strand
ugagggucugggcugugag

SEQ ID NO: 457 - RNA, 19 nt, Artificial Sequence
  siRNA strand
cucacagcccagacccuca

SEQ ID NO: 458 - RNA, 19 nt, Artificial Sequence
  siRNA strand
aagccguaguccagagagg

SEQ ID NO: 459 - RNA, 19 nt, Artificial Sequence
  siRNA strand
ccucucuggacuacggcuu

SEQ ID NO: 460 - RNA, 19 nt, Artificial Sequence
  siRNA strand
agaugccaccuccugccac

SEQ ID NO: 461 - RNA, 19 nt, Artificial Sequence
  siRNA strand
guggcaggagguggcaucu

SEQ ID NO: 462 - RNA, 19 nt, Artificial Sequence
  siRNA strand
uagaccaggggcuuccgaa

SEQ ID NO: 463 - RNA, 19 nt, Artificial Sequence
  siRNA strand
uucggaagccccuggucua

SEQ ID NO: 464 - RNA, 19 nt, Artificial Sequence
  siRNA strand
uccaaagggcagcugagcu

SEQ ID NO: 465 - RNA, 19 nt, Artificial Sequence
  siRNA strand
agcucagcugcccuuugga

SEQ ID NO: 466 - RNA, 19 nt, Artificial Sequence
  siRNA strand
aguuucucucauccaggcc

SEQ ID NO: 467 - RNA, 19 nt, Artificial Sequence
  siRNA strand
ggccuggaugagagaaacu

SEQ ID NO: 468 - RNA, 19 nt, Artificial Sequence
  siRNA strand
aagaugccaccuccugcca

SEQ ID NO: 469 - RNA, 19 nt, Artificial Sequence
  siRNA strand
uggcaggagguggcaucuu

SEQ ID NO: 470 - RNA, 19 nt, Artificial Sequence
  siRNA strand
uggaggccacagucacagu

SEQ ID NO: 471 - RNA, 19 nt, Artificial Sequence
  siRNA strand
acugugacuguggccucca

SEQ ID NO: 472 - RNA, 19 nt, Artificial Sequence
  siRNA strand
ucccaaguuagaccagggg

SEQ ID NO: 473 - RNA, 19 nt, Artificial Sequence
  siRNA strand
ccccuggucuaacuuggga

SEQ ID NO: 474 - RNA, 19 nt, Artificial Sequence
  siRNA strand
uauuccaaagggcagcuga

SEQ ID NO: 475 - RNA, 19 nt, Artificial Sequence
  siRNA strand
ucagcugcccuuuggaaua

SEQ ID NO: 476 - RNA, 19 nt, Artificial Sequence
  siRNA strand
acagaugugucgaccccga

SEQ ID NO: 477 - RNA, 19 nt, Artificial Sequence
  siRNA strand
ucggggucgacacaucugu

SEQ ID NO: 478 - RNA, 19 nt, Artificial Sequence
  siRNA strand
uggagcagacaucagggac

SEQ ID NO: 479 - RNA, 19 nt, Artificial Sequence
  siRNA strand
gucccugaugucugcucca

SEQ ID NO: 480 - RNA, 19 nt, Artificial Sequence
  siRNA strand
acccagcggucagcgauga

SEQ ID NO: 481 - RNA, 19 nt, Artificial Sequence
  siRNA strand
ucaucgcugaccgcugggu

SEQ ID NO: 482 - RNA, 19 nt, Artificial Sequence
  siRNA strand
uuauuccaaagggcagcug

SEQ ID NO: 483 - RNA, 23 nt, Artificial Sequence
  siRNA strand
uugaaggacaccucuccaggcca

SEQ ID NO: 484 - RNA, 21 nt, Artificial Sequence
  siRNA strand
uggccuggagagguguccuuc

SEQ ID NO: 485 - RNA, 23 nt, Artificial Sequence
  siRNA strand
aggaaauaccagaguagcacccc

SEQ ID NO: 486 - RNA, 21 nt, Artificial Sequence
  siRNA strand
ggggugcuacucugguauuuc

SEQ ID NO: 487 - RNA, 23 nt, Artificial Sequence
  siRNA strand
aguuucucucauccaggccguug

SEQ ID NO: 488 - RNA, 21 nt, Artificial Sequence
  siRNA strand
caacggccuggaugagagaaa

SEQ ID NO: 489 - RNA, 23 nt, Artificial Sequence
  siRNA strand
aagauccugggagaaguggcgau

SEQ ID NO: 490 - RNA, 21 nt, Artificial Sequence
  siRNA strand
aucgccacuucucccaggauc

SEQ ID NO: 491 - RNA, 23 nt, Artificial Sequence
  siRNA strand
acaagaugccaccuccugccacc

SEQ ID NO: 492 - RNA, 21 nt, Artificial Sequence
  siRNA strand
gguggcaggagguggcaucuu

SEQ ID NO: 493 - RNA, 23 nt, Artificial Sequence
  siRNA strand
acgucauacauggccagucgguc

SEQ ID NO: 494 - RNA, 21 nt, Artificial Sequence
  siRNA strand
gaccgacuggccauguaugac

SEQ ID NO: 495 - RNA, 23 nt, Artificial Sequence
  siRNA strand
aagccauagugcacccgcacacc

SEQ ID NO: 496 - RNA, 21 nt, Artificial Sequence
  siRNA strand
ggugugcgggugcacuauggc

SEQ ID NO: 497 - RNA, 23 nt, Artificial Sequence
  siRNA strand
acgcaguuucucucauccaggcc

SEQ ID NO: 498 - RNA, 21 nt, Artificial Sequence
  siRNA strand
ggccuggaugagagaaacugc

SEQ ID NO: 499 - RNA, 23 nt, Artificial Sequence
  siRNA strand
uuguacccuaggaaauaccagag

SEQ ID NO: 500 - RNA, 21 nt, Artificial Sequence
  siRNA strand
cucugguauuuccuaggguac

SEQ ID NO: 501 - RNA, 23 nt, Artificial Sequence
  siRNA strand
agaucccaaguuagaccaggggc

SEQ ID NO: 502 - RNA, 21 nt, Artificial Sequence
  siRNA strand
gccccuggucuaacuugggau

SEQ ID NO: 503 - RNA, 23 nt, Artificial Sequence
  siRNA strand
acggcaaaucauacuucugccuc

SEQ ID NO: 504 - RNA, 21 nt, Artificial Sequence
  siRNA strand
gaggcagaaguaugauuugcc

SEQ ID NO: 505 - RNA, 23 nt, Artificial Sequence
  siRNA strand
agcuaugucuuucacacuggcuu

SEQ ID NO: 506 - RNA, 21 nt, Artificial Sequence
  siRNA strand
aagccagugugaaagacauag

SEQ ID NO: 507 - RNA, 23 nt, Artificial Sequence
  siRNA strand
auacauggccagucggucccggc

SEQ ID NO: 508 - RNA, 21 nt, Artificial Sequence
  siRNA strand
gccgggaccgacuggccaugu

SEQ ID NO: 509 - RNA, 23 nt, Artificial Sequence
  siRNA strand
augugucgaccccgaaccuggag

SEQ ID NO: 510 - RNA, 21 nt, Artificial Sequence
  siRNA strand
cuccagguucggggucgacac

SEQ ID NO: 511 - RNA, 23 nt, Artificial Sequence
  siRNA strand
gaucccaaguuagaccaggggcu

SEQ ID NO: 512 - RNA, 21 nt, Artificial Sequence
  siRNA strand
agccccuggucuaacuuggga

SEQ ID NO: 513 - RNA, 23 nt, Artificial Sequence
  siRNA strand
uaagauccugggagaaguggcga

SEQ ID NO: 514 - RNA, 21 nt, Artificial Sequence
  siRNA strand
ucgccacuucucccaggaucu

SEQ ID NO: 515 - RNA, 23 nt, Artificial Sequence
  siRNA strand
uguacccuaggaaauaccagagu

SEQ ID NO: 516 - RNA, 21 nt, Artificial Sequence
  siRNA strand
acucugguauuuccuagggua

SEQ ID NO: 517 - RNA, 23 nt, Artificial Sequence
  siRNA strand
uguuaucacccagcggucagcga

SEQ ID NO: 518 - RNA, 21 nt, Artificial Sequence
  siRNA strand
ucgcugaccgcugggugauaa

SEQ ID NO: 519 - RNA, 23 nt, Artificial Sequence
  siRNA strand
ucucucauccaggccguuggggc

SEQ ID NO: 520 - RNA, 21 nt, Artificial Sequence
  siRNA strand
gccccaacggccuggaugaga

SEQ ID NO: 521 - RNA, 23 nt, Artificial Sequence
  siRNA strand
gaauacuugucccccugcuuggc

SEQ ID NO: 522 - RNA, 21 nt, Artificial Sequence
  siRNA strand
gccaagcagggggacaaguau

SEQ ID NO: 523 - RNA, 23 nt, Artificial Sequence
  siRNA strand
uucguacucggcccuguagggga

SEQ ID NO: 524 - RNA, 21 nt, Artificial Sequence
  siRNA strand
uccccuacagggccgaguacg

SEQ ID NO: 525 - RNA, 23 nt, Artificial Sequence
  siRNA strand
uagcuguagcgguaacaacccag

SEQ ID NO: 526 - RNA, 21 nt, Artificial Sequence
  siRNA strand
cuggguuguuaccgcuacagc

SEQ ID NO: 527 - RNA, 23 nt, Artificial Sequence
  siRNA strand
ugaaggacaccucuccaggccag

SEQ ID NO: 528 - RNA, 21 nt, Artificial Sequence
  siRNA strand
cuggccuggagagguguccuu

SEQ ID NO: 529 - RNA, 23 nt, Artificial Sequence
  siRNA strand
uacaagccauagugcacccgcac

SEQ ID NO: 530 - RNA, 21 nt, Artificial Sequence
  siRNA strand
gugcgggugcacuauggcuug

SEQ ID NO: 531 - RNA, 23 nt, Artificial Sequence
  siRNA strand
agacaagaugccaccuccugcca

SEQ ID NO: 532 - RNA, 21 nt, Artificial Sequence
  siRNA strand
uggcaggagguggcaucuugu

SEQ ID NO: 533 - RNA, 23 nt, Artificial Sequence
  siRNA strand
acuucguacucggcccuguaggg

SEQ ID NO: 534 - RNA, 21 nt, Artificial Sequence
  siRNA strand
cccuacagggccgaguacgaa

SEQ ID NO: 535 - RNA, 23 nt, Artificial Sequence
  siRNA strand
agaaugaaccagaagaagcaggu

SEQ ID NO: 536 - RNA, 21 nt, Artificial Sequence
  siRNA strand
accugcuucuucugguucauu

SEQ ID NO: 537 - RNA, 23 nt, Artificial Sequence
  siRNA strand
aguccuugaccccaucacaggca

SEQ ID NO: 538 - RNA, 21 nt, Artificial Sequence
  siRNA strand
ugccugugauggggucaagga

SEQ ID NO: 539 - RNA, 23 nt, Artificial Sequence
  siRNA strand
uagaccaggggcuuccgaagcug

SEQ ID NO: 540 - RNA, 23 nt, Artificial Sequence
  siRNA strand
uagaccaggggcuuccgaagcug

SEQ ID NO: 541 - RNA, 23 nt, Artificial Sequence
  siRNA strand
cagaucccaaguuagaccagggg

SEQ ID NO: 542 - RNA, 21 nt, Artificial Sequence
  siRNA strand
ccccuggucuaacuugggauc

SEQ ID NO: 543 - RNA, 23 nt, Artificial Sequence
  siRNA strand
uggagaaugaaccagaagaagca

SEQ ID NO: 544 - RNA, 21 nt, Artificial Sequence
  siRNA strand
ugcuucuucugguucauucuc

SEQ ID NO: 545 - RNA, 23 nt, Artificial Sequence
  siRNA strand
uuucucucauccaggccguuggg

SEQ ID NO: 546 - RNA, 21 nt, Artificial Sequence
  siRNA strand
cccaacggccuggaugagaga

SEQ ID NO: 547 - RNA, 23 nt, Artificial Sequence
  siRNA strand
ucguaguagcugugcaggcccuu

SEQ ID NO: 548 - RNA, 21 nt, Artificial Sequence
  siRNA strand
aagggccugcacagcuacuac

SEQ ID NO: 549 - RNA, 23 nt, Artificial Sequence
  siRNA strand
cauucccagaucccaaguuagac

SEQ ID NO: 550 - RNA, 21 nt, Artificial Sequence
  siRNA strand
gucuaacuugggaucugggaa

SEQ ID NO: 551 - RNA, 23 nt, Artificial Sequence
  siRNA strand
uuagaccaggggcuuccgaagcu

SEQ ID NO: 552 - RNA, 21 nt, Artificial Sequence
  siRNA strand
agcuucggaagccccuggucu

SEQ ID NO: 553 - RNA, 23 nt, Artificial Sequence
  siRNA strand
ugcagcuaugucuuucacacugg

SEQ ID NO: 554 - RNA, 21 nt, Artificial Sequence
  siRNA strand
ccagugugaaagacauagcug

SEQ ID NO: 555 - RNA, 23 nt, Artificial Sequence
  siRNA strand
agaugugucgaccccgaaccugg

SEQ ID NO: 556 - RNA, 21 nt, Artificial Sequence
  siRNA strand
ccagguucggggucgacacau

SEQ ID NO: 557 - RNA, 23 nt, Artificial Sequence
  siRNA strand
uagcgguaacaacccagcgugga

SEQ ID NO: 558 - RNA, 21 nt, Artificial Sequence
  siRNA strand
uccacgcuggguuguuaccgc

SEQ ID NO: 559 - RNA, 23 nt, Artificial Sequence
  siRNA strand
aauacuugucccccugcuuggca

SEQ ID NO: 560 - RNA, 21 nt, Artificial Sequence
  siRNA strand
ugccaagcagggggacaagua

SEQ ID NO: 561 - RNA, 23 nt, Artificial Sequence
  siRNA strand
ccacacagccuccuguucuggau

SEQ ID NO: 562 - RNA, 21 nt, Artificial Sequence
  siRNA strand
auccagaacaggaggcugugu

SEQ ID NO: 563 - RNA, 23 nt, Artificial Sequence
  siRNA strand
gugagggagaucugggaggugaa

SEQ ID NO: 564 - RNA, 21 nt, Artificial Sequence
  siRNA strand
uucaccucccagaucucccuc

SEQ ID NO: 565 - RNA, 23 nt, Artificial Sequence
  siRNA strand
ccauggccacucacccucggagg

SEQ ID NO: 566 - RNA, 21 nt, Artificial Sequence
  siRNA strand
ccuccgagggugaguggccau

SEQ ID NO: 567 - RNA, 23 nt, Artificial Sequence
  siRNA strand
gccacacagccuccuguucugga

SEQ ID NO: 568 - RNA, 21 nt, Artificial Sequence
  siRNA strand
uccagaacaggaggcugugug

SEQ ID NO: 569 - RNA, 23 nt, Artificial Sequence
  siRNA strand
ggccacucacccucggaggacac

SEQ ID NO: 570 - RNA, 21 nt, Artificial Sequence
  siRNA strand
guguccuccgagggugagugg

SEQ ID NO: 571 - RNA, 23 nt, Artificial Sequence
  siRNA strand
cccacagaugugucgaccccgaa

SEQ ID NO: 572 - RNA, 21 nt, Artificial Sequence
  siRNA strand
uucggggucgacacaucugug

SEQ ID NO: 573 - RNA, 23 nt, Artificial Sequence
  siRNA strand
ccccacagaugugucgaccccga

SEQ ID NO: 574 - RNA, 21 nt, Artificial Sequence
  siRNA strand
ucggggucgacacaucugugg

SEQ ID NO: 575 - RNA, 23 nt, Artificial Sequence
  siRNA strand
gccaugcuguccuccuggaagca

SEQ ID NO: 576 - RNA, 21 nt, Artificial Sequence
  siRNA strand
ugcuuccaggaggacagcaug

SEQ ID NO: 577 - RNA, 23 nt, Artificial Sequence
  siRNA strand
uguacccuaggaaauaccagagu

SEQ ID NO: 578 - RNA, 21 nt, Artificial Sequence
  siRNA strand
ucugguauuuccuaggguaca

SEQ ID NO: 579 - RNA, 23 nt, Artificial Sequence
  siRNA strand
aggaaauaccagaguagcacccc

SEQ ID NO: 580 - RNA, 21 nt, Artificial Sequence
  siRNA strand
ggugcuacucugguauuuccu

SEQ ID NO: 581 - RNA, 23 nt, Artificial Sequence
  siRNA strand
uguacccuaggaaauaccagagu

SEQ ID NO: 582 - RNA, 21 nt, Artificial Sequence
  siRNA strand
ucugguauuuccuaggguaca

SEQ ID NO: 583 - RNA, 23 nt, Artificial Sequence
  siRNA strand
uuguacccuaggaaauaccagag

SEQ ID NO: 584 - RNA, 21 nt, Artificial Sequence
  siRNA strand
cugguauuuccuaggguacaa

SEQ ID NO: 585 - RNA, 23 nt, Artificial Sequence
  siRNA strand
uuuguacccuaggaaauaccaga

SEQ ID NO: 586 - RNA, 21 nt, Artificial Sequence
  siRNA strand
ugguauuuccuaggguacaaa

SEQ ID NO: 587 - RNA, 23 nt, Artificial Sequence
  siRNA strand
ucuuguacccuaggaaauaccag

SEQ ID NO: 588 - RNA, 21 nt, Artificial Sequence
  siRNA strand
gguauuuccuaggguacaaga

SEQ ID NO: 589 - RNA, 19 nt, Artificial Sequence
  siRNA strand
uguacccuaggaaauacca

SEQ ID NO: 590 - RNA, 19 nt, Artificial Sequence
  siRNA strand
ugguauuuccuaggguaca

SEQ ID NO: 591 - RNA, 23 nt, Artificial Sequence
  siRNA strand
uccuuguacccuaggaaauacca

SEQ ID NO: 592 - RNA, 21 nt, Artificial Sequence
  siRNA strand
guauuuccuaggguacaagga

SEQ ID NO: 593 - RNA, 23 nt, Artificial Sequence
  siRNA strand
ucgccuuguacccuaggaaauac

SEQ ID NO: 594 - RNA, 21 nt, Artificial Sequence
  siRNA strand
auuuccuaggguacaaggcga

SEQ ID NO: 595 - RNA, 23 nt, Artificial Sequence
  siRNA strand
uccgccuuguacccuaggaaaua

SEQ ID NO: 596 - RNA, 21 nt, Artificial Sequence
  siRNA strand
uuuccuaggguacaaggcgga

SEQ ID NO: 597 - RNA, 23 nt, Artificial Sequence
  siRNA strand
aagauccugggagaaguggcgau

SEQ ID NO: 598 - RNA, 21 nt, Artificial Sequence
  siRNA strand
cgccacuucucccaggaucuu

SEQ ID NO: 599 - RNA, 23 nt, Artificial Sequence
  siRNA strand
uaagauccugggagaaguggcga

SEQ ID NO: 600 - RNA, 21 nt, Artificial Sequence
  siRNA strand
gccacuucucccaggaucuua

SEQ ID NO: 601 - RNA, 23 nt, Artificial Sequence
  siRNA strand
agaaugaaccagaagaagcaggu

SEQ ID NO: 602 - RNA, 21 nt, Artificial Sequence
  siRNA strand
cugcuucuucugguucauucu

SEQ ID NO: 603 - RNA, 23 nt, Artificial Sequence
  siRNA strand
uggagaaugaaccagaagaagca

SEQ ID NO: 604 - RNA, 21 nt, Artificial Sequence
  siRNA strand
cuucuucugguucauucucca

SEQ ID NO: 605 - RNA, 23 nt, Artificial Sequence
  siRNA strand
uucguacucggcccuguagggga

SEQ ID NO: 606 - RNA, 21 nt, Artificial Sequence
  siRNA strand
cccuacagggccgaguacgaa

SEQ ID NO: 607 - RNA, 23 nt, Artificial Sequence
  siRNA strand
acuucguacucggcccuguaggg

SEQ ID NO: 608 - RNA, 21 nt, Artificial Sequence
  siRNA strand
cuacagggccgaguacgaagu

SEQ ID NO: 609 - RNA, 23 nt, Artificial Sequence
  siRNA strand
agcuaugucuuucacacuggcuu

SEQ ID NO: 610 - RNA, 21 nt, Artificial Sequence
  siRNA strand
gccagugugaaagacauagcu

SEQ ID NO: 611 - RNA, 23 nt, Artificial Sequence
  siRNA strand
ugcagcuaugucuuucacacugg

SEQ ID NO: 612 - RNA, 21 nt, Artificial Sequence
  siRNA strand
agugugaaagacauagcugca

SEQ ID NO: 613 - RNA, 23 nt, Artificial Sequence
  siRNA strand
uagcgguaacaacccagcgugga

SEQ ID NO: 614 - RNA, 21 nt, Artificial Sequence
  siRNA strand
cacgcuggguuguuaccgcua

SEQ ID NO: 615 - RNA, 23 nt, Artificial Sequence
  siRNA strand
uagcuguagcgguaacaacccag

SEQ ID NO: 616 - RNA, 21 nt, Artificial Sequence
  siRNA strand
ggguuguuaccgcuacagcua

SEQ ID NO: 617 - RNA, 23 nt, Artificial Sequence
  siRNA strand
auacauggccagucggucccggc

SEQ ID NO: 618 - RNA, 21 nt, Artificial Sequence
  siRNA strand
cgggaccgacuggccauguau

SEQ ID NO: 619 - RNA, 23 nt, Artificial Sequence
  siRNA strand
acgucauacauggccagucgguc

SEQ ID NO: 620 - RNA, 21 nt, Artificial Sequence
  siRNA strand
ccgacuggccauguaugacgu

SEQ ID NO: 621 - RNA, 23 nt, Artificial Sequence
  siRNA strand
ucguaguagcugugcaggcccuu

SEQ ID NO: 622 - RNA, 21 nt, Artificial Sequence
  siRNA strand
gggccugcacagcuacuacga

SEQ ID NO: 623 - RNA, 23 nt, Artificial Sequence
  siRNA strand
acggcaaaucauacuucugccuc

SEQ ID NO: 624 - RNA, 21 nt, Artificial Sequence
  siRNA strand
ggcagaaguaugauuugccgu

SEQ ID NO: 625 - RNA, 23 nt, Artificial Sequence
  siRNA strand
ccacacagccuccuguucuggau

SEQ ID NO: 626 - RNA, 21 nt, Artificial Sequence
  siRNA strand
ccagaacaggaggcugugugg

SEQ ID NO: 627 - RNA, 23 nt, Artificial Sequence
  siRNA strand
gccacacagccuccuguucugga

SEQ ID NO: 628 - RNA, 21 nt, Artificial Sequence
  siRNA strand
cagaacaggaggcuguguggc

SEQ ID NO: 629 - RNA, 23 nt, Artificial Sequence
  siRNA strand
gugagggagaucugggaggugaa

SEQ ID NO: 630 - RNA, 21 nt, Artificial Sequence
  siRNA strand
caccucccagaucucccucac

SEQ ID NO: 631 - RNA, 23 nt, Artificial Sequence
  siRNA strand
uugagggagaucugggaggugaa

SEQ ID NO: 632 - RNA, 21 nt, Artificial Sequence
  siRNA strand
caccucccagaucucccucaa

SEQ ID NO: 633 - RNA, 23 nt, Artificial Sequence
  siRNA strand
aagccauagugcacccgcacacc

SEQ ID NO: 634 - RNA, 21 nt, Artificial Sequence
  siRNA strand
ugugcgggugcacuauggcuu

SEQ ID NO: 635 - RNA, 23 nt, Artificial Sequence
  siRNA strand
uacaagccauagugcacccgcac

SEQ ID NO: 636 - RNA, 21 nt, Artificial Sequence
  siRNA strand
gcgggugcacuauggcuugua

SEQ ID NO: 637 - RNA, 23 nt, Artificial Sequence
  siRNA strand
aggaacucuccagggcagggguc

SEQ ID NO: 638 - RNA, 21 nt, Artificial Sequence
  siRNA strand
ccccugcccuggagaguuccu

SEQ ID NO: 639 - RNA, 23 nt, Artificial Sequence
  siRNA strand
uaggaacucuccagggcaggggu

SEQ ID NO: 640 - RNA, 21 nt, Artificial Sequence
  siRNA strand
cccugcccuggagaguuccua

SEQ ID NO: 641 - RNA, 23 nt, Artificial Sequence
  siRNA strand
agaggaacucuccagggcagggg

SEQ ID NO: 642 - RNA, 21 nt, Artificial Sequence
  siRNA strand
ccugcccuggagaguuccucu

SEQ ID NO: 643 - RNA, 23 nt, Artificial Sequence
  siRNA strand
uagaggaacucuccagggcaggg

SEQ ID NO: 644 - RNA, 21 nt, Artificial Sequence
  siRNA strand
cugcccuggagaguuccucua

SEQ ID NO: 645 - RNA, 23 nt, Artificial Sequence
  siRNA strand
aguccuugaccccaucacaggca

SEQ ID NO: 646 - RNA, 21 nt, Artificial Sequence
  siRNA strand
ccugugauggggucaaggacu

SEQ ID NO: 647 - RNA, 23 nt, Artificial Sequence
  siRNA strand
uccaggccguuggggcaguccuu

SEQ ID NO: 648 - RNA, 21 nt, Artificial Sequence
  siRNA strand
ggacugccccaacggccugga

SEQ ID NO: 649 - RNA, 23 nt, Artificial Sequence
  siRNA strand
uauccaggccguuggggcagucc

SEQ ID NO: 650 - RNA, 21 nt, Artificial Sequence
  siRNA strand
acugccccaacggccuggaua

SEQ ID NO: 651 - RNA, 23 nt, Artificial Sequence
  siRNA strand
ucauccaggccguuggggcaguc

SEQ ID NO: 652 - RNA, 21 nt, Artificial Sequence
  siRNA strand
cugccccaacggccuggauga

SEQ ID NO: 653 - RNA, 23 nt, Artificial Sequence
  siRNA strand
uucauccaggccguuggggcagu

SEQ ID NO: 654 - RNA, 21 nt, Artificial Sequence
  siRNA strand
ugccccaacggccuggaugaa

SEQ ID NO: 655 - RNA, 23 nt, Artificial Sequence
  siRNA strand
ucucauccaggccguuggggcag

SEQ ID NO: 656 - RNA, 21 nt, Artificial Sequence
  siRNA strand
gccccaacggccuggaugaga

SEQ ID NO: 657 - RNA, 23 nt, Artificial Sequence
  siRNA strand
uucucauccaggccguuggggca

SEQ ID NO: 658 - RNA, 21 nt, Artificial Sequence
  siRNA strand
ccccaacggccuggaugagaa

SEQ ID NO: 659 - RNA, 23 nt, Artificial Sequence
  siRNA strand
ucucucauccaggccguuggggc

SEQ ID NO: 660 - RNA, 21 nt, Artificial Sequence
  siRNA strand
cccaacggccuggaugagaga

SEQ ID NO: 661 - RNA, 23 nt, Artificial Sequence
  siRNA strand
uuucucucauccaggccguuggg

SEQ ID NO: 662 - RNA, 21 nt, Artificial Sequence
  siRNA strand
caacggccuggaugagagaaa

SEQ ID NO: 663 - RNA, 23 nt, Artificial Sequence
  siRNA strand
aguuucucucauccaggccguug

SEQ ID NO: 664 - RNA, 21 nt, Artificial Sequence
  siRNA strand
acggccuggaugagagaaacu

SEQ ID NO: 665 - RNA, 23 nt, Artificial Sequence
  siRNA strand
acgcaguuucucucauccaggcc

SEQ ID NO: 666 - RNA, 21 nt, Artificial Sequence
  siRNA strand
ccuggaugagagaaacugcgu

SEQ ID NO: 667 - RNA, 23 nt, Artificial Sequence
  siRNA strand
uuggaggccacagucacagugcu

SEQ ID NO: 668 - RNA, 21 nt, Artificial Sequence
  siRNA strand
cacugugacuguggccuccaa

SEQ ID NO: 669 - RNA, 23 nt, Artificial Sequence
  siRNA strand
ggccacucacccucggaggacac

SEQ ID NO: 670 - RNA, 21 nt, Artificial Sequence
  siRNA strand
guccuccgagggugaguggcc

SEQ ID NO: 671 - RNA, 23 nt, Artificial Sequence
  siRNA strand
uauggccacucacccucggagga

SEQ ID NO: 672 - RNA, 21 nt, Artificial Sequence
  siRNA strand
cuccgagggugaguggccaua

SEQ ID NO: 673 - RNA, 23 nt, Artificial Sequence
  siRNA strand
ccauggccacucacccucggagg

SEQ ID NO: 674 - RNA, 21 nt, Artificial Sequence
  siRNA strand
uccgagggugaguggccaugg

SEQ ID NO: 675 - RNA, 23 nt, Artificial Sequence
  siRNA strand
augugucgaccccgaaccuggag

SEQ ID NO: 676 - RNA, 21 nt, Artificial Sequence
  siRNA strand
ccagguucggggucgacacau

SEQ ID NO: 677 - RNA, 23 nt, Artificial Sequence
  siRNA strand
agaugugucgaccccgaaccugg

SEQ ID NO: 678 - RNA, 21 nt, Artificial Sequence
  siRNA strand
agguucggggucgacacaucu

SEQ ID NO: 679 - RNA, 23 nt, Artificial Sequence
  siRNA strand
cccacagaugugucgaccccgaa

SEQ ID NO: 680 - RNA, 21 nt, Artificial Sequence
  siRNA strand
cggggucgacacaucuguggg

SEQ ID NO: 681 - RNA, 23 nt, Artificial Sequence
  siRNA strand
uccacagaugugucgaccccgaa

SEQ ID NO: 682 - RNA, 21 nt, Artificial Sequence
  siRNA strand
cggggucgacacaucugugga

SEQ ID NO: 683 - RNA, 23 nt, Artificial Sequence
  siRNA strand
ccccacagaugugucgaccccga

SEQ ID NO: 684 - RNA, 21 nt, Artificial Sequence
  siRNA strand
ggggucgacacaucugugggg

SEQ ID NO: 685 - RNA, 23 nt, Artificial Sequence
  siRNA strand
ucccacagaugugucgaccccga

SEQ ID NO: 686 - RNA, 21 nt, Artificial Sequence
  siRNA strand
ggggucgacacaucuguggga

SEQ ID NO: 687 - RNA, 23 nt, Artificial Sequence
  siRNA strand
uccccacagaugugucgaccccg

SEQ ID NO: 688 - RNA, 21 nt, Artificial Sequence
  siRNA strand
gggucgacacaucugugggga

SEQ ID NO: 689 - RNA, 23 nt, Artificial Sequence
  siRNA strand
uguuaucacccagcggucagcga

SEQ ID NO: 690 - RNA, 21 nt, Artificial Sequence
  siRNA strand
gcugaccgcugggugauaaca

SEQ ID NO: 691 - RNA, 23 nt, Artificial Sequence
  siRNA strand
gccaugcuguccuccuggaagca

SEQ ID NO: 692 - RNA, 21 nt, Artificial Sequence
  siRNA strand
cuuccaggaggacagcauggc

SEQ ID NO: 693 - RNA, 23 nt, Artificial Sequence
  siRNA strand
ugaaggacaccucuccaggccag

SEQ ID NO: 694 - RNA, 21 nt, Artificial Sequence
  siRNA strand
ggccuggagagguguccuuca

SEQ ID NO: 695 - RNA, 23 nt, Artificial Sequence
  siRNA strand
uugaaggacaccucuccaggcca

SEQ ID NO: 696 - RNA, 21 nt, Artificial Sequence
  siRNA strand
gccuggagagguguccuucaa

SEQ ID NO: 697 - RNA, 23 nt, Artificial Sequence
  siRNA strand
aauacuugucccccugcuuggca

SEQ ID NO: 698 - RNA, 21 nt, Artificial Sequence
  siRNA strand
ccaagcagggggacaaguauu

SEQ ID NO: 699 - RNA, 23 nt, Artificial Sequence
  siRNA strand
gaauacuugucccccugcuuggc

SEQ ID NO: 700 - RNA, 21 nt, Artificial Sequence
  siRNA strand
caagcagggggacaaguauuc

SEQ ID NO: 701 - RNA, 23 nt, Artificial Sequence
  siRNA strand
acaagaugccaccuccugccacc

SEQ ID NO: 702 - RNA, 21 nt, Artificial Sequence
  siRNA strand
uggcaggagguggcaucuugu

SEQ ID NO: 703 - RNA, 23 nt, Artificial Sequence
  siRNA strand
agacaagaugccaccuccugcca

SEQ ID NO: 704 - RNA, 21 nt, Artificial Sequence
  siRNA strand
gcaggagguggcaucuugucu

SEQ ID NO: 705 - RNA, 23 nt, Artificial Sequence
  siRNA strand
uagaccaggggcuuccgaagcug

SEQ ID NO: 706 - RNA, 21 nt, Artificial Sequence
  siRNA strand
gcuucggaagccccuggucua

SEQ ID NO: 707 - RNA, 23 nt, Artificial Sequence
  siRNA strand
uuagaccaggggcuuccgaagcu

SEQ ID NO: 708 - RNA, 21 nt, Artificial Sequence
  siRNA strand
cuucggaagccccuggucuaa

SEQ ID NO: 709 - RNA, 23 nt, Artificial Sequence
  siRNA strand
gaucccaaguuagaccaggggcu

SEQ ID NO: 710 - RNA, 21 nt, Artificial Sequence
  siRNA strand
ccccuggucuaacuugggauc

SEQ ID NO: 711 - RNA, 23 nt, Artificial Sequence
  siRNA strand
agaucccaaguuagaccaggggc

SEQ ID NO: 712 - RNA, 21 nt, Artificial Sequence
  siRNA strand
cccuggucuaacuugggaucu

SEQ ID NO: 713 - RNA, 23 nt, Artificial Sequence
  siRNA strand
cagaucccaaguuagaccagggg

SEQ ID NO: 714 - RNA, 21 nt, Artificial Sequence
  siRNA strand
ccuggucuaacuugggaucug

SEQ ID NO: 715 - RNA, 23 nt, Artificial Sequence
  siRNA strand
cauucccagaucccaaguuagac

SEQ ID NO: 716 - RNA, 21 nt, Artificial Sequence
  siRNA strand
cuaacuugggaucugggaaug

SEQ ID NO: 717 - RNA, 19 nt, Artificial Sequence
  siRNA strand
cuggacugggucuggcuca

SEQ ID NO: 718 - RNA, 19 nt, Artificial Sequence
  siRNA strand
ugagccagacccaguccag

SEQ ID NO: 719 - RNA, 19 nt, Artificial Sequence
  siRNA strand
accagagcuggacuggguc

SEQ ID NO: 720 - RNA, 19 nt, Artificial Sequence
  siRNA strand
gacccaguccagcucuggu

SEQ ID NO: 721 - RNA, 19 nt, Artificial Sequence
  siRNA strand
cagagggcaggcaccagag

SEQ ID NO: 722 - RNA, 19 nt, Artificial Sequence
  siRNA strand
cucuggugccugcccucug

SEQ ID NO: 723 - RNA, 19 nt, Artificial Sequence
  siRNA strand
ucagcucgcaccagagggc

SEQ ID NO: 724 - RNA, 19 nt, Artificial Sequence
  siRNA strand
gcccucuggugcgagcuga

SEQ ID NO: 725 - RNA, 19 nt, Artificial Sequence
  siRNA strand
ucucaggucagcucgcacc

SEQ ID NO: 726 - RNA, 19 nt, Artificial Sequence
  siRNA strand
ggugcgagcugaccugaga

SEQ ID NO: 727 - RNA, 19 nt, Artificial Sequence
  siRNA strand
ggaagugcaucucagguca

SEQ ID NO: 728 - RNA, 19 nt, Artificial Sequence
  siRNA strand
ugaccugagaugcacuucc

SEQ ID NO: 729 - RNA, 19 nt, Artificial Sequence
  siRNA strand
cacagaggagggaagugca

SEQ ID NO: 730 - RNA, 19 nt, Artificial Sequence
  siRNA strand
ugcacuucccuccucugug

SEQ ID NO: 731 - RNA, 19 nt, Artificial Sequence
  siRNA strand
gugccgagacagcucacag

SEQ ID NO: 732 - RNA, 19 nt, Artificial Sequence
  siRNA strand
cugugagcugucucggcac

SEQ ID NO: 733 - RNA, 19 nt, Artificial Sequence
  siRNA strand
ugcaagugggugccgagac

SEQ ID NO: 734 - RNA, 19 nt, Artificial Sequence
  siRNA strand
gucucggcacccacuugca

SEQ ID NO: 735 - RNA, 19 nt, Artificial Sequence
  siRNA strand
cggcagugacugcaagugg

SEQ ID NO: 736 - RNA, 19 nt, Artificial Sequence
  siRNA strand
ccacuugcagucacugccg

SEQ ID NO: 737 - RNA, 19 nt, Artificial Sequence
  siRNA strand
aacaucaggcggcagugac

SEQ ID NO: 738 - RNA, 19 nt, Artificial Sequence
  siRNA strand
gucacugccgccugauguu

SEQ ID NO: 739 - RNA, 19 nt, Artificial Sequence
  siRNA strand
aagaguaacaacaucaggc

SEQ ID NO: 740 - RNA, 19 nt, Artificial Sequence
  siRNA strand
gccugauguuguuacucuu

SEQ ID NO: 741 - RNA, 19 nt, Artificial Sequence
  siRNA strand
uuuuggaguggaagaguaa

SEQ ID NO: 742 - RNA, 19 nt, Artificial Sequence
  siRNA strand
uuacucuuccacuccaaaa

SEQ ID NO: 743 - RNA, 19 nt, Artificial Sequence
  siRNA strand
acgggcauccuuuuggagu

SEQ ID NO: 744 - RNA, 19 nt, Artificial Sequence
  siRNA strand
acuccaaaaggaugcccgu

SEQ ID NO: 745 - RNA, 19 nt, Artificial Sequence
  siRNA strand
ggggccucggccacgggca

SEQ ID NO: 746 - RNA, 19 nt, Artificial Sequence
  siRNA strand
ugcccguggccgaggcccc

SEQ ID NO: 747 - RNA, 19 nt, Artificial Sequence
  siRNA strand
accuggggggccucggcca

SEQ ID NO: 748 - RNA, 19 nt, Artificial Sequence
  siRNA strand
uggccgaggccccccaggu

SEQ ID NO: 749 - RNA, 19 nt, Artificial Sequence
  siRNA strand
cugcccgccagccaccugg

SEQ ID NO: 750 - RNA, 19 nt, Artificial Sequence
  siRNA strand
ccagguggcuggcgggcag

SEQ ID NO: 751 - RNA, 19 nt, Artificial Sequence
  siRNA strand
ccuccgucccccugcccgc

SEQ ID NO: 752 - RNA, 19 nt, Artificial Sequence
  siRNA strand
gcgggcagggggacggagg

SEQ ID NO: 753 - RNA, 19 nt, Artificial Sequence
  siRNA strand
cucgccaucaccuccgucc

SEQ ID NO: 754 - RNA, 19 nt, Artificial Sequence
  siRNA strand
ggacggaggugauggcgag

SEQ ID NO: 755 - RNA, 19 nt, Artificial Sequence
  siRNA strand
uccgcuuccucgccaucac

SEQ ID NO: 756 - RNA, 19 nt, Artificial Sequence
  siRNA strand
gugauggcgaggaagcgga

SEQ ID NO: 757 - RNA, 19 nt, Artificial Sequence
  siRNA strand
uccccuccggcuccgcuuc

SEQ ID NO: 758 - RNA, 19 nt, Artificial Sequence
  siRNA strand
gaagcggagccggagggga

SEQ ID NO: 759 - RNA, 19 nt, Artificial Sequence
  siRNA strand
cuugaacauccccuccggc

SEQ ID NO: 760 - RNA, 19 nt, Artificial Sequence
  siRNA strand
gccggaggggauguucaag

SEQ ID NO: 761 - RNA, 19 nt, Artificial Sequence
  siRNA strand
uccucacaggccuugaaca

SEQ ID NO: 762 - RNA, 19 nt, Artificial Sequence
  siRNA strand
uguucaaggccugugagga

SEQ ID NO: 763 - RNA, 19 nt, Artificial Sequence
  siRNA strand
ucucuuggaguccucacag

SEQ ID NO: 764 - RNA, 19 nt, Artificial Sequence
  siRNA strand
cugugaggacuccaagaga

SEQ ID NO: 765 - RNA, 19 nt, Artificial Sequence
  siRNA strand
cgggcuuuucucuuggagu

SEQ ID NO: 766 - RNA, 19 nt, Artificial Sequence
  siRNA strand
acuccaagagaaaagcccg

SEQ ID NO: 767 - RNA, 19 nt, Artificial Sequence
  siRNA strand
ggcggagguagccccgggc

SEQ ID NO: 768 - RNA, 19 nt, Artificial Sequence
  siRNA strand
gcccggggcuaccuccgcc

SEQ ID NO: 769 - RNA, 19 nt, Artificial Sequence
  siRNA strand
aggggcaccaggcggaggu

SEQ ID NO: 770 - RNA, 19 nt, Artificial Sequence
  siRNA strand
accuccgccuggugccccu

SEQ ID NO: 771 - RNA, 19 nt, Artificial Sequence
  siRNA strand
acaaacaggggcaccaggc

SEQ ID NO: 772 - RNA, 19 nt, Artificial Sequence
  siRNA strand
gccuggugccccuguuugu

SEQ ID NO: 773 - RNA, 19 nt, Artificial Sequence
  siRNA strand
agggccagcagcacaaaca

SEQ ID NO: 774 - RNA, 19 nt, Artificial Sequence
  siRNA strand
uguuugugcugcuggcccu

SEQ ID NO: 775 - RNA, 19 nt, Artificial Sequence
  siRNA strand
agcacgagcagggccagca

SEQ ID NO: 776 - RNA, 19 nt, Artificial Sequence
  siRNA strand
ugcuggcccugcucgugcu

SEQ ID NO: 777 - RNA, 19 nt, Artificial Sequence
  siRNA strand
cgccgaagccagcacgagc

SEQ ID NO: 778 - RNA, 19 nt, Artificial Sequence
  siRNA strand
gcucgugcuggcuucggcg

SEQ ID NO: 779 - RNA, 19 nt, Artificial Sequence
  siRNA strand
agaguagcacccccgccga

SEQ ID NO: 780 - RNA, 19 nt, Artificial Sequence
  siRNA strand
ucggcgggggugcuacucu

SEQ ID NO: 781 - RNA, 19 nt, Artificial Sequence
  siRNA strand
cagaguagcacccccgccg

SEQ ID NO: 782 - RNA, 19 nt, Artificial Sequence
  siRNA strand
cggcgggggugcuacucug

SEQ ID NO: 783 - RNA, 19 nt, Artificial Sequence
  siRNA strand
ccagaguagcacccccgcc

SEQ ID NO: 784 - RNA, 19 nt, Artificial Sequence
  siRNA strand
ggcgggggugcuacucugg

SEQ ID NO: 785 - RNA, 19 nt, Artificial Sequence
  siRNA strand
accagaguagcacccccgc

SEQ ID NO: 786 - RNA, 19 nt, Artificial Sequence
  siRNA strand
gcgggggugcuacucuggu

SEQ ID NO: 787 - RNA, 19 nt, Artificial Sequence
  siRNA strand
uaccagaguagcacccccg

SEQ ID NO: 788 - RNA, 19 nt, Artificial Sequence
  siRNA strand
cgggggugcuacucuggua

SEQ ID NO: 789 - RNA, 19 nt, Artificial Sequence
  siRNA strand
auaccagaguagcaccccc

SEQ ID NO: 790 - RNA, 19 nt, Artificial Sequence
  siRNA strand
gggggugcuacucugguau

SEQ ID NO: 791 - RNA, 19 nt, Artificial Sequence
  siRNA strand
aaauaccagaguagcaccc

SEQ ID NO: 792 - RNA, 19 nt, Artificial Sequence
  siRNA strand
gggugcuacucugguauuu

SEQ ID NO: 793 - RNA, 19 nt, Artificial Sequence
  siRNA strand
gaaauaccagaguagcacc

SEQ ID NO: 794 - RNA, 19 nt, Artificial Sequence
  siRNA strand
ggugcuacucugguauuuc

SEQ ID NO: 795 - RNA, 19 nt, Artificial Sequence
  siRNA strand
ggaaauaccagaguagcac

SEQ ID NO: 796 - RNA, 19 nt, Artificial Sequence
  siRNA strand
gugcuacucugguauuucc

SEQ ID NO: 797 - RNA, 19 nt, Artificial Sequence
  siRNA strand
uaggaaauaccagaguagc

SEQ ID NO: 798 - RNA, 19 nt, Artificial Sequence
  siRNA strand
gcuacucugguauuuccua

SEQ ID NO: 799 - RNA, 19 nt, Artificial Sequence
  siRNA strand
cuaggaaauaccagaguag

SEQ ID NO: 800 - RNA, 19 nt, Artificial Sequence
  siRNA strand
cuacucugguauuuccuag

SEQ ID NO: 801 - RNA, 19 nt, Artificial Sequence
  siRNA strand
ccuaggaaauaccagagua

SEQ ID NO: 802 - RNA, 19 nt, Artificial Sequence
  siRNA strand
uacucugguauuuccuagg

SEQ ID NO: 803 - RNA, 19 nt, Artificial Sequence
  siRNA strand
cccuaggaaauaccagagu

SEQ ID NO: 804 - RNA, 19 nt, Artificial Sequence
  siRNA strand
acucugguauuuccuaggg

SEQ ID NO: 805 - RNA, 19 nt, Artificial Sequence
  siRNA strand
acccuaggaaauaccagag

SEQ ID NO: 806 - RNA, 19 nt, Artificial Sequence
  siRNA strand
cucugguauuuccuagggu

SEQ ID NO: 807 - RNA, 19 nt, Artificial Sequence
  siRNA strand
guacccuaggaaauaccag

SEQ ID NO: 808 - RNA, 19 nt, Artificial Sequence
  siRNA strand
cugguauuuccuaggguac

SEQ ID NO: 809 - RNA, 19 nt, Artificial Sequence
  siRNA strand
cuuguacccuaggaaauac

SEQ ID NO: 810 - RNA, 19 nt, Artificial Sequence
  siRNA strand
guauuuccuaggguacaag

SEQ ID NO: 811 - RNA, 19 nt, Artificial Sequence
  siRNA strand
ccuuguacccuaggaaaua

SEQ ID NO: 812 - RNA, 19 nt, Artificial Sequence
  siRNA strand
uauuuccuaggguacaagg

SEQ ID NO: 813 - RNA, 19 nt, Artificial Sequence
  siRNA strand
gccuuguacccuaggaaau

SEQ ID NO: 814 - RNA, 19 nt, Artificial Sequence
  siRNA strand
auuuccuaggguacaaggc

SEQ ID NO: 815 - RNA, 19 nt, Artificial Sequence
  siRNA strand
cgccuuguacccuaggaaa

SEQ ID NO: 816 - RNA, 19 nt, Artificial Sequence
  siRNA strand
uuuccuaggguacaaggcg

SEQ ID NO: 817 - RNA, 19 nt, Artificial Sequence
  siRNA strand
ccgccuuguacccuaggaa

SEQ ID NO: 818 - RNA, 19 nt, Artificial Sequence
  siRNA strand
uuccuaggguacaaggcgg

SEQ ID NO: 819 - RNA, 19 nt, Artificial Sequence
  siRNA strand
cuccgccuuguacccuagg

SEQ ID NO: 820 - RNA, 19 nt, Artificial Sequence
  siRNA strand
ccuaggguacaaggcggag

SEQ ID NO: 821 - RNA, 19 nt, Artificial Sequence
  siRNA strand
ccuccgccuuguacccuag

SEQ ID NO: 822 - RNA, 19 nt, Artificial Sequence
  siRNA strand
cuaggguacaaggcggagg

SEQ ID NO: 823 - RNA, 19 nt, Artificial Sequence
  siRNA strand
accuccgccuuguacccua

SEQ ID NO: 824 - RNA, 19 nt, Artificial Sequence
  siRNA strand
uaggguacaaggcggaggu

SEQ ID NO: 825 - RNA, 19 nt, Artificial Sequence
  siRNA strand
caccuccgccuuguacccu

SEQ ID NO: 826 - RNA, 19 nt, Artificial Sequence
  siRNA strand
aggguacaaggcggaggug

SEQ ID NO: 827 - RNA, 19 nt, Artificial Sequence
  siRNA strand
ucaccuccgccuuguaccc

SEQ ID NO: 828 - RNA, 19 nt, Artificial Sequence
  siRNA strand
ggguacaaggcggagguga

SEQ ID NO: 829 - RNA, 19 nt, Artificial Sequence
  siRNA strand
aucaccuccgccuuguacc

SEQ ID NO: 830 - RNA, 19 nt, Artificial Sequence
  siRNA strand
gguacaaggcggaggugau

SEQ ID NO: 831 - RNA, 19 nt, Artificial Sequence
  siRNA strand
caucaccuccgccuuguac

SEQ ID NO: 832 - RNA, 19 nt, Artificial Sequence
  siRNA strand
guacaaggcggaggugaug

SEQ ID NO: 833 - RNA, 19 nt, Artificial Sequence
  siRNA strand
ccaucaccuccgccuugua

SEQ ID NO: 834 - RNA, 19 nt, Artificial Sequence
  siRNA strand
uacaaggcggaggugaugg

SEQ ID NO: 835 - RNA, 19 nt, Artificial Sequence
  siRNA strand
accaucaccuccgccuugu

SEQ ID NO: 836 - RNA, 19 nt, Artificial Sequence
  siRNA strand
acaaggcggaggugauggu

SEQ ID NO: 837 - RNA, 19 nt, Artificial Sequence
  siRNA strand
gaccaucaccuccgccuug

SEQ ID NO: 838 - RNA, 19 nt, Artificial Sequence
  siRNA strand
caaggcggaggugaugguc

SEQ ID NO: 839 - RNA, 19 nt, Artificial Sequence
  siRNA strand
ugaccaucaccuccgccuu

SEQ ID NO: 840 - RNA, 19 nt, Artificial Sequence
  siRNA strand
aaggcggaggugaugguca

SEQ ID NO: 841 - RNA, 19 nt, Artificial Sequence
  siRNA strand
cugaccaucaccuccgccu

SEQ ID NO: 842 - RNA, 19 nt, Artificial Sequence
  siRNA strand
aggcggaggugauggucag

SEQ ID NO: 843 - RNA, 19 nt, Artificial Sequence
  siRNA strand
uacaccuggcugaccauca

SEQ ID NO: 844 - RNA, 19 nt, Artificial Sequence
  siRNA strand
ugauggucagccaggugua

SEQ ID NO: 845 - RNA, 19 nt, Artificial Sequence
  siRNA strand
acugccugaguacaccugg

SEQ ID NO: 846 - RNA, 19 nt, Artificial Sequence
  siRNA strand
ccagguguacucaggcagu

SEQ ID NO: 847 - RNA, 19 nt, Artificial Sequence
  siRNA strand
uugaguacacgcagacugc

SEQ ID NO: 848 - RNA, 19 nt, Artificial Sequence
  siRNA strand
gcagucugcguguacucaa

SEQ ID NO: 849 - RNA, 19 nt, Artificial Sequence
  siRNA strand
guggcgauugaguacacgc

SEQ ID NO: 850 - RNA, 19 nt, Artificial Sequence
  siRNA strand
gcguguacucaaucgccac

SEQ ID NO: 851 - RNA, 19 nt, Artificial Sequence
  siRNA strand
auccugggagaaguggcga

SEQ ID NO: 852 - RNA, 19 nt, Artificial Sequence
  siRNA strand
ucgccacuucucccaggau

SEQ ID NO: 853 - RNA, 19 nt, Artificial Sequence
  siRNA strand
gcggguaagauccugggag

SEQ ID NO: 854 - RNA, 19 nt, Artificial Sequence
  siRNA strand
cucccaggaucuuacccgc

SEQ ID NO: 855 - RNA, 19 nt, Artificial Sequence
  siRNA strand
acuagauucccggcgggua

SEQ ID NO: 856 - RNA, 19 nt, Artificial Sequence
  siRNA strand
uacccgccgggaaucuagu

SEQ ID NO: 857 - RNA, 19 nt, Artificial Sequence
  siRNA strand
gaaggcacuagauucccgg

SEQ ID NO: 858 - RNA, 19 nt, Artificial Sequence
  siRNA strand
ccgggaaucuagugccuuc

SEQ ID NO: 859 - RNA, 19 nt, Artificial Sequence
  siRNA strand
uuucacugcggaaggcacu

SEQ ID NO: 860 - RNA, 19 nt, Artificial Sequence
  siRNA strand
agugccuuccgcagugaaa

SEQ ID NO: 861 - RNA, 19 nt, Artificial Sequence
  siRNA strand
ugggcuuuggcgguuucac

SEQ ID NO: 862 - RNA, 19 nt, Artificial Sequence
  siRNA strand
gugaaaccgccaaagccca

SEQ ID NO: 863 - RNA, 19 nt, Artificial Sequence
  siRNA strand
caucuucugggcuuuggcg

SEQ ID NO: 864 - RNA, 19 nt, Artificial Sequence
  siRNA strand
cgccaaagcccagaagaug

SEQ ID NO: 865 - RNA, 19 nt, Artificial Sequence
  siRNA strand
gcuccuugagcaucuucug

SEQ ID NO: 866 - RNA, 19 nt, Artificial Sequence
  siRNA strand
cagaagaugcucaaggagc

SEQ ID NO: 867 - RNA, 19 nt, Artificial Sequence
  siRNA strand
cuggugaugagcuccuuga

SEQ ID NO: 868 - RNA, 19 nt, Artificial Sequence
  siRNA strand
ucaaggagcucaucaccag

SEQ ID NO: 869 - RNA, 19 nt, Artificial Sequence
  siRNA strand
uucccaggcgggugcuggu

SEQ ID NO: 870 - RNA, 19 nt, Artificial Sequence
  siRNA strand
accagcacccgccugggaa

SEQ ID NO: 871 - RNA, 19 nt, Artificial Sequence
  siRNA strand
uuguaguaaguucccaggc

SEQ ID NO: 872 - RNA, 19 nt, Artificial Sequence
  siRNA strand
gccugggaacuuacuacaa

SEQ ID NO: 873 - RNA, 19 nt, Artificial Sequence
  siRNA strand
cuggaguuguaguaaguuc

SEQ ID NO: 874 - RNA, 19 nt, Artificial Sequence
  siRNA strand
gaacuuacuacaacuccag

SEQ ID NO: 875 - RNA, 19 nt, Artificial Sequence
  siRNA strand
aauagacggagcuggaguu

SEQ ID NO: 876 - RNA, 19 nt, Artificial Sequence
  siRNA strand
aacuccagcuccgucuauu

SEQ ID NO: 877 - RNA, 19 nt, Artificial Sequence
  siRNA strand
uccccaaaggaauagacgg

SEQ ID NO: 878 - RNA, 19 nt, Artificial Sequence
  siRNA strand
ccgucuauuccuuugggga

SEQ ID NO: 879 - RNA, 19 nt, Artificial Sequence
  siRNA strand
gugaggggucccuccccaa

SEQ ID NO: 880 - RNA, 19 nt, Artificial Sequence
  siRNA strand
uuggggagggaccccucac

SEQ ID NO: 881 - RNA, 19 nt, Artificial Sequence
  siRNA strand
agaagaagcaggugagggg

SEQ ID NO: 882 - RNA, 19 nt, Artificial Sequence
  siRNA strand
ccccucaccugcuucuucu

SEQ ID NO: 883 - RNA, 19 nt, Artificial Sequence
  siRNA strand
aaugaaccagaagaagcag

SEQ ID NO: 884 - RNA, 19 nt, Artificial Sequence
  siRNA strand
cugcuucuucugguucauu

SEQ ID NO: 885 - RNA, 19 nt, Artificial Sequence
  siRNA strand
gauuuggagaaugaaccag

SEQ ID NO: 886 - RNA, 19 nt, Artificial Sequence
  siRNA strand
cugguucauucuccaaauc

SEQ ID NO: 887 - RNA, 19 nt, Artificial Sequence
  siRNA strand
gugcucggggauuuggaga

SEQ ID NO: 888 - RNA, 19 nt, Artificial Sequence
  siRNA strand
ucuccaaauccccgagcac

SEQ ID NO: 889 - RNA, 19 nt, Artificial Sequence
  siRNA strand
aucagccggcggugcucgg

SEQ ID NO: 890 - RNA, 19 nt, Artificial Sequence
  siRNA strand
ccgagcaccgccggcugau

SEQ ID NO: 891 - RNA, 19 nt, Artificial Sequence
  siRNA strand
ucggggcucagcaucagcc

SEQ ID NO: 892 - RNA, 19 nt, Artificial Sequence
  siRNA strand
ggcugaugcugagccccga

SEQ ID NO: 893 - RNA, 19 nt, Artificial Sequence
  siRNA strand
ugcaccaccucggggcuca

SEQ ID NO: 894 - RNA, 19 nt, Artificial Sequence
  siRNA strand
ugagccccgagguggugca

SEQ ID NO: 895 - RNA, 19 nt, Artificial Sequence
  siRNA strand
accagcagugccugcacca

SEQ ID NO: 896 - RNA, 19 nt, Artificial Sequence
  siRNA strand
uggugcaggcacugcuggu

SEQ ID NO: 897 - RNA, 19 nt, Artificial Sequence
  siRNA strand
uccuccaccagcagugccu

SEQ ID NO: 898 - RNA, 19 nt, Artificial Sequence
  siRNA strand
aggcacugcugguggagga

SEQ ID NO: 899 - RNA, 19 nt, Artificial Sequence
  siRNA strand
uggacagcagcuccuccac

SEQ ID NO: 900 - RNA, 19 nt, Artificial Sequence
  siRNA strand
guggaggagcugcugucca

SEQ ID NO: 901 - RNA, 19 nt, Artificial Sequence
  siRNA strand
gagcuguugacuguggaca

SEQ ID NO: 902 - RNA, 19 nt, Artificial Sequence
  siRNA strand
uguccacagucaacagcuc

SEQ ID NO: 903 - RNA, 19 nt, Artificial Sequence
  siRNA strand
acggcagccgagcuguuga

SEQ ID NO: 904 - RNA, 19 nt, Artificial Sequence
  siRNA strand
ucaacagcucggcugccgu

SEQ ID NO: 905 - RNA, 19 nt, Artificial Sequence
  siRNA strand
uguaggggacggcagccga

SEQ ID NO: 906 - RNA, 19 nt, Artificial Sequence
  siRNA strand
ucggcugccguccccuaca

SEQ ID NO: 907 - RNA, 19 nt, Artificial Sequence
  siRNA strand
uaggcccucgggguccacu

SEQ ID NO: 908 - RNA, 19 nt, Artificial Sequence
  siRNA strand
aguggaccccgagggccua

SEQ ID NO: 909 - RNA, 19 nt, Artificial Sequence
  siRNA strand
uccaggaucacuaggcccu

SEQ ID NO: 910 - RNA, 19 nt, Artificial Sequence
  siRNA strand
agggccuagugauccugga

SEQ ID NO: 911 - RNA, 19 nt, Artificial Sequence
  siRNA strand
cuggcuuccaggaucacua

SEQ ID NO: 912 - RNA, 19 nt, Artificial Sequence
  siRNA strand
uagugauccuggaagccag

SEQ ID NO: 913 - RNA, 19 nt, Artificial Sequence
  siRNA strand
augucuuucacacuggcuu

SEQ ID NO: 914 - RNA, 19 nt, Artificial Sequence
  siRNA strand
aagccagugugaaagacau

SEQ ID NO: 915 - RNA, 19 nt, Artificial Sequence
  siRNA strand
aaugcagcuaugucuuuca

SEQ ID NO: 916 - RNA, 19 nt, Artificial Sequence
  siRNA strand
ugaaagacauagcugcauu

SEQ ID NO: 917 - RNA, 19 nt, Artificial Sequence
  siRNA strand
cagcguggaauucaaugca

SEQ ID NO: 918 - RNA, 19 nt, Artificial Sequence
  siRNA strand
ugcauugaauuccacgcug

SEQ ID NO: 919 - RNA, 19 nt, Artificial Sequence
  siRNA strand
cuggcccacguagcuguag

SEQ ID NO: 920 - RNA, 19 nt, Artificial Sequence
  siRNA strand
cuacagcuacgugggccag

SEQ ID NO: 921 - RNA, 19 nt, Artificial Sequence
  siRNA strand
cuggcccuggcccacguag

SEQ ID NO: 922 - RNA, 19 nt, Artificial Sequence
  siRNA strand
cuacgugggccagggccag

SEQ ID NO: 923 - RNA, 19 nt, Artificial Sequence
  siRNA strand
agccggaggaccuggcccu

SEQ ID NO: 924 - RNA, 19 nt, Artificial Sequence
  siRNA strand
agggccagguccuccggcu

SEQ ID NO: 925 - RNA, 19 nt, Artificial Sequence
  siRNA strand
gucaggccccuucagccgg

SEQ ID NO: 926 - RNA, 19 nt, Artificial Sequence
  siRNA strand
ccggcugaaggggccugac

SEQ ID NO: 927 - RNA, 19 nt, Artificial Sequence
  siRNA strand
aggccagguggucaggccc

SEQ ID NO: 928 - RNA, 19 nt, Artificial Sequence
  siRNA strand
gggccugaccaccuggccu

SEQ ID NO: 929 - RNA, 19 nt, Artificial Sequence
  siRNA strand
gcagcuggaggccaggugg

SEQ ID NO: 930 - RNA, 19 nt, Artificial Sequence
  siRNA strand
ccaccuggccuccagcugc

SEQ ID NO: 931 - RNA, 19 nt, Artificial Sequence
  siRNA strand
aggugccacaggcagcugg

SEQ ID NO: 932 - RNA, 19 nt, Artificial Sequence
  siRNA strand
ccagcugccuguggcaccu

SEQ ID NO: 933 - RNA, 19 nt, Artificial Sequence
  siRNA strand
ggcccugcaggugccacag

SEQ ID NO: 934 - RNA, 19 nt, Artificial Sequence
  siRNA strand
cuguggcaccugcagggcc

SEQ ID NO: 935 - RNA, 19 nt, Artificial Sequence
  siRNA strand
gguccuuggggcccugcag

SEQ ID NO: 936 - RNA, 19 nt, Artificial Sequence
  siRNA strand
cugcagggccccaaggacc

SEQ ID NO: 937 - RNA, 19 nt, Artificial Sequence
  siRNA strand
uugagcaugagguccuugg

SEQ ID NO: 938 - RNA, 19 nt, Artificial Sequence
  siRNA strand
ccaaggaccucaugcucaa

SEQ ID NO: 939 - RNA, 19 nt, Artificial Sequence
  siRNA strand
uccagccggaguuugagca

SEQ ID NO: 940 - RNA, 19 nt, Artificial Sequence
  siRNA strand
ugcucaaacuccggcugga

SEQ ID NO: 941 - RNA, 19 nt, Artificial Sequence
  siRNA strand
cagcguccacuccagccgg

SEQ ID NO: 942 - RNA, 19 nt, Artificial Sequence
  siRNA strand
ccggcuggaguggacgcug

SEQ ID NO: 943 - RNA, 19 nt, Artificial Sequence
  siRNA strand
ccggcacucugccagcguc

SEQ ID NO: 944 - RNA, 19 nt, Artificial Sequence
  siRNA strand
gacgcuggcagagugccgg

SEQ ID NO: 945 - RNA, 19 nt, Artificial Sequence
  siRNA strand
ucggucccggcacucugcc

SEQ ID NO: 946 - RNA, 19 nt, Artificial Sequence
  siRNA strand
ggcagagugccgggaccga

SEQ ID NO: 947 - RNA, 19 nt, Artificial Sequence
  siRNA strand
ucauacauggccagucggu

SEQ ID NO: 948 - RNA, 19 nt, Artificial Sequence
  siRNA strand
accgacuggccauguauga

SEQ ID NO: 949 - RNA, 19 nt, Artificial Sequence
  siRNA strand
ccggccacgucauacaugg

SEQ ID NO: 950 - RNA, 19 nt, Artificial Sequence
  siRNA strand
ccauguaugacguggccgg

SEQ ID NO: 951 - RNA, 19 nt, Artificial Sequence
  siRNA strand
ucuccaggggcccggccac

SEQ ID NO: 952 - RNA, 19 nt, Artificial Sequence
  siRNA strand
guggccgggccccuggaga

SEQ ID NO: 953 - RNA, 19 nt, Artificial Sequence
  siRNA strand
augagccucuucuccaggg

SEQ ID NO: 954 - RNA, 19 nt, Artificial Sequence
  siRNA strand
cccuggagaagaggcucau

SEQ ID NO: 955 - RNA, 19 nt, Artificial Sequence
  siRNA strand
gaggugaugagccucuucu

SEQ ID NO: 956 - RNA, 19 nt, Artificial Sequence
  siRNA strand
agaagaggcucaucaccuc

SEQ ID NO: 957 - RNA, 19 nt, Artificial Sequence
  siRNA strand
ugcagccguacaccgaggu

SEQ ID NO: 958 - RNA, 19 nt, Artificial Sequence
  siRNA strand
accucgguguacggcugca

SEQ ID NO: 959 - RNA, 19 nt, Artificial Sequence
  siRNA strand
uccuggcggcugcagccgu

SEQ ID NO: 960 - RNA, 19 nt, Artificial Sequence
  siRNA strand
acggcugcagccgccagga

SEQ ID NO: 961 - RNA, 19 nt, Artificial Sequence
  siRNA strand
accacgggcuccuggcggc

SEQ ID NO: 962 - RNA, 19 nt, Artificial Sequence
  siRNA strand
gccgccaggagcccguggu

SEQ ID NO: 963 - RNA, 19 nt, Artificial Sequence
  siRNA strand
agaaccuccaccacgggcu

SEQ ID NO: 964 - RNA, 19 nt, Artificial Sequence
  siRNA strand
agcccgugguggagguucu

SEQ ID NO: 965 - RNA, 19 nt, Artificial Sequence
  siRNA strand
ccgacgccagaaccuccac

SEQ ID NO: 966 - RNA, 19 nt, Artificial Sequence
  siRNA strand
guggagguucuggcgucgg

SEQ ID NO: 967 - RNA, 19 nt, Artificial Sequence
  siRNA strand
augauggcccccgacgcca

SEQ ID NO: 968 - RNA, 19 nt, Artificial Sequence
  siRNA strand
uggcgucgggggccaucau

SEQ ID NO: 969 - RNA, 19 nt, Artificial Sequence
  siRNA strand
cagacgaccgccaugaugg

SEQ ID NO: 970 - RNA, 19 nt, Artificial Sequence
  siRNA strand
ccaucauggcggucgucug

SEQ ID NO: 971 - RNA, 19 nt, Artificial Sequence
  siRNA strand
ccuucuuccagacgaccgc

SEQ ID NO: 972 - RNA, 19 nt, Artificial Sequence
  siRNA strand
gcggucgucuggaagaagg

SEQ ID NO: 973 - RNA, 19 nt, Artificial Sequence
  siRNA strand
cugugcaggcccuucuucc

SEQ ID NO: 974 - RNA, 19 nt, Artificial Sequence
  siRNA strand
ggaagaagggccugcacag

SEQ ID NO: 975 - RNA, 19 nt, Artificial Sequence
  siRNA strand
gucguaguagcugugcagg

SEQ ID NO: 976 - RNA, 19 nt, Artificial Sequence
  siRNA strand
ccugcacagcuacuacgac

SEQ ID NO: 977 - RNA, 19 nt, Artificial Sequence
  siRNA strand
agcacgaaggggucguagu

SEQ ID NO: 978 - RNA, 19 nt, Artificial Sequence
  siRNA strand
acuacgaccccuucgugcu

SEQ ID NO: 979 - RNA, 19 nt, Artificial Sequence
  siRNA strand
ugcacggagagcacgaagg

SEQ ID NO: 980 - RNA, 19 nt, Artificial Sequence
  siRNA strand
ccuucgugcucuccgugca

SEQ ID NO: 981 - RNA, 19 nt, Artificial Sequence
  siRNA strand
aagaccaccggcugcacgg

SEQ ID NO: 982 - RNA, 19 nt, Artificial Sequence
  siRNA strand
ccgugcagccgguggucuu

SEQ ID NO: 983 - RNA, 19 nt, Artificial Sequence
  siRNA strand
caggccuggaagaccaccg

SEQ ID NO: 984 - RNA, 19 nt, Artificial Sequence
  siRNA strand
cgguggucuuccaggccug

SEQ ID NO: 985 - RNA, 19 nt, Artificial Sequence
  siRNA strand
agguucacuucacaggccu

SEQ ID NO: 986 - RNA, 19 nt, Artificial Sequence
  siRNA strand
aggccugugaagugaaccu

SEQ ID NO: 987 - RNA, 19 nt, Artificial Sequence
  siRNA strand
uccagcgucagguucacuu

SEQ ID NO: 988 - RNA, 19 nt, Artificial Sequence
  siRNA strand
aagugaaccugacgcugga

SEQ ID NO: 989 - RNA, 19 nt, Artificial Sequence
  siRNA strand
gagccuguuguccagcguc

SEQ ID NO: 990 - RNA, 19 nt, Artificial Sequence
  siRNA strand
gacgcuggacaacaggcuc

SEQ ID NO: 991 - RNA, 19 nt, Artificial Sequence
  siRNA strand
ugggagucgagccuguugu

SEQ ID NO: 992 - RNA, 19 nt, Artificial Sequence
  siRNA strand
acaacaggcucgacuccca

SEQ ID NO: 993 - RNA, 19 nt, Artificial Sequence
  siRNA strand
cugaggacgcccugggagu

SEQ ID NO: 994 - RNA, 19 nt, Artificial Sequence
  siRNA strand
acucccagggcguccucag

SEQ ID NO: 995 - RNA, 19 nt, Artificial Sequence
  siRNA strand
uagcuggggaaguacgggg

SEQ ID NO: 996 - RNA, 19 nt, Artificial Sequence
  siRNA strand
ccccguacuuccccagcua

SEQ ID NO: 997 - RNA, 19 nt, Artificial Sequence
  siRNA strand
gcgaguaguagcuggggaa

SEQ ID NO: 998 - RNA, 19 nt, Artificial Sequence
  siRNA strand
uuccccagcuacuacucgc

SEQ ID NO: 999 - RNA, 19 nt, Artificial Sequence
  siRNA strand
uggguuuggggcgaguagu

SEQ ID NO: 1000 - RNA, 19 nt, Artificial Sequence
  siRNA strand
acuacucgccccaaaccca

SEQ ID NO: 1001 - RNA, 19 nt, Artificial Sequence
  siRNA strand
caggagcaguggguuuggg

SEQ ID NO: 1002 - RNA, 19 nt, Artificial Sequence
  siRNA strand
cccaaacccacugcuccug

SEQ ID NO: 1003 - RNA, 19 nt, Artificial Sequence
  siRNA strand
accgugaggugccaggagc

SEQ ID NO: 1004 - RNA, 19 nt, Artificial Sequence
  siRNA strand
gcuccuggcaccucacggu

SEQ ID NO: 1005 - RNA, 19 nt, Artificial Sequence
  siRNA strand
agagagggcaccgugaggu

SEQ ID NO: 1006 - RNA, 19 nt, Artificial Sequence
  siRNA strand
accucacggugcccucucu

SEQ ID NO: 1007 - RNA, 19 nt, Artificial Sequence
  siRNA strand
caagccguaguccagagag

SEQ ID NO: 1008 - RNA, 19 nt, Artificial Sequence
  siRNA strand
cucucuggacuacggcuug

SEQ ID NO: 1009 - RNA, 19 nt, Artificial Sequence
  siRNA strand
agagggccaagccguaguc

SEQ ID NO: 1010 - RNA, 19 nt, Artificial Sequence
  siRNA strand
gacuacggcuuggcccucu

SEQ ID NO: 1011 - RNA, 19 nt, Artificial Sequence
  siRNA strand
uaggcaucaaaccagaggg

SEQ ID NO: 1012 - RNA, 19 nt, Artificial Sequence
  siRNA strand
cccucugguuugaugccua

SEQ ID NO: 1013 - RNA, 19 nt, Artificial Sequence
  siRNA strand
cagugcauaggcaucaaac

SEQ ID NO: 1014 - RNA, 19 nt, Artificial Sequence
  siRNA strand
guuugaugccuaugcacug

SEQ ID NO: 1015 - RNA, 19 nt, Artificial Sequence
  siRNA strand
acuucugccuccucagugc

SEQ ID NO: 1016 - RNA, 19 nt, Artificial Sequence
  siRNA strand
gcacugaggaggcagaagu

SEQ ID NO: 1017 - RNA, 19 nt, Artificial Sequence
  siRNA strand
aaaucauacuucugccucc

SEQ ID NO: 1018 - RNA, 19 nt, Artificial Sequence
  siRNA strand
ggaggcagaaguaugauuu

SEQ ID NO: 1019 - RNA, 19 nt, Artificial Sequence
  siRNA strand
ugggugcacggcaaaucau

SEQ ID NO: 1020 - RNA, 19 nt, Artificial Sequence
  siRNA strand
augauuugccgugcaccca

SEQ ID NO: 1021 - RNA, 19 nt, Artificial Sequence
  siRNA strand
uccacuggcccugggugca

SEQ ID NO: 1022 - RNA, 19 nt, Artificial Sequence
  siRNA strand
ugcacccagggccagugga

SEQ ID NO: 1023 - RNA, 19 nt, Artificial Sequence
  siRNA strand
uucuggaucguccacuggc

SEQ ID NO: 1024 - RNA, 19 nt, Artificial Sequence
  siRNA strand
gccaguggacgauccagaa

SEQ ID NO: 1025 - RNA, 19 nt, Artificial Sequence
  siRNA strand
cuccuguucuggaucgucc

SEQ ID NO: 1026 - RNA, 19 nt, Artificial Sequence
  siRNA strand
ggacgauccagaacaggag

SEQ ID NO: 1027 - RNA, 19 nt, Artificial Sequence
  siRNA strand
aagccacacagccuccugu

SEQ ID NO: 1028 - RNA, 19 nt, Artificial Sequence
  siRNA strand
acaggaggcuguguggcuu

SEQ ID NO: 1029 - RNA, 19 nt, Artificial Sequence
  siRNA strand
ggaugcgcaagccacacag

SEQ ID NO: 1030 - RNA, 19 nt, Artificial Sequence
  siRNA strand
cuguguggcuugcgcaucc

SEQ ID NO: 1031 - RNA, 19 nt, Artificial Sequence
  siRNA strand
uagggcugcaggaugcgca

SEQ ID NO: 1032 - RNA, 19 nt, Artificial Sequence
  siRNA strand
ugcgcauccugcagcccua

SEQ ID NO: 1033 - RNA, 19 nt, Artificial Sequence
  siRNA strand
auccucucggcguagggcu

SEQ ID NO: 1034 - RNA, 19 nt, Artificial Sequence
  siRNA strand
agcccuacgccgagaggau

SEQ ID NO: 1035 - RNA, 19 nt, Artificial Sequence
  siRNA strand
accacggggauccucucgg

SEQ ID NO: 1036 - RNA, 19 nt, Artificial Sequence
  siRNA strand
ccgagaggauccccguggu

SEQ ID NO: 1037 - RNA, 19 nt, Artificial Sequence
  siRNA strand
ccggccguggccaccacgg

SEQ ID NO: 1038 - RNA, 19 nt, Artificial Sequence
  siRNA strand
ccgugguggccacggccgg

SEQ ID NO: 1039 - RNA, 19 nt, Artificial Sequence
  siRNA strand
auggugaucccggccgugg

SEQ ID NO: 1040 - RNA, 19 nt, Artificial Sequence
  siRNA strand
ccacggccgggaucaccau

SEQ ID NO: 1041 - RNA, 19 nt, Artificial Sequence
  siRNA strand
gugaaguugauggugaucc

SEQ ID NO: 1042 - RNA, 19 nt, Artificial Sequence
  siRNA strand
ggaucaccaucaacuucac

SEQ ID NO: 1043 - RNA, 19 nt, Artificial Sequence
  siRNA strand
aucugggaggugaaguuga

SEQ ID NO: 1044 - RNA, 19 nt, Artificial Sequence
  siRNA strand
ucaacuucaccucccagau

SEQ ID NO: 1045 - RNA, 19 nt, Artificial Sequence
  siRNA strand
ccggugagggagaucuggg

SEQ ID NO: 1046 - RNA, 19 nt, Artificial Sequence
  siRNA strand
cccagaucucccucaccgg

SEQ ID NO: 1047 - RNA, 19 nt, Artificial Sequence
  siRNA strand
acaccgggcccggugaggg

SEQ ID NO: 1048 - RNA, 19 nt, Artificial Sequence
  siRNA strand
cccucaccgggcccggugu

SEQ ID NO: 1049 - RNA, 19 nt, Artificial Sequence
  siRNA strand
agugcacccgcacaccggg

SEQ ID NO: 1050 - RNA, 19 nt, Artificial Sequence
  siRNA strand
cccggugugcgggugcacu

SEQ ID NO: 1051 - RNA, 19 nt, Artificial Sequence
  siRNA strand
uccgacugguuguacaagc

SEQ ID NO: 1052 - RNA, 19 nt, Artificial Sequence
  siRNA strand
gcuuguacaaccagucgga

SEQ ID NO: 1053 - RNA, 19 nt, Artificial Sequence
  siRNA strand
cagggguccgacugguugu

SEQ ID NO: 1054 - RNA, 19 nt, Artificial Sequence
  siRNA strand
acaaccagucggaccccug

SEQ ID NO: 1055 - RNA, 19 nt, Artificial Sequence
  siRNA strand
aacucuccagggcaggggu

SEQ ID NO: 1056 - RNA, 19 nt, Artificial Sequence
  siRNA strand
accccugcccuggagaguu

SEQ ID NO: 1057 - RNA, 19 nt, Artificial Sequence
  siRNA strand
aacagaggaacucuccagg

SEQ ID NO: 1058 - RNA, 19 nt, Artificial Sequence
  siRNA strand
ccuggagaguuccucuguu

SEQ ID NO: 1059 - RNA, 19 nt, Artificial Sequence
  siRNA strand
aguccauucacagaacaga

SEQ ID NO: 1060 - RNA, 19 nt, Artificial Sequence
  siRNA strand
ucuguucugugaauggacu

SEQ ID NO: 1061 - RNA, 19 nt, Artificial Sequence
  siRNA strand
agggacacagaguccauuc

SEQ ID NO: 1062 - RNA, 19 nt, Artificial Sequence
  siRNA strand
gaauggacucugugucccu

SEQ ID NO: 1063 - RNA, 19 nt, Artificial Sequence
  siRNA strand
aucacaggcagggacacag

SEQ ID NO: 1064 - RNA, 19 nt, Artificial Sequence
  siRNA strand
cugugucccugccugugau

SEQ ID NO: 1065 - RNA, 19 nt, Artificial Sequence
  siRNA strand
uugaccccaucacaggcag

SEQ ID NO: 1066 - RNA, 19 nt, Artificial Sequence
  siRNA strand
cugccugugauggggucaa

SEQ ID NO: 1067 - RNA, 19 nt, Artificial Sequence
  siRNA strand
guuggggcaguccuugacc

SEQ ID NO: 1068 - RNA, 19 nt, Artificial Sequence
  siRNA strand
ggucaaggacugccccaac

SEQ ID NO: 1069 - RNA, 19 nt, Artificial Sequence
  siRNA strand
cauccaggccguuggggca

SEQ ID NO: 1070 - RNA, 19 nt, Artificial Sequence
  siRNA strand
ugccccaacggccuggaug

SEQ ID NO: 1071 - RNA, 19 nt, Artificial Sequence
  siRNA strand
guuucucucauccaggccg

SEQ ID NO: 1072 - RNA, 19 nt, Artificial Sequence
  siRNA strand
cggccuggaugagagaaac

SEQ ID NO: 1073 - RNA, 19 nt, Artificial Sequence
  siRNA strand
ugcaaacgcaguuucucuc

SEQ ID NO: 1074 - RNA, 19 nt, Artificial Sequence
  siRNA strand
gagagaaacugcguuugca

SEQ ID NO: 1075 - RNA, 19 nt, Artificial Sequence
  siRNA strand
uggaauguggcucugcaaa

SEQ ID NO: 1076 - RNA, 19 nt, Artificial Sequence
  siRNA strand
uuugcagagccacauucca

SEQ ID NO: 1077 - RNA, 19 nt, Artificial Sequence
  siRNA strand
cuuugcacuggaauguggc

SEQ ID NO: 1078 - RNA, 19 nt, Artificial Sequence
  siRNA strand
gccacauuccagugcaaag

SEQ ID NO: 1079 - RNA, 19 nt, Artificial Sequence
  siRNA strand
ugugcuguccucuuugcac

SEQ ID NO: 1080 - RNA, 19 nt, Artificial Sequence
  siRNA strand
gugcaaagaggacagcaca

SEQ ID NO: 1081 - RNA, 19 nt, Artificial Sequence
  siRNA strand
agaugcaugugcuguccuc

SEQ ID NO: 1082 - RNA, 19 nt, Artificial Sequence
  siRNA strand
gaggacagcacaugcaucu

SEQ ID NO: 1083 - RNA, 19 nt, Artificial Sequence
  siRNA strand
accuugggcagugagaugc

SEQ ID NO: 1084 - RNA, 19 nt, Artificial Sequence
  siRNA strand
gcaucucacugcccaaggu

SEQ ID NO: 1085 - RNA, 19 nt, Artificial Sequence
  siRNA strand
cccaucacagaccuugggc

SEQ ID NO: 1086 - RNA, 19 nt, Artificial Sequence
  siRNA strand
gcccaaggucugugauggg

SEQ ID NO: 1087 - RNA, 19 nt, Artificial Sequence
  siRNA strand
aaucaggcugcccaucaca

SEQ ID NO: 1088 - RNA, 19 nt, Artificial Sequence
  siRNA strand
ugugaugggcagccugauu

SEQ ID NO: 1089 - RNA, 19 nt, Artificial Sequence
  siRNA strand
guugagacaaucaggcugc

SEQ ID NO: 1090 - RNA, 19 nt, Artificial Sequence
  siRNA strand
gcagccugauugucucaac

SEQ ID NO: 1091 - RNA, 19 nt, Artificial Sequence
  siRNA strand
ucgucgcugccguugagac

SEQ ID NO: 1092 - RNA, 19 nt, Artificial Sequence
  siRNA strand
gucucaacggcagcgacga

SEQ ID NO: 1093 - RNA, 19 nt, Artificial Sequence
  siRNA strand
uggcacugcucuucgucgc

SEQ ID NO: 1094 - RNA, 19 nt, Artificial Sequence
  siRNA strand
gcgacgaagagcagugcca

SEQ ID NO: 1095 - RNA, 19 nt, Artificial Sequence
  siRNA strand
accccuuccuggcacugcu

SEQ ID NO: 1096 - RNA, 19 nt, Artificial Sequence
  siRNA strand
agcagugccaggaaggggu

SEQ ID NO: 1097 - RNA, 19 nt, Artificial Sequence
  siRNA strand
ucccacauggcaccccuuc

SEQ ID NO: 1098 - RNA, 19 nt, Artificial Sequence
  siRNA strand
gaaggggugccauguggga

SEQ ID NO: 1099 - RNA, 19 nt, Artificial Sequence
  siRNA strand
aggugaaugucccacaugg

SEQ ID NO: 1100 - RNA, 19 nt, Artificial Sequence
  siRNA strand
ccaugugggacauucaccu

SEQ ID NO: 1101 - RNA, 19 nt, Artificial Sequence
  siRNA strand
ucacacuggaaggugaaug

SEQ ID NO: 1102 - RNA, 19 nt, Artificial Sequence
  siRNA strand
cauucaccuuccaguguga

SEQ ID NO: 1103 - RNA, 19 nt, Artificial Sequence
  siRNA strand
agcuccgguccucacacug

SEQ ID NO: 1104 - RNA, 19 nt, Artificial Sequence
  siRNA strand
cagugugaggaccggagcu

SEQ ID NO: 1105 - RNA, 19 nt, Artificial Sequence
  siRNA strand
ucuucacgcagcuccgguc

SEQ ID NO: 1106 - RNA, 19 nt, Artificial Sequence
  siRNA strand
gaccggagcugcgugaaga

SEQ ID NO: 1107 - RNA, 19 nt, Artificial Sequence
  siRNA strand
guugggcuucuucacgcag

SEQ ID NO: 1108 - RNA, 19 nt, Artificial Sequence
  siRNA strand
cugcgugaagaagcccaac

SEQ ID NO: 1109 - RNA, 19 nt, Artificial Sequence
  siRNA strand
ucacacugcggguugggcu

SEQ ID NO: 1110 - RNA, 19 nt, Artificial Sequence
  siRNA strand
agcccaacccgcaguguga

SEQ ID NO: 1111 - RNA, 19 nt, Artificial Sequence
  siRNA strand
cgggccgcccaucacacug

SEQ ID NO: 1112 - RNA, 19 nt, Artificial Sequence
  siRNA strand
cagugugaugggcggcccg

SEQ ID NO: 1113 - RNA, 19 nt, Artificial Sequence
  siRNA strand
gucccugcagucgggccgc

SEQ ID NO: 1114 - RNA, 19 nt, Artificial Sequence
  siRNA strand
gcggcccgacugcagggac

SEQ ID NO: 1115 - RNA, 19 nt, Artificial Sequence
  siRNA strand
auccgagccgucccugcag

SEQ ID NO: 1116 - RNA, 19 nt, Artificial Sequence
  siRNA strand
cugcagggacggcucggau

SEQ ID NO: 1117 - RNA, 19 nt, Artificial Sequence
  siRNA strand
ugcuccucauccgagccgu

SEQ ID NO: 1118 - RNA, 19 nt, Artificial Sequence
  siRNA strand
acggcucggaugaggagca

SEQ ID NO: 1119 - RNA, 19 nt, Artificial Sequence
  siRNA strand
acagucacagugcuccuca

SEQ ID NO: 1120 - RNA, 19 nt, Artificial Sequence
  siRNA strand
ugaggagcacugugacugu

SEQ ID NO: 1121 - RNA, 19 nt, Artificial Sequence
  siRNA strand
cuggaggccacagucacag

SEQ ID NO: 1122 - RNA, 19 nt, Artificial Sequence
  siRNA strand
cugugacuguggccuccag

SEQ ID NO: 1123 - RNA, 19 nt, Artificial Sequence
  siRNA strand
cuggaggggcccuggaggc

SEQ ID NO: 1124 - RNA, 19 nt, Artificial Sequence
  siRNA strand
gccuccagggccccuccag

SEQ ID NO: 1125 - RNA, 19 nt, Artificial Sequence
  siRNA strand
aacaaugcggcuggagggg

SEQ ID NO: 1126 - RNA, 19 nt, Artificial Sequence
  siRNA strand
ccccuccagccgcauuguu

SEQ ID NO: 1127 - RNA, 19 nt, Artificial Sequence
  siRNA strand
agcuccaccaacaaugcgg

SEQ ID NO: 1128 - RNA, 19 nt, Artificial Sequence
  siRNA strand
ccgcauuguugguggagcu

SEQ ID NO: 1129 - RNA, 19 nt, Artificial Sequence
  siRNA strand
ucggaggacacagcuccac

SEQ ID NO: 1130 - RNA, 19 nt, Artificial Sequence
  siRNA strand
guggagcuguguccuccga

SEQ ID NO: 1131 - RNA, 19 nt, Artificial Sequence
  siRNA strand
uggccacucacccucggag

SEQ ID NO: 1132 - RNA, 19 nt, Artificial Sequence
  siRNA strand
cuccgagggugaguggcca

SEQ ID NO: 1133 - RNA, 19 nt, Artificial Sequence
  siRNA strand
cugccauggccacucaccc

SEQ ID NO: 1134 - RNA, 19 nt, Artificial Sequence
  siRNA strand
gggugaguggccauggcag

SEQ ID NO: 1135 - RNA, 19 nt, Artificial Sequence
  siRNA strand
cuggaggcuggccugccau

SEQ ID NO: 1136 - RNA, 19 nt, Artificial Sequence
  siRNA strand
auggcaggccagccuccag

SEQ ID NO: 1137 - RNA, 19 nt, Artificial Sequence
  siRNA strand
ucgaccccgaaccuggagg

SEQ ID NO: 1138 - RNA, 19 nt, Artificial Sequence
  siRNA strand
ccuccagguucggggucga

SEQ ID NO: 1139 - RNA, 19 nt, Artificial Sequence
  siRNA strand
gaugugucgaccccgaacc

SEQ ID NO: 1140 - RNA, 19 nt, Artificial Sequence
  siRNA strand
gguucggggucgacacauc

SEQ ID NO: 1141 - RNA, 19 nt, Artificial Sequence
  siRNA strand
agggcccccccacagaugu

SEQ ID NO: 1142 - RNA, 19 nt, Artificial Sequence
  siRNA strand
acaucugugggggggcccu

SEQ ID NO: 1143 - RNA, 19 nt, Artificial Sequence
  siRNA strand
gcgaugagggcccccccac

SEQ ID NO: 1144 - RNA, 19 nt, Artificial Sequence
  siRNA strand
gugggggggcccucaucgc

SEQ ID NO: 1145 - RNA, 19 nt, Artificial Sequence
  siRNA strand
ucacccagcggucagcgau

SEQ ID NO: 1146 - RNA, 19 nt, Artificial Sequence
  siRNA strand
aucgcugaccgcuggguga

SEQ ID NO: 1147 - RNA, 19 nt, Artificial Sequence
  siRNA strand
gcuguuaucacccagcggu

SEQ ID NO: 1148 - RNA, 19 nt, Artificial Sequence
  siRNA strand
accgcugggugauaacagc

SEQ ID NO: 1149 - RNA, 19 nt, Artificial Sequence
  siRNA strand
cagugggcagcuguuauca

SEQ ID NO: 1150 - RNA, 19 nt, Artificial Sequence
  siRNA strand
ugauaacagcugcccacug

SEQ ID NO: 1151 - RNA, 19 nt, Artificial Sequence
  siRNA strand
uccuccuggaagcaguggg

SEQ ID NO: 1152 - RNA, 19 nt, Artificial Sequence
  siRNA strand
cccacugcuuccaggagga

SEQ ID NO: 1153 - RNA, 19 nt, Artificial Sequence
  siRNA strand
ggccaugcuguccuccugg

SEQ ID NO: 1154 - RNA, 19 nt, Artificial Sequence
  siRNA strand
ccaggaggacagcauggcc

SEQ ID NO: 1155 - RNA, 19 nt, Artificial Sequence
  siRNA strand
accguggaggccaugcugu

SEQ ID NO: 1156 - RNA, 19 nt, Artificial Sequence
  siRNA strand
acagcauggccuccacggu

SEQ ID NO: 1157 - RNA, 19 nt, Artificial Sequence
  siRNA strand
acgguccacagcaccgugg

SEQ ID NO: 1158 - RNA, 19 nt, Artificial Sequence
  siRNA strand
ccacggugcuguggaccgu

SEQ ID NO: 1159 - RNA, 19 nt, Artificial Sequence
  siRNA strand
uugcccaggaacacggucc

SEQ ID NO: 1160 - RNA, 19 nt, Artificial Sequence
  siRNA strand
ggaccguguuccugggcaa

SEQ ID NO: 1161 - RNA, 19 nt, Artificial Sequence
  siRNA strand
ugccacaccuugcccagga

SEQ ID NO: 1162 - RNA, 19 nt, Artificial Sequence
  siRNA strand
uccugggcaagguguggca

SEQ ID NO: 1163 - RNA, 19 nt, Artificial Sequence
  siRNA strand
agcgcgaguucugccacac

SEQ ID NO: 1164 - RNA, 19 nt, Artificial Sequence
  siRNA strand
guguggcagaacucgcgcu

SEQ ID NO: 1165 - RNA, 19 nt, Artificial Sequence
  siRNA strand
uccaggccagcgcgaguuc

SEQ ID NO: 1166 - RNA, 19 nt, Artificial Sequence
  siRNA strand
gaacucgcgcuggccugga

SEQ ID NO: 1167 - RNA, 19 nt, Artificial Sequence
  siRNA strand
aaggacaccucuccaggcc

SEQ ID NO: 1168 - RNA, 19 nt, Artificial Sequence
  siRNA strand
ggccuggagagguguccuu

SEQ ID NO: 1169 - RNA, 19 nt, Artificial Sequence
  siRNA strand
cucaccuugaaggacaccu

SEQ ID NO: 1170 - RNA, 19 nt, Artificial Sequence
  siRNA strand
agguguccuucaaggugag

SEQ ID NO: 1171 - RNA, 19 nt, Artificial Sequence
  siRNA strand
gagcaggcggcucaccuug

SEQ ID NO: 1172 - RNA, 19 nt, Artificial Sequence
  siRNA strand
caaggugagccgccugcuc

SEQ ID NO: 1173 - RNA, 19 nt, Artificial Sequence
  siRNA strand
uacgggugcaggagcaggc

SEQ ID NO: 1174 - RNA, 19 nt, Artificial Sequence
  siRNA strand
gccugcuccugcacccgua

SEQ ID NO: 1175 - RNA, 19 nt, Artificial Sequence
  siRNA strand
cucuucgugguacgggugc

SEQ ID NO: 1176 - RNA, 19 nt, Artificial Sequence
  siRNA strand
gcacccguaccacgaagag

SEQ ID NO: 1177 - RNA, 19 nt, Artificial Sequence
  siRNA strand
auggcuguccucuucgugg

SEQ ID NO: 1178 - RNA, 19 nt, Artificial Sequence
  siRNA strand
ccacgaagaggacagccau

SEQ ID NO: 1179 - RNA, 19 nt, Artificial Sequence
  siRNA strand
ucguagucauggcuguccu

SEQ ID NO: 1180 - RNA, 19 nt, Artificial Sequence
  siRNA strand
aggacagccaugacuacga

SEQ ID NO: 1181 - RNA, 19 nt, Artificial Sequence
  siRNA strand
agcagcgccacgucguagu

SEQ ID NO: 1182 - RNA, 19 nt, Artificial Sequence
  siRNA strand
acuacgacguggcgcugcu

SEQ ID NO: 1183 - RNA, 19 nt, Artificial Sequence
  siRNA strand
ucgagcugcagcagcgcca

SEQ ID NO: 1184 - RNA, 19 nt, Artificial Sequence
  siRNA strand
uggcgcugcugcagcucga

SEQ ID NO: 1185 - RNA, 19 nt, Artificial Sequence
  siRNA strand
accaccggguggucgagcu

SEQ ID NO: 1186 - RNA, 19 nt, Artificial Sequence
  siRNA strand
agcucgaccacccgguggu

SEQ ID NO: 1187 - RNA, 19 nt, Artificial Sequence
  siRNA strand
cggccgagcgcaccaccgg

SEQ ID NO: 1188 - RNA, 19 nt, Artificial Sequence
  siRNA strand
ccgguggugcgcucggccg

SEQ ID NO: 1189 - RNA, 19 nt, Artificial Sequence
  siRNA strand
cgcacggcggccgagcgca

SEQ ID NO: 1190 - RNA, 19 nt, Artificial Sequence
  siRNA strand
ugcgcucggccgccgugcg

SEQ ID NO: 1191 - RNA, 19 nt, Artificial Sequence
  siRNA strand
aggcagacggggcgcacgg

SEQ ID NO: 1192 - RNA, 19 nt, Artificial Sequence
  siRNA strand
ccgugcgccccgucugccu

SEQ ID NO: 1193 - RNA, 19 nt, Artificial Sequence
  siRNA strand
cgcgcgggcaggcagacgg

SEQ ID NO: 1194 - RNA, 19 nt, Artificial Sequence
  siRNA strand
ccgucugccugcccgcgcg

SEQ ID NO: 1195 - RNA, 19 nt, Artificial Sequence
  siRNA strand
aagaagugggagcgcgcgg

SEQ ID NO: 1196 - RNA, 19 nt, Artificial Sequence
  siRNA strand
ccgcgcgcucccacuucuu

SEQ ID NO: 1197 - RNA, 19 nt, Artificial Sequence
  siRNA strand
ccgggcucgaagaaguggg

SEQ ID NO: 1198 - RNA, 19 nt, Artificial Sequence
  siRNA strand
cccacuucuucgagcccgg

SEQ ID NO: 1199 - RNA, 19 nt, Artificial Sequence
  siRNA strand
agcagugcaggccgggcuc

SEQ ID NO: 1200 - RNA, 19 nt, Artificial Sequence
  siRNA strand
gagcccggccugcacugcu

SEQ ID NO: 1201 - RNA, 19 nt, Artificial Sequence
  siRNA strand
uaauccagcagugcaggcc

SEQ ID NO: 1202 - RNA, 19 nt, Artificial Sequence
  siRNA strand
ggccugcacugcuggauua

SEQ ID NO: 1203 - RNA, 19 nt, Artificial Sequence
  siRNA strand
cgccccagcccguaaucca

SEQ ID NO: 1204 - RNA, 19 nt, Artificial Sequence
  siRNA strand
uggauuacgggcuggggcg

SEQ ID NO: 1205 - RNA, 19 nt, Artificial Sequence
  siRNA strand
ucgcgcaaggcgccccagc

SEQ ID NO: 1206 - RNA, 19 nt, Artificial Sequence
  siRNA strand
gcuggggcgccuugcgcga

SEQ ID NO: 1207 - RNA, 19 nt, Artificial Sequence
  siRNA strand
auggggccgcccucgcgca

SEQ ID NO: 1208 - RNA, 19 nt, Artificial Sequence
  siRNA strand
ugcgcgagggcggccccau

SEQ ID NO: 1209 - RNA, 19 nt, Artificial Sequence
  siRNA strand
uugcugauggggccgcccu

SEQ ID NO: 1210 - RNA, 19 nt, Artificial Sequence
  siRNA strand
agggcggccccaucagcaa

SEQ ID NO: 1211 - RNA, 19 nt, Artificial Sequence
  siRNA strand
uucugcagagcguugcuga

SEQ ID NO: 1212 - RNA, 19 nt, Artificial Sequence
  siRNA strand
ucagcaacgcucugcagaa

SEQ ID NO: 1213 - RNA, 19 nt, Artificial Sequence
  siRNA strand
ugcacauccacuuucugca

SEQ ID NO: 1214 - RNA, 19 nt, Artificial Sequence
  siRNA strand
ugcagaaaguggaugugca

SEQ ID NO: 1215 - RNA, 19 nt, Artificial Sequence
  siRNA strand
aucaacugcacauccacuu

SEQ ID NO: 1216 - RNA, 19 nt, Artificial Sequence
  siRNA strand
aaguggaugugcaguugau

SEQ ID NO: 1217 - RNA, 19 nt, Artificial Sequence
  siRNA strand
guccugugggaucaacugc

SEQ ID NO: 1218 - RNA, 19 nt, Artificial Sequence
  siRNA strand
gcaguugaucccacaggac

SEQ ID NO: 1219 - RNA, 19 nt, Artificial Sequence
  siRNA strand
ucgcugcacagguccugug

SEQ ID NO: 1220 - RNA, 19 nt, Artificial Sequence
  siRNA strand
cacaggaccugugcagcga

SEQ ID NO: 1221 - RNA, 19 nt, Artificial Sequence
  siRNA strand
uagcgauagaccucgcugc

SEQ ID NO: 1222 - RNA, 19 nt, Artificial Sequence
  siRNA strand
gcagcgaggucuaucgcua

SEQ ID NO: 1223 - RNA, 19 nt, Artificial Sequence
  siRNA strand
ucaccugguagcgauagac

SEQ ID NO: 1224 - RNA, 19 nt, Artificial Sequence
  siRNA strand
gucuaucgcuaccagguga

SEQ ID NO: 1225 - RNA, 19 nt, Artificial Sequence
  siRNA strand
caugcguggcgucaccugg

SEQ ID NO: 1226 - RNA, 19 nt, Artificial Sequence
  siRNA strand
ccaggugacgccacgcaug

SEQ ID NO: 1227 - RNA, 19 nt, Artificial Sequence
  siRNA strand
cggcacacagcaugcgugg

SEQ ID NO: 1228 - RNA, 19 nt, Artificial Sequence
  siRNA strand
ccacgcaugcugugugccg

SEQ ID NO: 1229 - RNA, 19 nt, Artificial Sequence
  siRNA strand
ugcgguagccggcacacag

SEQ ID NO: 1230 - RNA, 19 nt, Artificial Sequence
  siRNA strand
cugugugccggcuaccgca

SEQ ID NO: 1231 - RNA, 19 nt, Artificial Sequence
  siRNA strand
uccuucuugcccuugcggu

SEQ ID NO: 1232 - RNA, 19 nt, Artificial Sequence
  siRNA strand
accgcaagggcaagaagga

SEQ ID NO: 1233 - RNA, 19 nt, Artificial Sequence
  siRNA strand
ugacaggcauccuucuugc

SEQ ID NO: 1234 - RNA, 19 nt, Artificial Sequence
  siRNA strand
gcaagaaggaugccuguca

SEQ ID NO: 1235 - RNA, 19 nt, Artificial Sequence
  siRNA strand
cugagucacccugacaggc

SEQ ID NO: 1236 - RNA, 19 nt, Artificial Sequence
  siRNA strand
gccugucagggugacucag

SEQ ID NO: 1237 - RNA, 19 nt, Artificial Sequence
  siRNA strand
agcggaccaccugagucac

SEQ ID NO: 1238 - RNA, 19 nt, Artificial Sequence
  siRNA strand
gugacucaggugguccgcu

SEQ ID NO: 1239 - RNA, 19 nt, Artificial Sequence
  siRNA strand
uugcacaccagcggaccac

SEQ ID NO: 1240 - RNA, 19 nt, Artificial Sequence
  siRNA strand
gugguccgcuggugugcaa

SEQ ID NO: 1241 - RNA, 19 nt, Artificial Sequence
  siRNA strand
cugagugccuugcacacca

SEQ ID NO: 1242 - RNA, 19 nt, Artificial Sequence
  siRNA strand
uggugugcaaggcacucag

SEQ ID NO: 1243 - RNA, 19 nt, Artificial Sequence
  siRNA strand
accagcggccacugagugc

SEQ ID NO: 1244 - RNA, 19 nt, Artificial Sequence
  siRNA strand
gcacucaguggccgcuggu

SEQ ID NO: 1245 - RNA, 19 nt, Artificial Sequence
  siRNA strand
cccgccaggaaccagcggc

SEQ ID NO: 1246 - RNA, 19 nt, Artificial Sequence
  siRNA strand
gccgcugguuccuggcggg

SEQ ID NO: 1247 - RNA, 19 nt, Artificial Sequence
  siRNA strand
cugaccagccccgccagga

SEQ ID NO: 1248 - RNA, 19 nt, Artificial Sequence
  siRNA strand
uccuggcggggcuggucag

SEQ ID NO: 1249 - RNA, 19 nt, Artificial Sequence
  siRNA strand
caggccccagcugaccagc

SEQ ID NO: 1250 - RNA, 19 nt, Artificial Sequence
  siRNA strand
gcuggucagcuggggccug

SEQ ID NO: 1251 - RNA, 19 nt, Artificial Sequence
  siRNA strand
ccggccacagcccaggccc

SEQ ID NO: 1252 - RNA, 19 nt, Artificial Sequence
  siRNA strand
gggccugggcuguggccgg

SEQ ID NO: 1253 - RNA, 19 nt, Artificial Sequence
  siRNA strand
aguuaggccggccacagcc

SEQ ID NO: 1254 - RNA, 19 nt, Artificial Sequence
  siRNA strand
ggcuguggccggccuaacu

SEQ ID NO: 1255 - RNA, 19 nt, Artificial Sequence
  siRNA strand
uagacgccgaaguaguuag

SEQ ID NO: 1256 - RNA, 19 nt, Artificial Sequence
  siRNA strand
cuaacuacuucggcgucua

SEQ ID NO: 1257 - RNA, 19 nt, Artificial Sequence
  siRNA strand
gaugcggguguagacgccg

SEQ ID NO: 1258 - RNA, 19 nt, Artificial Sequence
  siRNA strand
cggcgucuacacccgcauc

SEQ ID NO: 1259 - RNA, 19 nt, Artificial Sequence
  siRNA strand
acaccugugaugcgggugu

SEQ ID NO: 1260 - RNA, 19 nt, Artificial Sequence
  siRNA strand
acacccgcaucacaggugu

SEQ ID NO: 1261 - RNA, 19 nt, Artificial Sequence
  siRNA strand
uccagcugaucacaccugu

SEQ ID NO: 1262 - RNA, 19 nt, Artificial Sequence
  siRNA strand
acaggugugaucagcugga

SEQ ID NO: 1263 - RNA, 19 nt, Artificial Sequence
  siRNA strand
acuugcuggauccagcuga

SEQ ID NO: 1264 - RNA, 19 nt, Artificial Sequence
  siRNA strand
ucagcuggauccagcaagu

SEQ ID NO: 1265 - RNA, 19 nt, Artificial Sequence
  siRNA strand
cucaggucaccacuugcug

SEQ ID NO: 1266 - RNA, 19 nt, Artificial Sequence
  siRNA strand
cagcaaguggugaccugag

SEQ ID NO: 1267 - RNA, 19 nt, Artificial Sequence
  siRNA strand
ggggcaguuccucagguca

SEQ ID NO: 1268 - RNA, 19 nt, Artificial Sequence
  siRNA strand
ugaccugaggaacugcccc

SEQ ID NO: 1269 - RNA, 19 nt, Artificial Sequence
  siRNA strand
uuugcaggggggcaguucc

SEQ ID NO: 1270 - RNA, 19 nt, Artificial Sequence
  siRNA strand
ggaacugccccccugcaaa

SEQ ID NO: 1271 - RNA, 19 nt, Artificial Sequence
  siRNA strand
ggugggcccugcuuugcag

SEQ ID NO: 1272 - RNA, 19 nt, Artificial Sequence
  siRNA strand
cugcaaagcagggcccacc

SEQ ID NO: 1273 - RNA, 19 nt, Artificial Sequence
  siRNA strand
uccaggaggugggcccugc

SEQ ID NO: 1274 - RNA, 19 nt, Artificial Sequence
  siRNA strand
gcagggcccaccuccugga

SEQ ID NO: 1275 - RNA, 19 nt, Artificial Sequence
  siRNA strand
gcucucugaguccaggagg

SEQ ID NO: 1276 - RNA, 19 nt, Artificial Sequence
  siRNA strand
ccuccuggacucagagagc

SEQ ID NO: 1277 - RNA, 19 nt, Artificial Sequence
  siRNA strand
uugcccugggcucucugag

SEQ ID NO: 1278 - RNA, 19 nt, Artificial Sequence
  siRNA strand
cucagagagcccagggcaa

SEQ ID NO: 1279 - RNA, 19 nt, Artificial Sequence
  siRNA strand
ugcuuggcaguugcccugg

SEQ ID NO: 1280 - RNA, 19 nt, Artificial Sequence
  siRNA strand
ccagggcaacugccaagca

SEQ ID NO: 1281 - RNA, 19 nt, Artificial Sequence
  siRNA strand
cccgccagaauacuugucc

SEQ ID NO: 1282 - RNA, 19 nt, Artificial Sequence
  siRNA strand
ggacaaguauucuggcggg

SEQ ID NO: 1283 - RNA, 19 nt, Artificial Sequence
  siRNA strand
cucccccaccccccgccag

SEQ ID NO: 1284 - RNA, 19 nt, Artificial Sequence
  siRNA strand
cuggcggggggugggggag

SEQ ID NO: 1285 - RNA, 19 nt, Artificial Sequence
  siRNA strand
ccugcucucucccccaccc

SEQ ID NO: 1286 - RNA, 19 nt, Artificial Sequence
  siRNA strand
gggugggggagagagcagg

SEQ ID NO: 1287 - RNA, 19 nt, Artificial Sequence
  siRNA strand
accacagggccugcucucu

SEQ ID NO: 1288 - RNA, 19 nt, Artificial Sequence
  siRNA strand
agagagcaggcccuguggu

SEQ ID NO: 1289 - RNA, 19 nt, Artificial Sequence
  siRNA strand
accuccugccaccacaggg

SEQ ID NO: 1290 - RNA, 19 nt, Artificial Sequence
  siRNA strand
cccugugguggcaggaggu

SEQ ID NO: 1291 - RNA, 19 nt, Artificial Sequence
  siRNA strand
gagacaagaugccaccucc

SEQ ID NO: 1292 - RNA, 19 nt, Artificial Sequence
  siRNA strand
ggagguggcaucuugucuc

SEQ ID NO: 1293 - RNA, 19 nt, Artificial Sequence
  siRNA strand
ucagggacgagacaagaug

SEQ ID NO: 1294 - RNA, 19 nt, Artificial Sequence
  siRNA strand
caucuugucucgucccuga

SEQ ID NO: 1295 - RNA, 19 nt, Artificial Sequence
  siRNA strand
acuggagcagacaucaggg

SEQ ID NO: 1296 - RNA, 19 nt, Artificial Sequence
  siRNA strand
cccugaugucugcuccagu

SEQ ID NO: 1297 - RNA, 19 nt, Artificial Sequence
  siRNA strand
ccugccaucacuggagcag

SEQ ID NO: 1298 - RNA, 19 nt, Artificial Sequence
  siRNA strand
cugcuccagugauggcagg

SEQ ID NO: 1299 - RNA, 19 nt, Artificial Sequence
  siRNA strand
uucuccauccuccugccau

SEQ ID NO: 1300 - RNA, 19 nt, Artificial Sequence
  siRNA strand
auggcaggaggauggagaa

SEQ ID NO: 1301 - RNA, 19 nt, Artificial Sequence
  siRNA strand
ugcuggcacuucuccaucc

SEQ ID NO: 1302 - RNA, 19 nt, Artificial Sequence
  siRNA strand
ggauggagaagugccagca

SEQ ID NO: 1303 - RNA, 19 nt, Artificial Sequence
  siRNA strand
ugacccccagcugcuggca

SEQ ID NO: 1304 - RNA, 19 nt, Artificial Sequence
  siRNA strand
ugccagcagcuggggguca

SEQ ID NO: 1305 - RNA, 19 nt, Artificial Sequence
  siRNA strand
gacgucuugacccccagcu

SEQ ID NO: 1306 - RNA, 19 nt, Artificial Sequence
  siRNA strand
agcugggggucaagacguc

SEQ ID NO: 1307 - RNA, 19 nt, Artificial Sequence
  siRNA strand
uccucaggggacgucuuga

SEQ ID NO: 1308 - RNA, 19 nt, Artificial Sequence
  siRNA strand
ucaagacguccccugagga

SEQ ID NO: 1309 - RNA, 19 nt, Artificial Sequence
  siRNA strand
ugggccuggguccucaggg

SEQ ID NO: 1310 - RNA, 19 nt, Artificial Sequence
  siRNA strand
cccugaggacccaggccca

SEQ ID NO: 1311 - RNA, 19 nt, Artificial Sequence
  siRNA strand
agaagggcugggugugggc

SEQ ID NO: 1312 - RNA, 19 nt, Artificial Sequence
  siRNA strand
gcccacacccagcccuucu

SEQ ID NO: 1313 - RNA, 19 nt, Artificial Sequence
  siRNA strand
auugggaggcagaagggcu

SEQ ID NO: 1314 - RNA, 19 nt, Artificial Sequence
  siRNA strand
agcccuucugccucccaau

SEQ ID NO: 1315 - RNA, 19 nt, Artificial Sequence
  siRNA strand
aggagagagaauugggagg

SEQ ID NO: 1316 - RNA, 19 nt, Artificial Sequence
  siRNA strand
ccucccaauucucucuccu

SEQ ID NO: 1317 - RNA, 19 nt, Artificial Sequence
  siRNA strand
aaggggacggaggagagag

SEQ ID NO: 1318 - RNA, 19 nt, Artificial Sequence
  siRNA strand
cucucuccuccguccccuu

SEQ ID NO: 1319 - RNA, 19 nt, Artificial Sequence
  siRNA strand
aguggaggaaggggacgga

SEQ ID NO: 1320 - RNA, 19 nt, Artificial Sequence
  siRNA strand
uccguccccuuccuccacu

SEQ ID NO: 1321 - RNA, 19 nt, Artificial Sequence
  siRNA strand
uaggcagcaguggaggaag

SEQ ID NO: 1322 - RNA, 19 nt, Artificial Sequence
  siRNA strand
cuuccuccacugcugccua

SEQ ID NO: 1323 - RNA, 19 nt, Artificial Sequence
  siRNA strand
acugccuugcauuaggcag

SEQ ID NO: 1324 - RNA, 19 nt, Artificial Sequence
  siRNA strand
cugccuaaugcaaggcagu

SEQ ID NO: 1325 - RNA, 19 nt, Artificial Sequence
  siRNA strand
ugcugagccacugccuugc

SEQ ID NO: 1326 - RNA, 19 nt, Artificial Sequence
  siRNA strand
gcaaggcaguggcucagca

SEQ ID NO: 1327 - RNA, 19 nt, Artificial Sequence
  siRNA strand
cauucuugcugcugagcca

SEQ ID NO: 1328 - RNA, 19 nt, Artificial Sequence
  siRNA strand
uggcucagcagcaagaaug

SEQ ID NO: 1329 - RNA, 19 nt, Artificial Sequence
  siRNA strand
uguagaaccagcauucuug

SEQ ID NO: 1330 - RNA, 19 nt, Artificial Sequence
  siRNA strand
caagaaugcugguucuaca

SEQ ID NO: 1331 - RNA, 19 nt, Artificial Sequence
  siRNA strand
uccucgggauguagaacca

SEQ ID NO: 1332 - RNA, 19 nt, Artificial Sequence
  siRNA strand
ugguucuacaucccgagga

SEQ ID NO: 1333 - RNA, 19 nt, Artificial Sequence
  siRNA strand
accucagacacuccucggg

SEQ ID NO: 1334 - RNA, 19 nt, Artificial Sequence
  siRNA strand
cccgaggagugucugaggu

SEQ ID NO: 1335 - RNA, 19 nt, Artificial Sequence
  siRNA strand
aguggggcgcaccucagac

SEQ ID NO: 1336 - RNA, 19 nt, Artificial Sequence
  siRNA strand
gucugaggugcgccccacu

SEQ ID NO: 1337 - RNA, 19 nt, Artificial Sequence
  siRNA strand
ccucuguacagaguggggc

SEQ ID NO: 1338 - RNA, 19 nt, Artificial Sequence
  siRNA strand
gccccacucuguacagagg

SEQ ID NO: 1339 - RNA, 19 nt, Artificial Sequence
  siRNA strand
ccaaacagccucuguacag

SEQ ID NO: 1340 - RNA, 19 nt, Artificial Sequence
  siRNA strand
cuguacagaggcuguuugg

SEQ ID NO: 1341 - RNA, 19 nt, Artificial Sequence
  siRNA strand
ggcaaggcugcccaaacag

SEQ ID NO: 1342 - RNA, 19 nt, Artificial Sequence
  siRNA strand
cuguuugggcagccuugcc

SEQ ID NO: 1343 - RNA, 19 nt, Artificial Sequence
  siRNA strand
ucugcucucuggaggcaag

SEQ ID NO: 1344 - RNA, 19 nt, Artificial Sequence
  siRNA strand
cuugccuccagagagcaga

SEQ ID NO: 1345 - RNA, 19 nt, Artificial Sequence
  siRNA strand
cuggaaucugcucucugga

SEQ ID NO: 1346 - RNA, 19 nt, Artificial Sequence
  siRNA strand
uccagagagcagauuccag

SEQ ID NO: 1347 - RNA, 19 nt, Artificial Sequence
  siRNA strand
ggcuuccgaagcuggaauc

SEQ ID NO: 1348 - RNA, 19 nt, Artificial Sequence
  siRNA strand
gauuccagcuucggaagcc

SEQ ID NO: 1349 - RNA, 19 nt, Artificial Sequence
  siRNA strand
augggagcaccuuccauuc

SEQ ID NO: 1350 - RNA, 19 nt, Artificial Sequence
  siRNA strand
gaauggaaggugcucccau

SEQ ID NO: 1351 - RNA, 19 nt, Artificial Sequence
  siRNA strand
uccccuccgaugggagcac

SEQ ID NO: 1352 - RNA, 19 nt, Artificial Sequence
  siRNA strand
gugcucccaucggagggga

SEQ ID NO: 1353 - RNA, 19 nt, Artificial Sequence
  siRNA strand
cucugaggguccccuccga

SEQ ID NO: 1354 - RNA, 19 nt, Artificial Sequence
  siRNA strand
ucggaggggacccucagag

SEQ ID NO: 1355 - RNA, 19 nt, Artificial Sequence
  siRNA strand
gucuccagggcucugaggg

SEQ ID NO: 1356 - RNA, 19 nt, Artificial Sequence
  siRNA strand
cccucagagcccuggagac

SEQ ID NO: 1357 - RNA, 19 nt, Artificial Sequence
  siRNA strand
aggcccaccuggcagucuc

SEQ ID NO: 1358 - RNA, 19 nt, Artificial Sequence
  siRNA strand
gagacugccaggugggccu

SEQ ID NO: 1359 - RNA, 19 nt, Artificial Sequence
  siRNA strand
aguggcagcaggcccaccu

SEQ ID NO: 1360 - RNA, 19 nt, Artificial Sequence
  siRNA strand
aggugggccugcugccacu

SEQ ID NO: 1361 - RNA, 19 nt, Artificial Sequence
  siRNA strand
uuuuggcuuacaguggcag

SEQ ID NO: 1362 - RNA, 19 nt, Artificial Sequence
  siRNA strand
cugccacuguaagccaaaa

SEQ ID NO: 1363 - RNA, 19 nt, Artificial Sequence
  siRNA strand
cccaccuuuuggcuuacag

SEQ ID NO: 1364 - RNA, 19 nt, Artificial Sequence
  siRNA strand
cuguaagccaaaagguggg

SEQ ID NO: 1365 - RNA, 19 nt, Artificial Sequence
  siRNA strand
ggagucaggacuuccccac

SEQ ID NO: 1366 - RNA, 19 nt, Artificial Sequence
  siRNA strand
guggggaaguccugacucc

SEQ ID NO: 1367 - RNA, 19 nt, Artificial Sequence
  siRNA strand
caaggacccuggagucagg

SEQ ID NO: 1368 - RNA, 19 nt, Artificial Sequence
  siRNA strand
ccugacuccaggguccuug

SEQ ID NO: 1369 - RNA, 19 nt, Artificial Sequence
  siRNA strand
agggguggggcaaggaccc

SEQ ID NO: 1370 - RNA, 19 nt, Artificial Sequence
  siRNA strand
ggguccuugccccaccccu

SEQ ID NO: 1371 - RNA, 19 nt, Artificial Sequence
  siRNA strand
uggcaggcagggguggggc

SEQ ID NO: 1372 - RNA, 19 nt, Artificial Sequence
  siRNA strand
gccccaccccugccugcca

SEQ ID NO: 1373 - RNA, 19 nt, Artificial Sequence
  siRNA strand
ugagggcccagguggcagg

SEQ ID NO: 1374 - RNA, 19 nt, Artificial Sequence
  siRNA strand
ccugccaccugggcccuca

SEQ ID NO: 1375 - RNA, 19 nt, Artificial Sequence
  siRNA strand
cugggcugugagggcccag

SEQ ID NO: 1376 - RNA, 19 nt, Artificial Sequence
  siRNA strand
cugggcccucacagcccag

SEQ ID NO: 1377 - RNA, 19 nt, Artificial Sequence
  siRNA strand
gugagggucugggcuguga

SEQ ID NO: 1378 - RNA, 19 nt, Artificial Sequence
  siRNA strand
ucacagcccagacccucac

SEQ ID NO: 1379 - RNA, 19 nt, Artificial Sequence
  siRNA strand
gagcucaccucccagugag

SEQ ID NO: 1380 - RNA, 19 nt, Artificial Sequence
  siRNA strand
cucacugggaggugagcuc

SEQ ID NO: 1381 - RNA, 19 nt, Artificial Sequence
  siRNA strand
aagggcagcugagcucacc

SEQ ID NO: 1382 - RNA, 19 nt, Artificial Sequence
  siRNA strand
ggugagcucagcugcccuu

SEQ ID NO: 1383 - RNA, 19 nt, Artificial Sequence
  siRNA strand
aucaggcagcuuuauucca

SEQ ID NO: 1384 - RNA, 19 nt, Artificial Sequence
  siRNA strand
uggaauaaagcugccugau

SEQ ID NO: 1385 - RNA, 3197 nt, Homo sapiens
guucuugagccagacccaguccagcucuggugccugcccucuggugcgagcugaccugagaugcacuucccuccucugugagcugucucggcacccacuugcagucacugccgccugauguuguuacucuuccacuccaaaaggaugcccguggccgaggccccccagguggcuggcgggcagggggacggaggugauggcgaggaagcggagccggaggggauguucaaggccugugaggacuccaagagaaaagcccggggcuaccuccgccuggugccccuguuugugcugcuggcccugcucgugcuggcuucggcgggggugcuacucugguauuuccuaggguacaaggcggaggugauggucagccagguguacucaggcagucugcguguacucaaucgccacuucucccaggaucuuacccgccgggaaucuagugccuuccgcagugaaaccgccaaagcccagaagaugcucaaggagcucaucaccagcacccgccugggaacuuacuacaacuccagcuccgucuauuccuuuggggagggaccccucaccugcuucuucugguucauucuccaaauccccgagcaccgccggcugaugcugagccccgagguggugcaggcacugcugguggaggagcugcuguccacagucaacagcucggcugccguccccuacagggccgaguacgaaguggaccccgagggccuagugauccuggaagccagugugaaagacauagcugcauugaauuccacgcuggguuguuaccgcuacagcuacgugggccagggccagguccuccggcugaaggggccugaccaccuggccuccagcugccuguggcaccugcagggccccaaggaccucaugcucaaacuccggcuggaguggacgcuggcagagugccgggaccgacuggccauguaugacguggccgggccccuggagaagaggcucaucaccucgguguacggcugcagccgccaggagcccgugguggagguucuggcgucgggggccaucauggcggucgucuggaagaagggccugcacagcuacuacgaccccuucgugcucuccgugcagccgguggucuuccaggccugugaagugaaccugacgcuggacaacaggcucgacucccagggcguccucagcaccccguacuuccccagcuacuacucgccccaaacccacugcuccuggcaccucacggugcccucucuggacuacggcuuggcccucugguuugaugccuaugcacugaggaggcagaaguaugauuugccgugcacccagggccaguggacgauccagaacaggaggcuguguggcuugcgcauccugcagcccuacgccgagaggauccccgugguggccacggccgggaucaccaucaacuucaccucccagaucucccucaccgggcccggugugcgggugcacuauggcuuguacaaccagucggaccccugcccuggagaguuccucuguucugugaauggacucugugucccugccugugauggggucaaggacugccccaacggccuggaugagagaaacugcguuugcagagccacauuccagugcaaagaggacagcacaugcaucucacugcccaaggucugugaugggcagccugauugucucaacggcagcgacgaagagcagugccaggaaggggugccaugugggacauucaccuuccagugugaggaccggagcugcgugaagaagcccaacccgcagugugaugggcggcccgacugcagggacggcucggaugaggagcacugugacuguggccuccagggccccuccagccgcauuguugguggagcuguguccuccgagggugaguggccauggcaggccagccuccagguucggggucgacacaucugugggggggcccucaucgcugaccgcugggugauaacagcugcccacugcuuccaggaggacagcauggccuccacggugcuguggaccguguuccugggcaagguguggcagaacucgcgcuggccuggagagguguccuucaaggugagccgccugcuccugcacccguaccacgaagaggacagccaugacuacgacguggcgcugcugcagcucgaccacccgguggugcgcucggccgccgugcgccccgucugccugcccgcgcgcucccacuucuucgagcccggccugcacugcuggauuacgggcuggggcgccuugcgcgagggcggccccaucagcaacgcucugcagaaaguggaugugcaguugaucccacaggaccugugcagcgaggucuaucgcuaccaggugacgccacgcaugcugugugccggcuaccgcaagggcaagaaggaugccugucagggugacucaggugguccgcuggugugcaaggcacucaguggccgcugguuccuggcggggcuggucagcuggggccugggcuguggccggccuaacuacuucggcgucuacacccgcaucacaggugugaucagcuggauccagcaaguggugaccugaggaacugccccccugcaaagcagggcccaccuccuggacucagagagcccagggcaacugccaagcagggggacaaguauucuggcggggggugggggagagagcaggcccugugguggcaggagguggcaucuugucucgucccugaugucugcuccagugauggcaggaggauggagaagugccagcagcugggggucaagacguccccugaggacccaggcccacacccagcccuucugccucccaauucucucuccuccguccccuuccuccacugcugccuaaugcaaggcaguggcucagcagcaagaaugcugguucuacaucccgaggagugucugaggugcgccccacucuguacagaggcuguuugggcagccuugccuccagagagcagauuccagcuucggaagccccuggucuaacuugggaucugggaauggaaggugcucccaucggaggggacccucagagcccuggagacugccaggugggccugcugccacuguaagccaaaagguggggaaguccugacuccaggguccuugccccaccccugccugccaccugggcccucacagcccagacccucacugggaggugagcucagcugcccuuuggaauaaagcugccugauccaa

SEQ ID NO: 1386 - RNA, 19 nt, Artificial Sequence
  siRNA strand - sequence modified as per summary sequence table at the end of the description
ucaccugcuucuucugguu

SEQ ID NO: 1387 - RNA, 19 nt, Artificial Sequence
  siRNA strand - sequence modified as per summary sequence table at the end of the description
ucaccugcuucuucugguu

(19) United States
(12) Patent Application Publication
(10) Pub. No.: US 2024/0240187 A1
(43) Pub. Date: Jul. 18, 2024

(54) SIRNA TARGETING TMPRSS6 FOR THE TREATMENT OF MYELOPROLIFERATIVE DISORDERS

(71) Applicants: SILENCE THERAPEUTICS GMBH, Berlin (DE); The Walter and Eliza Hall Institute of Medical Research, Melbourne, Victoria (AU)

(72) Inventors: Cavan Bennett, Melbourne, Victoria (AU); Sant-Rayn Pasricha, Melbourne, Victoria (AU); Ashley Ng, Melbourne, Victoria (AU); Sibylle Dames, Berlin (DE); Ute Schaeper, Berlin (DE)

(21) Appl. No.: 18/288,427

(22) PCT Filed: Apr. 26, 2022
(86) PCT No.: PCT/EP2022/060998

§ 371 Date: Oct. 26, 2023

(30) Foreign Application Priority Data
Apr. 27, 2021 (EP) 21170774.0

(51) Int. Cl. C12N 15/113; A61P 7/00

(52) U.S. Cl. CPC C12N 15/1137; A61P 7/00; C12N 2310/11; C12N 2310/315; C12N 2310/351

(57) ABSTRACT

The invention relates to a Matriptase-2 (MT2) and/or TMPRSS6 inhibitor for the prevention, decrease of the risk of suffering from or treatment of a myeloproliferative disorder.

### FIELD OF THE INVENTION

The invention relates to a Matriptase-2 (MT2) and/or TMPRSS6 inhibitor for the prevention, decrease of the risk of suffering from or treatment of a myeloproliferative disorder.

### BACKGROUND

Polycythaemia vera (PV), essential thrombocythaemia (ET) and primary myelofibrosis (PMF) are Philadelphia chromosome (BCR-ABL) negative myeloproliferative neoplasms. PV is characterized by bone marrow erythroid and megakaryocytic hyperplasia, erythrocytosis, fatigue, aquagenic pruritus, microvascular symptoms, and symptomatic splenomegaly (reviewed by Ginzburg et al., Leukemia. 2018 October; 32(10):2105-2116). The disease is caused by a driver mutation in the haematopoietic stem cells. Most commonly, the gene defect involves Janus kinase 2 (JAK2). JAK2 is a non-receptor tyrosine kinase that transduces the erythropoietin receptor (EpoR) as well as granulocyte-colony stimulating factor and thrombopoietin receptor signalling. Activation of JAK2 triggers multiple signalling pathways regulating erythroid precursor cell survival, proliferation and differentiation. The most common JAK2 driver mutation is JAK2 V617F, which results in constitutive, erythropoietin independent JAK2/STAT signalling and upregulation of genes downstream of the JAK2/STAT pathway (reviewed by Ginzburg et al., Leukemia. 2018 October;32(10):2105-2116). The vast majority of PV patients (96%) are JAK2 V617F positive, 2-3% of them harbour mutations in exon 12 of JAK2 and in some rare occasions, mutations were identified in genes that function as negative regulators of JAK2, indicating the predominant function of JAK2 activation in the aetiology of PV. In addition, the JAK2 V617F mutation is found in ˜ 50% of ET and PMF patients. JAK2 V617F positive ET patients generally have higher haemoglobin (HGB) levels and lower platelet counts compared to JAK2 V617F negative ET patients, which points to a prominent role of JAK2 activation in promoting erythropoiesis. As a result of chronic hyperproliferation of erythroid cells and erythrocytosis, patients with PV have elevated haemoglobin (HGB), haematocrit (HCT) and red blood cell mass, which puts them at increased risk for arterial and venous thrombosis. Indeed, thrombosis is the most immediate health threat in PV patients (Spivak J L Blood. 2019 Jul. 25; 134(4):341-352).

### Current Treatment Options

Aspirin is recommended for primary thrombosis prevention in all PV patients with haematocrit levels >45% without contra indication. This recommendation has however been challenged because of bleeding risks (Valgimigli M, European Heart Journal (2019) 40, 618-620). Patients are commonly treated by phlebotomy (i.e., removal of blood) to normalize haemoglobin and haematocrit levels and may receive aspirin to further reduce the risk of thrombosis. However, patients with PV frequently have iron deficiency when they are diagnosed, even prior to the onset of therapeutic phlebotomy (Thiele et al., Pathol Res Pract. 2001; 197(2):77-84). Iron deficiency is further exacerbated by repeated phlebotomy. While iron deficiency is beneficial to attenuate the accelerated erythropoiesis (production of red blood cells), it can also cause cognitive impairment, fatigue and decline in physical performance. This is due to the fact that iron is not only required for erythropoiesis, as an essential component of haem and haemoglobin, but is also an essential component of myoglobin and oxygen storage proteins in high-oxygen consuming tissues such as skeletal muscle and heart muscle. Iron is also important for the energy metabolism because it is a component of several enzymes involved in the energy metabolism, such as the haem-containing enzymes cytochrome c and the non-haem containing enzymes NADH dehydrogenase and aconitase. Other enzymes such as nitric oxide synthase, various enzymes involved in the generation of adenine triphosphate (ATP) as well as in DNA replication and repair also require iron. However, despite the wide-ranging impact of iron deficiency, PV patients cannot be treated by iron repletion, as this would increase erythropoiesis. This would reverse the effects of phlebotomy (also called venesection), and thus again enhance the risk of thrombotic events. As a result, patients must currently live with the effects of systemic iron deficiency. Due to its nature, venesection causes fluid shifts and is associated with vaso-vagal reactions including dizziness and fainting. Patients with PV have a long median survival (˜14 years from diagnosis), and during much of this time, many patients are treated with venesection and aspirin alone.

Cytoreductive therapies such as hydroxyurea, interferon alpha, busulfan or JAK2 inhibitors (e.g., Ruxolitinib) can be effective as second line therapies in normalizing haemoglobin and haematocrit levels, and thereby reducing the need for phlebotomy. However, even though patients have a clinical response to cytoreductive therapy, it is not curative and does not change the natural history of the disease. Patients who are <60 years old are therefore in general maintained on phlebotomy (Tefferi et al., Blood Cancer J. 2018 Jan. 10; 8(1).) and the impact of systemic iron deficiency is not addressed. Patients who are unable to tolerate venesection are administered cytoreductive therapies due to lack of alternative options.

Another therapy in development is the use of mini-hepcidins. These are peptide-derived hepcidin agonists. Hepcidin is a peptide hormone predominantly produced in the liver. It is a negative regulator of gastro-intestinal iron absorption and controls the release of iron into the circulation from iron storage cells such as macrophages in the spleen. Hepcidin regulates iron balance in the body by blocking the cellular release of iron via the only known cellular iron export protein, ferroportin. Elevated hepcidin levels can as a result induce iron restriction, which is a reduction in iron availability within the body (McDonald et al., American Journal of Physiology, 2015, vol 08 no. 7, C539-C547). Mini-hepcidins are engineered to reproduce the iron restriction effect of hepcidin. It was shown in a murine model of PV that administration of mini-hepcidins twice weekly by subcutaneous injection was effective in reducing haemoglobin and haematocrit levels (Casu et al., Blood. 2016 Jul. 14; 128(2): 265-276.). Similarly, PTG300, a hepcidin mimetic in clinical development decreased serum iron and transferrin saturation in healthy volunteers (62^{nd} ASH Annual Meeting and Exposition, Abstract #482). It was shown in a clinical phase 2 study with PV patients that treatment with PTG300 reduces the requirement for phlebotomy to maintain haematocrit levels in a range below 45% (62^{nd} ASH Annual Meeting and Exposition, Abstract #482).

### New Treatment Option: Inhibiting Matriptase-2/TMPRSS6

The inventors have surprisingly found a different route of treatment of at least some of the symptoms of myeloproliferative disorders such as PV. They have found that an inhibitor of the protein Matriptase-2 (MT2) can be used for such treatment. MT2 is the protein product of the TMPRSS6 gene and is a type II transmembrane serine protease that plays a critical role in the regulation of iron homeostasis. MT2 is a negative regulator for synthesis induction of the peptide hormone hepcidin. TMPRSS6 is primarily expressed in the liver, although high levels of TMPRSS6 mRNA are also found in the kidney, with lower levels in the uterus and much lower amounts detected in many other tissues (Ramsay et al., Haematologica (2009), 94(*6), 84-849).

Particularly inhibition of MT2 by reducing the expression of the TMPRSS6 gene appears to be a promising approach. Inhibition of TMPRSS6 expression can be attained in several ways. One way is the use of an inhibitory nucleic acid such as a siRNA or an antisense oligonucleotide (ASOs). These are short nucleic acids that inhibit the formation of proteins by causing targeted degradation of the mRNA molecules that encode these proteins. Such gene silencing agents are becoming increasingly important for therapeutic applications in medicine. For the pharmaceutical development of such nucleic acids, it is among others necessary that they can be synthesised economically, are metabolically stable, are specifically targeted to a tissue, are able to enter cells and function within acceptable limits of toxicity.

Double-stranded RNAs (dsRNA) able to bind through complementary base pairing to expressed mRNAs have been shown to block gene expression (Fire et al., 1998, Nature. 1998 Feb. 19; 391(6669):806-11 and Elbashir et al., 2001, Nature. 2001 May 24; 411(6836):494-8) by a mechanism that has been termed “RNA interference (RNAi)”. Short dsRNAs direct gene specific, post transcriptional silencing in many organisms, including vertebrates, and have become a useful tool for studying gene function. RNAi is mediated by the RNA induced silencing complex (RISC), a sequence specific, multi component nuclease that degrades messenger RNAs having sufficient complementary or homology to the silencing trigger loaded into the RISC complex.

### Advantages of Targeting MT2/TMPRSS6

Targeting MT2 or TMPRSS6 has advantages compared to all the treatment options listed above.

Phlebotomy lowers haematocrit and haemoglobin levels by removing red blood cells from the circulation and decreasing iron available for further erythropoiesis. However, each 500 ml phlebotomy removes ˜250 mg of iron from the body, thus leading to iron deficiency in the patients. Iron deficiency is associated with decreased quality of life through fatigue and impaired cognition. This could be avoided by the new treatment method, as iron restriction through elevation of endogenous hepcidin levels via a MT2 inhibitor, such as a TMPRSS6 siRNA, leads to a certain extent to redistribution of iron within the body, rather than eliminating it. This allows to limit serum iron availability for erythropoiesis but maintains peripheral iron stores. As such, the unwanted effects of systemic iron deficiency are likely to be much less severe after MT2 or TMPRSS6 inhibition, preferably via RNAi therapy, than with phlebotomy. This would significantly improve the quality of life of PV patients.

Phlebotomy is also associated with side effects linked to fluid shifts, including dizziness, nausea and vasovagal syncope. Therapies based on MT2 inhibition, such as TMPRSS6 siRNA therapy, are unlikely to affect fluid levels compartments and these complications would therefore likely not arise with this therapeutic approach. This is particularly important for low risk PV patients who cannot tolerate venesection and are therefore stratified to cytoreductive therapies due to lack of alternative options.

The risk for thrombotic events in PV patients is expected to be reduced by MT2 inhibition, for example with a TMPRSS6 siRNA, as haematocrit levels are reduced without further depleting the iron stores. Iron stores may even be restored over time with such a therapy. Furthermore, since MT2 or TMPRSS6 inhibition and the resulting elevation of the hepcidin level does not affect other lineages in haematopoiesis (other than iron restricted erythropoiesis) MT2 inhibitors, such as TMPRSS6 siRNAs, are unlikely to contribute to or increase the risk of bleeding events or other safety events when patients are treated concomitantly with Aspirin.

Currently available hepcidin agonists able to induce iron restriction have a short half-life, which requires frequent dosing (once or twice a week) by subcutaneous administration to reduce haematocrit levels by iron restriction (Casu et al., Blood. 2016 Jul. 14; 128(2): 265-276.; 62nASH Annual Meeting and Exposition, Abstract #482). In contrast, at least some types of MT2 inhibitors, such as TMPRSS6 siRNAs, are expected to require less frequent dosing (possibly no more than about once every 3 to 5 weeks) to reduce haemoglobin and haematocrit levels by iron restriction in vivo (Altamura et al., Hemasphere. 2019 December; 3(6): e301., Vadolas et al., 2021, Br. J. Hematology, in press). In addition, PTG300 was recently shown to lack efficacy in β-thalassaemia patients due to the oscillating effect on iron metabolism (EHA Library. Lal A. 06/12/20; 295117; S298). This suggests that high and stable elevation of hepcidin levels that could be attained by MT2 inhibitors, such as TMPRSS siRNAs, would be advantageous to provide a long and durable effect on iron metabolism and thus delivering the therapeutic effect. Also, since hepcidin agonists are modified peptides and require repeated administration, there is a risk that these foreign peptides could cause an immune reaction directed against them and potentially to the endogenous hepcidin (Zuckerman et al., Current Pharmaceutical Biotechnology, Volume 3, Number 4, 2002, pp. 349-360 (12)). In contrast, the risk of an immune response as a result of treatment with at least some MT2 inhibitors, such as TMPRSS6 siRNAs, which act to raise endogenous hepcidin levels, is unlikely.

Cytoreductive therapy is also associated with certain limitations that can be avoided by at least partial replacement through specific MT2 inhibition. Cytoreductive therapy is usually recommended for all high-risk PV patients along with low doses of aspirin and phlebotomy, as well as for low risk patients intolerant to phlebotomy. However, since this therapy is not specifically targeted, it affects many other cell lineages and is associated with many unwanted side effects. Hydroxycarbamide (hydroxyurea) is the first line cytoreductive therapy and acts at the level of DNA replication and therefore affects other tissues with high DNA replication such as the skin. Many patients are resistant or intolerant to hydroxycarbamide (Alvaez-Larran et al., Br J Haematol, 2016 March; 172 (5):789-93) and therefore require alternative therapies. Interferon-α and pegylated interferon-α-2a, which is better tolerated, are traditionally second line (now also first line) cytoreductive agents. Interferon agents reduce blood counts and have anti-clonal activity, but limiting side effects occur in 8-10% of patients (Kiladjian et al, Blood, 2006; Quintas-Cardama et al., J Clin Oncol, 2009). Recently, the JAK1/2 inhibitor Ruxolitinib has been approved for patients intolerant or resistant to hydroxycarbamide. Ruxolitinib has been shown to improve blood counts and decrease spleen size (Vannucchi et al., NEJM, 2015) but is currently reserved as a second/third line therapy for hydroxycarbamide intolerant/resistant patients.

The use of an MT2 inhibitor, such as a TMPRSS6 siRNA, may delay the need for use of cytoreductive therapies in low-risk patients. This is because iron restriction in the erythron reduces erythropoiesis drive, which may allow patients to meet their haematocrit goal ($45% for males and ≤42% for females) with a reduced phlebotomy need. For high-risk patients for whom aspirin and phlebotomy treatment does not reduce haematocrits to target levels, the use of an MT2 inhibitor, such as a TMPRSS6 siRNA, may be used instead, alone or in combination with a cytoreductive therapy. This could lead to the elimination or at least a reduction of the dose-limiting side effects of the cytoreductive therapy and thereby reduce morbidity and improve quality of life for patients.

Current treatments of myeloproliferative disorders such as polycythaemia vera all have drawbacks. There is therefore a clear need in the art for new ways of treating such disorders. The invention addresses this need. The use of an MT2 inhibitor, such as a GalNAc-conjugated TMPRSS6 siRNA, presents a promising therapeutic strategy for myeloproliferative disorders such as polycythaemia vera.

### SUMMARY OF THE INVENTION

One aspect of the invention is an inhibitor of Matriptase-2 (MT2) function and/or expression, for use in the prevention, decrease of the risk of suffering from or treatment of a myeloproliferative disorder, as well as associated diagnostic or therapeutic methods.

One aspect of the invention is a nucleic acid that is capable of inhibiting expression of TMPRSS6, for use in the prevention, decrease of the risk of suffering from or treatment of a myeloproliferative disorder, as well as associated diagnostic or therapeutic methods.

One aspect of the invention is a double-stranded nucleic acid that is capable of inhibiting expression of TMPRSS6, for use in the prevention, decrease of the risk of suffering from or treatment of a myeloproliferative disorder, as well as associated diagnostic or therapeutic methods.

One aspect of the invention is a composition comprising a nucleic acid as described herein and a solvent, preferably water, and/or a delivery vehicle and/or a physiologically acceptable excipient and/or a carrier and/or a salt and/or a diluent and/or a buffer and/or a preservative and/or a further therapeutic agent selected from an oligonucleotide, a small molecule, a monoclonal antibody, a polyclonal antibody and a peptide.

One aspect relates to an inhibitor, a nucleic acid and/or a composition as disclosed herein and a further therapeutic agent selected from an oligonucleotide, a small molecule, a monoclonal antibody, a polyclonal antibody and a peptide.

One aspect relates to the use of an inhibitor, a nucleic acid and/or a composition as disclosed herein in the prevention, decrease of the risk of suffering from or treatment of a myeloproliferative disorder.

One aspect relates to a method of preventing, decreasing the risk of suffering from, or treating a myeloproliferative disorder comprising administering a pharmaceutically effective amount of an inhibitor, a nucleic acid and/or a composition as disclosed herein to an individual in need of treatment.

One aspect is the use of an inhibitor or nucleic acid or composition as disclosed herein in the manufacture of a medicament for treating a myeloproliferative disorder.

One aspect relates to an inhibitor, a nucleic acid or a composition as disclosed herein in the treatment of a myeloproliferative disorder or in an associated diagnostic or therapeutic methods, wherein the myeloproliferative disorder is:
a) a Philadelphia chromosome (BCR-ABL) negative myeloproliferative neoplasm;
b) one or several of polycythaemia vera (PV), essential thrombocythaemia (ET) and primary myelofibrosis (PMF); or
c) polycythaemia vera (PV).

### DETAILED DESCRIPTION OF THE INVENTION

The inventors have surprisingly found that at least some of the symptoms of myeloproliferative disorders can be treated by inhibiting Matriptase-2 (MT2). Accordingly, the present invention relates to an inhibitor of Matriptase-2 (MT2) function and/or expression, for use in the prevention, decrease of the risk of suffering from or treatment of a myeloproliferative disorder, as well as associated diagnostic or therapeutic methods.

In one embodiment, the inhibitor is capable of inhibiting the function or the expression of Matriptase-2 (MT2) in vivo, preferably in the liver or in hepatocytes.

In one embodiment, an inhibitor of Matriptase-2 (MT2) function and/or expression is a compound that is able to:
a) prevent the protein Matriptase-2 (MT2) from carrying out its function, preferably repression of hepcidin expression, such as for example by binding to the protein so as to prevent it from binding to its usual interaction partner(s);
b) disrupt the interaction of Matriptase-2 (MT2) with its usual interaction partner(s), to prevent it from repressing expression of hepcidin;
c) prevent Matriptase-2 (MT2) from down-regulating the expression of hepcidin; and/or d) reduce the expression level of Matriptase-2 (MT2), for example by preventing production of the protein, by increasing the degradation level of Matriptase-2 (MT2) and/or by increasing the degradation rate of the mRNA that encodes the Matriptase-2 (MT2) protein.

In one embodiment, the Matriptase-2 (MT2) inhibitor reduces the expression of Matriptase-2 (MT2) by 10%, 20%, 30%, 40%, 50%, 55%, 60%, 65%, 70%, 75%, 80%, 85%, 90%, 95%, 96%, 97%, 98%, 99% or 100%. In one embodiment, the Matriptase-2 (MT2) inhibitor reduces the expression of the TMPRSS6 mRNA, such as for example SEQ ID NO: 1385, by 10%, 20%, 30%, 40%, 50%, 55%, 60%, 65%, 70%, 75%, 80%, 85%, 90%, 95%, 96%, 97%, 98%, 99% or 100%. These reduced expression levels may be measured in a cell or a hepatocyte or the liver of a subject treated with the inhibitor. The reduced expression levels of Matriptase-2 may result in increased hepcidin levels which may be measured in serum or plasma. In one embodiment, the Matriptase-2 (MT2) inhibitor is a nucleic acid, a monoclonal antibody, a polyclonal antibody, a small molecule, a peptide, or a protein.

In one embodiment, the Matriptase-2 (MT2) inhibitor is a nucleic acid, wherein the nucleic acid comprises at least one strand with a sequence that is at least partially complementary to a portion of SEQ ID NO: 1385 (the TMPRSS6 mRNA sequence that encodes Matriptase-2 (MT2) shown in FIG. 3). The length of the sequence and the level of complementarity of the sequence are in one embodiment sufficient to enable the nucleic acid to inhibit the expression of Matriptase-2 (MT2). Examples of sequence lengths that are sufficient to allow inhibition are typically between 13 and 35 nucleotides, such as 13, 14, 15, 16, 17, 18, 19, 20, 21, 22, 23, 24, 25, 26, 27, 28, 29, 30, 31, 32, 33, 34 or 35 nucleotides or nucleotide analogues. Examples of the level of complementarity required for inhibition are full complementarity, partial complementarity with one, two, three, four, five, six, seven, eight, nine or ten nucleotides or nucleotide analogues that are not complementary to SEQ ID NO: 1385.

In one embodiment, the Matriptase-2 (MT2) inhibitor is a nucleic acid, wherein the nucleic acid has at least one modified nucleotide, such as a 2′-OMe or 2′-F modified nucleotide and/or the nucleic acid has at least one phosphorothioate (PS) or phosphorodithioate (PS2) internucleotide linkage.

In one embodiment, the Matriptase-2 (MT2) inhibitor is a nucleic acid, wherein the nucleic acid is conjugated to a ligand.

In one embodiment, the Matriptase-2 (MT2) inhibitor is a nucleic acid, wherein the nucleic acid reduces the expression of Matriptase-2 (MT2) by RNA interference or RNase H-mediated degradation of the TMPRSS6 mRNA.

In one embodiment, the Matriptase-2 (MT2) inhibitor is a nucleic acid, wherein the nucleic acid is a siRNA or an ASO.

In one embodiment, the Matriptase-2 (MT2) inhibitor is a nucleic acid, wherein the nucleic acid is an ASO, and preferably an ASO as disclosed in WO2016161429.

In one embodiment, the Matriptase-2 (MT2) inhibitor is a nucleic acid, wherein the nucleic acid is single-stranded or double-stranded.

All embodiments relating to nucleic acids disclosed below also apply to Matriptase-2 (MT2) inhibitor that are nucleic acids, as far as they are compatible. The skilled person will be able to determine whether the embodiments disclosed below are compatible.

One embodiment of the invention is a nucleic acid that is capable of inhibiting expression of TMPRSS6, for use in the prevention, decrease of the risk of suffering from or treatment of a myeloproliferative disorder.

One embodiment of the invention is a double-stranded nucleic acid that is capable of inhibiting expression of TMPRSS6, for use in the prevention, decrease of the risk of suffering from or treatment of a myeloproliferative disorder.

In one embodiment, the nucleic acid is capable of inhibiting expression of TMPRSS6 in vivo, preferably in the liver or in hepatocytes.

In one embodiment, the double-stranded nucleic acid comprises a sequence that is homologous to and/or complementary to a portion of an expressed RNA transcript of TMPRSS6 such as SEQ ID NO: 1385.

In one embodiment, the double-stranded nucleic acid comprises a first strand and a second strand.

In one embodiment, the first strand and the second strand of the double-stranded nucleic acid are at least partially complementary to each other.

In one embodiment of the double-stranded nucleic acid, the first strand comprises a sequence that is at least partially complementary to an equivalent portion of SEQ ID NO: 1385.

In one embodiment, the first strand comprises a sequence of at least 15 nucleotides differing by no more than 3 nucleotides from any one of the sequences selected from SEQ ID NO: 6, 8, 10, 12, 14, 16, 18, 20, 22, 24, 26, 28, 30, 32, 34 and 36.

These nucleic acids among others have the advantage of being active in various species that are relevant for pre-clinical and clinical development and/or of having few relevant off-target effects. Having few relevant off-target effects means that a nucleic acid specifically inhibits the intended target and does not significantly inhibit other genes or inhibits only one or few other genes at a therapeutically acceptable level.

In one embodiment, the first strand sequence comprises, or essentially consists of, a sequence of at least 16, more preferably at least 17, yet more preferably at least 18 and most preferably all 19 nucleotides differing by no more than 3 nucleotides, preferably by no more than 2 nucleotides, more preferably by no more than 1 nucleotide, and most preferably not differing by any nucleotide from any one of the sequences selected from SEQ ID NO: 6, 8, 10, 12, 14, 16, 18, 20, 22, 24, 26, 28, 30, 32, 34 and 36.

In one embodiment, the first strand sequence of the nucleic acid consists of one of the sequences selected from SEQ ID NO: 6, 8, 10, 12, 14, 16, 18, 20, 22, 24, 26, 28, 30, 32, 34 and 36. The sequence may however be modified by a number of nucleic acid modifications that do not change the identity of the nucleotide. For example, modifications of the backbone or sugar residues of the nucleic acid do not change the identity of the nucleotide because the base itself remains the same as in the reference sequence.

A nucleic acid that comprises a sequence according to a reference sequence herein means that the nucleic acid comprises a sequence of contiguous nucleotides in the order as defined in the reference sequence.

When reference is made herein to a sequence comprising or consisting of a number of nucleotides that are not shown to be modified in that sequence, the reference also encompasses the same nucleotide sequence in which one, several, such as two, three, four, five, six, seven or more, including all, nucleotides are modified by modifications such as 2′-OMe, 2′-F, are linked to a ligand or a linker, have a 3′ end or 5′ end modification or any other modification. It also encompasses sequences in which two or more nucleotides are linked to each other by the natural phosphodiester linkage or by any other linkage such as a phosphorothioate or a phosphorodithioate linkage.

A double-stranded nucleic acid is a nucleic acid in which the first strand and the second strand hybridise to each other over at least part of their lengths and are therefore capable of forming a duplex region under physiological conditions, such as in PBS at 37° C. at a concentration of 1 UM of each strand. The first and second strand are preferably able to hybridise to each other and therefore to form a duplex region over a region of at least 15 nucleotides, preferably 16, 17, 18 or 19 nucleotides. This duplex region comprises nucleotide base parings between the two strands, preferably based on Watson-Crick base pairing and/or wobble base pairing (such as GU base pairing). All the nucleotides of the two strands within a duplex region do not have to base pair to each other to form a duplex region. A certain number of mismatches, deletions or insertions between the nucleotide sequences of the two strands are acceptable. Overhangs on either end of the first or second strand or unpaired nucleotides at either end of the double-stranded nucleic acid are also possible. The double-stranded nucleic acid is preferably a stable double-stranded nucleic acid under physiological conditions, and preferably has a melting temperature (Tm) of 45° C. or more, 50° C. or more, 55° C. or more, 60° C. or more, 65° C. or more, 70° C. or more, 75° C. or more, 80° C. or more, or 85° C. or more, for example in PBS at a concentration of 1 μM of each strand.

The first strand and the second strand are preferably capable of forming a duplex region (i.e., are complementary to each other) over i) at least a portion of their lengths, preferably over at least 15 nucleotides of both of their lengths, ii) over the entire length of the first strand, iii) over the entire length of the second strand or iv) over the entire length of both the first and the second strand. Strands being complementary to each other over a certain length means that the strands are able to base pair to each other, either via Watson-Crick or wobble base pairing, over that length. Each nucleotide of the length does not necessarily have to be able to base pair with its counterpart in the other strand over the entire given length as long as a stable double-stranded nucleotide under physiological conditions can be formed. It is however preferred, in certain embodiments, if each nucleotide of the length can base pair with its counterpart in the other strand over the entire given length.

A certain number of mismatches, deletions or insertions between the first strand and the target sequence, or between the first strand and the second strand can be tolerated in the context of the siRNA and even have the potential in certain cases to increase RNA interference (e.g., inhibition) activity.

The inhibition activity of the nucleic acids according to the present invention relies on the formation of a duplex region between all or a portion of a strand of the nucleic acid and a portion of a target nucleic acid. The portion of the target nucleic acid that forms a duplex region with the strand of the nucleic acid, defined as beginning with the first base pair formed between the first strand and the target sequence and ending with the last base pair formed between the first strand and the target sequence, inclusive, is the target nucleic acid sequence or simply, target sequence. In the case of double-stranded nucleic acids, the duplex region formed between the first strand and the second strand need not be the same as the duplex region formed between the first strand and the target sequence. That is, the second strand may have a sequence different from the target sequence; however, the first strand must be able to form a duplex structure with both the second strand and the target sequence, at least under physiological conditions.

The complementarity between the first strand and the target sequence may be perfect (i.e., 100% identity with no nucleotide mismatches or insertions or deletions in the first strand as compared to the target sequence).

The complementarity between the first strand and the target sequence may not be perfect. The complementarity may be from about 70% to about 100%. More specifically, the complementarity may be at least 70%, 80%, 85%, 90% or 95% and intermediate values.

The identity between the first strand and the complementary sequence of the target sequence may range from about 75% to about 100%. More specifically, the complementarity may be at least 75%, 80%, 85%, 90% or 95% and intermediate values, provided a nucleic acid is capable of reducing or inhibiting the expression of TMPRSS6.

A nucleic acid having less than 100% complementarity between the first strand and the target sequence may be able to reduce the expression of TMPRSS6 to the same level as a nucleic acid having perfect complementarity between the first strand and the target sequence.

Alternatively, it may be able to reduce expression of TMPRSS6 to a level that is 15%, 20%, 25%, 30%, 35%, 40%, 45%, 50%, 55%, 60%, 65%, 70%, 75%, 80%, 85%, 90%, 95%, 98%, 99% or 100% of the level of reduction achieved by the nucleic acid with perfect complementarity.

In one aspect, a nucleic acid of the present invention is a nucleic acid wherein
(a) the first strand sequence comprises a sequence differing by no more than 3 nucleotides from any one of the first strand sequences of Table 1 and/or wherein the second strand sequence comprises a sequence differing by no more than 3 nucleotides from the second strand sequence in the same line of the table;
(b) the first strand sequence comprises a sequence differing by no more than 2 nucleotides from any one of the first strand sequences of Table 1 and/or wherein the second strand sequence comprises a sequence differing by no more than 2 nucleotides from the second strand sequence in the same line of the table;
(c) the first strand sequence comprises a sequence differing by no more than 1 nucleotide from any one of the first strand sequences of Table 1 and/or wherein the second strand sequence comprises a sequence differing by no more than 1 nucleotide from the second strand sequence in the same line of the table;
(d) the first strand sequence comprises a sequence corresponding to nucleotides 2 to 17 from the 5′ end of any one of the first strand sequences of Table 1 and/or wherein the second strand sequence comprises a sequence corresponding to nucleotides 2 to 17 from the 5′ end of the second strand sequence in the same line of the table;
(e) the first strand sequence comprises a sequence corresponding to nucleotides 2 to 18 from the 5′ end of any one of the first strand sequences of Table 1 and/or wherein the second strand sequence comprises a sequence corresponding to nucleotides 2 to 18 from the 5′ end of the second strand sequence in the same line of the table;
(f) the first strand sequence comprises a sequence corresponding to nucleotides 2 to 19 from the 5′ end of any one of the first strand sequences of Table 1 and/or wherein the second strand sequence comprises a sequence corresponding to nucleotides 2 to 19 from the 5′ end of the second strand sequence in the same line of the table;
(g) the first strand sequence comprises a sequence corresponding to nucleotides 2 to 19 from the 5′ end of any one of the first strand sequences of Table 1 and/or wherein the second strand sequence comprises a sequence corresponding to nucleotides 1 to 18 from the 5′ end of the second strand sequence in the same line of the table;
(h) the first strand sequence comprises a sequence of any one of the first strand sequences of Table 1 and/or wherein the second strand sequence comprises a sequence of the second strand sequence in the same line of the table; or
(i) the first strand sequence consists of any one of the first strand sequences of Table 1 and/or wherein the second strand sequence consists of the sequence of the second strand sequence in the same line of the table;
   wherein Table 1 is:

TABLE 1

| First strand sequence | Second strand sequence |
|---|---|
| (SEQ ID NO:) | (SEQ ID NO:) |
| 6 | 7 |
| 8 | 9 |
| 10 | 11 |
| 12 | 13 |
| 14 | 15 |
| 16 | 17 |
| 18 | 19 |
| 20 | 21 |
| 22 | 23 |
| 24 | 25 |
| 26 | 27 |
| 28 | 29 |
| 30 | 31 |
| 32 | 33 |
| 34 | 35 |
| 36 | 37 |

In one aspect of the double-stranded nucleic acid, the first strand sequence comprises, consists essentially of, or consists of the sequence of SEQ ID NO: 6 and/or the second strand sequence comprises, consists essentially of, or consists of the sequence of SEQ ID NO: 7.

In one aspect, if the 5′-most nucleotide of the first strand is a nucleotide other than A or U, this nucleotide is replaced by an A or U. preferably, if the 5′-most nucleotide of the first strand is a nucleotide other than a U, this nucleotide is replaced by U, and more preferably by U with a 5′ (E)-vinylphosphonate, in the sequence.

In one aspect of the double-stranded nucleic acid, there is a mismatch between the first nucleotide at the 5′ end of the first strand and the corresponding nucleotide (the nucleotide with which it would form a base pair if there was no mismatch) in the second strand. For example, the 5′ nucleotide of the first strand may be U and the corresponding nucleotide in the second strand may be any nucleotide other than A. In this case, the two nucleotides are unable to form a classical Watson-Crick base pair and there is a mismatch between the two nucleotides.

When a double-stranded nucleic acid of the invention does not comprise the entire sequence of a reference first strand and/or second strand sequence as for example given in Table 1, or one or both strands differ from the corresponding reference sequence by one, two or three nucleotides, this nucleic acid preferably retains at least 30%, more preferably at least 50%, more preferably at least 70%, more preferably at least 80%, even more preferably at least 90%, yet more preferably at least 95% and most preferably 100% of the TMPRSS6 inhibition activity compared to the inhibition activity of the corresponding nucleic acid that comprises the entire first strand and second strand reference sequences in a comparable experiment.

In one aspect, the double-stranded nucleic acid is a nucleic acid wherein the first strand sequence comprises, consists essentially of, or consists of the sequence of SEQ ID NO: 6 and/or wherein the second strand sequence comprises, consists essentially of, or consists of, a sequence of at least 15, preferably at least 16, more preferably at least 17, yet more preferably at least 18 and most preferably all nucleotides of the sequence of SEQ ID NO: 7.

In one aspect, the nucleic acid is a double-stranded nucleic acid capable of inhibiting expression of TMPRSS6, wherein the nucleic acid comprises a first nucleic acid strand and a second nucleic acid strand, wherein the first strand is capable of hybridising under physiological conditions to a nucleic acid of a sequence selected from SEQ ID NO: 7, 9, 11, 13, 15, 17, 19, 21, 23, 25, 27, 29, 31, 33, 35 and 37; and wherein the second strand is capable of hybridising under physiological conditions to the first strand to form a duplex region.

Nucleic acids that are capable of hybridising under physiological conditions are nucleic acids that are capable of forming base pairs, preferably Watson-Crick or wobble base-pairs, between at least a portion of the opposed nucleotides in the strands so as to form at least a duplex region. Such a double-stranded nucleic acid is preferably a stable double-stranded nucleic acid under physiological conditions (for example in PBS at 37° C. at a concentration of 1 UM of each strand), meaning that under such conditions, the two strands stay hybridised to each other. The Tm of the double-stranded nucleotide is preferably 45° C. or more, preferably 50° C. or more and more preferably 55° C. or more.

In one aspect of the present invention, the double-stranded nucleic acid capable of inhibiting the expression of TMPRSS6, comprises a first sequence of at least 15, preferably at least 16, more preferably at least 17, yet more preferably at least 18 and most preferably all nucleotides differing by no more than 3 nucleotides, preferably no more than 2 nucleotides, more preferably no more than 1 nucleotide and most preferably not differing by any nucleotide from any of the sequences of Table 4, the first sequence being able to hybridise to a target gene transcript (such as an mRNA, preferably SEQ ID NO: 1385) under physiological conditions. In addition, or alternatively, the nucleic acid comprises a second sequence of at least 15, preferably, at least 16, more preferably at least 17, yet more preferably at least 18 and most preferably all nucleotides differing by no more than 3 nucleotides, preferably no more than 2 nucleotides, more preferably no more than 1 nucleotide and most preferably not differing by any nucleotide from any of the sequences of Table 4, wherein the second sequence is able to hybridise to the first sequence under physiological conditions and preferably wherein the nucleic acid is an siRNA that is capable of inhibiting TMPRSS6 expression via the RNAi pathway.

In one aspect of the present invention, the double-stranded nucleic acid capable of inhibiting the expression of TMPRSS6, is any double-stranded nucleic acid as disclosed in Table 2, provided that the double-stranded nucleic acid is capable of inhibiting expression of TMPRSS6, preferably in vivo, in hepatocytes and/or in the liver. These nucleic acids are all siRNAs with unmodified or modified nucleotide modifications. Some of them are conjugates comprising GalNAc moieties that can be specifically targeted to cells with GalNAc receptors, such as hepatocytes. In one aspect, these nucleic acids have modifications, such as 2′ nucleotide modifications and/or internucleotide modifications, and/or are attached to a ligand that targets them to the liver, such as a GalNAc-comprising ligand, so as to be able to reduce the expression of TMPRSS6 in the liver of a subject. Possible modifications that provide the necessary properties to the nucleic acids of Table 2 are disclosed herein or for example in WO2018185240, WO2012135246 and WO2014190157.

In one aspect of the double-stranded nucleic acid that is capable of inhibiting expression of TMPRSS6, the nucleic acid comprises or consists of a first strand and a second strand and preferably the first strand comprises a sequence sufficiently complementary to a TMPRSS6 mRNA, such as SEQ ID NO: 1385, so as to mediate RNA interference.

The inhibitors or nucleic acids described herein may be capable of inhibiting the expression of TMPRSS6, preferably in vivo, in hepatocytes and/or in the liver. The inhibitors or nucleic acids may be capable of inhibiting TMPRSS6 expression completely, resulting in 0% remaining expression upon treatment with the nucleic acids. The inhibitors or nucleic acids may be capable of partially inhibiting TMPRSS6 expression. Partial inhibition means that TMPRSS6 expression is decreased by 15%, 20%, 30%, 40%, 50%, 60%, 70%, 75%, 80%, 85%, 90%, 95% or more, or intermediate values, as compared to the absence of the nucleic acids under comparable conditions. The level of inhibition may be measured by comparing a treated sample with an untreated sample or with a sample treated with a control, such as for example an inhibitor or a siRNA that does not target TMPRSS6. Inhibition may be measured by measuring TMPRSS6 mRNA and/or protein levels or levels of a biomarker or indicator that correlates with Matriptase-2 (MT2) presence or activity. It may be measured in cells that may have been treated with an inhibitor or nucleic acid described herein. Alternatively, or in addition, inhibition may be measured in cells, such as hepatocytes, or tissue, such as liver tissue, or an organ, such as the liver, or in a body fluid such as blood, serum, lymph or in any other body part or fluid that has been taken from a subject previously treated with an inhibitor or a nucleic acid disclosed herein. Preferably, inhibition of TMPRSS6 expression is determined by comparing the TMPRSS6 mRNA level measured in TMPRSS6-expressing cells after 24 or 48 hours treatment with a nucleic acid or other inhibitor disclosed herein under ideal conditions (see the examples for appropriate concentrations and conditions) to the TMPRSS6 mRNA level measured in control cells that were untreated or mock treated or treated with a control nucleic acid or other inhibitor under the same or at least comparable conditions.

One aspect of the present invention relates to a double-stranded nucleic acid, wherein the first strand and the second strand are present on a single strand of a nucleic acid that loops around so that the first strand and the second strand are able to hybridise to each other and to thereby form a double-stranded nucleic acid with a duplex region.

In one embodiment, the first strand and the second strand of the nucleic acid are separate strands. The two separate strands are preferably each 17-25 nucleotides in length, more preferably 18-25 nucleotides in length. The two strands may be of the same or different lengths. The first strand may be 17-25 nucleotides in length, preferably it may be 18-24 nucleotides in length, it may be 18, 19, 20, 21, 22, 23 or 24 nucleotides in length. Most preferably, the first strand is 19 nucleotides in length. The second strand may independently be 17-25 nucleotides in length, preferably it may be 18-24 nucleotides in length, it may be 18, 19, 20, 21, 22, 23 or 24 nucleotides in length. More preferably, the second strand is 18 or 19 or 20 nucleotides in length, and most preferably it is 19 nucleotides in length.

In one embodiment, the first strand and the second strand of the nucleic acid form a duplex region of 17-25 nucleotides in length. More preferably, the duplex region is 18-24 nucleotides in length. The duplex region may be 17, 18, 19, 20, 21, 22, 23, 24 or 25 nucleotides in length.

In the most preferred embodiment, the duplex region is 18 or 19 nucleotides in length. The duplex region is defined here as the region between and including the 5′-most nucleotide of the first strand that is base paired to a nucleotide of the second strand to the 3′-most nucleotide of the first strand that is base paired to a nucleotide of the second strand. The duplex region may comprise nucleotides in either or both strands that are not base-paired to a nucleotide in the other strand. It may comprise one, two, three or four such nucleotides on the first strand and/or on the second strand. However, preferably, the duplex region consists of 17-25 consecutive nucleotide base pairs. That is to say that it preferably comprises 17-25 consecutive nucleotides on both of the strands that all base pair to a nucleotide in the other strand. More preferably, the duplex region consists of 18 or 19 consecutive nucleotide base pairs, most preferably 19.

In each of the embodiments of a double-stranded nucleic acid with two separate strands disclosed herein, the nucleic acid may be blunt ended at both ends; have an overhang at one end and a blunt end at the other end; or have an overhang at both ends.

The double-stranded nucleic acid may have an overhang at one end and a blunt end at the other end. The nucleic acid may have an overhang at both ends. The nucleic acid may be blunt ended at both ends. The nucleic acid may be blunt ended at the end with the 5′ end of the first strand and the 3′ end of the second strand or at the 3′ end of the first strand and the 5′ end of the second strand.

The double-stranded nucleic acid may comprise an overhang at a 3′ or 5′ end. The nucleic acid may have a 3′ overhang on the first strand. The nucleic acid may have a 3′ overhang on the second strand. The nucleic acid may have a 5′ overhang on the first strand. The nucleic acid may have a 5′ overhang on the second strand. The nucleic acid may have an overhang at both the 5′ end and 3′ end of the first strand. The nucleic acid may have an overhang at both the 5′ end and 3′ end of the second strand. The nucleic acid may have a 5′ overhang on the first strand and a 3′ overhang on the second strand. The nucleic acid may have a 3′ overhang on the first strand and a 5′ overhang on the second strand. The nucleic acid may have a 3′ overhang on the first strand and a 3′ overhang on the second strand. The nucleic acid may have a 5′ overhang on the first strand and a 5′ overhang on the second strand.

An overhang at the 3′ end or 5′ end of the second strand or the first strand may consist of 1, 2, 3, 4 and 5 nucleotides in length. Optionally, an overhang may consist of 1 or 2 nucleotides, which may or may not be modified.

In one embodiment, the nucleic acid is an siRNA. siRNAs are short interfering or short silencing RNAs that are able to inhibit the expression of a target gene through the RNA interference (RNAi) pathway. Inhibition occurs through targeted degradation of mRNA transcripts of the target gene after transcription. The siRNA forms part of the RISC complex. The RISC complex specifically targets the target RNA by sequence complementarity of a strand (the antisense strand, which is the first strand in double-stranded nucleic acids) with the target sequence.

In on embodiment, the nucleic acid is capable of inhibiting TMPRSS6. The inhibition is preferably mediated by the RNA interference (RNAi) mechanism. Preferably, the nucleic acid mediates RNA interference (i.e., it is capable of inhibiting its target) with an efficacy of at least 50% inhibition, more preferably at least 70%, more preferably at least 80%, even more preferably at least 90%, yet more preferably at least 95% and most preferably 100% inhibition. The inhibition efficacy is preferably measured by comparing the TMPRSS6 mRNA level in cells, such as hepatocytes, treated with a TMPRSS6 specific siRNA to the TMPRSS6 mRNA level in cells treated with a control in a comparable experiment. The control can be a treatment with a non-TMPRSS6 targeting siRNA or without a siRNA.

The nucleic acid, or at least the first strand of the nucleic acid, is therefore preferably able to be incorporated into the RISC complex. As a result, the nucleic acid, or at least the first strand of the nucleic acid, is therefore able to guide the RISC complex to a specific target RNA with which the nucleic acid, or at least the first strand of the nucleic acid, is at least partially complementary. The RISC complex then specifically cleaves this target RNA and as a result leads to inhibition of the expression of the gene from which the RNA stems.

One embodiment is a nucleic acid wherein the first strand comprises, consists essentially of, or consists of SEQ ID NO: 3 and/or the second strand comprises, consists essentially of, or consists of SEQ ID NO: 1386 for use in the prevention, decrease of the risk of suffering from or treatment of a myeloproliferative disorder. This nucleic acid may be further conjugated to a ligand, preferably at the 5′ end of the second strand. More preferred is a nucleic acid wherein the first strand comprises, consists essentially of, or consists of SEQ ID NO: 3 and/or the second strand comprises, consists essentially of, or consists of SEQ ID NO: 4. Most preferred in this case is an siRNA that consists of SEQ ID NO: 3 and SEQ ID NO: 4. One aspect of the invention is EU401 for use in the prevention, decrease of the risk of suffering from or treatment of a myeloproliferative disorder, preferably polycythaemia vera, as well as associated diagnostic or therapeutic methods.

One embodiment is a nucleic acid wherein the first strand comprises, consists essentially of, or consists of SEQ ID NO: 3 and/or the second strand comprises, consists essentially of, or consists of SEQ ID NO: 1387 for use in the prevention, decrease of the risk of suffering from or treatment of a myeloproliferative disorder. This nucleic acid may be further conjugated to a ligand, preferably at the 5′ end of the second strand. More preferred is a nucleic acid wherein the first strand comprises, consists essentially of, or consists of SEQ ID NO: 3 and/or the second strand comprises, consists essentially of, or consists of SEQ ID NO: 5. Most preferred in this case is an siRNA that consists of SEQ ID NO: 3 and SEQ ID NO: 5. One aspect of the invention is EU402 for use in the prevention, decrease of the risk of suffering from or treatment of a myeloproliferative disorder, preferably polycythaemia vera, as well as associated diagnostic or therapeutic methods.

One aspect of the present invention relates to a Matriptase-2 (MT2) inhibitor such as an siRNA, an antibody, a small molecule, a peptide, a protein or any other agent that reduces the level of

Matriptase-2 (MT2) in the liver or blocks its activity, for use in the treatment of a myeloproliferative disorder, preferably polycythaemia vera.

### Nucleic Acid Modifications

Nucleic acids discussed herein include unmodified RNA as well as RNA which has been modified, e.g., to improve efficacy or stability. Unmodified RNA refers to a molecule in which the components of the nucleic acid, namely sugars, bases, and phosphate moieties, are the same or essentially the same as those which occur in nature, for example as occur naturally in the human body. The term “modified nucleotide” as used herein refers to a nucleotide in which one or more of the components of the nucleotide, namely the sugar, base, and phosphate moiety, is/are different from those which occur in nature. The term “modified nucleotide” also refers in certain cases to molecules that are not nucleotides in the strict sense of the term because they lack, or have a substitute of, an essential component of a nucleotide, such as the sugar, base or phosphate moiety. A nucleic acid comprising such modified nucleotides is still to be understood as being a nucleic acid, even if one or more of the nucleotides of the nucleic acid has been replaced by a modified nucleotide that lacks, or has a substitution of, an essential component of a nucleotide.

Modifications of the nucleic acid of the present invention generally provide a powerful tool in overcoming potential limitations including, but not limited to, in vitro and in vivo stability and bioavailability inherent to native RNA molecules. The nucleic acids according to the invention may be modified by chemical modifications. Modified nucleic acids can also minimise the possibility of inducing interferon activity in humans. Modifications can further enhance the functional delivery of a nucleic acid to a target cell. The modified nucleic acids of the present invention may comprise one or more chemically modified ribonucleotides. Modified nucleotides can be present in either or both of the first strand or the second strand when the nucleic acid has a first and a second strand. A ribonucleotide may comprise a chemical modification of the base, sugar or phosphate moieties. The ribonucleic acid may be modified by substitution with or insertion of analogues of nucleic acids or bases.

Throughout the description of the invention, “same or common modification” means the same modification to any nucleotide, be that A, G, C or U modified with a group such as a methyl group (2′-OMe) or a fluoro group (2′-F). For example, 2′-F-dU, 2′-F-dA, 2′-F-dC, 2′-F-dG are all considered to be the same or common modification, as are 2′-OMe-rU, 2′-OMe-rA; 2′-OMe-rC; 2′-OMe-rG. In contrast, a 2′-F modification is a different modification compared to a 2′-OMe modification.

In one embodiment, at least one nucleotide of the nucleic acid is a modified nucleotide, preferably a non-naturally occurring nucleotide such as preferably a 2′-F modified nucleotide. When the nucleic acid has a first and a second strand any of the first and/or second strand of the nucleic acid can have at least one modified nucleotide.

A modified nucleotide can be a nucleotide with a modification of the sugar group. The 2′ hydroxyl group (OH) can be modified or replaced with a number of different “oxy” or “deoxy” substituents.

Examples of “oxy”-2′ hydroxyl group modifications include alkoxy or aryloxy (OR, e.g., R=H, alkyl (such as methyl), cycloalkyl, aryl, aralkyl, heteroaryl or sugar); polyethyleneglycols (PEG), O(CH₂CH₂O)ₙCH₂CH₂OR; “locked” nucleic acids (LNA) in which the 2′ hydroxyl is connected, e.g., by a methylene bridge, to the 4′ carbon of the same ribose sugar; O-AMINE (AMINE=NH₂, alkylamino, dialkylamino, heterocyclyl, arylamino, diaryl amino, heteroaryl amino, or diheteroaryl amino, ethylene diamine or polyamino) and aminoalkoxy, O(CH₂)ₙAMINE, (e.g., AMINE=NH₂, alkylamino, dialkylamino, heterocyclyl, arylamino, diaryl amino, heteroaryl amino, or diheteroaryl amino, ethylene diamine or polyamino).

“Deoxy” modifications include hydrogen, halogen, amino (e.g., NH₂, alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂, alkylamino, dialkylamino, heterocyclyl, arylamino, diaryl amino, heteroaryl amino, or diheteroaryl amino), —NHC(O)R (R=alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino functionality. Other substituents of certain embodiments include 2′-methoxyethyl, 2′-OCH₃, 2′-O-allyl, 2′-C-allyl, and 2′-fluoro.

The sugar group can also contain one or more carbons that possess the opposite stereochemical configuration than that of the corresponding carbon in ribose. Thus, a modified nucleotide may contain a sugar such as arabinose.

Modified nucleotides can also include “abasic” sugars, which lack a nucleobase at C—1′. These abasic sugars can further contain modifications at one or more of the constituent sugar atoms.

The 2′ modifications may be used in combination with one or more phosphate internucleoside linker modifications (e.g., phosphorothioate or phosphorodithioate).

One or more nucleotides of a nucleic acid of the present invention may be modified. The nucleic acid may comprise at least one modified nucleotide. When the nucleic acid is a double-stranded nucleic acid, the modified nucleotide may be in the first strand. The modified nucleotide may be in the second strand. The modified nucleotide may be in the duplex region. The modified nucleotide may be outside the duplex region, i.e., in a single-stranded region. The modified nucleotide may be on the first strand and may be outside the duplex region. The modified nucleotide may be on the second strand and may be outside the duplex region. The 3′-terminal nucleotide of the first strand may be a modified nucleotide. The 3′-terminal nucleotide of the second strand may be a modified nucleotide. The 5′-terminal nucleotide of the first strand may be a modified nucleotide. The 5′-terminal nucleotide of the second strand may be a modified nucleotide.

A nucleic acid of the invention may have 1 modified nucleotide or a nucleic acid of the invention may have about 2-4 modified nucleotides, or a nucleic acid may have about 4-6 modified nucleotides, about 6-8 modified nucleotides, about 8-10 modified nucleotides, about 10-12 modified nucleotides, about 12-14 modified nucleotides, about 14-16 modified nucleotides about 16-18 modified nucleotides, about 18-20 modified nucleotides, about 20-22 modified nucleotides, about 22-24 modified nucleotides, about 24-26 modified nucleotides or about 26-28 modified nucleotides. In each case the nucleic acid comprising said modified nucleotides retains at least 50% of its activity as compared to the same nucleic acid but without said modified nucleotides or vice versa. The nucleic acid may retain 55%, 60%, 65%, 70%, 75%, 80%, 85%, 90%, 95% or 100% and intermediate values of its activity as compared to the same nucleic acid but without said modified nucleotides, or may have more than 100% of the activity of the same nucleic acid without said modified nucleotides.

The modified nucleotide may be a purine or a pyrimidine. At least half of the purines may be modified. At least half of the pyrimidines may be modified. All of the purines may be modified. All of the pyrimidines may be modified. The modified nucleotides may be selected from the group consisting of a 3′ terminal deoxy thymine (dT) nucleotide, a 2′-O-methyl (2′-OMe) modified nucleotide, a 2′ modified nucleotide, a 2′ deoxy modified nucleotide, a locked nucleotide, an abasic nucleotide, a 2′ amino modified nucleotide, a 2′ alkyl modified nucleotide, a 2′-deoxy-2′-fluoro (2′-F) modified nucleotide, a morpholino nucleotide, a phosphoramidate, a non-natural base comprising nucleotide, a nucleotide comprising a 5′-phosphorothioate group, a nucleotide comprising a 5′ phosphate or 5′ phosphate mimic and a terminal nucleotide linked to a cholesteryl derivative or a dodecanoic acid bisdecylamide group.

The nucleic acid may comprise a nucleotide comprising a modified base, wherein the base is selected from 2-aminoadenosine, 2,6-diaminopurine, inosine, pyridin-4-one, pyridin-2-one, phenyl, pseudouracil, 2,4,6-trimethoxy benzene, 3-methyl uracil, dihydrouridine, naphthyl, aminophenyl, 5-alkylcytidine (e.g., 5-methylcytidine), 5-alkyluridine (e.g., ribothymidine), 5-halouridine (e.g., 5-bromouridine), 6-azapyrimidine, 6-alkylpyrimidine (e.g. 6-methyluridine), propyne, quesosine, 2-thiouridine, 4-thiouridine, wybutosine, wybutoxosine, 4-acetylcytidine, 5-(carboxyhydroxymethyl)uridine, 5′-carboxymethylaminomethyl-2-thiouridine, 5-carboxymethylaminomethyluridine, beta-D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, beta-D-mannosylqueosine, uridine-5-oxyacetic acid and 2-thiocytidine.

Many of the modifications described herein and that occur within a nucleic acid will be repeated within a polynucleotide molecule, such as a modification of a base, or a phosphate moiety, or a non-linking O of a phosphate moiety. In some cases, the modification will occur at all of the possible positions/nucleotides in the polynucleotide but in many cases it will not. A modification may only occur at a 3′ or 5′ terminal position, may only occur in a terminal region, such as at a position on a terminal nucleotide or in the last 2, 3, 4, 5, or 10 nucleotides of a strand. A modification may occur in a double-strand region, a single-strand region, or in both. A modification may occur only in the double-strand region of a nucleic acid of the invention or may only occur in a single-strand region of a nucleic acid of the invention. A phosphorothioate or phosphorodithioate modification at a non-linking O position may only occur at one or both termini, may only occur in a terminal region, e.g., at a position on a terminal nucleotide or in the last 2, 3, 4 or 5 nucleotides of a strand, or may occur in duplex and/or in single-strand regions, preferably at termini. The 5′ end and/or 3′ end may be phosphorylated.

Stability of a double-stranded nucleic acid of the invention may be increased by including particular bases in overhangs, or by including modified nucleotides, in single-strand overhangs, e.g., in a 5′ or 3′ overhang, or in both. Purine nucleotides may be included in overhangs. All or some of the bases in a 3′ or 5′ overhang may be modified. Modifications can include the use of modifications at the 2′ OH group of the ribose sugar, the use of deoxyribonucleotides, instead of ribonucleotides, and modifications in the phosphate group, such as phosphorothioate or phosphorodithioate modifications. Overhangs need not be homologous with the target sequence.

Nucleases can hydrolyse nucleic acid phosphodiester bonds. However, chemical modifications to nucleic acids can confer improved properties, and, can render oligoribonucleotides more stable to nucleases.

Modified nucleic acids, as used herein, can include one or more of:
(i) alteration, e.g., replacement, of one or both of the non-linking phosphate oxygens and/or of one or more of the linking phosphate oxygens (referred to as linking even if at the 5′ and 3′ terminus of the nucleic acid of the invention);
(ii) alteration, e.g., replacement, of a constituent of the ribose sugar, e.g., of the 2′ hydroxyl on the ribose sugar;
(iii) replacement of the phosphate moiety with “dephospho” linkers;
(iv) modification or replacement of a naturally occurring base;
(v) replacement or modification of the ribose-phosphate backbone; and
(vi) modification of the 3′ end or 5′ end of the first strand and/or the second strand when the nucleic acid as a first and a second strand, e.g., removal, modification or replacement of a terminal phosphate group or conjugation of a moiety, e.g., a fluorescently labelled moiety, to either the 3′ or 5′ end of one or both strands.

The terms replacement, modification and alteration indicate a difference from a naturally occurring molecule.

Specific modifications are discussed in more detail below.

The nucleic acid may comprise one or more nucleotides that are modified. Such modifications can occur on the second and/or first strands when the nuclei acid has a first and a second strand. Alternating nucleotides may be modified, to form modified nucleotides.

Alternating as described herein means to occur one after another in a regular way. In other words, alternating means to occur in turn repeatedly. For example, if one nucleotide is modified, the next contiguous nucleotide is not modified and the following contiguous nucleotide is modified and so on. One nucleotide may be modified with a first modification, the next contiguous nucleotide may be modified with a second modification and the following contiguous nucleotide is modified with the first modification and so on, where the first and second modifications are different.

In one aspect of the double-stranded nucleic acid, at least nucleotides 2 and 14 of the first strand are modified, preferably by a first common modification, the nucleotides being numbered consecutively starting with nucleotide number 1 at the 5′ end of the first strand. The first modification is preferably 2′-F.

In one aspect of a double-stranded nucleic acid, at least one, several or preferably all the even-numbered nucleotides of the first strand are modified, preferably by a first common modification, the nucleotides being numbered consecutively starting with nucleotide number 1 at the 5′ end of the first strand. The first modification is preferably 2′-F.

In one aspect of the double-stranded nucleic acid, at least one, several or preferably all the odd-numbered nucleotides of the first strand are modified, the nucleotides being numbered consecutively starting with nucleotide number 1 at the 5′ end of the first strand. Preferably, they are modified by a second modification. This second modification is preferably different from the first modification if the nucleic acid also comprises a first modification, for example of nucleotides 2 and 14 or of all the even-numbered nucleotides of the first strand. The first modification is preferably any 2′ ribose modification that is of the same size or smaller in volume than a 2′—OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′—OH group can for example be a 2′-F, 2′-H, 2′-halo, or 2′—NH₂. The second modification is preferably any 2′ ribose modification that is larger in volume than a 2′—OH group. A 2′ ribose modification that is larger in volume than a 2′—OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first modification is preferably 2′-F and/or the second modification is preferably 2′-OMe.

In the context of this disclosure, the size or volume of a substituent, such as a 2′ ribose modification, is preferably measured as the van der Waals volume.

In one aspect of the double-stranded nucleic acid, at least one, several or preferably all the nucleotides of the second strand in a position corresponding to an even-numbered nucleotide of the first strand are modified, preferably by a third modification. Preferably in the same nucleic acid nucleotides 2 and 14 or all the even numbered nucleotides of the first strand are modified with a first modification. In addition, or alternatively, the odd-numbered nucleotides of the first strand are modified with a second modification. Preferably, the third modification is different from the first modification and/or the third modification is the same as the second modification. The first modification is preferably any 2′ ribose modification that is of the same size or smaller in volume than a 2′—OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′—OH group can for example be a 2′-F, 2′-H, 2′-halo, or 2′—NH₂. The second and/or third modification is preferably any 2′ ribose modification that is larger in volume than a 2′—OH group. A 2′ ribose modification that is larger in volume than a 2′—OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first modification is preferably 2′-F and/or the second and/or third modification is/are preferably 2′-OMe. The nucleotides on the first strand are numbered consecutively starting with nucleotide number 1 at the 5′ end of the first strand.

A nucleotide of the second strand that is in a position corresponding, for example, to an even-numbered nucleotide of the first strand is a nucleotide of the second strand that is base-paired to an even-numbered nucleotide of the first strand, or would be base-paired if they were complementary.

In one aspect of the double-stranded nucleotide, at least one, several or preferably all the nucleotides of the second strand in a position corresponding to an odd-numbered nucleotide of the first strand are modified, preferably by a fourth modification. Preferably in the same nucleic acid nucleotides 2 and 14 or all the even numbered nucleotides of the first strand are modified with a first modification. In addition, or alternatively, the odd-numbered nucleotides of the first strand are modified with a second modification. In addition, or alternatively, all the nucleotides of the second strand in a position corresponding to an even-numbered nucleotide of the first strand are modified with a third modification. The fourth modification is preferably different from the second modification and preferably different from the third modification and the fourth modification is preferably the same as the first modification. The first and/or fourth modification is preferably any 2′ ribose modification that is of the same size or smaller in volume than a 2′—OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′—OH group can for example be a 2′-F, 2′-H, 2′-halo, or 2′—NH₂. The second and/or third modification is preferably any 2′ ribose modification that is larger in volume than a 2′—OH group. A 2′ ribose modification that is larger in volume than a 2′-OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first and/or the fourth modification is/are preferably a 2′-OMe modification and/or the second and/or third modification is/are preferably a 2′-F modification. The nucleotides on the first strand are numbered consecutively starting with nucleotide number 1 at the 5′ end of the first strand.

In one aspect of the double-stranded nucleic acid, the nucleotide/nucleotides of the second strand in a position corresponding to nucleotide 11 or nucleotide 13 or nucleotides 11 and 13 or nucleotides 11-13 of the first strand is/are modified by a fourth modification. Preferably, all the nucleotides of the second strand other than the nucleotide/nucleotides in a position corresponding to nucleotide 11 or nucleotide 13 or nucleotides 11 and 13 or nucleotides 11-13 of the first strand is/are modified by a third modification. Preferably in the same nucleic acid nucleotides 2 and 14 or all the even numbered nucleotides of the first strand are modified with a first modification. In addition, or alternatively, the odd-numbered nucleotides of the first strand are modified with a second modification. The fourth modification is preferably different from the second modification and preferably different from the third modification and the fourth modification is preferably the same as the first modification. The first and/or fourth modification is preferably any 2′ ribose modification that is of the same size or smaller in volume than a 2′-OH group, or a locked nucleic acid (LNA), or an unlocked nucleic acid (UNA), or a 2′-Fluoroarabino Nucleic Acid (FANA) modification. A 2′ ribose modification that is of the same size or smaller in volume than a 2′—OH group can for example be a 2′-F, 2′-H, 2′-halo, or 2′-NH₂. The second and/or third modification is preferably any 2′ ribose modification that is larger in volume than a 2′—OH group. A 2′ ribose modification that is larger in volume than a 2′—OH group can for example be a 2′-OMe, 2′-O-MOE (2′-O-methoxyethyl), 2′-O-allyl or 2′-O-alkyl, with the proviso that the nucleic is capable of reducing the expression of the target gene to at least the same extent as the same nucleic acid without the modification(s) under comparable conditions. The first and/or the fourth modification is/are preferably a 2′-OMe modification and/or the second and/or third modification is/are preferably a 2′-F modification. The nucleotides on the first strand are numbered consecutively starting with nucleotide number 1 at the 5′ end of the first strand.

In one aspect of the double-stranded nucleic acid, all the even-numbered nucleotides of the first strand are modified by a first modification, all the odd-numbered nucleotides of the first strand are modified by a second modification, all the nucleotides of the second strand in a position corresponding to an even-numbered nucleotide of the first strand are modified by a third modification, all the nucleotides of the second strand in a position corresponding to an odd-numbered nucleotide of the first strand are modified by a fourth modification, wherein the first and/or fourth modification is/are 2′-F and/or the second and/or third modification is/are 2′-OMe.

In one aspect of the double-stranded nucleic acid, all the even-numbered nucleotides of the first strand are modified by a first modification, all the odd-numbered nucleotides of the first strand are modified by a second modification, all the nucleotides of the second strand in positions corresponding to nucleotides 11-13 of the first strand are modified by a fourth modification, all the nucleotides of the second strand other than the nucleotides corresponding to nucleotides 11-13 of the first strand are modified by a third modification, wherein the first and fourth modification are 2′-F and the second and third modification are 2′-OMe. In one embodiment in this aspect, the 3′ terminal nucleotide of the second strand is an inverted RNA nucleotide (i.e., the nucleotide is linked to the 3′ end of the strand through its 3′ carbon, rather than through its 5′ carbon as would normally be the case). When the 3′ terminal nucleotide of the second strand is an inverted RNA nucleotide, the inverted RNA nucleotide is preferably an unmodified nucleotide in the sense that it does not comprise any modifications compared to the natural nucleotide counterpart. Specifically, the inverted RNA nucleotide is preferably a 2′-OH nucleotide. Preferably, in this aspect when the 3′ terminal nucleotide of the second strand is an inverted RNA nucleotide, the nucleic acid is blunt-ended at least at the end that comprises the 5′ end of the first strand.

One aspect of the present invention is a double-stranded nucleic acid as disclosed herein for inhibiting expression of the TMPRSS6 gene, wherein said first strand includes modified nucleotides or unmodified nucleotides at a plurality of positions in order to facilitate processing of the nucleic acid by RISC.

In one aspect, “facilitate processing by RISC” means that the nucleic acid can be processed by RISC, for example any modification present will permit the nucleic acid to be processed by RISC and preferably, will be beneficial to processing by RISC, suitably such that siRNA activity can take place.

One aspect is a double-stranded nucleic acid as disclosed herein, wherein the nucleotides at positions 2 and 14 from the 5′ end of the first strand are not modified with a 2′-OMe modification, and the nucleotide/nucleotides on the second strand which corresponds to position 11 or position 13 or positions 11 and 13 or positions 11, 12 and 13 of the first strand is/are not modified with a 2′-OMe modification (in other words, they are naturally occurring nucleotides or are modified with a modification other than 2′-OMe).

In one aspect of the double-stranded nucleic acid, the nucleotide on the second strand which corresponds to position 13 of the first strand is the nucleotide that forms a base pair with position 13 (from the 5′ end) of the first strand.

In one aspect of the double-stranded nucleic acid, the nucleotide on the second strand which corresponds to position 11 of the first strand is the nucleotide that forms a base pair with position 11 (from the 5′ end) of the first strand.

In one aspect of the double-stranded nucleic acid, the nucleotide on the second strand which corresponds to position 12 of the first strand is the nucleotide that forms a base pair with position 12 (from the 5′ end) of the first strand.

For example, in a 19-mer nucleic acid which is double-stranded and blunt ended, position 13 (from the 5′ end) of the first strand would pair with position 7 (from the 5′ end) of the second strand. Position 11 (from the 5′ end) of the first strand would pair with position 9 (from the 5′ end) of the second strand. This nomenclature may be applied to other positions of the second strand.

In one aspect of the double-stranded nucleic acid, in the case of a partially complementary first and second strand, the nucleotide on the second strand that “corresponds to” a position on the first strand may not necessarily form a base pair if that position is the position in which there is a mismatch, but the principle of the nomenclature still applies.

One aspect is a double-stranded nucleic acid as disclosed herein, wherein the nucleotides at positions 2 and 14 from the 5′ end of the first strand are not modified with a 2′-OMe modification, and the nucleotides on the second strand which correspond to position 11, or 13, or 11 and 13, or 11-13 of the first strand are modified with a 2′-F modification.

One aspect is a double-stranded nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are not modified with a 2′-OMe modification.

One aspect is a double-stranded nucleic acid as disclosed herein, wherein the nucleotides at positions 2 and 14 from the 5′ end of the first strand are modified with a 2′-F modification, and the nucleotides on the second strand which correspond to position 11, or 13, or 11 and 13, or 11-13 of the first strand are modified with a 2′-F modification.

One aspect is a double-stranded nucleic acid as disclosed herein, wherein greater than 50% of the nucleotides of the first and/or second strand comprise a 2′-OMe modification, such as greater than 55%, 60%, 65%, 70%, 75%, 80%, or 85%, or more, of the first and/or second strand comprise a 2′-OMe modification.

One aspect is a double-stranded nucleic acid as disclosed herein, wherein greater than 50% of the nucleotides of the first and/or second strand comprise a naturally occurring RNA modification, such as wherein greater than 55%, 60%, 65%, 70%, 75%, 80%, or 85% or more of the first and/or second strands comprise such a modification. Suitable naturally occurring modifications include, as well as 2′-OMe, other 2′ sugar modifications, in particular a 2′-H modification resulting in a DNA nucleotide.

One aspect is a double-stranded nucleic acid as disclosed herein, comprising no more than 20%, such as no more than 15% such as no more than 10%, of nucleotides which have 2′ modifications that are not 2′-OMe modifications on the first and/or second strand.

One aspect is a double-stranded nucleic acid as disclosed herein, wherein the number of nucleotides in the first and/or second strand with a 2′-modification that is not a 2′-OMe modification is no more than 7, more preferably no more than 5, and most preferably no more than 3.

One aspect is a nucleic acid as disclosed herein, comprising no more than 20%, (such as no more than 15% or no more than 10%) of 2′-F modifications. When the nucleic acid is a double-stranded nucleic acid, the nucleic acid preferably comprises no more than 20%, (such as no more than 15% or no more than 10%) of 2′-F modifications on the first and/or second strand.

One aspect is a double-stranded nucleic acid as disclosed herein, wherein the number of nucleotides in the first and/or second strand with a 2′-F modification is no more than 7, more preferably no more than 5, and most preferably no more than 3.

One aspect is a double-stranded nucleic acid as disclosed herein, wherein all nucleotides are modified with a 2′-OMe modification except positions 2 and 14 from the 5′ end of the first strand and the nucleotides on the second strand which correspond to position 11, or 13, or 11 and 13, or 11-13 of the first strand. Preferably the nucleotides that are not modified with 2′-OMe are modified with fluoro at the 2′ position (2′-F modification).

In one embodiment, all nucleotides of the nucleic acid are modified at the 2′ position of the sugar. Preferably, these nucleotides are modified with a 2′-F modification where the modification is not a 2′-OMe modification.

In one aspect, the double-stranded nucleic acid is modified on the first strand with alternating 2′-OMe modifications and 2-F modifications, and positions 2 and 14 (starting from the 5′ end) are modified with 2′-F. Preferably the second strand is modified with 2′-F modifications at nucleotides on the second strand which correspond to position 11, or 13, or 11 and 13, or 11-13 of the first strand. Preferably the second strand is modified with 2′-F modifications at positions 11-13 counting from the 3′ end starting at the first position of the complementary (double-stranded) region, and the remaining modifications are naturally occurring modifications, preferably 2′-OMe. The complementary region at least in this case starts at the first position of the second strand that has a corresponding nucleotide in the first strand, regardless of whether the two nucleotides are able to base pair to each other.

In one aspect of the nucleic acid, each of the nucleotides of the nucleic acid is a modified nucleotide. In one aspect, when the nucleic acid is double stranded, each of the nucleotides of the nucleic acid of the first strand and/or of the second strand is a modified nucleotide.

Unless specifically stated otherwise, herein the nucleotides of the first strand are numbered contiguously starting with nucleotide number 1 at the 5′ end of the first strand. Nucleotides of the second strand are numbered contiguously starting with nucleotide number 1 at the 3′ end of the second strand.

An “odd numbered” nucleotide is a nucleotide numbered with an odd number in a strand in which the nucleotides are numbered contiguously starting either from the indicated end or from the 5′ end of the strand if the end from which the nucleotides are numbered is not indicated. An “even numbered” nucleotide is a nucleotide numbered with an even number in a strand in which the nucleotides are numbered contiguously starting either from the indicated end or from the 5′ end of the strand if the end from which the nucleotides are numbered is not indicated.

One or more nucleotides on the first and/or second strand of a double-stranded nucleic acid as disclosed herein may be modified, to form modified nucleotides. One or more of the odd-numbered nucleotides of the first strand may be modified. One or more of the even-numbered nucleotides of the first strand may be modified by at least a second modification, wherein the at least second modification is different from the modification on the one or more odd nucleotides. At least one of the one or more modified even numbered-nucleotides may be adjacent to at least one of the one or more modified odd-numbered nucleotides.

A plurality of odd-numbered nucleotides in the first strand of a double-stranded nucleic acid as disclosed herein may be modified. A plurality of even-numbered nucleotides in the first strand may be modified by a second modification. The first strand may comprise adjacent nucleotides that are modified by a common modification. The first strand may also comprise adjacent nucleotides that are modified by a second different modification (i.e., the first strand may comprise nucleotides that are adjacent to each other and modified by a first modification as well as other nucleotides that are adjacent to each other and modified by a second modification that is different to the first modification).

One or more of the odd-numbered nucleotides of the second strand (wherein the nucleotides are numbered contiguously starting with nucleotide number 1 at the 3′ end of the second strand) of the double-stranded nucleic acids disclosed herein may be modified by a modification that is different to the modification of the odd-numbered nucleotides on the first strand (wherein the nucleotides are numbered contiguously starting with nucleotide number 1 at the 5′ end of the first strand) and/or one or more of the even-numbered nucleotides of the second strand may be modified by the same modification of the odd-numbered nucleotides of the first strand. At least one of the one or more modified even-numbered nucleotides of the second strand may be adjacent to the one or more modified odd-numbered nucleotides. A plurality of odd-numbered nucleotides of the second strand may be modified by a common modification and/or a plurality of even-numbered nucleotides may be modified by the same modification that is present on the first stand odd-numbered nucleotides. A plurality of odd-numbered nucleotides on the second strand may be modified by a modification that is different from the modification of the first strand odd-numbered nucleotides.

The second strand of a double-stranded nucleic acid as disclosed herein may comprise adjacent nucleotides that are modified by a common modification, which may be a modification that is different from the modification of the odd-numbered nucleotides of the first strand.

In the double-stranded nucleic acids of the invention, each of the odd-numbered nucleotides in the first strand and each of the even-numbered nucleotides in the second strand may be modified with a common modification and each of the even-numbered nucleotides may be modified in the first strand with a different modification and each of the odd-numbered nucleotides may be modified in the second strand with the different modification.

The double-stranded nucleic acids of the invention may have the modified nucleotides of the first strand shifted by at least one nucleotide relative to the unmodified or differently modified nucleotides of the second strand.

One or more or each of the odd numbered-nucleotides of the double-stranded nucleic acids disclosed herein may be modified in the first strand and one or more or each of the even-numbered nucleotides may be modified in the second strand. One or more or each of the alternating nucleotides on either or both strands may be modified by a second modification. One or more or each of the even-numbered nucleotides may be modified in the first strand and one or more or each of the even-numbered nucleotides may be modified in the second strand. One or more or each of the alternating nucleotides on either or both strands may be modified by a second modification. One or more or each of the odd-numbered nucleotides may be modified in the first strand and one or more of the odd-numbered nucleotides may be modified in the second strand by a common modification. One or more or each of the alternating nucleotides on either or both strands may be modified by a second modification. One or more or each of the even-numbered nucleotides may be modified in the first strand and one or more or each of the odd-numbered nucleotides may be modified in the second strand by a common modification. One or more or each of the alternating nucleotides on either or both strands may be modified by a second modification.

The nucleic acids of the invention may comprise single- or double-stranded constructs that comprise at least two regions of alternating modifications in one or both strands, if two strands are present. These alternating regions can comprise up to about 12 nucleotides but preferably comprise from about 3 to about 10 nucleotides. The regions of alternating nucleotides may be located at the termini of one or both strands of the nucleic acid of the invention. The nucleic acid may comprise from 4 to about 10 nucleotides of alternating nucleotides at each of the termini (3′ and 5′) and these regions may be separated by from about 5 to about 12 contiguous unmodified or differently or commonly modified nucleotides.

The odd numbered nucleotides of the first strand of the double-stranded nucleic acids disclosed herein may be modified and the even numbered nucleotides may be modified with a second modification. The second strand may comprise adjacent nucleotides that are modified with a common modification, which may be the same as the modification of the odd-numbered nucleotides of the first strand. One or more nucleotides of the second strand may also be modified with the second modification. One or more nucleotides with the second modification may be adjacent to each other and to nucleotides having a modification that is the same as the modification of the odd-numbered nucleotides of the first strand. The first strand may also comprise phosphorothioate linkages between the two nucleotides at the 3′ end and at the 5′ end or a phosphorodithioate linkage between the two nucleotides at the 3′ end. The second strand may comprise a phosphorothioate or phosphorodithioate linkage between the two nucleotides at the 5′ end. The second strand may also be conjugated to a ligand at the 5′ end.

The double-stranded nucleic acids of the invention may comprise a first strand comprising adjacent nucleotides that are modified with a common modification. One or more such nucleotides may be adjacent to one or more nucleotides which may be modified with a second modification. One or more nucleotides with the second modification may be adjacent. The second strand may comprise adjacent nucleotides that are modified with a common modification, which may be the same as one of the modifications of one or more nucleotides of the first strand. One or more nucleotides of the second strand may also be modified with the second modification. One or more nucleotides with the second modification may be adjacent. The first strand may also comprise phosphorothioate linkages between the two nucleotides at the 3′ end and at the 5′ end or a phosphorodithioate linkage between the two nucleotides at the 3′ end. The second strand may comprise a phosphorothioate or phosphorodithioate linkage between the two nucleotides at the 3′ end. The second strand may also be conjugated to a ligand at the 5′ end.

When the nucleotides of the double-stranded nucleic acid disclosed herein are numbered from 5′ to 3′ on the first strand and 3′ to 5′ on the second strand, nucleotides 1, 3, 5, 7, 9, 11, 13, 15, 17, 19, 21, 23 and 25 may be modified by a modification on the first strand. The nucleotides numbered 2, 4, 6, 8, 10, 12, 14, 16, 18, 20, 22 and 24 may be modified by a second modification on the first strand. The nucleotides numbered 1, 3, 5, 7, 9, 11, 13, 15, 17, 19, 21, 23 may be modified by a modification on the second strand. The nucleotides numbered 2, 4, 6, 8, 10, 12, 14, 16, 18, 20, 22 and 24 may be modified by a second modification on the second strand.

The nucleotides numbered 2, 4, 6, 8, 10, 12, 14, 16, 18, 20, 22 and 24 may be modified by a modification on the first strand. The nucleotides numbered 1, 3, 5, 7, 9, 11, 13, 15, 17, 19, 21, 23 may be modified by a second modification on the first strand. The nucleotides numbered 1, 3, 5, 7, 9, 11, 13, 15, 17, 19, 21, 23 may be modified by a modification on the second strand. The nucleotides numbered 2, 4, 6, 8, 10, 12, 14, 16, 18, 20, 22 and 24 may be modified by a second modification on the second strand.

Clearly, if the first and/or the second strand are shorter than 25 nucleotides in length, such as 19 nucleotides in length, there are no nucleotides numbered 20, 21, 22, 23, 24 and 25 to be modified. The skilled person understands the description above to apply to shorter strands, accordingly.

One or more modified nucleotides on the first strand of a double-stranded nucleic acid as disclosed herein may be paired with modified nucleotides on the second strand having a common modification. One or more modified nucleotides on the first strand may be paired with modified nucleotides on the second strand having a different modification. One or more modified nucleotides on the first strand may be paired with unmodified nucleotides on the second strand. One or more modified nucleotides on the second strand may be paired with unmodified nucleotides on the first strand. In other words, the alternating nucleotides can be aligned on the two strands such as, for example, all the modifications in the alternating regions of the second strand are paired with identical modifications in the first strand or alternatively the modifications can be offset by one nucleotide with the common modifications in the alternating regions of one strand pairing with dissimilar modifications (i.e. a second or further modification) in the other strand. Another option is to have dissimilar modifications in each of the strands.

The modifications on the first strand of a double-stranded nucleic acid as disclosed herein may be shifted by one nucleotide relative to the modified nucleotides on the second strand, such that common modified nucleotides are not paired with each other.

The modification and/or modifications of the nucleic acids disclosed herein may each and individually be selected from the group consisting of 3′ terminal deoxy thymine, 2′-OMe, a 2′ deoxy modification, a 2′ amino modification, a 2′ alkyl modification, a morpholino modification, a phosphoramidate modification, 5′-phosphorothioate group modification, a 5′ phosphate or 5′ phosphate mimic modification and a cholesteryl derivative or a dodecanoic acid bisdecylamide group modification and/or the modified nucleotide may be any one of a locked nucleotide, an abasic nucleotide or a non-natural base comprising nucleotide.

At least one modification may be 2′-OMe and/or at least one modification may be 2′-F. Further modifications as described herein may be present, preferably on the first and/or second strand of a double-stranded nucleic acid.

The nucleic acid of the invention may comprise an inverted RNA nucleotide at one or several of the strand ends. Such inverted nucleotides provide stability to the nucleic acid. Preferably, the nucleic acid comprises at least an inverted nucleotide at the 3′ end. When the nucleic acid is double-stranded it may comprise at least an inverted nucleotide at the 3′ end of the first and/or the second strand and/or at the 5′ end of the second strand. More preferably, the double-stranded nucleic acid comprises an inverted nucleotide at the 3′ end of the second strand. Most preferably, the double-stranded nucleic acid comprises an inverted RNA nucleotide at the 3′ end of the second strand and this nucleotide is preferably an inverted A. An inverted nucleotide is a nucleotide that is linked to the 3′ end of a nucleic acid through its 3′ carbon, rather than its 5′ carbon as would normally be the case or is linked to the 5′ end of a nucleic acid through its 5′ carbon, rather than its 3′ carbon as would normally be the case. The inverted nucleotide is preferably present at an end of a strand not as an overhang but opposite a corresponding nucleotide in the other strand. Accordingly, the nucleic acid is preferably blunt-ended at the end that comprises the inverted RNA nucleotide. An inverted RNA nucleotide being present at the end of a strand preferably means that the last nucleotide at this end of the strand is the inverted RNA nucleotide. A nucleic acid with such a nucleotide is stable and easy to synthesise. The inverted RNA nucleotide is preferably an unmodified nucleotide in the sense that it does not comprise any modifications compared to the natural nucleotide counterpart. Specifically, the inverted RNA nucleotide is preferably a 2′-OH nucleotide.

Nucleic acids of the invention may comprise one or more nucleotides modified at the 2′ position with a 2′-H, and therefore having a DNA nucleotide within the nucleic acid. Double-stranded nucleic acids of the invention may comprise DNA nucleotides at positions 2 and/or 14 of the first strand counting from the 5′ end of the first strand. Nucleic acids may comprise DNA nucleotides on the second strand which correspond to position 11, or 13, or 11 and 13, or 11-13 of the first strand.

In one aspect there is no more than one DNA nucleotide per nucleic acid of the invention.

Nucleic acids of the invention may comprise one or more LNA nucleotides. Double-stranded nucleic acids of the invention may comprise LNA nucleotides at positions 2 and/or 14 of the first strand counting from the 5′ end of the first strand. Nucleic acids may comprise LNA on the second strand which correspond to position 11, or 13, or 11 and 13, or 11-13 of the first strand.

Some representative modified nucleic acid sequences of the present invention are shown in the examples. These examples are meant to be representative and not limiting.

In one embodiment, the nucleic acid may comprise a first modification and a second or further modification which are each and individually selected from the group comprising 2′-OMe modification and 2′-F modification. The nucleic acid may comprise a modification that is 2′-OMe that may be a first modification, and a second modification that is 2′-F. The nucleic acid of the invention may also include a phosphorothioate or phosphorodithioate modification and/or a deoxy modification which may be present in or between the terminal 2 or 3 nucleotides of each or any end of a strand or of both strands if the nucleic acid has two strands.

In one aspect of the double-stranded nucleic acid, at least one nucleotide of the first and/or second strand is a modified nucleotide, wherein if the first strand comprises at least one modified nucleotide:
(i) at least one or both of the nucleotides 2 and 14 of the first strand is/are modified by a first modification; and/or
(ii) at least one, several, or all the even-numbered nucleotides of the first strand is/are modified by a first modification; and/or
(iii) at least one, several, or all the odd-numbered nucleotides of the first strand is/are modified by a second modification; and/or wherein if the second strand comprises at least one modified nucleotide:
(iv) at least one, several, or all the nucleotides of the second strand in a position corresponding to an even-numbered nucleotide of the first strand is/are modified by a third modification; and/or
(v) at least one, several, or all the nucleotides of the second strand in a position corresponding to an odd-numbered nucleotide of the first strand is/are modified by a fourth modification; and/or
(vi) at least one, several, or all the nucleotides of the second strand in a position corresponding to nucleotide 11 or nucleotide 13 or nucleotides 11 and 13 or nucleotides 11-13 of the first strand is/are modified by a fourth modification; and/or
(vii) at least one, several, or all the nucleotides of the second strand in a position other than the position corresponding to nucleotide 11 or nucleotide 13 or nucleotides 11 and 13 or nucleotides 11-13 of the first strand is/are modified by a third modification;
wherein the nucleotides on the first strand are numbered consecutively starting with nucleotide number 1 at the 5′ end of the first strand;
wherein the modifications are preferably at least one of the following:
(a) the first modification is preferably different from the second and from the third modification;
(b) the first modification is preferably the same as the fourth modification;
(c) the second and the third modification are preferably the same modification;
(d) the first modification is preferably a 2′-F modification;
(e) the second modification is preferably a 2′-OMe modification;
(f) the third modification is preferably a 2′-OMe modification; and/or
(g) the fourth modification is preferably a 2′-F modification; and wherein optionally the nucleic acid is conjugated to a ligand.

One aspect is a double-stranded nucleic acid that is capable of inhibiting expression of TMPRSS6 for use in the prevention, decrease of the risk of suffering from or treatment of a myeloproliferative disorder, preferably polycythaemia vera, wherein the nucleic acid comprises a first strand and a second strand, wherein the first strand sequence preferably comprises a sequence of at least 15 nucleotides differing by no more than 3 nucleotides from any one of the sequences selected from SEQ ID NO: 6, 8, 10, 12, 14, 16, 18, 20, 22, 24, 26, 28, 30, 32, 34 and 36, wherein all the even-numbered nucleotides of the first strand are modified by a first modification, all the odd-numbered nucleotides of the first strand are modified by a second modification, all the nucleotides of the second strand in a position corresponding to an even-numbered nucleotide of the first strand are modified by a third modification, all the nucleotides of the second strand in a position corresponding to an odd-numbered nucleotide of the first strand are modified by a fourth modification, wherein the first and fourth modification are 2′-F and the second and third modification are 2′-OMe.

One aspect is a double-stranded nucleic acid that is capable of inhibiting expression of TMPRSS6 for use in the prevention, decrease of the risk of suffering from or treatment of a myeloproliferative disorder, preferably polycythaemia vera, wherein the nucleic acid comprises a first strand and a second strand, wherein the first strand sequence preferably comprises a sequence of at least 15 nucleotides differing by no more than 3 nucleotides from any one of the sequences selected from SEQ ID NO: 6, 8, 10, 12, 14, 16, 18, 20, 22, 24, 26, 28, 30, 32, 34 and 36, wherein all the even-numbered nucleotides of the first strand are modified by a first modification, all the odd-numbered nucleotides of the first strand are modified by a second modification, all the nucleotides of the second strand in positions corresponding to nucleotides 11-13 of the first strand are modified by a fourth modification, all the nucleotides of the second strand other than the nucleotides corresponding to nucleotides 11-13 of the first strand are modified by a third modification, wherein the first and fourth modification are 2′-F and the second and third modification are 2′-OMe.

The 3′ and 5′ ends of an oligonucleotide can be modified. Such modifications can be at the 3′ end or the 5′ end or both ends of the molecule. They can include modification or replacement of an entire terminal phosphate or of one or more of the atoms of the phosphate group. For example, the 3′ and 5′ ends of an oligonucleotide can be conjugated to other functional molecular entities such as labelling moieties, e.g., fluorophores (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs). These spacers or linkers can include e.g., —(CH₂)ₙ—, —(CH₂)ₙN—, —(CH₂)ₙO—, —(CH₂)ₙS—, —(CH₂CH₂O)ₙCH₂CH₂O— (e.g., n=3 or 6), abasic sugars, amide, carboxy, amine, oxyamine, oxyimine, thioether, disulfide, thiourea, sulfonamide, or morpholino, or biotin and fluorescein reagents. The 3′ end can be an —OH group.

Other examples of terminal modifications include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases, EDTA, lipophilic carriers (e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles).

Terminal modifications can also be useful for monitoring distribution, and in such cases the groups to be added may include fluorophores, e.g., fluorescein or an Alexa dye. Terminal modifications can also be useful for enhancing uptake, useful modifications for this include cholesterol. Terminal modifications can also be useful for cross-linking an RNA agent to another moiety.

Terminal modifications can be added for a number of reasons, including to modulate activity or to modulate resistance to degradation. Terminal modifications useful for modulating activity include modification of the 5′ end with phosphate or phosphate analogues. Nucleic acids of the invention, on the first or second strand, may be 5′ phosphorylated or include a phosphoryl analogue at the 5′ prime terminus. 5′-phosphate modifications include those which are compatible with RISC mediated gene silencing. Suitable modifications include: 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-guanosine cap (7-methylated or non-methylated) (7m-G-O-5′-(HO)(O)P—O—(HO)(O)P—O—P(HO)(O)—O-5′); 5′-adenosine cap (Appp), and any modified or unmodified nucleotide cap structure (N—O-5′-(HO)(O)P—O—(HO)(O)P—O—P(HO)(O)—O-5′); 5′-monothiophosphate (phosphorothioate; (HO)₂(S)P—O-5′); 5′-monodithiophosphate (phosphorodithioate; (HO)(HS)(S)P—O-5′), 5′-phosphorothiolate ((HO)₂(O)P—S-5′); any additional combination of oxygen/sulfur replaced monophosphate, diphosphate and triphosphates (e.g., 5′-alpha-thiotriphosphate, 5′-gamma-thiotriphosphate, etc.), 5′-phosphoramidates ((HO)₂(O)P—NH-5′, (HO)(NH₂)(O)P—O-5′), 5′-alkylphosphonates (alkyl=methyl, ethyl, isopropyl, propyl, etc., e.g., RP(OH)(O)—O-5′-(wherein R is an alkyl), (OH)₂(O)P-5′-CH₂—), 5′ vinylphosphonate, 5′-alkyletherphosphonates (alkylether═methoxymethyl (MeOCH₂—), ethoxymethyl, etc., e.g., RP(OH)(O)—O-5′-) (wherein R is an alkylether)).

Certain moieties may be linked to the 5′ terminus of a strand, such as at the 5′ terminus of the first strand or the second strand when the nucleic acid has a first and a second strand. These include abasic ribose moiety, abasic deoxyribose moiety, modifications abasic ribose and abasic deoxyribose moieties including 2′-O alkyl modifications; inverted abasic ribose and abasic deoxyribose moieties and modifications thereof, C6-imino-Pi; a mirror nucleotide including L-DNA and L-RNA; 5′OMe nucleotide; and nucleotide analogues including 4′,5′-methylene nucleotide; 1-(β-D-erythrofuranosyl)nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 12-aminododecyl phosphate; hydroxypropyl phosphate; 1,5-anhydrohexitol nucleotide; alpha-nucleotide; threo-pentofuranosyl nucleotide; acyclic 3′,4′-seco nucleotide; 3,4-dihydroxybutyl nucleotide; 3,5-dihydroxypentyl nucleotide, 5′-5′-inverted abasic moiety; 1,4-butanediol phosphate; 5′-amino; and bridging or non-bridging methylphosphonate and 5′-mercapto moieties.

In each sequence described herein, a C-terminal “—OH” moiety may be substituted for a C-terminal “—NH₂” moiety, and vice-versa.

The invention also provides a nucleic acid according to any aspect of the invention described herein, wherein the nucleic acid has a terminal 5′ (E)-vinylphosphonate nucleotide at its 5′ end. In this embodiment, when the nucleic acid is double-stranded the first strand of the nucleic acid has a terminal 5′ (E)-vinylphosphonate nucleotide at its 5′ end. This terminal 5′ (E)-vinylphosphonate nucleotide is preferably linked to the second nucleotide in the first strand by a phosphodiester linkage.

The nucleic acid, or the first strand of the nucleic acid when the nucleic acid is double-stranded, may comprise formula (I):

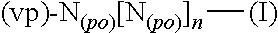

where ‘(vp)-’ is the 5′ (E)-vinylphosphonate, ‘N’ is a nucleotide, ‘po’ is a phosphodiester linkage, and n is from 1 to (the total number of nucleotides in the strand—2), preferably wherein n is from 1 to (the total number of nucleotides in the strand −3), more preferably wherein n is from 1 to (the total number of nucleotides in the strand −4).

In one embodiment, the terminal 5′ (E)-vinylphosphonate nucleotide is an RNA nucleotide, preferably a (vp)-U.

A terminal 5′ (E)-vinylphosphonate nucleotide is a nucleotide wherein the natural phosphate group at the 5′-end has been replaced with a E-vinylphosphonate, in which the bridging 5′-oxygen atom of the terminal nucleotide of the 5′ phosphorylated strand is replaced with a methynyl (—CH═) group:

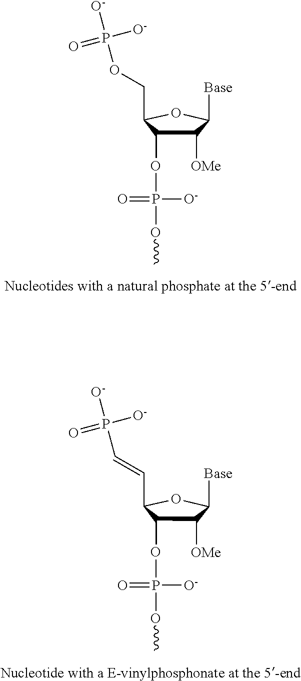

A 5′ (E)-vinylphosphonate is a 5′ phosphate mimic. A biological mimic is a molecule that is capable of carrying out the same function as and is structurally very similar to the original molecule that is being mimicked. In the context of the present invention, 5′ (E)-vinylphosphonate mimics the function of a normal 5′ phosphate, e.g. enabling efficient RISC loading. In addition, because of its slightly altered structure, 5′ (E) vinylphosphonate is capable of stabilizing the 5′-end nucleotide by protecting it from dephosphorylation by enzymes such as phosphatases.

In one aspect of the double-stranded nucleic acid, the first strand has a terminal 5′ (E)-vinylphosphonate nucleotide at its 5′ end, the terminal 5′ (E)-vinylphosphonate nucleotide is linked to the second nucleotide in the first strand by a phosphodiester linkage and the first strand comprises a) more than 1 phosphodiester linkage; b) phosphodiester linkages between at least the terminal three 5′ nucleotides and/or c) phosphodiester linkages between at least the terminal four 5′ nucleotides.

In one aspect, the nucleic acid comprises at least one phosphorothioate (ps) and/or at least one phosphorodithioate (ps2) linkage between two nucleotides.

In one aspect of the double-stranded nucleic acid, the first strand and/or the second strand of the nucleic acid comprises at least one phosphorothioate (ps) and/or at least one phosphorodithioate (ps2) linkage between two nucleotides.

In one aspect, the nucleic acid comprises more than one phosphorothioate and/or more than one phosphorodithioate linkage.

In one aspect of the double-stranded nucleic acid, the first strand and/or the second strand of the nucleic acid comprises more than one phosphorothioate and/or more than one phosphorodithioate linkage.

In one aspect of the double-stranded nucleic acid, the first strand and/or the second strand comprises a phosphorothioate or phosphorodithioate linkage between the terminal two 3′ nucleotides or phosphorothioate or phosphorodithioate linkages between the terminal three 3′ nucleotides. Preferably, the linkages between the other nucleotides in the first strand and/or the second strand are phosphodiester linkages.

In one aspect, the nucleic acid comprises a phosphorothioate linkage between the terminal two 5′ nucleotides or a phosphorothioate linkages between the terminal three 5′ nucleotides.

In one aspect of the double-stranded nucleic acid, the first strand and/or the second strand of the nucleic acid comprises a phosphorothioate linkage between the terminal two 5′ nucleotides or a phosphorothioate linkages between the terminal three 5′ nucleotides.

In one aspect of the double-stranded nucleic acid, the nucleic acid comprises one or more phosphorothioate or phosphorodithioate modifications on one or more of the terminal ends of the first and/or the second strand. Optionally, each or either end of the first strand may comprise one or two or three phosphorothioate or phosphorodithioate modified nucleotides (internucleotide linkage). Optionally, each or either end of the second strand may comprise one or two or three phosphorothioate or phosphorodithioate modified nucleotides (internucleotide linkages).

In one aspect of the double-stranded nucleic acid, the nucleic acid comprises a phosphorothioate linkage between the terminal two or three 3′ nucleotides and/or 5′ nucleotides of the first and/or the second strand. Preferably, the nucleic acid comprises a phosphorothioate linkage between each of the terminal three 3′ nucleotides and the terminal three 5′ nucleotides of the first strand and of the second strand. Preferably, all remaining linkages between nucleotides of the first and/or of the second strand are phosphodiester linkages.

In one aspect of the double-stranded nucleic acid, the nucleic acid comprises a phosphorodithioate linkage between each of the two, three or four terminal nucleotides at the 3′ end of the first strand and/or comprises a phosphorodithioate linkage between each of the two, three or four terminal nucleotides at the 3′ end of the second strand and/or a phosphorodithioate linkage between each of the two, three or four terminal nucleotides at the 5′ end of the second strand and comprises a linkage other than a phosphorodithioate linkage between the two, three or four terminal nucleotides at the 5′ end of the first strand.

In one aspect of the double-stranded nucleic acid, the nucleic acid comprises a phosphorothioate linkage between the terminal three 3′ nucleotides and the terminal three 5′ nucleotides of the first strand and of the second strand. Preferably, all remaining linkages between nucleotides of the first and/or of the second strand are phosphodiester linkages.

In one aspect of the double-stranded nucleic acid, the nucleic acid:
(i) has a phosphorothioate linkage between the terminal three 3′ nucleotides and the terminal three 5′ nucleotides of the first strand;
(ii) is conjugated to a triantennary ligand either on the 3′ end nucleotide or on the 5′ end nucleotide of the second strand;
(iii) has a phosphorothioate linkage between the terminal three nucleotides of the second strand at the end opposite to the one conjugated to the triantennary ligand; and
(iv) optionally all remaining linkages between nucleotides of the first and/or of the second strand are phosphodiester linkages.

In one aspect of the double-stranded nucleic acid, the nucleic acid:
(i) has a terminal 5′ (E)-vinylphosphonate nucleotide at the 5′ end of the first strand;
(ii) has a phosphorothioate linkage between the terminal three 3′ nucleotides on the first and second strand and between the terminal three 5′ nucleotides on the second strand or it has a phosphorodithioate linkage between the terminal two 3′ nucleotides on the first and second strand and between the terminal two 5′ nucleotides on the second strand; and
(iii) optionally all remaining linkages between nucleotides of the first and/or of the second strand are phosphodiester linkages.

The use of a phosphorodithioate linkage in the nucleic acid of the invention reduces the variation in the stereochemistry of a population of nucleic acid molecules compared to molecules comprising a phosphorothioate in that same position. Phosphorothioate linkages introduce chiral centres and it is difficult to control which non-linking oxygen is substituted for sulphur. The use of a phosphorodithioate ensures that no chiral centre exists in that linkage and thus reduces or eliminates any variation in the population of nucleic acid molecules, depending on the number of phosphorodithioate and phosphorothioate linkages used in the nucleic acid molecule.

In one aspect of the double-stranded nucleic acid, the nucleic acid comprises a phosphorodithioate linkage between the two terminal nucleotides at the 3′ end of the first strand and a phosphorodithioate linkage between the two terminal nucleotides at the 3′ end of the second strand and a phosphorodithioate linkage between the two terminal nucleotides at the 5′ end of the second strand and comprises a linkage other than a phosphorodithioate linkage between the two, three or four terminal nucleotides at the 5′ end of the first strand. Preferably, the first strand has a terminal 5′ (E)-vinylphosphonate nucleotide at its 5′ end. This terminal 5′ (E)-vinylphosphonate nucleotide is preferably linked to the second nucleotide in the first strand by a phosphodiester linkage. Preferably, all the linkages between the nucleotides of both strands other than the linkage between the two terminal nucleotides at the 3′ end of the first strand and the linkages between the two terminal nucleotides at the 3′ end and at the 5′ end of the second strand are phosphodiester linkages.

In one aspect of the double-stranded nucleic acid, the nucleic acid comprises a phosphorothioate linkage between each of the three terminal 3′ nucleotides and/or between each of the three terminal 5′ nucleotides on the first strand, and/or between each of the three terminal 3′ nucleotides and/or between each of the three terminal 5′ nucleotides of the second strand when there is no phosphorodithioate linkage present at that end. No phosphorodithioate linkage being present at an end means that the linkage between the two terminal nucleotides, or preferably between the three terminal nucleotides of the nucleic acid end in question are linkages other than phosphorodithioate linkages.

In one aspect of the double-stranded nucleic acid, all the linkages of the nucleic acid between the nucleotides of both strands other than the linkage between the two terminal nucleotides at the 3′ end of the first strand and the linkages between the two terminal nucleotides at the 3′ end and at the 5′ end of the second strand are phosphodiester linkages.

Other phosphate linkage modifications are possible.

The phosphate linker can also be modified by replacement of a linking oxygen with nitrogen (bridged phosphoroamidates), sulfur (bridged phosphorothioates) and carbon (bridged methylenephosphonates). The replacement can occur at a terminal oxygen. Replacement of the non-linking oxygens with nitrogen is possible.

The phosphate groups can also individually be replaced by non-phosphorus containing connectors.

Examples of moieties which can replace the phosphate group include siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino. In certain embodiments, replacements may include the methylenecarbonylamino and methylenemethylimino groups.

The phosphate linker and ribose sugar may be replaced by nuclease resistant nucleotides. Examples include the morpholino, cyclobutyl, pyrrolidine and peptide nucleic acid (PNA) nucleoside surrogates. In certain embodiments, PNA surrogates may be used.

In one aspect, the nucleic acid, which is preferably an siRNA that inhibits expression of TMPRSS6, preferably via RNAi, comprises one or more or all of:
(i) a modified nucleotide;
(ii) a modified nucleotide other than a 2′-OMe modified nucleotide at positions 2 and 14 from the 5′ end of the first strand, preferably a 2′-F modified nucleotide;
(iii) each of the odd-numbered nucleotides of the first strand as numbered starting from one at the 5′ end of the first strand are 2′-OMe modified nucleotides;
(iv) each of the even-numbered nucleotides of the first strand as numbered starting from one at the 5′ end of the first strand are 2′-F modified nucleotides;
(v) the second strand nucleotide corresponding to position 11 and/or 13 or 11-13 of the first strand is modified by a modification other than a 2′-OMe modification, preferably wherein one or both or all of these positions comprise a 2′-F modification;
(vi) an inverted nucleotide, preferably a 3′-3′ linkage at the 3′ end of the second strand;
(vii) one or more phosphorothioate linkages;
(viii) one or more phosphorodithioate linkages; and/or
(ix) the first strand has a terminal 5′ (E)-vinylphosphonate nucleotide at its 5′ end, in which case the terminal 5′ (E)-vinylphosphonate nucleotide is preferably a uridine and is preferably linked to the second nucleotide in the first strand by a phosphodiester linkage.

All the features of the nucleic acids can be combined with all other aspects of the invention disclosed herein.

### Ligands

The inhibitors or nucleic acids of the invention may be conjugated to a ligand. Efficient delivery of oligonucleotides, in particular double-stranded nucleic acids of the invention, to cells in vivo is important and requires specific targeting and substantial protection from the extracellular environment, preferably serum proteins. One method of achieving specific targeting is to conjugate a ligand to the nucleic acid. In some embodiments, the ligand helps in targeting the nucleic acid to a target cell which has a cell surface receptor that binds to and internalises the conjugated ligand. In such embodiments, there is a need to conjugate appropriate ligands for the desired receptor molecules in order for the conjugated molecules to be taken up by the target cells by mechanisms such as different receptor-mediated endocytosis pathways or functionally analogous processes. In other embodiments, a ligand which can mediate internalization of the nucleic acid into a target cell by mechanisms other than receptor mediated endocytosis may alternatively be conjugated to a nucleic acid of the invention for cell or tissue specific targeting.

One example of a conjugate that mediates receptor mediated endocytosis is the asialoglycoprotein receptor complex (ASGP-R) which has high affinity to the GalNAc moiety described herein. The ASGP-R complex is composed of varying ratios of multimers of membrane ASGR1 and ASGR2 receptors, which are highly abundant on hepatocytes. One of the first disclosures of the use of triantennary cluster glycosides as conjugated ligands was in U.S. Pat. No. 5,885,968. Conjugates having three GalNAc ligands and comprising phosphate groups are known and are described in Dubber et al. (Bioconjug. Chem. 2003 January-February; 14(1):239-46.). The ASGP-R complex shows a 50-fold higher affinity for N-Acetyl-D-Galactosamine (GalNAc) than D-Gal.

The ASGP-R complex recognizes specifically terminal β-galactosyl subunits of glycosylated proteins or other oligosaccharides (Weigel, P. H. et. al., Biochim. Biophys. Acta. 2002 Sep. 19; 1572(2-3):341-63) and can be used for delivering a drug to the liver's hepatocytes expressing the receptor complex by covalent coupling of galactose or galactosamine to the drug substance (Ishibashi, S.; et. al., J Biol. Chem. 1994 Nov. 11; 269(45):27803-6). Furthermore, the binding affinity can be significantly increased by the multi-valency effect, which is achieved by the repetition of the targeting moiety (Biessen E A, et al., J Med Chem. 1995 Apr. 28; 38(9):1538-46).

The ASGP-R complex is a mediator for an active uptake of terminal β-galactosyl containing glycoproteins to the cell's endosomes. Thus, the ASGPR is highly suitable for targeted delivery of drug candidates conjugated to such ligands like, e.g., nucleic acids into receptor-expressing cells (Akinc et al., Mol Ther. 2010 July; 18(7):1357-64).

More generally the ligand can comprise a saccharide that is selected to have an affinity for at least one type of receptor on a target cell. In particular, the receptor is on the surface of a mammalian liver cell, for example, the hepatic asialoglycoprotein receptor complex described before (ASGP-R).

The saccharide may be selected from N-acetyl galactosamine, mannose, galactose, glucose, glucosamine and fucose. The saccharide may be N-acetyl galactosamine (GalNAc).

A ligand for use in the present invention may therefore comprise (i) one or more N-acetyl galactosamine (GalNAc) moieties and derivatives thereof, and (ii) a linker, wherein the linker conjugates the GalNAc moieties to a nucleic acid as defined in any preceding aspects. The linker may be a monovalent structure or bivalent or trivalent or tetravalent branched structure. The nucleotides may be modified as defined herein.

The ligand may therefore comprise GalNAc.

In one aspect, the nucleic acid is conjugated to a ligand comprising a compound of formula (II):

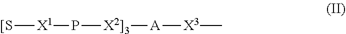

wherein:
S represents a saccharide, preferably wherein the saccharide is N-acetyl galactosamine;
X¹ represents C₃-C₆ alkylene or (—CH₂—CH₂—O)ₘ(—CH₂)₂— wherein m is 1, 2, or 3;
P is a phosphate or modified phosphate, preferably a thiophosphate;
X² is alkylene or an alkylene ether of the formula (—CH₂)ₙ—O—CH₂— where n=1-6;
A is a branching unit;
X³ represents a bridging unit;
wherein a nucleic acid according to the present invention is conjugated to X³ via a phosphate or modified phosphate, preferably a thiophosphate.

In formula (II), the branching unit “A” preferably branches into three in order to accommodate three saccharide ligands. The branching unit is preferably covalently attached to the remaining tethered portions of the ligand and the nucleic acid. The branching unit may comprise a branched aliphatic group comprising groups selected from alkyl, amide, disulphide, polyethylene glycol, ether, thioether and hydroxyamino groups. The branching unit may comprise groups selected from alkyl and ether groups.

The branching unit A may have a structure selected from:

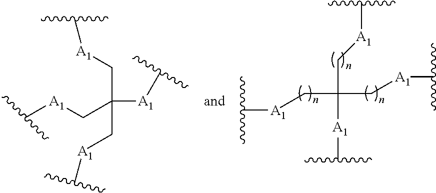

wherein each A₁ independently represents O, S, C═O or NH; and each n independently represents an integer from 1 to 20.

The branching unit may have a structure selected from:

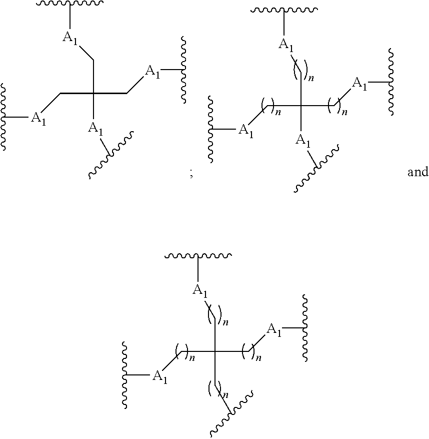

wherein each A₁ independently represents O, S, C═O or NH; and each n independently represents an integer from 1 to 20.

The branching unit may have a structure selected from:

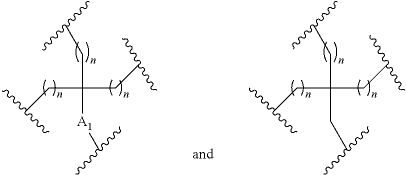

wherein A₁ is O, S, C═O or NH; and each n independently represents an integer from 1 to 20. The branching unit may have the structure:

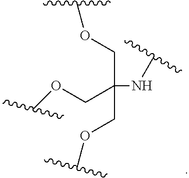 [SMILES: *NC(CO*)(CO*)CO*]

The branching unit may have the structure:

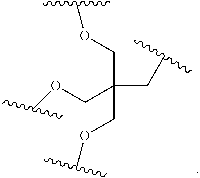 [SMILES: *CC(CO*)(CO*)CO*]

The branching unit may have the structure:

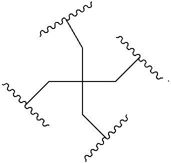 [SMILES: *CC(C*)(C*)C*]

Alternatively, the branching unit A may have a structure selected from:

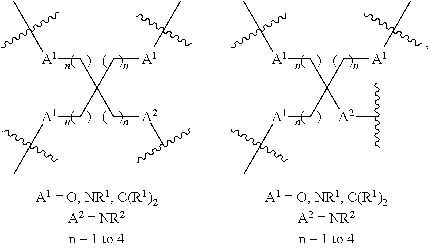

wherein:
R1 is hydrogen or C1-C10 alkylene;
and R2 is C1-C10 alkylene.

Optionally, the branching unit consists of only a carbon atom.

The “X³” portion is a bridging unit. The bridging unit is linear and is covalently bound to the branching unit and the nucleic acid.

X³ may be selected from —C₁-C₂₀ alkylene-, —C₂-C₂₀ alkenylene-, an alkylene ether of formula —(C₁-C₂₀ alkylene)-O—(C₁-C₂₀ alkylene)-, —C(O)—C₁-C₂₀ alkylene-, —C₀-C₄ alkylene(Cy)C₀-C₄ alkylene—wherein Cy represents a substituted or unsubstituted 5 or 6 membered cycloalkylene, arylene, heterocyclylene or heteroarylene ring, —C₁-C₄ alkylene-NHC(O)—C₁-C₄ alkylene-, —C₁-C₄ alkylene-C(O)NH—C₁-C₄ alkylene-, —C₁-C₄ alkylene-SC(O)—C₁-C₄ alkylene-, —C₁-C₄ alkylene-C(O)S—C₁-C₄ alkylene-, —C₁-C₄ alkylene-OC(O)—C₁-C₄ alkylene-, —C₁-C₄ alkylene-C(O)O—C₁-C₄ alkylene-, and —C₁-C₆ alkylene-S—S—C₁-C₆ alkylene —.

X³ may be an alkylene ether of formula —(C₁-C₂₀ alkylene)-O—(C₁-C₂₀ alkylene)—. X³ may be an alkylene ether of formula —(C₁-C₂₀ alkylene)-O—(C₄-C₂₀ alkylene)-, wherein said (C₄-C₂₀ alkylene) is linked to Z. X³ may be selected from the group consisting of —CH₂—O—C₃H₆-, —CH₂—O—C₄H₈-, —CH₂—O—C₆H₁₂—and —CH₂—O—C₈H₁₆-, especially—CH₂—O—C₄H₈-, —CH₂—O—C₆H₁₂—and —CH₂—O—C₈H₁₆-, wherein in each case the —CH₂— group is linked to A.

In one aspect, the nucleic acid is conjugated to a ligand comprising a compound of formula (III):

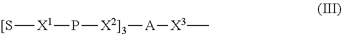

wherein:
S represents a saccharide, preferably GalNAc;
X¹ represents C₃-C₆ alkylene or (—CH₂—CH₂—O)ₘ(—CH₂)₂— wherein m is 1, 2, or 3;
P is a phosphate or modified phosphate, preferably a thiophosphate;
X² is C₁-C₈ alkylene;
A is a branching unit selected from:

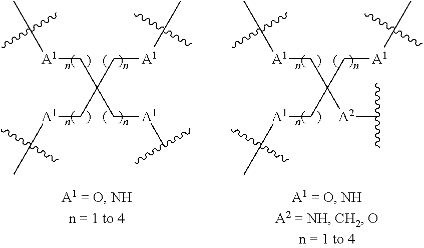

X³ is a bridging unit;
wherein a nucleic acid according to the present invention is conjugated to X³ via a phosphate or a modified phosphate, preferably a thiophosphate.

The branching unit A may have the structure:

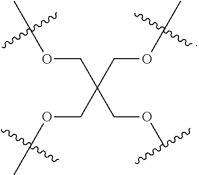 [SMILES: *OCC(CO*)(CO*)CO*]

The branching unit A may have the structure:

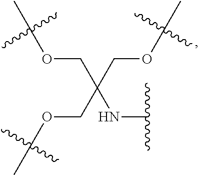

wherein X³ is attached to the nitrogen atom.

X³ may be C₁-C₂₀ alkylene. Preferably, X³ is selected from the group consisting of —C₃H₆—, —C₄H₈—, —C₆H₁₂— and —C₈H₁₆—, especially —C₄H₈—, —C₆H₁₂— and —C₈H₁₆—.

In one aspect, the nucleic acid is conjugated to a ligand comprising a compound of formula (IV):

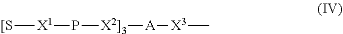

wherein:
S represents a saccharide, preferably GalNAc;
X¹ represents C₃-C₆ alkylene or (—CH₂—CH₂—O)ₘ(—CH₂)₂— wherein m is 1, 2, or 3;
P is a phosphate or modified phosphate, preferably a thiophosphate;
X² is an alkylene ether of formula —C₃H₆—O—CH₂—;
A is a branching unit;
X³ is an alkylene ether of formula selected from the group consisting of —CH₂—O—CH₂—, —CH₂—O—C₂H₄—, —CH₂—O—C₃H₆—, —CH₂—O—C₄H₈—, —CH₂—O—C₅H₁₀—, —CH₂—O—C₆H₁₂—, —CH₂—O—C₇H₁₄—, and —CH₂—O—C₈H₁₆—, wherein in each case the —CH₂— group is linked to A, and wherein X³ is conjugated to a nucleic acid according to the present invention by a phosphate or modified phosphate, preferably a thiophosphate.

The branching unit may comprise carbon. Preferably, the branching unit is a carbon.

X³ may be selected from the group consisting of —CH₂—O—C₄H₈—, —CH₂—O—C₅H₁₀—, —CH₂—O—C₆H₁₂—, —CH₂—O—C₇H₁₄—, and —CH₂—O—C₈H₁₆—. Preferably, X³ is selected from the group consisting of —CH₂—O—C₄H₈—, —CH₂—O—C₆H₁₂— and —CH₂—O—C₈H₁₆.

X¹ may be (—CH₂—CH₂—O)(—CH₂)₂—. X¹ may be (—CH₂—CH₂—O)₂(—CH₂)₂—. X¹ may be (—CH₂—CH₂—O)₃(—CH₂)₂—. Preferably, X¹ is (—CH₂—CH₂—O)₂(—CH₂)₂—. Alternatively, X¹ represents C₃-C₆ alkylene. X¹ may be propylene. X¹ may be butylene. X¹ may be pentylene. X¹ may be hexylene. Preferably the alkyl is a linear alkylene. In particular, X¹ may be butylene.

X² represents an alkylene ether of formula —C₃H₆—O—CH₂—i.e. C₃ alkoxy methylene, or —CH₂CH₂CH₂OCH₂—.

For any of the above aspects, when P represents a modified phosphate group, P can be represented by:

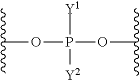

wherein Y¹ and Y² each independently represent ═O, ═S, —O—, —OH, —SH, —BH₃, —OCH₂CO₂, OCH₂CO₂R^{x}, —OCH₂C(S)OR^{x}, and —OR^{x}, wherein R^{x} represents C₁-C₆ alkyl and wherein indicates attachment to the remainder of the compound.

By modified phosphate it is meant a phosphate group wherein one or more of the non-linking oxygens is replaced. Examples of modified phosphate groups include phosphorothioate, phosphorodithioates, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulphur. One, each or both non-linking oxygens in the phosphate group can be independently any one of S, Se, B, C, H, N, or OR (R is alkyl or aryl).

The phosphate can also be modified by replacement of a linking oxygen with nitrogen (bridged phosphoroamidates), sulfur (bridged phosphorothioates) and carbon (bridged methylenephosphonates). The replacement can occur at a terminal oxygen. Replacement of the non-linking oxygens with nitrogen is possible.

For example, Y¹ may represent —OH and Y² may represent=O or ═S; or
Y¹ may represent —O— and Y² may represent=O or ═S;
Y¹ may represent=O and Y² may represent —CH₃, —SH, —OR^{x}, or —BH₃
Y¹ may represent ═S and Y² may represent —CH₃, OR^{x} or —SH.

It will be understood by the skilled person that in certain instances there will be delocalisation between Y¹ and Y².

In one embodiment, the modified phosphate group is a thiophosphate group. Thiophosphate groups include bithiophosphate (i.e. where Y¹ represents ═S and Y² represents—S—) and monothiophosphate (i.e. where Y¹ represents —O— and Y² represents ═S, or where Y¹ represents=O and Y² represents—S—). Preferably, P is a monothiophosphate. The inventors have found that conjugates having thiophosphate groups in replacement of phosphate groups have improved potency and duration of action in vivo.

P may also be an ethylphosphate (i.e. where Y¹ represents=O and Y² represents OCH₂CH₃).

The saccharide may be selected to have an affinity for at least one type of receptor on a target cell. In particular, the receptor is on the surface of a mammalian liver cell, for example, the hepatic asialoglycoprotein receptor complex (ASGP-R).

For any of the above or below aspects, the saccharide may be selected from N-acetyl with one or more of galactosamine, mannose, galactose, glucose, glucosamine and fructose. Typically a ligand to be used in the present invention may include N-acetyl galactosamine (GalNAc). Preferably the compounds of the invention may have 3 ligands, which will each preferably include N-acetyl galactosamine.

“GalNAc” refers to 2-(Acetylamino)-2-deoxy-D-galactopyranose, commonly referred to in the literature as N-acetyl galactosamine. Reference to “GalNAc” or “N-acetyl galactosamine” includes both the β— form: 2-(Acetylamino)-2-deoxy-β-D-galactopyranose and the α-form: 2-(Acetylamino)-2-deoxy-α-D-galactopyranose. In certain embodiments, both the β-form: 2-(Acetylamino)-2-deoxy-β-D-galactopyranose and α-form: 2-(Acetylamino)-2-deoxy-α-D-galactopyranose may be used interchangeably. Preferably, the compounds of the invention comprise the β-form, 2-(Acetylamino)-2-deoxy-β-D-galactopyranose.

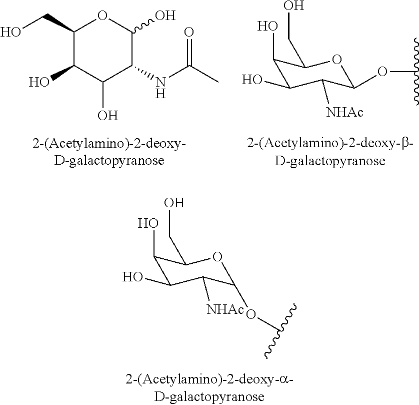

In one aspect, the nucleic acid is a conjugated nucleic acid, wherein the nucleic acid is conjugated to a triantennary ligand with one of the following structures:

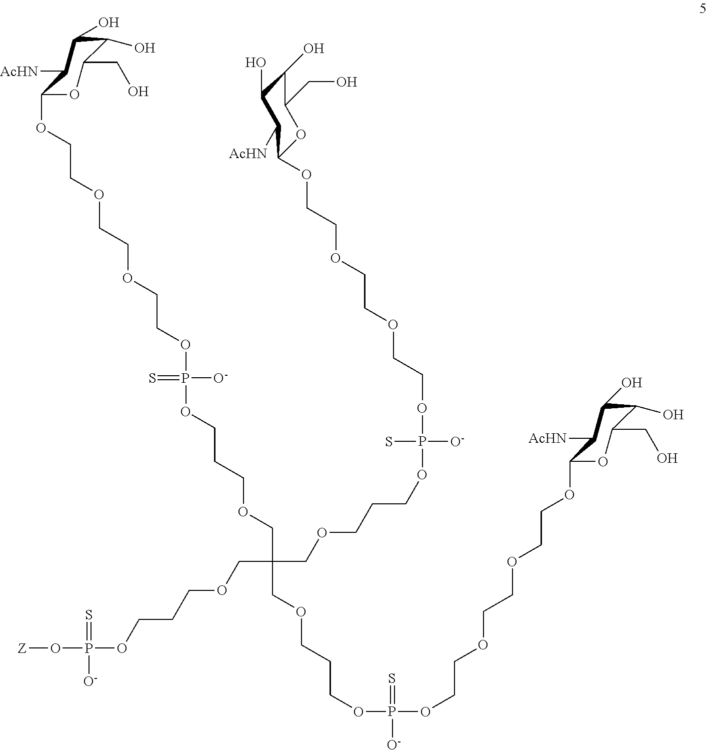

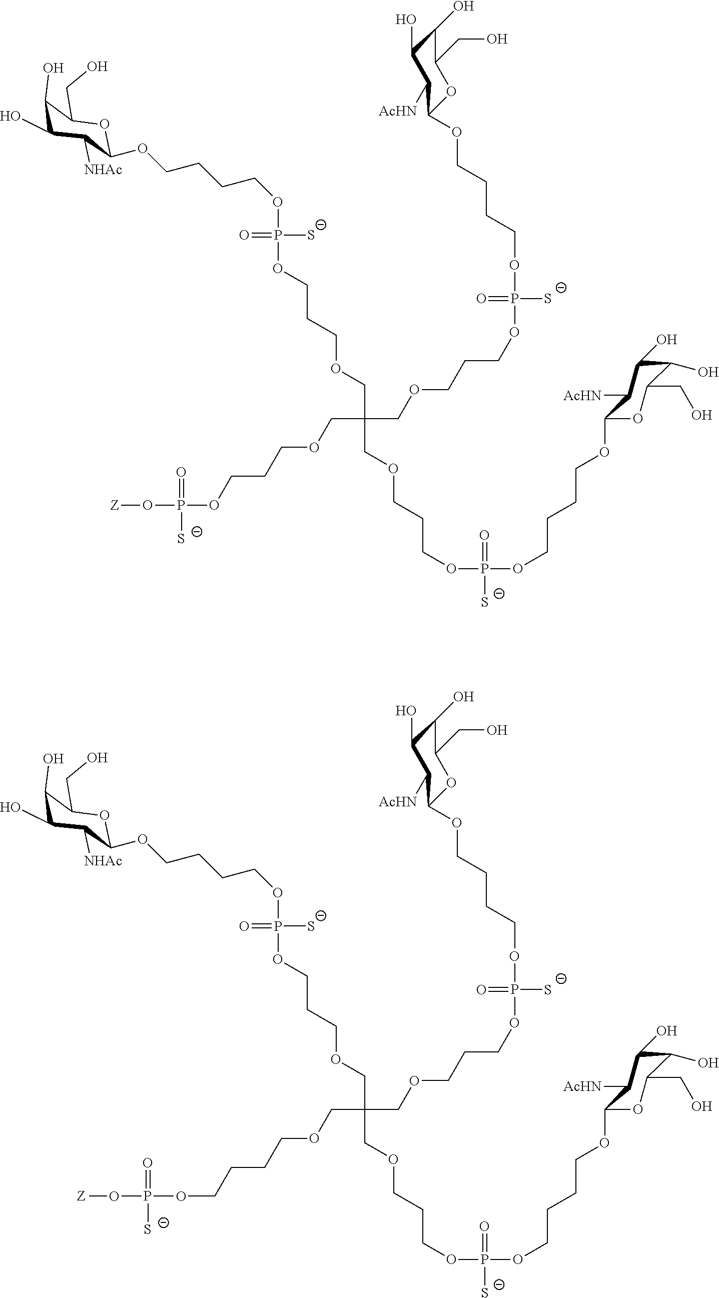

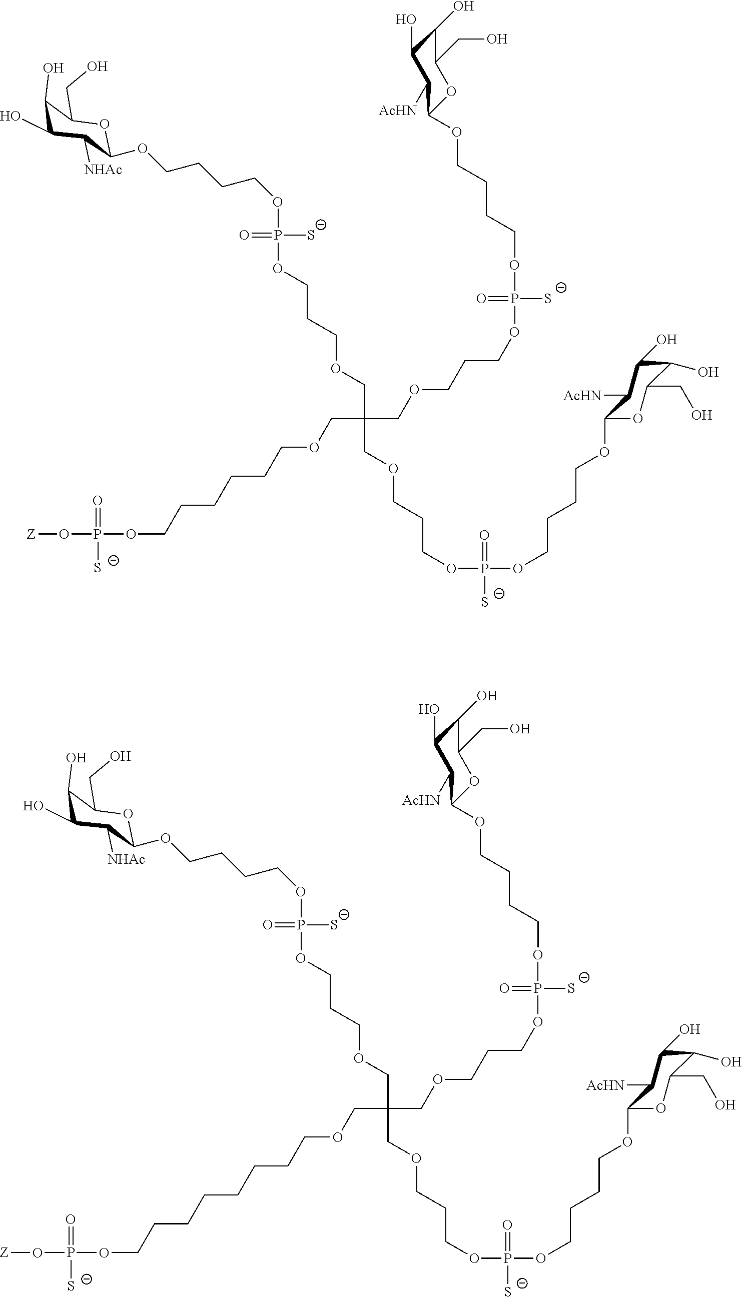

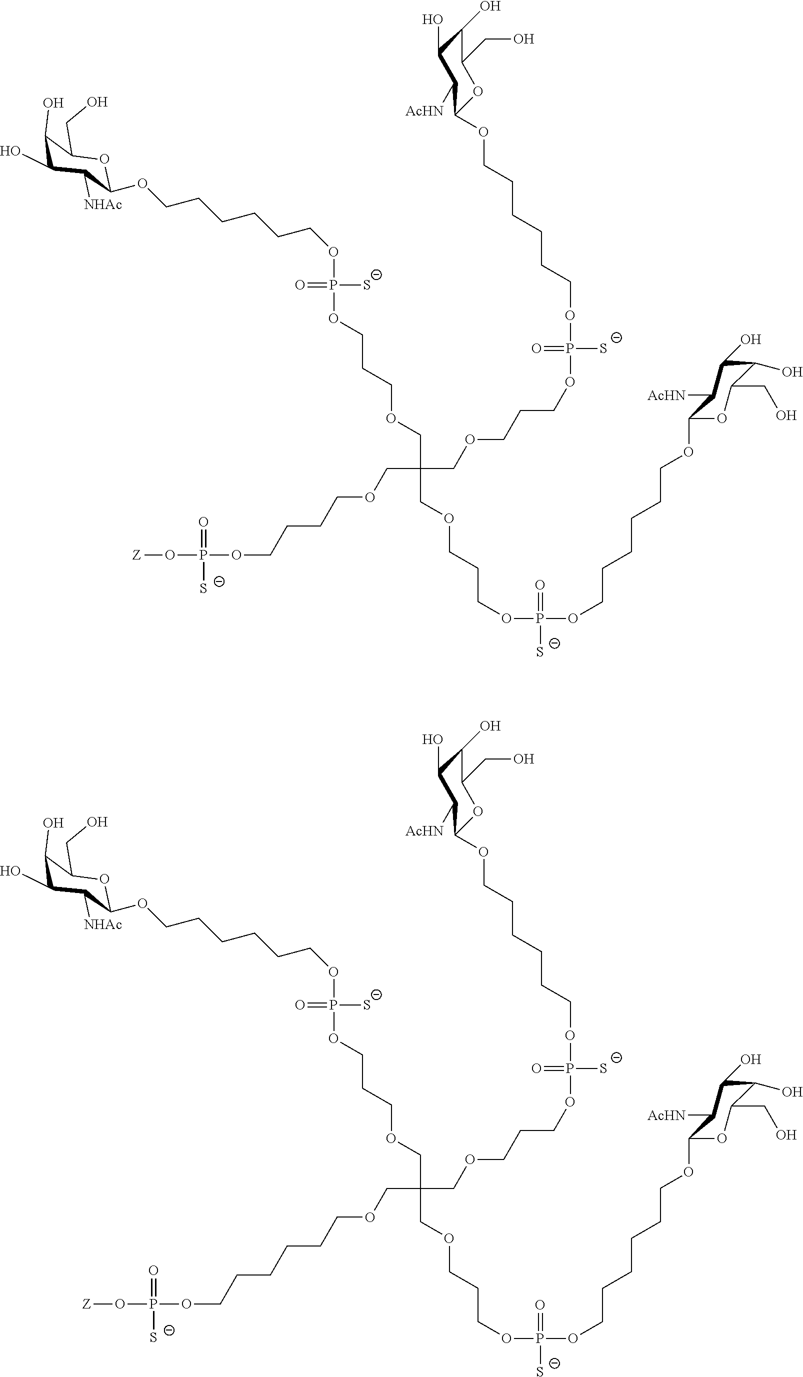

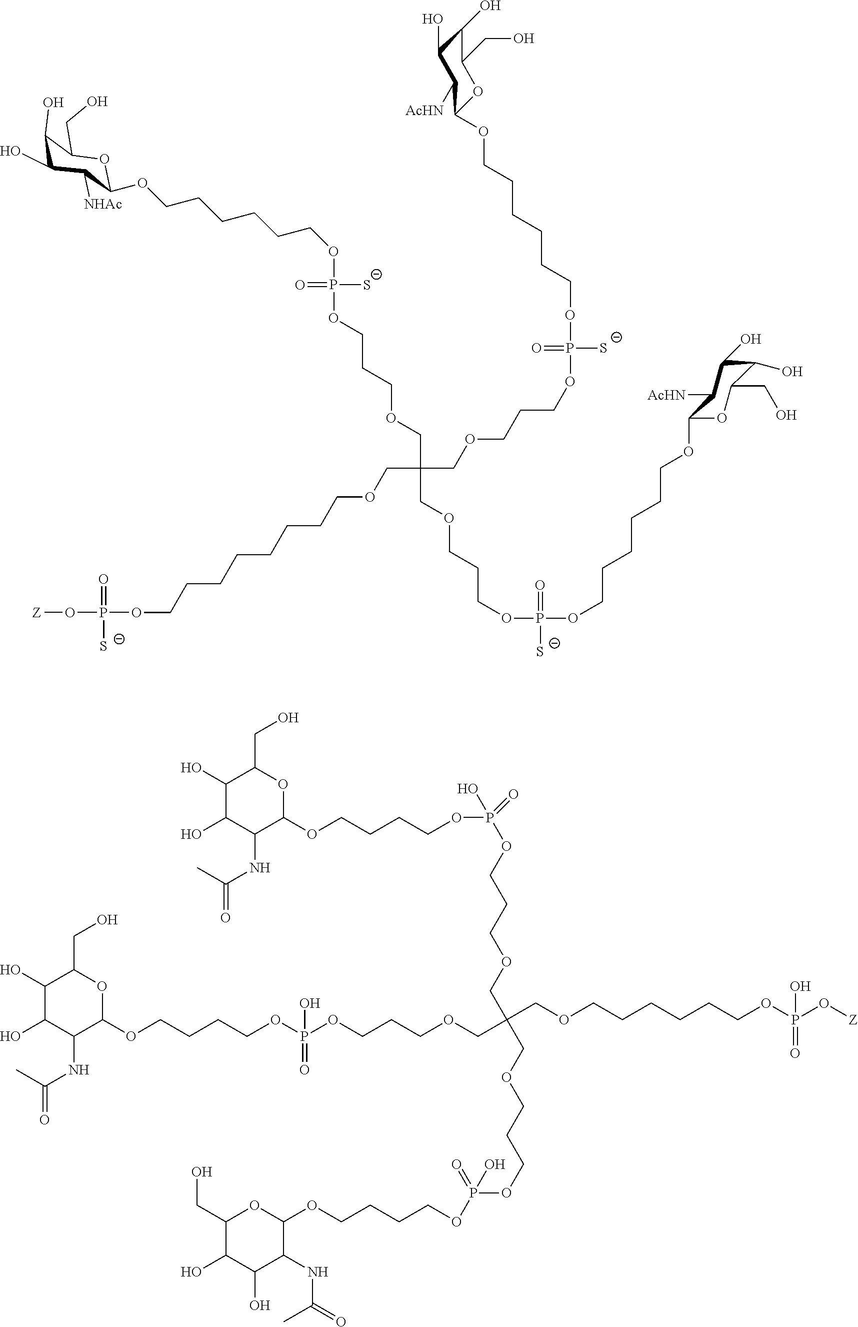

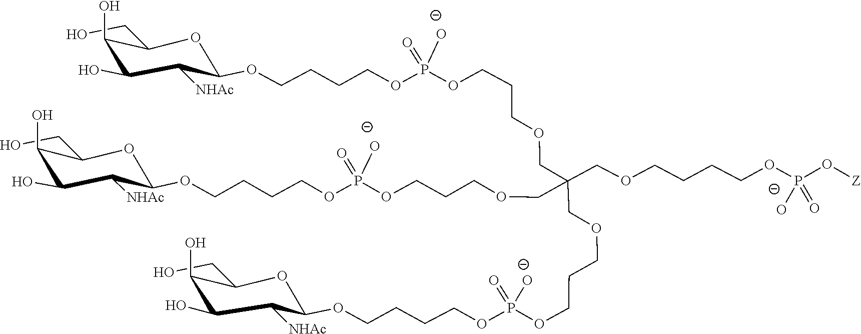

wherein Z is any nucleic acid as defined herein.

In one embodiment, the nucleic acid is a conjugated nucleic acid, wherein the nucleic acid is conjugated to a triantennary ligand with the following structures:

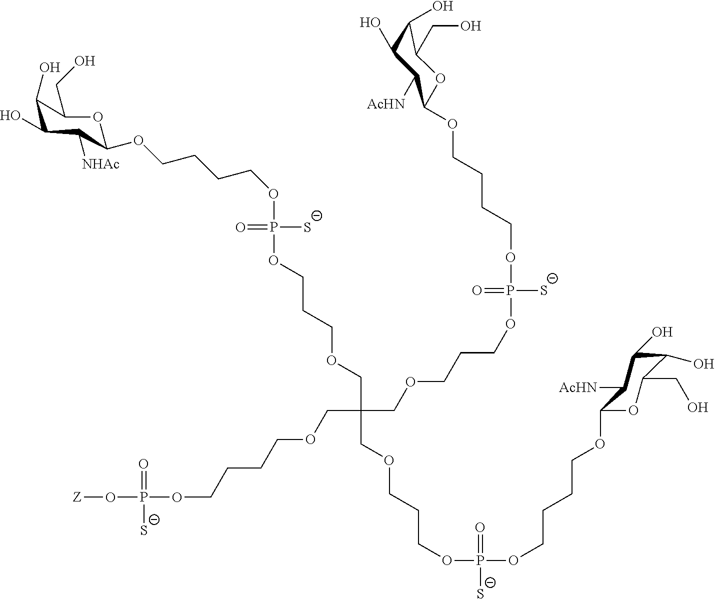

wherein Z is any nucleic acid as defined herein and wherein the terminal phosphorothioate group of the ligand moiety is bonded to the 5′position of the 5′terminal nucleotide of the second strand of the nucleic acid (which is denoted by the “Z”) or wherein the terminal phosphorothioate group of the ligand moiety is bonded to the 3′position of the 3′terminal nucleotide of the second strand of the nucleic acid (“Z”).

In certain embodiments, the nucleic acid (which is denoted by the “Z”) is conjugated to the (triantennary) ligand via the phosphate or thiophosphate group of the ligand moiety which links the ligand to the 5 position of the 5′terminal nucleotide of the second strand of the nucleic acid or which links the ligand to the 3 position of the 3 terminal nucleotide of the second strand of the nucleic acid.

A ligand of formula (II), (III) or (IV) or any one of the triantennary ligands disclosed herein can be attached at a nucleic acid end or to a nucleotide that is not at the end of the nucleic acid. In the case of a double-stranded nucleic acid, the ligand can be attached at the 3′-end of the first (antisense) strand and/or at any of the 3′ and/or 5′ end of the second (sense) strand. The nucleic acid can comprise more than one ligand of formula (II), (III) or (IV) or any one of the triantennary ligands disclosed herein. However, a single ligand of formula (II), (III) or (IV) or any one of the triantennary ligands disclosed herein is preferred because a single such ligand is sufficient for efficient targeting of the nucleic acid to the target cells. Preferably in that case, at least the last two, preferably at least the last three and more preferably at least the last four nucleotides at the end of the nucleic acid to which the ligand is attached are linked by a phosphodiester linkage.

In one embodiment, in the case of a double-stranded nucleic acid, the 5′-end of the first (antisense) strand is not attached to a ligand of formula (II), (III) or (IV) or any one of the triantennary ligands disclosed herein, since a ligand in this position can potentially interfere with the biological activity of the nucleic acid.

A nucleic acid with a single ligand of formula (II), (III) or (IV) or any one of the triantennary ligands disclosed herein at the 5′ end of a strand is easier and therefore cheaper to synthesise than the same nucleic acid with the same ligand at the 3′ end. Preferably therefore, a single ligand of any of formulae (II), (III) or (IV) or any one of the triantennary ligands disclosed herein is covalently attached to (conjugated with) the 5′ end a nucleic acid strand, and preferably to the 5′ end of the second strand when the nucleic acid is double-stranded.

In one aspect of a double-stranded nucleic acid, the first strand of the nucleic acid is a compound of formula (V):

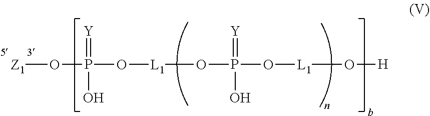

wherein b is preferably 0 or 1; and the second strand is a compound of formula (VI):

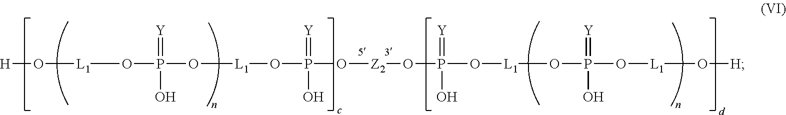

wherein:
c and d are independently preferably 0 or 1;
Z₁ and Z₂ are respectively the first and second strand of the nucleic acid;
Y is independently O or S;
n is independently 0, 1, 2 or 3; and
L₁ is a linker to which a ligand is attached, wherein L₁ is the same or different in formulae
(V) and (VI), and is the same or different within formulae (V) and (VI) when L₁ is present more than once within the same formula, wherein L₁ is preferably of formula (VII); and wherein b+c+d is preferably 2 or 3.

In one embodiment, L₁ in formulae (V) and (VI) is of formula (VII):

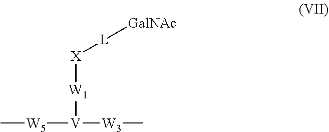

wherein:
L is selected from the group comprising, or preferably consisting of:
   —(CH₂)ᵣ—C(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to X of formula (VII), or if X is absent, to W₁ of formula (VII), or if W₁ is absent, to V of formula (VII);
W₁, W₃ and W₅ are individually absent or selected from the group comprising, or preferably consisting of:
   —(CH₂)ᵣ—, wherein r=1-7;
   —(CH₂)ₛ—O—(CH₂)ₛ—, wherein s is independently 0-5;
   —(CH₂)ₜ—S—(CH₂)ₜ—, wherein t is independently 0-5;
X is absent or is selected from the group comprising, or preferably consisting of: NH, NCH₃ or NC₂H₅;
V is selected from the group comprising, or preferably consisting of:

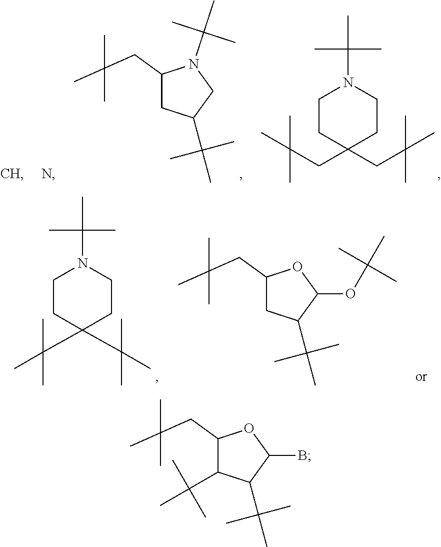

wherein B, if present, is a modified or natural nucleobase.

In one aspect, the first strand is a compound of formula (VIII)

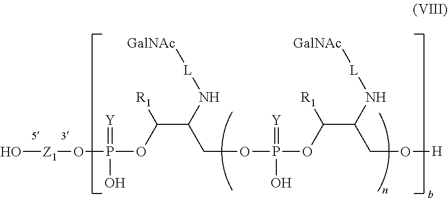

wherein b is preferably 0 or 1; and
the second strand is a compound of formula (IX):

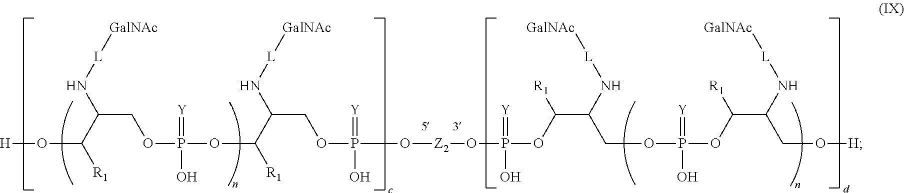

wherein c and d are independently preferably 0 or 1;
   wherein:
Z₁ and Z₂ are respectively the first and second strand of the nucleic acid;
Y is independently O or S;
R₁ is H or methyl;
n is independently preferably 0, 1, 2 or 3; and
L is the same or different in formulae (VIII) and (IX), and is the same or different within formulae (VIII) and (IX) when L is present more than once within the same formula, and is selected from the group comprising, or preferably consisting of:
   —(CH₂)ᵣ—C(O)—, wherein r=2-12;
   —(CH₂—CH₂—O)ₛ—CH₂—C(O)—, wherein s=1-5;
   —(CH₂)ₜ—CO—NH—(CH₂)ₜ— NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to the NH group (of the linker, not of the targeting ligand);
   and wherein b+c+d is preferably 2 or 3.

In one aspect, the first strand of the nucleic acid is a compound of formula (X):

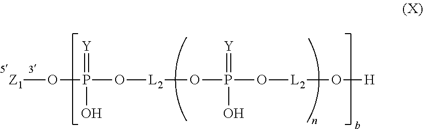

wherein b is preferably 0 or 1; and
the second strand is a compound of formula (XI):

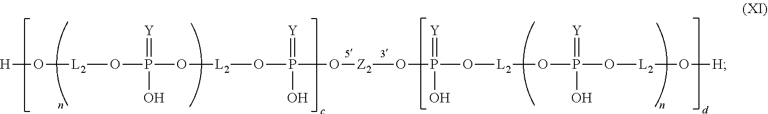

wherein:

c and d are independently preferably 0 or 1;
Z₁ and Z₂ are respectively the first and second RNA strand of the nucleic;
Y is independently O or S;
n is independently preferably 0, 1, 2 or 3; and
L₂ is the same or different in formulae (X) and (XI) and is the same or different in moieties bracketed by b, c and d, and is selected from the group comprising, or preferably consisting of:

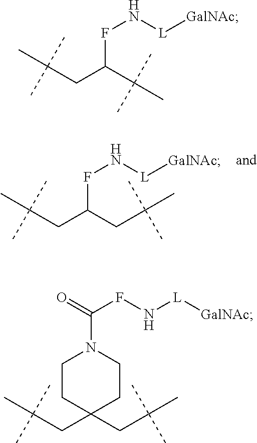

or
n is 0 and L₂ is:

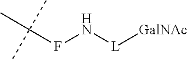

and the terminal OH group is absent such that the following moiety is formed:

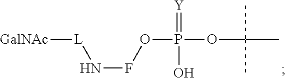

wherein:
F is a saturated branched or unbranched (such as unbranched) C₁-8alkyl (e.g. C₁₆alkyl) chain wherein one of the carbon atoms is optionally replaced with an oxygen atom provided that said oxygen atom is separated from another heteroatom (e.g. an O or N atom) by at least 2 carbon atoms;
L is the same or different in formulae (X) and (XI) and is selected from the group comprising, or preferably consisting of:
   —(CH₂)ᵣ—C(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12; and
wherein the terminal C(O), if present, is attached to the NH group (of the linker, not of the targeting ligand);
   and wherein b+c+d is preferably 2 or 3.

In one aspect, b is 0, c is 1 and d is 1; b is 1, c is 0 and d is 1; b is 1, c is 1 and d is 0; or b is 1, c is 1 and d is 1 in any of the nucleic acids of formulae (V) and (VI) or (VIII) and (IX) or (X) and (XI). Preferably, b is 0, c is 1 and d is 1; b is 1, c is 0 and d is 1; or b is 1, c is 1 and d is 1. Most preferably, b is 0, c is 1 and d is 1.

In one aspect, Y is O in any of the nucleic acids of formulae (V) and (VI) or (VIII) and (IX) or (X) and (XI). In another aspect, Y is S. In a particular aspect, Y is independently selected from O or S in the different positions in the formulae.

In one aspect, R₁ is H or methyl in any of the nucleic acids of formulae (VIII) and (IX). In one aspect, R₁ is H. In another aspect, R₁ is methyl.

In one aspect, n is 0, 1, 2 or 3 in any of the nucleic acids of formulae (V) and (VI) or (VIII) and (IX) or (X) and (XI). Preferably, n is 0.

Examples of F moieties in any of the nucleic acids of formulae (X) and (XI) include (CH₂)₁₋₆e.g. (CH₂)₁₋₄e.g. CH₂, (CH₂)₄, (CH₂)₅ or (CH₂)₆, or CH₂O(CH₂)₂₋₃, e.g. CH₂O(CH₂)CH₃.

In one aspect, L₂ in formulae (X) and (XI) is:

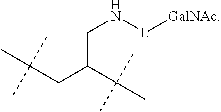

In one aspect, L₂ is:

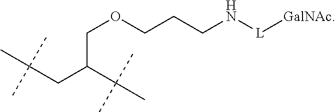

In one aspect, L₂ is:

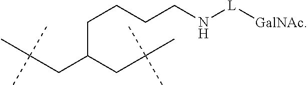

In one aspect, L₂ is:

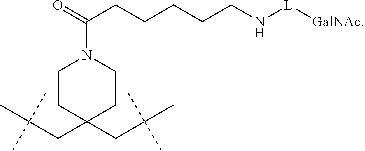

In one aspect, n is 0 and L₂ is:

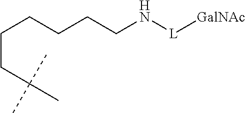

and the terminal OH group is absent such that the following moiety is formed:

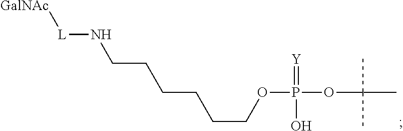

wherein Y is O or S.

In one aspect, L in the nucleic acids of formulae (V) and (VI) or (VIII) and (IX) or (X) and (XI), is selected from the group comprising, or preferably consisting of:
—(CH₂)ᵣ—C(O)—, wherein r=2-12;
   —(CH₂—CH₂—O)ₛ—CH₂—C(O)—, wherein s=1-5;
   —(CH₂)ₜ—CO—NH—(CH₂)ₜ—NH—C(O)—, wherein t is independently 1-5;
   —(CH₂)ᵤ—CO—NH—(CH₂)ᵤ—C(O)—, wherein u is independently 1-5; and
   —(CH₂)ᵥ—NH—C(O)—, wherein v is 2-12;
   wherein the terminal C(O) is attached to the NH group.

In one embodiment, L is —(CH₂)—C(O)—, wherein r=2-12, more preferably r=2-6 even more preferably, r=4 or 6 e.g. 4.

In one embodiment, L is:

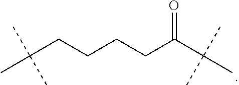

Within the moiety bracketed by b, c and d, L₂ in the nucleic acids of formulae (X) and (XI) is typically the same. Between moieties bracketed by b, c and d, L₂ may be the same or different. In an embodiment, L₂ in the moiety bracketed by c is the same as the L₂ in the moiety bracketed by d. In an embodiment, L₂ in the moiety bracketed by c is not the same as L₂ in the moiety bracketed by d. In an embodiment, the L₂ in the moieties bracketed by b, c and d is the same, for example when the linker moiety is a serinol-derived linker moiety.

Serinol derived linker moieties may be based on serinol in any stereochemistry i.e. derived from L-serine isomer, D-serine isomer, a racemic serine or other combination of isomers. In a preferred aspect of the invention, the serinol-GalNAc moiety (SerGN) has the following stereochemistry:

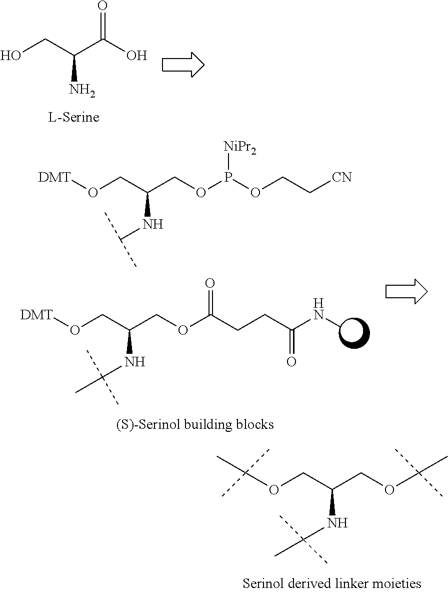

i.e. is based on an (S)-serinol-amidite or (S)-serinol succinate solid supported building block derived from L-serine isomer.

In one aspect, the first strand of the nucleic acid is a compound of formula (VIII) and the second strand of the nucleic acid is a compound of formula (IX), wherein:
b is 0;
c and d are 1,
n is 0,
Z₁ and Z₂ are respectively the first and second strand of the nucleic acid,
Y is S,
R₁ is H, and
L is —(CH₂)₄—C(O)—, wherein the terminal C(O) of L is attached to the N atom of the linker (ie not a possible N atom of a targeting ligand).

In another aspect, the first strand of the nucleic acid is a compound of formula (V) and the second strand of the nucleic acid is a compound of formula (VI), wherein:
b is 0,
c and d are 1,
n is 0,
Z₁ and Z₂ are respectively the first and second strand of the nucleic acid,
Y is S,
L₁ is of formula (VII), wherein:
   W₁ is —CH₂—O—(CH₂)₃—,
   W₃ is —CH₂—,
   W₅ is absent,
   V is CH,
   X is NH, and
   L is —(CH₂)₄—C(O)— wherein the terminal C(O) of L is attached to the N atom of X in formula (VII).

In another aspect, the first strand of the nucleic acid is a compound of formula (V) and the second strand of the nucleic acid is a compound of formula (VI), wherein:
b is 0,
c and d are 1,
n is 0,
Z₁ and Z₂ are respectively the first and second strand of the nucleic acid,
Y is S,
L₁ is of formula (VII), wherein:
   W₁, W₃ and W₅ are absent,
   V is

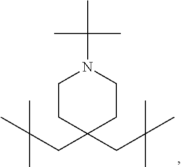 [SMILES: CC(C)(C)CC1(CC(C)(C)C)CCN(C(C)(C)C)CC1]

X is absent, and
L is —(CH₂)₄—C(O)—NH—(CH₂)₅—C(O)—, wherein the terminal C(O) of L is attached to the N atom of V in formula (VII).

In one aspect, the nucleic acid is conjugated to a triantennary ligand with the following structure:

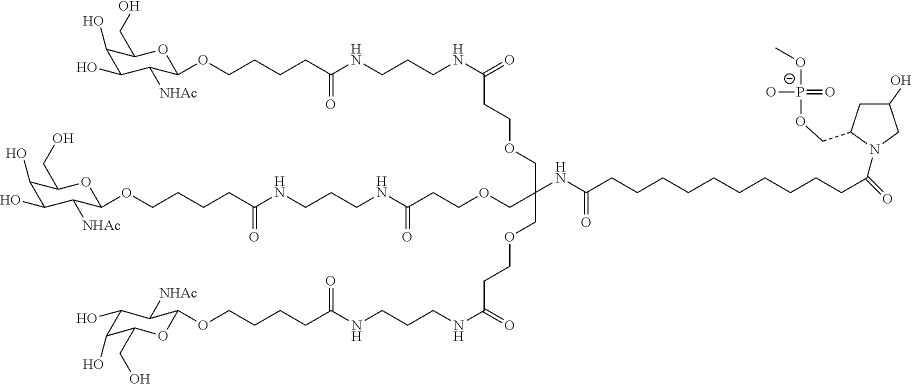

wherein the nucleic acid is conjugated to the ligand via the phosphate group of the ligand a) to the last nucleotide at the 5′ end of the nucleic acid strand, preferably the 5′ end of the second strand when the nucleic acid is double-stranded; b) to the last nucleotide at the 3′ end of the nucleic acid strand, preferably the 3′ end of the second the second strand when the nucleic acid is double-stranded; or c) to the last nucleotide at the 3′ end of the first strand of a double-stranded nucleic acid.

In one aspect of the nucleic acid, the cells that are targeted by the nucleic acid with a ligand are hepatocytes.

In any one of the above ligands where GalNAc is present, the GalNAc may be substituted for any other targeting ligand, such as those mentioned herein, preferably mannose, galactose, glucose, glucosamine and fucose.

In one aspect, the nucleic acid is conjugated to a ligand that comprises a lipid, and more preferably, a ligand that comprises a cholesterol.

Many nucleic acids that are capable of inhibiting expression of TMPRSS6 as well as experimental data for these nucleic acids are disclosed in WO2018185240, WO2012135246 and WO2014190157. Any nucleic acid disclosed in any of these documents that is capable of inhibiting expression of TMPRSS6 as appropriate are also part of the invention. These documents are hereby incorporated by reference.

### Compositions, Uses and Methods

The present invention also provides compositions comprising an inhibitor or nucleic acid of the invention. The inhibitors, nucleic acids and compositions may be used as medicaments or as diagnostic agents, alone or in combination with other agents. For example, one or more inhibitor(s) or nucleic acid(s) of the invention can be combined with a delivery vehicle (e.g., liposomes) and/or excipients, such as carriers, diluents. Other agents such as preservatives and stabilizers can also be added. Pharmaceutically acceptable salts or solvates of any of the inhibitors or nucleic acids of the invention are likewise within the scope of the present invention. Methods for the delivery of nucleic acids are known in the art and within the knowledge of the person skilled in the art.

Compositions disclosed herein are preferably pharmaceutical compositions. Such compositions are suitable for administration to a subject.

In one aspect, the composition comprises an inhibitor or a nucleic acid disclosed herein, or a pharmaceutically acceptable salt or solvate thereof, and a solvent (preferably water) and/or a delivery vehicle and/or a physiologically acceptable excipient and/or a carrier and/or a salt and/or a diluent and/or a buffer and/or a preservative.

Pharmaceutically acceptable carriers or diluents include those used in formulations suitable for oral, rectal, nasal or parenteral (including subcutaneous, intramuscular, intravenous, intradermal, and transdermal) administration. The formulations may conveniently be presented in unit dosage form and may be prepared by any of the methods well known in the art of pharmacy. Subcutaneous or transdermal modes of administration may be particularly suitable for the compounds described herein.

The therapeutically effective amount of an inhibitor or a nucleic acid of the present invention will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy, and may depend on such factors as weight, diet, concurrent medication and other factors, well known to those skilled in the medical arts. The dosage sizes and dosing regimen most appropriate for human use may be guided by the results obtained by the present invention, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject. Such considerations are known to the skilled person.

Inhibitors or nucleic acids of the present invention, or salts thereof, may be formulated as pharmaceutical compositions prepared for storage or administration, which typically comprise a therapeutically effective amount of an inhibitor or nucleic acid of the invention, or a salt thereof, in a pharmaceutically acceptable carrier.

The inhibitor or nucleic acid or conjugated nucleic acid of the present invention can also be administered in combination with other therapeutic compounds, either administrated separately or simultaneously, e.g., as a combined unit dose. The invention also includes a composition comprising one or more nucleic acids according to the present invention in a physiologically/pharmaceutically acceptable excipient, such as a stabilizer, preservative, diluent, buffer, and the like.

In one aspect, the composition comprises an inhibitor or nucleic acid disclosed herein and a further therapeutic agent selected from the group comprising an oligonucleotide, a small molecule, a monoclonal antibody, a polyclonal antibody, a peptide and a protein.

In certain embodiments, two or more different inhibitors of the invention may be administered simultaneously or sequentially.

In certain embodiments, two or more nucleic acids of the invention with different sequences may be administered simultaneously or sequentially.

In another aspect, the present invention provides a composition, e.g., a pharmaceutical composition, comprising one or a combination of different inhibitors or nucleic acids of the invention and at least one pharmaceutically acceptable carrier.

Dosage levels for the medicament and compositions of the invention can be determined by those skilled in the art by experimentation. In one aspect for nucleic acids, a unit dose may contain between about 0.01 mg/kg and about 100 mg/kg body weight of nucleic acid or conjugated nucleic acid. Alternatively, the dose can be from 10 mg/kg to 25 mg/kg body weight, or 1 mg/kg to 10 mg/kg body weight, or 0.05 mg/kg to 5 mg/kg body weight, or 0.1 mg/kg to 5 mg/kg body weight, or 0.1 mg/kg to 1 mg/kg body weight, or 0.1 mg/kg to 0.5 mg/kg body weight, or 0.5 mg/kg to 1 mg/kg body weight. Alternatively, the dose can be from about 0.5 mg/kg to about 10 mg/kg body weight, or about 0.6 mg/kg to about 8 mg/kg body weight, or about 0.7 mg/kg to about 7 mg/kg body weight, or about 0.8 mg/kg to about 6 mg/kg body weight, or about 0.9 mg/kg to about 5.5 mg/kg body weight, or about 1 mg/kg to about 5 mg/kg body weight, or about 2 mg/kg to about 5 mg/kg body weight, or about 3 mg/kg to about 5 mg/kg body weight, or about 1 mg/kg body weight, or about 3 mg/kg body weight, or about 5 mg/kg body weight, wherein “about” is a deviation of up to 30%, preferably up to 20%, more preferably up to 10%, yet more preferably up to 5% and most preferably 0% from the indicated value. Dosage levels may also be calculated via other parameters such as, e.g., body surface area.

The dosage and frequency of administration may vary depending on whether the treatment is therapeutic or prophylactic (e.g., preventative), and may be adjusted during the course of treatment. In certain prophylactic applications, a relatively low dosage is administered at relatively infrequent intervals over a relatively long period of time. Some subjects may continue to receive treatment over their lifetime. In certain therapeutic applications, a relatively high dosage at relatively short intervals is sometimes required until progression of the disease is reduced or until the patient shows partial or complete amelioration of symptoms of disease. Thereafter, the patient may be switched to a suitable prophylactic dosing regimen.

Actual dosage levels of an inhibitor or a nucleic acid of the invention alone or in combination with one or more other active ingredients in the pharmaceutical compositions of the present invention may be varied so as to obtain an amount of the active ingredient which is effective to achieve the desired therapeutic response for a particular patient, composition, and mode of administration, without causing deleterious side effects to the subject or patient. A selected dosage level will depend upon a variety of factors, such as pharmacokinetic factors, including the activity of the particular inhibitor or nucleic acid or composition employed, the route of administration, the time of administration, the rate of excretion of the particular inhibitor or nucleic acid being employed, the duration of the treatment, other drugs, compounds and/or materials used in combination with the particular compositions employed, the age, sex, weight, condition, general health and prior medical history of the subject or patient being treated, and similar factors well known in the medical arts.

The pharmaceutical composition may be a sterile injectable aqueous suspension or solution, or in a lyophilised form.

The pharmaceutical compositions can be in unit dosage form. In such form, the composition is divided into unit doses containing appropriate quantities of the active component. The unit dosage form can be a packaged preparation, the package containing discrete quantities of the preparations, for example, packeted tablets, capsules, and powders in vials or ampoules. The unit dosage form can also be a capsule, cachet, or tablet itself, or it can be the appropriate number of any of these packaged forms. It may be provided in single dose injectable form, for example in the form of a pen. Compositions may be formulated for any suitable route and means of administration.

The pharmaceutical compositions and medicaments of the present invention may be administered to a mammalian subject in a pharmaceutically effective dose. The mammal may be selected from a human, a non-human primate, a simian or prosimian, a dog, a cat, a horse, cattle, a pig, a goat, a sheep, a mouse, a rat, a hamster, a hedgehog and a guinea pig, or other species of relevance. On this basis, “Matriptase-2”, “MT2” and “TMPRSS6” as used herein denotes nucleic acid or protein in any of the above-mentioned species, if expressed therein naturally or artificially, but preferably this wording denotes human nucleic acids or proteins.

Pharmaceutical compositions of the invention may be administered alone or in combination with one or more other therapeutic or diagnostic agents. A combination therapy may include an inhibitor or nucleic acid of the present invention combined with at least one other therapeutic agent selected based on the particular patient, disease or condition to be treated. Examples of other such agents include, inter alia, a therapeutically active small molecule or polypeptide, a single chain antibody, a classical antibody or fragment thereof, or a nucleic acid molecule which modulates gene expression of one or more additional genes, and similar modulating therapeutics which may complement or otherwise be beneficial in a therapeutic or prophylactic treatment regimen.

Pharmaceutical compositions are typically sterile and stable under the conditions of manufacture and storage. The composition may be formulated as a solution, microemulsion, liposome, or other ordered structure suitable to high drug concentration. The carrier may be a solvent or dispersion medium containing, for example, water, alcohol such as ethanol, polyol (e.g., glycerol, propylene glycol, and liquid polyethylene glycol), or any suitable mixtures. The proper fluidity may be maintained, for example, by the use of a coating such as lecithin, by the maintenance of the required particle size in the case of dispersion and by use of surfactants according to formulation chemistry well known in the art. In certain embodiments, isotonic agents, e.g., sugars, polyalcohols such as mannitol, sorbitol, or sodium chloride may be desirable in the composition. Prolonged absorption of injectable compositions may be brought about by including in the composition an agent that delays absorption for example, monostearate salts and gelatine.

One aspect of the invention is an inhibitor or nucleic acid or a composition disclosed herein for use as a medicament. The nucleic acid or composition is preferably for use in the prevention, decrease of the risk of suffering from, or treatment of a myeloproliferative disorder.

The present invention provides an inhibitor or nucleic acid for use, alone or in combination with one or more additional therapeutic agents in a pharmaceutical composition, for treatment or prophylaxis of conditions, diseases and disorders responsive to inhibition of Matriptase 2 (MT2) or TMPRSS6 expression.

One aspect of the invention is the use of an inhibitor or a nucleic acid or a composition as disclosed herein in the prevention, decrease of the risk of suffering from, or treatment of a myeloproliferative disorder.

Inhibitors, nucleic acids and pharmaceutical compositions of the invention may be used in the treatment of a variety of conditions, disorders or diseases. Treatment with an inhibitor or nucleic acid of the invention in certain cases leads to in vivo Matriptase-2 (MT2) depletion, preferably in the liver. As such, inhibitors or nucleic acids of the invention, and compositions comprising them, will be useful in methods for treating a variety of pathological disorders in which inhibiting the expression of Matriptase-2 (MT2) may be beneficial, such as, inter alia, myeloproliferative disorders. The present invention provides methods for treating myeloproliferative disorders comprising the step of administering to a subject in need thereof a therapeutically effective amount of an inhibitor, nucleic acid or composition of the invention.

The invention thus provides methods of treatment or prevention of a myeloproliferative disorder, the method comprising the step of administering to a subject (e.g., a patient) in need thereof a therapeutically effective amount of an inhibitor or nucleic acid or pharmaceutical composition of the invention.

The most desirable therapeutically effective amount is an amount that will produce a desired efficacy of a particular treatment selected by one of skill in the art for a given subject in need thereof. This amount will vary depending upon a variety of factors understood by the skilled worker, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through experimentation, namely by monitoring a subject's response to administration of a compound and adjusting the dosage accordingly. See, e.g., Remington: The Science and Practice of Pharmacy 21st Ed., Univ. of Sciences in Philadelphia (USIP), Lippincott Williams & Wilkins, Philadelphia, PA, 2005.

In certain embodiments, nucleic acids and pharmaceutical compositions of the invention may be used to treat or prevent a myeloproliferative disorder.

In certain embodiments, the present invention provides methods for treating a myeloproliferative disorder in a mammalian subject, such as a human, the method comprising the step of administering to a subject in need thereof a therapeutically effective amount of an inhibitor or a nucleic acid or a composition as disclosed herein.

Administration of a “therapeutically effective dosage” of an inhibitor or nucleic acid or composition of the invention may result in a decrease in severity of disease symptoms, an increase in frequency and duration of disease symptom-free periods, or a prevention of impairment or disability due to the disease affliction.

Inhibitors or nucleic acids or compositions of the invention may be beneficial in treating or diagnosing myeloproliferative disorders that may be diagnosed or treated using the methods described herein. Treatment and diagnosis of other myeloproliferative disorders are also considered to fall within the scope of the present invention.

One aspect of the invention is a method of preventing, decreasing the risk of suffering from, or treating a myeloproliferative disorder, comprising administering a pharmaceutically effective dose or amount of an inhibitor or a nucleic acid or a composition disclosed herein to an individual in need of treatment, preferably wherein the inhibitor or nucleic acid or composition is administered to the subject subcutaneously, intravenously or by oral, rectal, pulmonary, intramuscular or intraperitoneal administration. Preferably, it is administered subcutaneously.

Inhibitor or nucleic acids or compositions disclosed herein may be for use in a regimen comprising treatments once or twice weekly, every week, every two weeks, every three weeks, every four weeks, every five weeks, every six weeks, every seven weeks, every eight weeks, every nine weeks, every ten weeks, every eleven weeks, every twelve weeks, every three months, every four months, every five months, every six months or in regimens with varying dosing frequency such as combinations of the before-mentioned intervals. The inhibitor or nucleic acid or composition may be for use subcutaneously, intravenously or using any other application routes such as oral, rectal, pulmonary, intramuscular or intraperitoneal. Preferably, it is for use subcutaneously.

An exemplary treatment regime is administration once every two weeks, once every three weeks, once every four weeks, once a month, once every two or three months or once every three, four, five or six or more months. Dosages may be selected and readjusted by the skilled health care professional as required to maximize therapeutic benefit for a particular subject, e.g., patient. The inhibitors or nucleic acids will typically be administered on multiple occasions. Intervals between single dosages can be, for example, 2-5 days, weekly, bi-weekly, monthly, every two or three months, every four or five months, every six months, or yearly. Intervals between administrations can also be irregular, based on nucleic acid target gene product levels for example in the blood or liver of the subject or patient.

In cells and/or subjects treated with or receiving an inhibitor or nucleic acid or composition as disclosed herein, Matriptase-2 (MT2) protein and/or TMPRSS6 mRNA expression may be inhibited compared to untreated cells and/or subjects by a range from 15% up to 100% but at least about 30%, 35%, 40%, 45%, 50%, 55%, 60%, 65%, 70%, 75%, 80%, 85%, 90%, 95%, 98%, or 100% or intermediate values. The level of inhibition may allow treatment of a myeloproliferative disorder or may serve to further investigate the functions and physiological roles of the TMPRSS6 gene products. The level of inhibition is preferably measured in the liver or in the blood or in the kidneys, preferably in the liver, of the subject treated with the inhibitor or nucleic acid or composition.

One aspect is the use of an inhibitor or nucleic acid or composition as disclosed herein in the manufacture of a medicament for treating a myeloproliferative disorder such as those as listed below or additional pathologies where inhibition of Matriptase-2 (MT2) or TMPRSS6 expression is desired. A medicament is a pharmaceutical composition.

Each of the inhibitors or nucleic acids of the invention and pharmaceutically acceptable salts and solvates thereof constitutes an individual embodiment of the invention.

One aspect of the invention is a method of treating or preventing a myeloproliferative disorder, as described herein, comprising administering a pharmaceutically effective dose or amount of a double-stranded nucleic acid, as described herein, to an individual in need of treatment, wherein the nucleic acid is administered to the subject subcutaneously, intravenously or by oral, rectal, pulmonary, intramuscular or intraperitoneal administration. In one embodiment, it is administered subcutaneously.

Also included in the invention is a method of treating or preventing a myeloproliferative disorder, such as those listed below, comprising administration of a composition comprising an inhibitor or nucleic acid or composition as described herein, to an individual in need of treatment (to improve such pathologies). The inhibitor or nucleic acid or composition may be administered in a regimen comprising treatments twice every week, once every week, every two weeks, every three weeks, every four weeks, every five weeks, every six weeks, every seven weeks, or every eight to twelve or more weeks or in regimens with varying dosing frequency such as combinations of the before-mentioned intervals. The inhibitor or nucleic acid or conjugated nucleic acid or composition may be for use subcutaneously or intravenously or other application routes such as oral, rectal or intraperitoneal.

One aspect is the use of a double-stranded nucleic acid, as described herein, in the manufacture of a medicament for treating or preventing a myeloproliferative disorder, as described herein. A medicament is a pharmaceutical composition.

One aspect is the use of a composition, as described herein, in the manufacture of a medicament for treating or preventing a myeloproliferative disorder, as described herein. A medicament is a pharmaceutical composition. In one embodiment the composition is a pharmaceutical composition.

An inhibitor or nucleic acid of the invention may be administered by any appropriate administration pathway known in the art, including but not limited to aerosol, enteral, nasal, ophthalmic, oral, parenteral, rectal, vaginal, or transdermal (e.g., topical administration of a cream, gel or ointment, or by means of a transdermal patch). “Parenteral administration” is typically associated with injection at or in communication with the intended site of action, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal administration.

The use of a chemical modification pattern of the nucleic acids confers nuclease stability in serum and makes for example subcutaneous application route feasible.

Solutions or suspensions used for intradermal or subcutaneous application typically include one or more of: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and/or tonicity adjusting agents such as, e.g., sodium chloride or dextrose. The pH can be adjusted with acids or bases, such as hydrochloric acid or sodium hydroxide, or buffers with citrate, phosphate, acetate and the like. Such preparations may be enclosed in ampoules, disposable syringes or multiple dose vials made of glass or plastic.

Sterile injectable solutions may be prepared by incorporating an inhibitor or a nucleic acid in the required amount in an appropriate solvent with one or a combination of ingredients described above, as required, followed by sterilization microfiltration. Dispersions may be prepared by incorporating the active compound into a sterile vehicle that contains a dispersion medium and optionally other ingredients, such as those described above. In the case of sterile powders for the preparation of sterile injectable solutions, the methods of preparation are vacuum drying and freeze-drying (lyophilization) that yield a powder of the active ingredient in addition to any additional desired ingredient from a sterile-filtered solution thereof.

When a therapeutically effective amount of an inhibitor or nucleic acid or composition of the invention is administered by, e.g., intravenous, cutaneous or subcutaneous injection, the inhibitor or nucleic acid will be in the form of a pyrogen-free, parenterally acceptable aqueous solution. Methods for preparing parenterally acceptable solutions, taking into consideration appropriate pH, isotonicity, stability, and the like, are within the skill in the art. A preferred pharmaceutical composition for intravenous, cutaneous, or subcutaneous injection will contain, in addition to an inhibitor or nucleic acid, an isotonic vehicle such as sodium chloride injection, Ringer's injection, dextrose injection, dextrose and sodium chloride injection, lactated Ringer's injection, or other vehicle as known in the art. A pharmaceutical composition of the present invention may also contain stabilizers, preservatives, buffers, antioxidants, or other additives well known to those of skill in the art.

The amount of inhibitor or nucleic acid which can be combined with a carrier material to produce a single dosage form will vary depending on a variety of factors, including the subject being treated, and the particular mode of administration. In general, it will be an amount of the composition that produces an appropriate therapeutic effect under the particular circumstances. Generally, out of one hundred percent, this amount will range from about 0.01% to about 99% of inhibitor or nucleic acid, from about 0.1% to about 70%, or from about 1% to about 30% of inhibitor or nucleic acid in combination with a pharmaceutically acceptable carrier.

The inhibitor or nucleic acid may be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Dosage regimens may be adjusted to provide the optimum desired response (e.g., a therapeutic response). For example, a dose may be administered, several divided doses may be administered over time, or the dose may be proportionally reduced or increased as indicated by the particular circumstances of the therapeutic situation, on a case by case basis. It is especially advantageous to formulate parenteral compositions in dosage unit forms for ease of administration and uniformity of dosage when administered to the subject or patient. As used herein, a dosage unit form refers to physically discrete units suitable as unitary dosages for the subjects to be treated; each unit containing a predetermined quantity of active compound calculated to produce a desired therapeutic effect. The specification for the dosage unit forms of the invention depend on the specific characteristics of the active compound and the particular therapeutic effect(s) to be achieved and the treatment and sensitivity of any individual patient.

The inhibitors or nucleic acids or compositions of the present invention can be produced using routine methods in the art including chemical synthesis, such as solid phase chemical synthesis.

Inhibitors or nucleic acids or compositions of the invention may be administered with one or more of a variety of medical devices known in the art. For example, in one embodiment, an inhibitor or nucleic acid of the invention may be administered with a needleless hypodermic injection device. Examples of well-known implants and modules useful in the present invention are in the art, including e.g., implantable micro-infusion pumps for controlled rate delivery; devices for administering through the skin; infusion pumps for delivery at a precise infusion rate; variable flow implantable infusion devices for continuous drug delivery; and osmotic drug delivery systems. These and other such implants, delivery systems, and modules are known to those skilled in the art.

In certain embodiments, the inhibitor or nucleic acid or composition of the invention may be formulated to ensure a desired distribution in vivo. To target a therapeutic compound or composition of the invention to a particular in vivo location, they can be formulated, for example, in liposomes which may comprise one or more moieties that are selectively transported into specific cells or organs, thus enhancing targeted drug delivery.

The invention is characterized by high specificity at the molecular and tissue-directed delivery level. The inhibitors or nucleic acids of the invention are highly specific for their targets. The sequences of the nucleic acids of the invention for example are highly specific for their target, meaning that they do not inhibit the expression of genes that they are not designed to target or only minimally inhibit the expression of genes that they are not designed to target and/or only inhibit the expression of a low number of genes that they are not designed to target. A further level of specificity is achieved when nucleic acids are linked to a ligand that is specifically recognised and internalised by a particular cell type. This is for example the case when a nucleic acid is linked to a ligand comprising GalNAc moieties, which are specifically recognised and internalised by hepatocytes. This leads to the nucleic acid inhibiting the expression of their target only in the cells that are targeted by the ligand to which they are linked. These two levels of specificity potentially confer a better safety profile than the currently available treatments. In certain embodiments, the present invention thus provides nucleic acids of the invention linked to a ligand comprising one or more GalNAc moieties, or comprising one or more other moieties that confer cell-type or tissue-specific internalisation of the nucleic acid thereby conferring additional specificity of target gene knockdown by RNA interference.

The inhibitors or nucleic acids as described herein may be formulated with a lipid in the form of a liposome. Such a formulation may be described in the art as a lipoplex. The composition with a lipid/liposome may be used to assist with delivery of the inhibitor or nucleic acid of the invention to the target cells. The lipid delivery system herein described may be used as an alternative to a conjugated ligand. The modifications herein described may be present when using the inhibitor or nucleic acid of the invention with a lipid delivery system or with a ligand conjugate delivery system.

Such a lipoplex may comprise a lipid composition comprising:
i) a cationic lipid, or a pharmaceutically acceptable salt thereof;
ii) a steroid;
iii) a phosphatidylethanolamine phospholipid; and/or
iv) a PEGylated lipid.

The cationic lipid may be an amino cationic lipid.

The content of the cationic lipid component may be from about 55 mol % to about 65 mol % of the overall lipid content of the composition. Preferably, the cationic lipid component is about 59 mol % of the overall lipid content of the composition.

The compositions can further comprise a steroid. The steroid may be cholesterol. The content of the steroid may be from about 26 mol % to about 35 mol % of the overall lipid content of the lipid composition. More preferably, the content of steroid may be about 30 mol % of the overall lipid content of the lipid composition.

The phosphatidylethanolamine phospholipid may be selected from the group consisting of 1,2-diphytanoyl-sn-glycero-3-phosphoethanolamine (DPhyPE), 1,2-dioleoyl-sn-glycero-3-phosphoethanolamine (DOPE), 1,2-distearoyl-sn-glycero-3-phosphoethanolamine (DSPE), 1,2-Dilauroyl-sn-glycero-3-phosphoethanolamine (DLPE), 1,2-Dimyristoyl-sn-glycero-3-phosphoethanolamine (DMPE), 1,2-Dipalmitoyl-sn-glycero-3-phosphoethanolamine (DPPE), 1,2-Dilinoleoyl-sn-glycero-3-phosphoethanolamine (DLoPE), 1-Palmitoyl-2-oleoyl-sn-glycero-3-phosphoethanolamine (POPE), 1,2-Dierucoyl-sn-glycero-3-phosphoethanolamine (DEPE), 1,2-Disqualeoyl-sn-glycero-3-phosphoethanolamine (DSQPE) and 1-Stearoyl-2-linoleoyl-sn-glycero-3-phosphoethanolamine (SLPE). The content of the phospholipid may be about 10 mol % of the overall lipid content of the composition.

The PEGylated lipid may be selected from the group consisting of 1,2-dimyristoyl-sn-glycerol, methoxypolyethylene glycol (DMG-PEG) and C16-Ceramide-PEG. The content of the PEGylated lipid may be about 1 to 5 mol % of the overall lipid content of the composition.

The content of the cationic lipid component in the composition may be from about 55 mol % to about 65 mol % of the overall lipid content of the lipid composition, preferably about 59 mol % of the overall lipid content of the lipid composition.

The composition may have a molar ratio of the components of i):ii): iii): iv) selected from 55:34:10:1; 56:33:10:1; 57:32:10:1; 58:31:10:1; 59:30:10:1; 60:29:10:1; 61:28:10:1; 62:27:10:1; 63:26:10:1; 64:25:10:1; and 65:24:10:1.

Neutral liposome compositions may be formed from, for example, dimyristoyl phosphatidylcholine (DMPC) or dipalmitoyl phosphatidylcholine (DPPC). Anionic liposome compositions may be formed from dimyristoyl phosphatidylglycerol, while anionic fusogenic liposomes may be formed primarily from dioleoyl phosphatidylethanolamine (DOPE). Another type of liposomal composition may be formed from phosphatidylcholine (PC) such as, for example, soybean PC, and egg PC. Another type is formed from mixtures of phospholipid and/or phosphatidylcholine and/or cholesterol.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells. DOTMA analogues can also be used to form liposomes.

Derivatives and analogues of lipids described herein may also be used to form liposomes.

A liposome containing an inhibitor or a nucleic acid can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The nucleic acid preparation is then added to the micelles that include the lipid component. The cationic groups on the lipid interact with the nucleic acid and condense around the nucleic acid to form a liposome. After condensation, the detergent is removed, e.g., by dialysis, to yield a liposomal preparation of nucleic acid.

If necessary, a carrier compound that assists in condensation can be added during the condensation reaction, e.g., by controlled addition. For example, the carrier compound can be a polymer other than a nucleic acid (e.g., spermine or spermidine). pH can also be adjusted to favour condensation.

Inhibitor or nucleic acid formulations of the present invention may include a surfactant. In one embodiment, the nucleic acid is formulated as an emulsion that includes a surfactant.

A surfactant that is not ionized is a non-ionic surfactant. Examples include non-ionic esters, such as ethylene glycol esters, propylene glycol esters, glyceryl esters etc., nonionic alkanolamides, and ethers such as fatty alcohol ethoxylates, propoxylated alcohols, and ethoxylated/propoxylated block polymers.

A surfactant that carries a negative charge when dissolved or dispersed in water is an anionic surfactant. Examples include carboxylates, such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates.

A surfactant that carries a positive charge when dissolved or dispersed in water is a cationic surfactant. Examples include quaternary ammonium salts and ethoxylated amines.

A surfactant that has the ability to carry either a positive or negative charge is an amphoteric surfactant. Examples include acrylic acid derivatives, substituted alkylamides, N-alkylbetaines and phosphatides.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic. A micelle may be formed by mixing an aqueous solution of the inhibitor or nucleic acid, an alkali metal alkyl sulphate, and at least one micelle forming compound.

Exemplary micelle forming compounds include lecithin, hyaluronic acid, pharmaceutically acceptable salts of hyaluronic acid, glycolic acid, lactic acid, chamomile extract, cucumber extract, oleic acid, linoleic acid, linolenic acid, monoolein, monooleates, monolaurates, borage oil, evening of primrose oil, menthol, trihydroxy oxo cholanyl glycine and pharmaceutically acceptable salts thereof, glycerol, polyglycerol, lysine, polylysine, triolein, polyoxyethylene ethers and analogues thereof, polidocanol alkyl ethers and analogues thereof, chenodeoxycholate, deoxycholate, and mixtures thereof.

Phenol and/or m-cresol may be added to the mixed micellar composition to act as a stabiliser and preservative. An isotonic agent such as glycerine may as be added.

An inhibitor or nucleic acid preparation may be incorporated into a particle such as a microparticle. Microparticles can be produced by spray-drying, lyophilisation, evaporation, fluid bed drying, vacuum drying, or a combination of these methods.

Throughout the description, a reference to “an inhibitor or a nucleic acid” or similar disclosures should not be interpreted as meaning that the inhibitor is not a nucleic acid. An inhibitor can be a nucleic acid such as a siRNA or an ASO.

### Indications

In certain embodiments, an inhibitor or nucleic acid or composition described herein is for use or is used in a method of prevention, decrease of the risk of suffering from or treatment of a myeloproliferative disorder, wherein the myeloproliferative disorder is on or several of:
a) Philadelphia chromosome (BCR-ABL) negative myeloproliferative neoplasm;
b) one or several of polycythaemia vera (PV), essential thrombocythaemia (ET) and primary myelofibrosis (PMF);
c) polycythaemia vera (PV)
d) an elevated risk of thrombosis
e) an elevated haematocrit level in the blood;
f) an elevated haemoglobin level in the blood;
g) an elevated red blood cell mass in the blood;
h) elevated erythropoiesis;
i) an elevated level of mature red cell population in the bone marrow;
j) a reduced level of progenitor red blood cells in the bone marrow; and
k) bone marrow erythroid and/or megakaryocytic hyperplasia.

Each such disease, condition, disorder or symptom is envisioned to be a separate embodiment with respect to uses of an inhibitor, nucleic acid or pharmaceutical composition according to the invention.

In certain embodiments, a myeloproliferative disorder, such as polycythaemia vera (PV) is characterised by one or several of:
a) an elevated risk of thrombosis;
b) polycythaemia vera (PV) symptoms;
c) a JAK2 mutation;
d) the JAK2 mutation V617F;
e) a mutation in JAK2 exon 12;
f) a mutation in a negative regulator of JAK2;
g) constitutive, erythropoietin independent JAK2/STAT signalling;
h) is an acquired disorder;
i) is an inherited disorder;
j) elevated haematocrit level in the blood;
k) elevated haemoglobin level in the blood;
I) elevated red blood cell mass;
m) elevated erythropoiesis;
n) an elevated level of mature red cell population in the bone marrow;
o) a reduced level of progenitor red blood cells in the bone marrow; and
p) bone marrow erythroid and/or megakaryocytic hyperplasia.

In one embodiment, the JAK2 mutation is present in haematopoietic stem cells.

In one embodiment, the myeloproliferative disorder is JAK2 positive polycythaemia vera (PV). JAK2 positive polycythaemia vera (PV) is characterised by one or more activating mutation(s) in the JAK2 (Janus kinase 2) gene/protein. One example of such an activating mutation is the V617F mutation. The JAK2 (V617F) (exon 14) mutation is found in 95% of PV cases. About 5% of the PV patients exhibit a mutation in exon 12 (McMullin M F, Wilkins B S, Harrison C N. Management of polycythaemia vera: a critical review of current data. Br J Haematol. 2016; 172(3):337-349; McMullin M F, Harrison C N, Ali S, et al., A guideline for the diagnosis and management of polycythaemia vera. A British Society for Haematology Guideline. Br J Haematol. 2019; 184(2):176-191).

In one embodiment, the myeloproliferative disorder is JAK2 positive polycythaemia vera (PV) characterised by a V617F mutation of JAK2.

In one embodiment, the myeloproliferative disorder is JAK2 positive polycythaemia vera (PV) characterised by one or more mutation(s) in exon 12 of JAK2 gene. Examples of mutation(s) in exon 12 of JAK2 gene are described for example in Li et al. (Blood 2008; 111(7): 3863-3866) and in Kondo et al. (Leukemia & Lymphoma 2008; 49(9): 1784-1791).

Other examples of mutation(s) in exon 12 of JAK2 gene are F537-K539delinsL, H538QK539L, K539L, N542-E543del, which are further described in Scott et al., New England Journal of Medicine 2008; 356(5): 459-468. Further mutations in exon 12 of JAK2 gene are described in Scott, American Journal of Hematology 2011; 86: 668-676: F533IK539L, F537IK539L, H538QK539L, H538DK539LI504S, K539L, K539LL545V, 1540T, D544G, L545S, F547L, F547V, F537-K539delinsK, F537-K539del, F537-K539delinsL, H538del, H538-K539del, H538-K539delinsF, H538-K539delinsI, H538-K539delinsL, 1540-N542delinsS,1540-N542delinsK, 1540-N543delinsKK, 1540-N543delinsMK, 1540-D544delinsMK, 1540S, R541-E543delinsK, R541-E543delinsK, R541-D544del, N542-D544delinsN, E543-D544del, D544-L545del, V536-1546dup11, V536-F547dup12, [V536, F37-1546dup10],[F537-1546dup10, F547L], [F547L, 1540-F547dup8].

Testing for JAK2 V617F in peripheral blood is sensitive (Takahashi et al., Blood 2013; 122:3784-3786). Assays which can be used for detection of JAK2 mutations are described, for example, in Bench et al. (British Journal of Haematology 2013; 160: 25-34).

In one embodiment, the myeloproliferative disorder is HFE positive polycythaemia vera (PV). HFE positive polycythaemia vera (PV) is characterised by one or more mutation(s) in the HFE (homeostatic iron regulator) gene/protein resulting in a loss of HFE function. The most common HFE mutations are C282Y, H63D and S65C.

In one embodiment, the myeloproliferative disorder is HFE positive polycythaemia vera (PV) characterised by a homozygous C282Y mutation.

In one embodiment, the myeloproliferative disorder is HFE positive polycythemia vera (PV) characterised by a heterozygous C282Y mutation.

In one embodiment, the myeloproliferative disorder is HFE positive polycythaemia vera (PV) characterised by a homozygous H63D mutation.

In one embodiment, the myeloproliferative disorder is HFE positive polycythaemia vera (PV) characterised by a heterozygous H63D mutation.

In one embodiment, the myeloproliferative disorder is HFE positive polycythaemia vera (PV) characterised by a homozygous S65C mutation.

In one embodiment, the myeloproliferative disorder is HFE positive polycythaemia vera (PV) characterised by a heterozygous S65C mutation.

In one embodiment, the myeloproliferative disorder is JAK2 positive and HFE positive polycythaemia vera (PV).

In one embodiment, the myeloproliferative disorder is JAK2 positive and HFE positive PV characterised by a V617F mutation of JAK2 and by a homozygous C282Y mutation of HFE.

In one embodiment, the myeloproliferative disorder is JAK2 positive and HFE positive PV characterised by a V617F mutation of JAK2 and by a heterozygous C282Y mutation of HFE.

In one embodiment, the myeloproliferative disorder is JAK2 positive and HFE positive PV characterised by a V617F mutation of JAK2 and by a heterozygous H63D mutation of HFE.

In one embodiment, the myeloproliferative disorder is JAK2 positive and HFE positive PV characterised by a V617F mutation of JAK2 and by a homozygous H63D mutation of HFE.

In one embodiment, the myeloproliferative disorder is JAK2 positive and HFE positive PV characterised by a V617F mutation of JAK2 and by a heterozygous S65C mutation of HFE.

In one embodiment, the myeloproliferative disorder is JAK2 positive and HFE positive PV characterised by a V617F mutation of JAK2 and by a homozygous S65C mutation of HFE.

In one embodiment, the myeloproliferative disorder is LNK positive PV.

LNK (Lymphocyte Adaptor Protein, also known as SH2B3 adaptor protein) positive polycythaemia vera (PV) is characterised by one or more mutation(s) in the SH2B3 gene resulting in a loss of LNK function.

LNK (SH2B3) belongs to a family of adaptor proteins that contain a proline-rich N-terminal dimerization domain, a pleckstrin homology domain (PH), an Src homology-2 domain (SH2), and a conserved C-terminal tyrosine residue. By binding to cytokine receptors and JAK2 through the SH2 domain, LNK inhibits downstream signaling pathways. LNK mutations are found mainly in exon 2 that, together with exons 3 and 4, encodes the PH domain. Mutations in exon 7 encoding the SH2 domain, and in exon 8 encoding the C-terminal portion, have recently been reported in a few cases, also in association with JAK2V617F mutation (Ha et al., Am J Hematol. 2011; 86(10): 866-868). Examples of LNK (SH2B3) mutations are: E208Q, P155L, S213R, T274A (Spolverini et al., Haematologica 2013; 98(9): e101-e102).

In one embodiment, the myeloproliferative disorder is LNK positive PV characterised by a E208Q mutation of LNK.

In one embodiment, the myeloproliferative disorder is LNK positive PV characterised by a P155L mutation of LNK.

In one embodiment, the myeloproliferative disorder is LNK positive PV characterised by a S213R mutation of LNK.

In one embodiment, the myeloproliferative disorder is LNK positive PV characterised by a T274A mutation of LNK.

An elevated haematocrit or haemoglobin level is a level that lies above the level expected in a healthy subject, such as a level that is elevated by 5% or more, 10% or more, 15% or more, 20% or more, 25% or more, 30% or more, 35% or more or 40% or more as compared to the level expected in a corresponding healthy subject. The expected level of haematocrit or haemoglobin in a healthy subject can vary depending on age, sex and other factors such as pregnancy, altitude and more. The person skilled in the art will be able to determine for a given subject whether their haemoglobin and haematocrit levels are elevated relative to the level expected in a corresponding healthy subject. For haematocrits, the level expected in a healthy subject is generally 45% or less for male subjects and 42% or less for female subjects (the percentage is the number of millilitres of red blood cells per 100 millilitres of blood).

In one embodiment, the myeloproliferative disorder is PV and the subject treated has a haematocrit level of more than 49% (men) or of more than 48% (women) (Barbui et al., Blood Cancer J. 2018 February; 8(2): 15).

In one embodiment, the inhibitor, nucleic acid or compositions of the invention is for use or is used in a method of treatment to:
a) reduce the risk of thrombosis;
b) reduce the level of haematocrits in the blood;
c) reduce the level of haemoglobin in the blood; and/or
d) reduce erythropoiesis.

In one embodiment, the use of an inhibitor, nucleic acid or composition disclosed herein reduces the risk of thrombosis in a subject treated with the inhibitor, nucleic acid or composition to the corresponding level expected in a healthy subject. Alternatively, it reduces the risk of thrombosis in a subject treated with the inhibitor, nucleic acid or composition such that the difference between the risk of thrombosis in the subject before treatment and the corresponding level expected in a healthy subject is at least temporarily reduced by 10%, 20%, 30%, 40%, 50%, 60%, 70%, 80%, 90% or 95%.

In one embodiment, the use of an inhibitor, nucleic acid or composition disclosed herein reduces the level of haematocrits in the blood of a subject treated with the inhibitor, nucleic acid or composition to the corresponding level expected in a healthy subject. Alternatively, it reduces the level of haematocrits in the blood of a subject treated with the inhibitor, nucleic acid or composition such that the difference between the level of haematocrits in the blood in a subject before treatment and the corresponding level expected in a healthy subject is at least temporarily reduced by 10%, 20%, 30%, 40%, 50%, 60%, 70%, 80%, 90% or 95%.

In one embodiment, the use of an inhibitor, nucleic acid or composition disclosed herein reduces the level of haemoglobin in the blood of a subject treated with the inhibitor, nucleic acid or composition to the corresponding level expected in a healthy subject. Alternatively, it reduces the level of haemoglobin in the blood of a subject treated with the inhibitor, nucleic acid or composition such that the difference between the level of haemoglobin in the blood in a subject before treatment and the corresponding level expected in a healthy subject is at least temporarily reduced by 10%, 20%, 30%, 40%, 50%, 60%, 70%, 80%, 90% or 95%.

In one embodiment, the myeloproliferative disorder is PV and the subject treated has a haemoglobin level of more than 16.5 g/dL (men) or of more than 16.0 g/dL (women) (Barbui et al., Blood Cancer J. 2018 February; 8(2): 15).

In one embodiment, the myeloproliferative disorder is PV and the bone marrow biopsy of the subject treated shows hypercellularity for age with trilineage growth (panmyelosis) including prominent erythroid, granulocytic and megakaryocytic proliferation with pleomorphic, mature megakaryocytes (differences in size) (Barbui et al., Blood Cancer J. 2018 February; 8(2): 15).

In one embodiment, the use of an inhibitor, nucleic acid or composition disclosed herein reduces the level of erythropoiesis in a subject treated with the inhibitor, nucleic acid or composition to the corresponding level expected in a healthy subject. Alternatively, it reduces the level of erythropoiesis in a subject treated with the inhibitor, nucleic acid or composition such that the difference between the level of erythropoiesis in a subject before treatment and the corresponding level expected in a healthy subject is at least temporarily reduced by 10%, 20%, 30%, 40%, 50%, 60%, 70%, 80%, 90% or 95%.

In one embodiment, the myeloproliferative disorder is PV characterized by an increased red cell mass. An “increased red cell mass” is a red cell mass which is more than 25% above mean normal predicted value (Barbui et al., Blood Cancer J. 2018 February; 8(2): 15).

It is evident that an appropriate dosage regimen of an inhibitor, nucleic acid or composition is necessary to achieve these outcomes. The skilled person will be able to determine the dosage regimen necessary to achieve these outcomes for a given subject.

### Combination Therapies

In certain embodiments, the inhibitors or nucleic acids or compositions described herein are for use or are used in a method of prevention, decrease of the risk of suffering from or treatment of a myeloproliferative disorder in combination with one or several of:
a) a cytoreductive therapy;
b) phlebotomy;
c) a hepcidin agonist;
d) a hepcidin mimetic, such as PTG300;
e) aspirin; and
f) an anticoagulant.

In one embodiment, a cytoreductive therapy is a therapy with hydroxyurea, hydroxycarbamide, interferon-α, pegylated interferon-α-2a, busulfan, a JAK2 inhibitor or Ruxolitinib,

Treatments that are used in combination are treatments that are administered at least twice, three times, four times, five times or more within 90 days or less, 80 days or less, 70 days or less, 60 days or less, 50 days or less, 40 days or less, 30 days or less, 20 days or less, 15 days or less, 10 days or less, 9 days or less, 8 days or less, 7 days or less, 6 days or less, 5 days or less, 4 days or less, 3 days or less, 48 hours or less, 36 hours or less, 24 hours or less, 12 hours or less, 6 hours or less, 3 hours or less, 2 hours or less, 1 hour or less, 30 minutes or less, 15 minutes or less, 10 minutes or less or 5 minutes or less from each other.

### Definitions

As used herein, the terms “inhibit”, “down-regulate”, or “reduce” with respect to gene expression mean that the expression of the gene, or the level of RNA molecules or equivalent RNA molecules encoding one or more proteins or protein subunits (e.g., mRNA), or the activity of one or more proteins or protein subunits, is reduced below that observed either in the absence of the nucleic acid or conjugated nucleic acid of the invention or as compared to that obtained with an siRNA molecule with no known homology to the human transcript (herein termed non-silencing control). Such control may be conjugated and modified in an analogous manner to the molecule of the invention and delivered into the target cell by the same route. The expression after treatment with the nucleic acid of the invention may be reduced to 95%, 90%, 80%, 70%, 60%, 50%, 40%, 30%, 20%, 15%, 10%, 5% or 0% or to intermediate values, or less than that observed in the absence of the nucleic acid or conjugated nucleic acid. The expression may be measured in the cells to which the nucleic acid is applied. Alternatively, especially if the nucleic acid is administered to a subject, the level can be measured in a different group of cells or in a tissue or an organ or in a body fluid such as blood or plasma. The level of inhibition is preferably measured in conditions that have been selected because they show the greatest effect of the nucleic acid on the target mRNA level in cells treated with the nucleic acid in vitro. The level of inhibition may for example be measured after 24 hours or 48 hours of treatment with a nucleic acid at a concentration of between 0.038 nM—10 μM, preferably 1 nM, 10 nM or 100 nM. These conditions may be different for different nucleic acid sequences or for different types of nucleic acids, such as for nucleic acids that are unmodified or modified or conjugated to a ligand or not. Examples of suitable conditions for determining levels of inhibition are described in the examples.

By nucleic acid it is meant a nucleic acid comprising one or two strands comprising nucleotides and that is able to interfere with gene expression. Inhibition may be complete or partial and result in down regulation of gene expression in a targeted manner. The nucleic acid may comprise two separate polynucleotide strands; the first strand, which may also be a guide strand; and a second strand, which may also be a passenger strand. The first strand and the second strand may be part of the same polynucleotide molecule that is self-complementary which ‘folds’ back to form a double-stranded molecule. The nucleic acid may be an siRNA molecule.

The nucleic acid may comprise ribonucleotides, modified ribonucleotides, deoxynucleotides, deoxyribonucleotides, or nucleotide analogues non-nucleotides that are able to mimic nucleotides such that they may ‘pair’ with the corresponding base on the target sequence or a complementary strand. The nucleic acid may further comprise a double-stranded nucleic acid portion or duplex region formed by all or a portion of the first strand (also known in the art as a guide strand) and all or a portion of the second strand (also known in the art as a passenger strand). The duplex region is defined as beginning with the first base pair formed between the first strand and the second strand and ending with the last base pair formed between the first strand and the second strand, inclusive.

By duplex region it is meant the region in two complementary or substantially complementary oligonucleotides that form base pairs with one another, either by Watson-Crick base pairing or any other manner that allows for a duplex between oligonucleotide strands that are complementary or substantially complementary. For example, an oligonucleotide strand having 21 nucleotide units can base pair with another oligonucleotide of 21 nucleotide units, yet only 19 nucleotides on each strand are complementary or substantially complementary, such that the “duplex region” consists of 19 base pairs. The remaining base pairs may exist as 5′ and 3′ overhangs, or as single-stranded regions. Further, within the duplex region, 100% complementarity is not required; substantial complementarity is allowable within a duplex region. Substantial complementarity refers to complementarity between the strands such that they are capable of annealing under biological conditions. Techniques to empirically determine if two strands are capable of annealing under biological conditions are well known in the art. Alternatively, two strands can be synthesised and added together under biological conditions to determine if they anneal to one another. The portion of the first strand and second strand that form at least one duplex region may be fully complementary or are at least partially complementary to each other. Depending on the length of a nucleic acid, a perfect match in terms of base complementarity between the first strand and the second strand is not necessarily required. However, the first and second strands must be able to hybridise under physiological conditions.

As used herein, the terms “non-pairing nucleotide analogue” means a nucleotide analogue which includes a non-base pairing moiety including but not limited to: 6 des amino adenosine (Nebularine), 4-Me-indole, 3-nitropyrrole, 5-nitroindole, Ds, Pa, N3-Me ribo U, N3-Me riboT,

N3-Me dC, N3-Me-dT, N1-Me-dG, N1-Me-dA, N3-ethyl-dC, and N3-Me dC. In some embodiments the non-base pairing nucleotide analogue is a ribonucleotide. In other embodiments it is a deoxyribonucleotide.

As used herein, the term, “terminal functional group” includes without limitation a halogen, alcohol, amine, carboxylic, ester, amide, aldehyde, ketone, and ether groups.

An “overhang” as used herein has its normal and customary meaning in the art, i.e. a single-stranded portion of a nucleic acid that extends beyond the terminal nucleotide of a complementary strand in a double-strand nucleic acid. The term “blunt end” includes double-stranded nucleic acid whereby both strands terminate at the same position, regardless of whether the terminal nucleotide(s) are base-paired. The terminal nucleotide of a first strand and a second strand at a blunt end may be base paired. The terminal nucleotide of a first strand and a second strand at a blunt end may not be paired. The terminal two nucleotides of a first strand and a second strand at a blunt end may be base-paired. The terminal two nucleotides of a first strand and a second strand at a blunt end may not be paired.

The term “serinol-derived linker moiety” means the linker moiety comprises the following structure:

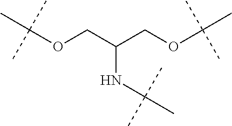

An O atom of said structure typically links to an RNA strand and the N atom typically links to the targeting ligand.

“Matriptase-2” or “MT2” in the context of the present invention relates to human “Transmembrane protease serine 6” (UniProt ID Q8IU80), encoded by the gene TMPRSS6 (NCBI Gene ID: 164656).

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

As used herein, “treating” or “treatment” and grammatical variants thereof refer to an approach for obtaining beneficial or desired clinical results. The term may refer to slowing the onset or rate of development of a condition, disorder or disease, reducing or alleviating symptoms associated with it, generating a complete or partial regression of the condition, or some combination of any of the above. For the purposes of this invention, beneficial or desired clinical results include, but are not limited to, reduction or alleviation of symptoms, diminishment of extent of disease, stabilization (i.e., not worsening) of state of disease, delay or slowing of disease progression, amelioration or palliation of the disease state, and remission (whether partial or total), whether detectable or undetectable. “Treatment” can also mean prolonging survival relative to expected survival time if not receiving treatment. A subject (e.g., a human) in need of treatment may thus be a subject already afflicted with the disease or disorder in question. The term “treatment” includes inhibition or reduction of an increase in severity of a pathological state or symptoms relative to the absence of treatment, and is not necessarily meant to imply complete or even partial cessation of the relevant disease, disorder or condition. “Treatment” of a disease, disorder or condition may be limited to reducing the extent of one or more symptom of the disease, disorder or condition.

As used herein, the terms “preventing” and grammatical variants thereof refer to an approach for preventing the development of, or altering the pathology of, a condition, disease or disorder. Accordingly, “prevention” may refer to prophylactic or preventive measures. For the purposes of this invention, beneficial or desired clinical results include, but are not limited to, prevention or slowing of symptoms, progression or development of a disease, whether detectable or undetectable. A subject (e.g., a human) in need of prevention may thus be a subject not yet afflicted with the disease or disorder in question. The term “prevention” includes slowing the onset of disease relative to the absence of treatment, and is not necessarily meant to imply permanent prevention of the relevant disease, disorder or condition. Thus “preventing” or “prevention” of a condition may in certain contexts refer to reducing the risk of developing the condition, or preventing or delaying the development of symptoms associated with the condition.

As used herein, an “effective amount,” “therapeutically effective amount” or “effective dose” is an amount of a composition (e.g., a therapeutic composition or agent) that produces at least one desired therapeutic effect in a subject, such as preventing or treating a target condition or beneficially alleviating a symptom associated with the condition.

As used herein, the term “pharmaceutically acceptable salt” refers to a salt that is not harmful to a patient or subject to which the salt in question is administered. It may be a salt chosen, e.g., among acid addition salts and basic salts. Examples of acid addition salts include chloride salts, citrate salts and acetate salts. Examples of basic salts include salts wherein the cation is selected from alkali metal cations, such as sodium or potassium ions, alkaline earth metal cations, such as calcium or magnesium ions, as well as substituted ammonium ions, such as ions of the type N(R¹)(R²)(R³)(R⁴)+, wherein R¹, R², R³ and R⁴ independently will typically designate hydrogen, optionally substituted C1-6-alkyl groups or optionally substituted C2-6-alkenyl groups. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, PA, USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). A “pharmaceutically acceptable salt” retains qualitatively a desired biological activity of the parent compound without imparting any undesired effects relative to the compound. Examples of pharmaceutically acceptable salts include acid addition salts and base addition salts. Acid addition salts include salts derived from nontoxic inorganic acids, such as hydrochloric, nitric, phosphorous, phosphoric, sulfuric, hydrobromic, hydroiodic and the like, or from nontoxic organic acids such as aliphatic mono- and di-carboxylic acids, phenyl-substituted alkanoic acids, hydroxy alkanoic acids, aromatic acids, aliphatic and aromatic sulfonic acids and the like. Base addition salts include salts derived from alkaline earth metals, such as sodium, potassium, magnesium, calcium and the like, as well as from nontoxic organic amines, such as N, N′-dibenzylethylenediamine, N-methylglucamine, chloroprocaine, choline, diethanolamine, ethylenediamine, procaine and the like.

The term “pharmaceutically acceptable carrier” includes any of the standard pharmaceutical carriers. Pharmaceutically acceptable carriers for therapeutic use are well known in the pharmaceutical art, and are described, for example, in Remington's Pharmaceutical Sciences, Mack Publishing Co. (A. R. Gennaro edit. 1985). For example, sterile saline and phosphate-buffered saline at slightly acidic or physiological pH may be used. Exemplary pH buffering agents include phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. The term further encompasses any agents listed in the US Pharmacopeia for use in animals, including humans. A “pharmaceutically acceptable carrier” includes any and all physiologically acceptable, i.e., compatible, solvents, dispersion media, coatings, antimicrobial agents, isotonic and absorption delaying agents, and the like. In certain embodiments, the carrier is suitable for intravenous, intramuscular, subcutaneous, parenteral, spinal or epidermal administration (e.g., by injection or infusion). Depending on selected route of administration, the nucleic acid may be coated in a material or materials intended to protect the compound from the action of acids and other natural inactivating conditions to which the nucleic acid may be exposed when administered to a subject by a particular route of administration.

The term “solvate” in the context of the present invention refers to a complex of defined stoichiometry formed between a solute (in casu, a nucleic acid compound or pharmaceutically acceptable salt thereof according to the invention) and a solvent. The solvent in this connection may, for example, be water or another pharmaceutically acceptable, typically small-molecular organic species, such as, but not limited to, acetic acid or lactic acid. When the solvent in question is water, such a solvate is normally referred to as a hydrate.

The invention will now be described with reference to the following non-limiting Figures and Examples.

### BRIEF DESCRIPTION OF THE FIGURES

FIG. 1 shows the effect on different parameters of treating polycythaemia vera mice models or control mice with a TMPRSS6 siRNA GalNAc conjugate.

FIG. 2 shows the effect on different parameters of treating polycythaemia vera mice models or control mice with a TMPRSS6 siRNA GalNAc conjugate.

FIG. 3 shows SEQ ID NO: 1385: the TMPRSS6 mRNA sequence: NM_153609.4 Homo sapiens transmembrane serine protease 6 (TMPRSS6), transcript variant 2, mRNA.

FIG. 4 shows a dose dependent reduction of TMPRSS6 mRNA levels in primary hepatocytes by TMPRSS6 siRNA conjugates EU401 and EU402 by receptor mediated uptake.

### EXAMPLES

### Example 1—Synthesis of Building Blocks

Synthesis of (vp)-mU-phos was performed as described in Prakash, Nucleic Acids Res. 2015, 43(6), 2993-3011 and Haraszti, Nucleic Acids Res. 2017, 45(13), 7581-7592. Synthesis of the phosphoramidite derivatives of ST41 (ST41-phos), ST43 (ST43-phos) as well as ST23 (ST23-phos) and similar can be performed as described in WO2017/174657.

### Example 2—Oligonucleotide Synthesis

Example compounds were synthesised according to methods described below and known to the person skilled in the art. Assembly of the oligonucleotide chain and linker building blocks was performed by solid phase synthesis applying phosphoramidite methodology.

Downstream cleavage, deprotection and purification followed standard procedures that are known in the art.

Oligonucleotide syntheses was performed on an AKTA oligopilot 10 using commercially available 2′O-Methyl RNA and 2′Fluoro-2′Deoxy RNA base loaded CPG solid support and phosphoramidites (all standard protection, ChemGenes, LinkTech) were used.

Ancillary reagents were purchased from EMP Biotech. Synthesis was performed using a 0.1 M solution of the phosphoramidite in dry acetonitrile (<20 ppm H₂O) and benzylthiotetrazole (BTT) was used as activator (0.3M in acetonitrile). Coupling time was 10 min. A Cap/OX/Cap or Cap/Thio/Cap cycle was applied (Cap: AC₂O/NMI/Lutidine/Acetonitrile, Oxidizer: 0.05M 12 in pyridine/H₂O). Phosphorothioates were introduced using commercially available thiolation reagent 50 mM EDITH in acetonitrile (Link technologies). DMT cleavage was achieved by treatment with 3% dichloroacetic acid in toluene. Upon completion of the programmed synthesis cycles a diethylamine (DEA) wash was performed. All oligonucleotides were synthesized in DMT-off mode.

Tri-antennary GalNAc clusters (ST23/ST43 or ST23/ST41) were introduced by successive coupling of the branching trebler amidite derivative (ST43-phos or ST41-phos) followed by the GalNAc amidite (ST23-phos). Attachment of (vp)-mU moiety was achieved by use of (vp)-mU-phos in the last synthesis cycle. The (vp)-mU-phos does not provide a hydroxy group suitable for further synthesis elongation and therefore, does not possess an DMT-group. Hence coupling of (vp)-mU-phos results in synthesis termination.

For the removal of the methyl esters masking the vinylphosphonate, the CPG carrying the fully assembled oligonucleotide was dried under reduced pressure and transferred into a 20 ml PP syringe reactor for solid phase peptide synthesis equipped with a disc frit (Carl Roth GmbH). The CPG was then brought into contact with a solution of 250 μL TMSBr and 177 L pyridine in CH₂Cl₂ (0.5 ml/μmol solid support bound oligonucleotide) at room temperature and the reactor was sealed with a Luer cap. The reaction vessels were slightly agitated over a period of 2×15 min, the excess reagent discarded, and the residual CPG washed 2× with 10 ml acetonitrile. Further downstream processing did not alter from any other example compound.

The single strands were cleaved off the CPG by 40% aq. methylamine treatment (90 min, RT). The resulting crude oligonucleotide was purified by ion exchange chromatography (Resource Q, 6 ml, GE Healthcare) on a AKTA Pure HPLC System using a sodium chloride gradient. Product containing fractions were pooled, desalted on a size exclusion column (Zetadex, EMP Biotech) and lyophilised until further use.

All final single-stranded products were analysed by AEX-HPLC to prove their purity. Identity of the respective single-stranded products was proved by LC-MS analysis.

### Example 3—Double-Strand Formation

Individual single strands were dissolved in a concentration of 60 OD/ml in H₂O. Both individual oligonucleotide solutions were added together in a reaction vessel. For easier reaction monitoring a titration was performed. The first strand was added in 25% excess over the second strand as determined by UV-absorption at 260 nm. The reaction mixture was heated to 80° C. for 5 min and then slowly cooled to RT. Double-strand formation was monitored by ion pairing reverse phase HPLC. From the UV-area of the residual single strand the needed amount of the second strand was calculated and added to the reaction mixture. The reaction was heated to 80° C. again and slowly cooled to RT. This procedure was repeated until less than 10% of residual single strand was detected.

### Example 4

Inhibition of TMPRSS6 expression by TMPRSS6 siRNA treatment in a rodent model for polycythaemia vera (PV) leads to an increase of hepcidin levels and a reduction of serum iron levels.

A rodent model for polycythemia vera (PV) was established by transplantation of murine haematopoietic stem cells that carry one inducible human JAK2V617F allele and a Cre recombinase transgene under the control of the Tamoxifen inducible promoter (CreERT2) into preconditioned wild type Ly5.1/J recipient mice. Control animals received haematopoietic stem cells that carry only the JAK2V617F knock-in allele (JAK2 KI control). 46 days after transplantation, engraftment was confirmed by blood analysis. 49 days after transplantation, expression of the JAK2V617F transgene was activated in the PV mouse model by administration of Tamoxifen (4.2 mg by oral gavage in 10% ethanol/90% corn oil) on two consecutive days. The different genotypes (JAK2 KI control and PV) were each randomized into three groups and treated on Day 56, Day 77 and Day 98 after transplantation with either the vehicle (PBS), 5 mg GalNAc conjugated TMPRSS6 siRNA (EU401) or 5 mg non-targeting control siRNA molecule (EU400) per kg body weight. On Day 105 after transplantation all animals were humanely euthanized and tissue and blood samples collected for analyses. Liver tissue samples were stored in nucleic acid preserving media (RNAlater) and total RNA extracted to measure target gene expression by qRT-PCR. Serum samples were prepared from terminal blood samples and hepcidin levels were determined by ELISA assay and serum iron levels were determined using an Abbott ARCHITECT analyzer with the MULTIGENT Iron assay. Tissue non-haem iron levels were determined by the Ferrozine method from shock-frozen liver samples.

Treatment with EU401 reduces hepatic Tmprss6 mRNA expression in the control mice as well as in the rodent model for polycythaemia vera (PV). EU401 treatment also raises hepatic Hamp1 mRNA levels and serum hepcidin levels and reduces serum iron levels in control mice and in the PV mouse model. Hepatic iron levels were not significantly affected by the treatment with EU401.

Statistics: Experimental data are presented as bar chart with median±95% CI. Distribution-free Mann-Whitney U tests were used for the pairwise comparisons. Data was considered statistically significant, if the P-value was ≤ 0.05 (ns: P >0.05; * P≤ 0.05; ** P≤ 0.01; *** P≤ 0.001, *** P≤ 0.0001). The test statistics were not corrected for multiple testing.

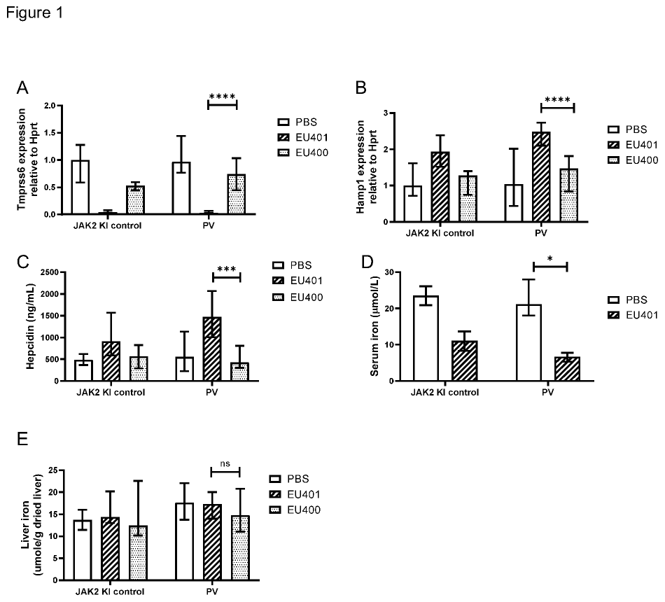

Results are shown in FIG. 1A) Tmprss6 mRNA levels in liver, B) Hamp1 mRNA level in liver, C) Hepcidin level in serum, D) Iron levels in serum, E) Iron levels in liver tissue. Tmprss6 and Hamp1 mRNA levels are depicted normalized to the house keeping gene Hprt and presented as fold change vs PBS treated JAK2 KI control (set to 1). siRNAs are listed in Table 2.

Similar results to those obtained with EU401 are expected with EU402 and possibly other inhibitors of MP2 function or expression.

### Example 5

Reduction of haemoglobin and haematocrit by GalNAc-conjugated TMPRSS6 siRNA in an animal model for polycythaemia vera.

A rodent model for polycythaemia vera (PV) was set up and treated with vehicle (PBS), EU401 and EU400 as described in Example 4. At the end of the study, terminal blood samples and bone marrow were collected. Full blood cell counts were determined by automatic haemocytometer (Advia 2120i) and red blood cell maturation was assessed in the bone marrow by flow cytometry as previously described (Chen et al., 2009, PNAS, 106(41): 17413-17418).

Treatment with EU401 reduces haemoglobin and haematocrit in the blood of the rodent model for polycythaemia vera (PV) and in the control mice. EU401 treatment also reduces the mean corpuscular volume in the PV model and in the control mice, when compared to the corresponding control group of the same genotype that received either the vehicle or EU400. EU401 resets red blood cell maturation in the bone marrow of the PV mouse model by normalizing the mature red cell population and expanding progenitor populations.

Statistics: Experimental data are presented as bar chart with median+95% CI. Distribution-free Mann-Whitney U tests were used for the pairwise comparisons. Data was considered statistically significant, if the P-value was ≤ 0.05 (ns: P >0.05; * P≤ 0.05; ** P≤ 0.01; *** P≤ 0.001, *** P≤ 0.0001). The test statistics were not corrected for multiple testing. The proportion of erythroid progenitor populations are presented by stacked bar chart with mean±SD.

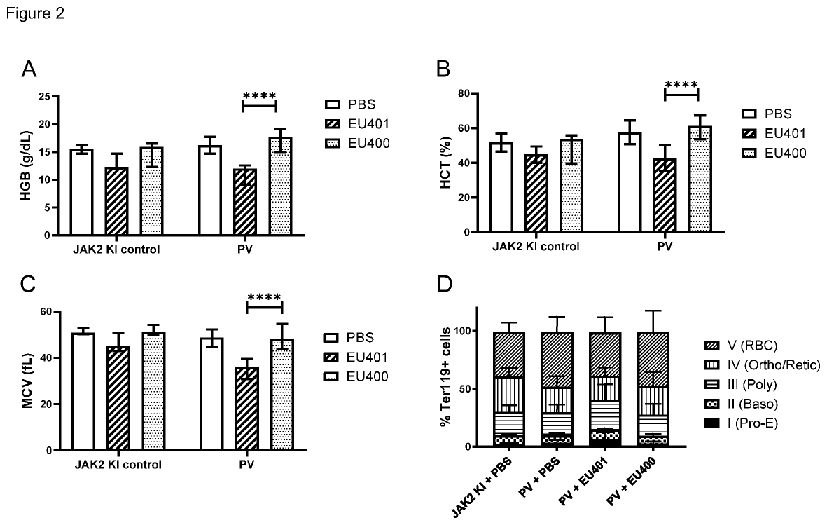

Results are shown in FIG. 2A) Haemoglobin (HGB), B) Haematocrit (HCT), C) Mean corpuscular volume (MCV) in red blood cells, D) Red blood cell maturation in the bone marrow. siRNAs are listed in Table 2.

Similar results to those obtained with EU401 are expected with EU402 and possibly other inhibitors of MP2 function or expression.

### Example 6

Reduction of TMPRSS6 mRNA levels in primary hepatocytes

Primary mouse hepatocytes were seeded in a 96 well plate at a density of 25,000 cells per well. After plating, cells were incubated with the TMPRSS6 siRNA conjugates EU401 and EU402 in the cell culture medium at different concentrations (100 nM, 33 nM, 11 nM, 3.7, 1.2 nM, 0.41 nM and 0.14 nM). The following day cells were lysed for RNA extraction and TMPRSS6 and Actin mRNA levels were determined by Taqman qRT-PCR. Values obtained for TMPRSS6 mRNA were normalized to values generated for the house keeping gene, Actin, and related to mean of untreated sample (ut) set at 1-fold target gene expression. Each bar represents mean+/−SD from three biological replicates. siRNA conjugates EU401 and EU402 used in this study are further described in Table 2 and Table 4.

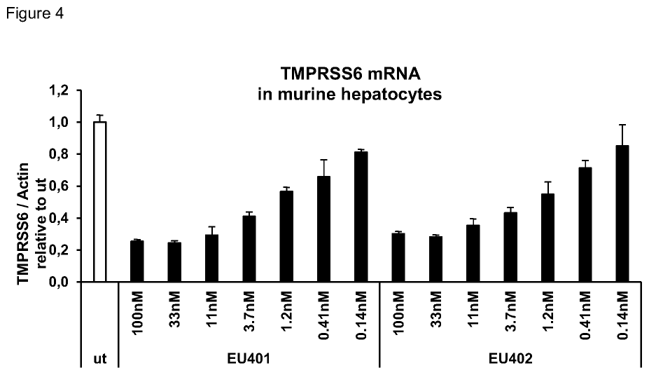

The results shown in FIG. 4 demonstrate a dose dependent reduction of TMPRSS6 mRNA levels in primary hepatocytes by TMPRSS6 siRNA conjugates EU401 and EU402 by receptor mediated uptake.

### Summary Tables

### Summary Duplex Table

TABLE 2

| | Single | | Single | | Single |
|---|---|---|---|---|---|
| Duplex | Strands | Duplex | Strands | Duplex | Strands |
| EU400 | EU400A | AL1-214 | AL1-214A | AL2-222 | AL2-222A |
| | EU400B | | AL1-214B | | AL2-222B |
| EU401 | EU401A | AL1-215 | AL1-215A | AL2-223 | AL2-223A |
| | EU401B | | AL1-215B | | AL2-223B |
| EU402 | EU401A | AL1-216 | AL1-216A | AL2-224 | AL2-224A |
| | EU402B | | AL1-216B | | AL2-224B |
| TMPRSS6-hc-1 | TMPRSS6-hc-1A | AL1-217 | AL1-217A | AL2-225 | AL2-225A |
| | TMPRSS6-hc-1B | | AL1-217B | | AL2-225B |
| TMPRSS6-h-2 | TMPRSS6-h-2A | AL1-218 | AL1-218A | AL2-226 | AL2-226A |
| | TMPRSS6-h-2B | | AL1-218B | | AL2-226B |
| TMPRSS6-h-3 | TMPRSS6-h-3A | AL1-219 | AL1-219A | AL2-227 | AL2-227A |
| | TMPRSS6-h-3B | | AL1-219B | | AL2-227B |
| TMPRSS6-hc-4 | TMPRSS6-hc-4A | AL1-220 | AL1-220A | AL2-228 | AL2-228A |
| | TMPRSS6-hc-4B | | AL1-220B | | AL2-228B |
| TMPRSS6-h-5 | TMPRSS6-h-5A | AL1-221 | AL1-221A | AL2-229 | AL2-229A |
| | TMPRSS6-h-5B | | AL1-221B | | AL2-229B |
| TMPRSS6-h-6 | TMPRSS6-h-6A | AL1-222 | AL1-222A | AL2-230 | AL2-230A |
| | TMPRSS6-h-6B | | AL1-222B | | AL2-230B |
| TMPRSS6-h-7 | TMPRSS6-h-7A | AL1-223 | AL1-223A | AL2-231 | AL2-231A |
| | TMPRSS6-h-7B | | AL1-223B | | AL2-231B |
| TMPRSS6-hcmr-8 | TMPRSS6-hcmr-8A | AL2-1 | AL2-1A | AL2-232 | AL2-232A |
| | TMPRSS6-hcmr-8B | | AL2-1B | | AL2-232B |
| TMPRSS6-hcm-9 | TMPRSS6-hcm-9A | AL2-2 | AL2-2A | AL2-233 | AL2-233A |
| | TMPRSS6-hcm-9B | | AL2-2B | | AL2-233B |
| TMPRSS6-hc-10 | TMPRSS6-hc-10A | AL2-3 | AL2-3A | AL2-234 | AL2-234A |
| | TMPRSS6-hc-10B | | AL2-3B | | AL2-234B |
| TMPRSS6-hc-11 | TMPRSS6-hc-11A | AL2-4 | AL2-4A | AL2-235 | AL2-235A |
| | TMPRSS6-hc-11B | | AL2-4B | | AL2-235B |
| TMPRSS6-hcm-12 | TMPRSS6-hcm-12A | AL2-5 | AL2-5A | AL2-236 | AL2-236A |
| | TMPRSS6-hcm-12B | | AL2-5B | | AL2-236B |
| TMPRSS6-hc-13 | TMPRSS6-hc-13A | AL2-6 | AL2-6A | AL2-237 | AL2-237A |
| | TMPRSS6-hc-13B | | AL2-6B | | AL2-237B |
| TMPRSS6-hcmr-14 | TMPRSS6-hcmr-14A | AL2-7 | AL2-7A | AL2-238 | AL2-238A |
| | TMPRSS6-hcmr-14B | | AL2-7B | | AL2-238B |
| TMPRSS6-hcmr-15 | TMPRSS6-hcmr-15A | AL2-8 | AL2-8A | AL2-239 | AL2-239A |
| | TMPRSS6-hcmr-15B | | AL2-8B | | AL2-239B |
| AL1-1 | AL1-1A | AL2-9 | AL2-9A | AL2-240 | AL2-240A |
| | AL1-1B | | AL2-9B | | AL2-240B |
| AL1-2 | AL1-2A | AL2-10 | AL2-10A | AL2-241 | AL2-241A |
| | AL1-2B | | AL2-10B | | AL2-241B |
| AL1-3 | AL1-3A | AL2-11 | AL2-11A | AL2-242 | AL2-242A |
| | AL1-3B | | AL2-11B | | AL2-242B |
| AL1-4 | AL1-4A | AL2-12 | AL2-12A | AL2-243 | AL2-243A |
| | AL1-4B | | AL2-12B | | AL2-243B |
| AL1-5 | AL1-5A | AL2-13 | AL2-13A | AL2-244 | AL2-244A |
| | AL1-5B | | AL2-13B | | AL2-244B |
| AL1-6 | AL1-6A | AL2-14 | AL2-14A | AL2-245 | AL2-245A |
| | AL1-6B | | AL2-14B | | AL2-245B |
| AL1-7 | AL1-7A | AL2-15 | AL2-15A | AL2-246 | AL2-246A |
| | AL1-7B | | AL2-15B | | AL2-246B |
| AL1-8 | AL1-8A | AL2-16 | AL2-16A | AL2-247 | AL2-247A |
| | AL1-8B | | AL2-16B | | AL2-247B |
| AL1-9 | AL1-9A | AL2-17 | AL2-17A | AL2-248 | AL2-248A |
| | AL1-9B | | AL2-17B | | AL2-248B |
| AL1-10 | AL1-10A | AL2-18 | AL2-18A | AL2-249 | AL2-249A |
| | AL1-10B | | AL2-18B | | AL2-249B |
| AL1-11 | AL1-11A | AL2-19 | AL2-19A | AL2-250 | AL2-250A |
| | AL1-11B | | AL2-19B | | AL2-250B |
| AL1-12 | AL1-12A | AL2-20 | AL2-20A | AL2-251 | AL2-251A |
| | AL1-12B | | AL2-20B | | AL2-251B |
| AL1-13 | AL1-13A | AL2-21 | AL2-21A | AL2-252 | AL2-252A |
| | AL1-13B | | AL2-21B | | AL2-252B |
| AL1-14 | AL1-14A | AL2-22 | AL2-22A | AL2-253 | AL2-253A |
| | AL1-14B | | AL2-22B | | AL2-253B |
| AL1-15 | AL1-15A | AL2-23 | AL2-23A | AL2-254 | AL2-254A |
| | AL1-15B | | AL2-23B | | AL2-254B |
| AL1-16 | AL1-16A | AL2-24 | AL2-24A | AL2-255 | AL2-255A |
| | AL1-16B | | AL2-24B | | AL2-255B |
| AL1-17 | AL1-17A | AL2-25 | AL2-25A | AL2-256 | AL2-256A |
| | AL1-17B | | AL2-25B | | AL2-256B |
| AL1-18 | AL1-18A | AL2-26 | AL2-26A | AL2-257 | AL2-257A |
| | AL1-18B | | AL2-26B | | AL2-257B |
| AL1-19 | AL1-19A | AL2-27 | AL2-27A | AL2-258 | AL2-258A |
| | AL1-19B | | AL2-27B | | AL2-258B |
| AL1-20 | AL1-20A | AL2-28 | AL2-28A | AL2-259 | AL2-259A |
| | AL1-20B | | AL2-28B | | AL2-259B |
| AL1-21 | AL1-21A | AL2-29 | AL2-29A | AL2-260 | AL2-260A |
| | AL1-21B | | AL2-29B | | AL2-260B |
| AL1-22 | AL1-22A | AL2-30 | AL2-30A | AL2-261 | AL2-261A |
| | AL1-22B | | AL2-30B | | AL2-261B |
| AL1-23 | AL1-23A | AL2-31 | AL2-31A | AL2-262 | AL2-262A |
| | AL1-23B | | AL2-31B | | AL2-262B |
| AL1-24 | AL1-24A | AL2-32 | AL2-32A | AL2-263 | AL2-263A |
| | AL1-24B | | AL2-32B | | AL2-263B |
| AL1-25 | AL1-25A | AL2-33 | AL2-33A | AL2-264 | AL2-264A |
| | AL1-25B | | AL2-33B | | AL2-264B |
| AL1-26 | AL1-26A | AL2-34 | AL2-34A | AL2-265 | AL2-265A |
| | AL1-26B | | AL2-34B | | AL2-265B |
| AL1-27 | AL1-27A | AL2-35 | AL2-35A | AL2-266 | AL2-266A |
| | AL1-27B | | AL2-35B | | AL2-266B |
| AL1-28 | AL1-28A | AL2-36 | AL2-36A | AL2-267 | AL2-267A |
| | AL1-28B | | AL2-36B | | AL2-267B |
| AL1-29 | AL1-29A | AL2-37 | AL2-37A | AL2-268 | AL2-268A |
| | AL1-29B | | AL2-37B | | AL2-268B |
| AL1-30 | AL1-30A | AL2-38 | AL2-38A | AL2-269 | AL2-269A |
| | AL1-30B | | AL2-38B | | AL2-269B |
| AL1-31 | AL1-31A | AL2-39 | AL2-39A | AL2-270 | AL2-270A |
| | AL1-31B | | AL2-39B | | AL2-270B |
| AL1-32 | AL1-32A | AL2-40 | AL2-40A | AL2-271 | AL2-271A |
| | AL1-32B | | AL2-40B | | AL2-271B |
| AL1-33 | AL1-33A | AL2-41 | AL2-41A | AL2-272 | AL2-272A |
| | AL1-33B | | AL2-41B | | AL2-272B |
| AL1-34 | AL1-34A | AL2-42 | AL2-42A | AL2-273 | AL2-273A |
| | AL1-34B | | AL2-42B | | AL2-273B |
| AL1-35 | AL1-35A | AL2-43 | AL2-43A | AL2-274 | AL2-274A |
| | AL1-35B | | AL2-43B | | AL2-274B |
| AL1-36 | AL1-36A | AL2-44 | AL2-44A | AL2-275 | AL2-275A |
| | AL1-36B | | AL2-44B | | AL2-275B |
| AL1-37 | AL1-37A | AL2-45 | AL2-45A | AL2-276 | AL2-276A |
| | AL1-37B | | AL2-45B | | AL2-276B |
| AL1-38 | AL1-38A | AL2-46 | AL2-46A | AL2-277 | AL2-277A |
| | AL1-38B | | AL2-46B | | AL2-277B |
| AL1-39 | AL1-39A | AL2-47 | AL2-47A | AL2-278 | AL2-278A |
| | AL1-39B | | AL2-47B | | AL2-278B |
| AL1-40 | AL1-40A | AL2-48 | AL2-48A | AL2-279 | AL2-279A |
| | AL1-40B | | AL2-48B | | AL2-279B |
| AL1-41 | AL1-41A | AL2-49 | AL2-49A | AL2-280 | AL2-280A |
| | AL1-41B | | AL2-49B | | AL2-280B |
| AL1-42 | AL1-42A | AL2-50 | AL2-50A | AL2-281 | AL2-281A |
| | AL1-42B | | AL2-50B | | AL2-281B |
| AL1-43 | AL1-43A | AL2-51 | AL2-51A | AL2-282 | AL2-282A |
| | AL1-43B | | AL2-51B | | AL2-282B |
| AL1-44 | AL1-44A | AL2-52 | AL2-52A | AL2-283 | AL2-283A |
| | AL1-44B | | AL2-52B | | AL2-283B |
| AL1-45 | AL1-45A | AL2-53 | AL2-53A | AL2-284 | AL2-284A |
| | AL1-45B | | AL2-53B | | AL2-284B |
| AL1-46 | AL1-46A | AL2-54 | AL2-54A | AL2-285 | AL2-285A |
| | AL1-46B | | AL2-54B | | AL2-285B |
| AL1-47 | AL1-47A | AL2-55 | AL2-55A | AL2-286 | AL2-286A |
| | AL1-47B | | AL2-55B | | AL2-286B |
| AL1-48 | AL1-48A | AL2-56 | AL2-56A | AL2-287 | AL2-287A |
| | AL1-48B | | AL2-56B | | AL2-287B |
| AL1-49 | AL1-49A | AL2-57 | AL2-57A | AL2-288 | AL2-288A |
| | AL1-49B | | AL2-57B | | AL2-288B |
| AL1-50 | AL1-50A | AL2-58 | AL2-58A | AL2-289 | AL2-289A |
| | AL1-50B | | AL2-58B | | AL2-289B |
| AL1-51 | AL1-51A | AL2-59 | AL2-59A | AL2-290 | AL2-290A |
| | AL1-51B | | AL2-59B | | AL2-290B |
| AL1-52 | AL1-52A | AL2-60 | AL2-60A | AL2-291 | AL2-291A |
| | AL1-52B | | AL2-60B | | AL2-291B |
| AL1-53 | AL1-53A | AL2-61 | AL2-61A | AL2-292 | AL2-292A |
| | AL1-53B | | AL2-61B | | AL2-292B |
| AL1-54 | AL1-54A | AL2-62 | AL2-62A | AL2-293 | AL2-293A |
| | AL1-54B | | AL2-62B | | AL2-293B |
| AL1-55 | AL1-55A | AL2-63 | AL2-63A | AL2-294 | AL2-294A |
| | AL1-55B | | AL2-63B | | AL2-294B |
| AL1-56 | AL1-56A | AL2-64 | AL2-64A | AL2-295 | AL2-295A |
| | AL1-56B | | AL2-64B | | AL2-295B |
| AL1-57 | AL1-57A | AL2-65 | AL2-65A | AL2-296 | AL2-296A |
| | AL1-57B | | AL2-65B | | AL2-296B |
| AL1-58 | AL1-58A | AL2-66 | AL2-66A | AL2-297 | AL2-297A |
| | AL1-58B | | AL2-66B | | AL2-297B |
| AL1-59 | AL1-59A | AL2-67 | AL2-67A | AL2-298 | AL2-298A |
| | AL1-59B | | AL2-67B | | AL2-298B |
| AL1-60 | AL1-60A | AL2-68 | AL2-68A | AL2-299 | AL2-299A |
| | AL1-60B | | AL2-68B | | AL2-299B |
| AL1-61 | AL1-61A | AL2-69 | AL2-69A | AL2-300 | AL2-300A |
| | AL1-61B | | AL2-69B | | AL2-300B |
| AL1-62 | AL1-62A | AL2-70 | AL2-70A | AL2-301 | AL2-301A |
| | AL1-62B | | AL2-70B | | AL2-301B |
| AL1-63 | AL1-63A | AL2-71 | AL2-71A | AL2-302 | AL2-302A |
| | AL1-63B | | AL2-71B | | AL2-302B |
| AL1-64 | AL1-64A | AL2-72 | AL2-72A | AL2-303 | AL2-303A |
| | AL1-64B | | AL2-72B | | AL2-303B |
| AL1-65 | AL1-65A | AL2-73 | AL2-73A | AL2-304 | AL2-304A |
| | AL1-65B | | AL2-73B | | AL2-304B |
| AL1-66 | AL1-66A | AL2-74 | AL2-74A | AL2-305 | AL2-305A |
| | AL1-66B | | AL2-74B | | AL2-305B |
| AL1-67 | AL1-67A | AL2-75 | AL2-75A | AL2-306 | AL2-306A |
| | AL1-67B | | AL2-75B | | AL2-306B |
| AL1-68 | AL1-68A | AL2-76 | AL2-76A | AL2-307 | AL2-307A |
| | AL1-68B | | AL2-76B | | AL2-307B |
| AL1-69 | AL1-69A | AL2-77 | AL2-77A | AL2-308 | AL2-308A |
| | AL1-69B | | AL2-77B | | AL2-308B |
| AL1-70 | AL1-70A | AL2-78 | AL2-78A | AL2-309 | AL2-309A |
| | AL1-70B | | AL2-78B | | AL2-309B |
| AL1-71 | AL1-71A | AL2-79 | AL2-79A | AL2-310 | AL2-310A |
| | AL1-71B | | AL2-79B | | AL2-310B |
| AL1-72 | AL1-72A | AL2-80 | AL2-80A | AL2-311 | AL2-311A |
| | AL1-72B | | AL2-80B | | AL2-311B |
| AL1-73 | AL1-73A | AL2-81 | AL2-81A | AL2-312 | AL2-312A |
| | AL1-73B | | AL2-81B | | AL2-312B |
| AL1-74 | AL1-74A | AL2-82 | AL2-82A | AL2-313 | AL2-313A |
| | AL1-74B | | AL2-82B | | AL2-313B |
| AL1-75 | AL1-75A | AL2-83 | AL2-83A | AL2-314 | AL2-314A |
| | AL1-75B | | AL2-83B | | AL2-314B |
| AL1-76 | AL1-76A | AL2-84 | AL2-84A | AL2-315 | AL2-315A |
| | AL1-76B | | AL2-84B | | AL2-315B |
| AL1-77 | AL1-77A | AL2-85 | AL2-85A | AL2-316 | AL2-316A |
| | AL1-77B | | AL2-85B | | AL2-316B |
| AL1-78 | AL1-78A | AL2-86 | AL2-86A | AL2-317 | AL2-317A |
| | AL1-78B | | AL2-86B | | AL2-317B |
| AL1-79 | AL1-79A | AL2-87 | AL2-87A | AL2-318 | AL2-318A |
| | AL1-79B | | AL2-87B | | AL2-318B |
| AL1-80 | AL1-80A | AL2-88 | AL2-88A | AL2-319 | AL2-319A |
| | AL1-80B | | AL2-88B | | AL2-319B |
| AL1-81 | AL1-81A | AL2-89 | AL2-89A | AL2-320 | AL2-320A |
| | AL1-81B | | AL2-89B | | AL2-320B |
| AL1-82 | AL1-82A | AL2-90 | AL2-90A | AL2-321 | AL2-321A |
| | AL1-82B | | AL2-90B | | AL2-321B |
| AL1-83 | AL1-83A | AL2-91 | AL2-91A | AL2-322 | AL2-322A |
| | AL1-83B | | AL2-91B | | AL2-322B |
| AL1-84 | AL1-84A | AL2-92 | AL2-92A | AL2-323 | AL2-323A |
| | AL1-84B | | AL2-92B | | AL2-323B |
| AL1-85 | AL1-85A | AL2-93 | AL2-93A | AL2-324 | AL2-324A |
| | AL1-85B | | AL2-93B | | AL2-324B |
| AL1-86 | AL1-86A | AL2-94 | AL2-94A | AL2-325 | AL2-325A |
| | AL1-86B | | AL2-94B | | AL2-325B |
| AL1-87 | AL1-87A | AL2-95 | AL2-95A | AL2-326 | AL2-326A |
| | AL1-87B | | AL2-95B | | AL2-326B |
| AL1-88 | AL1-88A | AL2-96 | AL2-96A | AL2-327 | AL2-327A |
| | AL1-88B | | AL2-96B | | AL2-327B |
| AL1-89 | AL1-89A | AL2-97 | AL2-97A | AL2-328 | AL2-328A |
| | AL1-89B | | AL2-97B | | AL2-328B |
| AL1-90 | AL1-90A | AL2-98 | AL2-98A | AL2-329 | AL2-329A |
| | AL1-90B | | AL2-98B | | AL2-329B |
| AL1-91 | AL1-91A | AL2-99 | AL2-99A | AL2-330 | AL2-330A |
| | AL1-91B | | AL2-99B | | AL2-330B |
| AL1-92 | AL1-92A | AL2-100 | AL2-100A | AL2-331 | AL2-331A |
| | AL1-92B | | AL2-100B | | AL2-331B |
| AL1-93 | AL1-93A | AL2-101 | AL2-101A | AL2-332 | AL2-332A |
| | AL1-93B | | AL2-101B | | AL2-332B |
| AL1-94 | AL1-94A | AL2-102 | AL2-102A | AL2-333 | AL2-333A |
| | AL1-94B | | AL2-102B | | AL2-333B |
| AL1-95 | AL1-95A | AL2-103 | AL2-103A | AL2-334 | AL2-334A |
| | AL1-95B | | AL2-103B | | AL2-334B |
| AL1-96 | AL1-96A | AL2-104 | AL2-104A | AL2-335 | AL2-335A |
| | AL1-96B | | AL2-104B | | AL2-335B |
| AL1-97 | AL1-97A | AL2-105 | AL2-105A | AL2-336 | AL2-336A |
| | AL1-97B | | AL2-105B | | AL2-336B |
| AL1-98 | AL1-98A | AL2-106 | AL2-106A | AL2-337 | AL2-337A |
| | AL1-98B | | AL2-106B | | AL2-337B |
| AL1-99 | AL1-99A | AL2-107 | AL2-107A | AL2-338 | AL2-338A |
| | AL1-99B | | AL2-107B | | AL2-338B |
| AL1-100 | AL1-100A | AL2-108 | AL2-108A | AL2-339 | AL2-339A |
| | AL1-100B | | AL2-108B | | AL2-339B |
| AL1-101 | AL1-101A | AL2-109 | AL2-109A | AL2-340 | AL2-340A |
| | AL1-101B | | AL2-109B | | AL2-340B |
| AL1-102 | AL1-102A | AL2-110 | AL2-110A | AL2-341 | AL2-341A |
| | AL1-102B | | AL2-110B | | AL2-341B |
| AL1-103 | AL1-103A | AL2-111 | AL2-111A | AL2-342 | AL2-342A |
| | AL1-103B | | AL2-111B | | AL2-342B |
| AL1-104 | AL1-104A | AL2-112 | AL2-112A | AL2-343 | AL2-343A |
| | AL1-104B | | AL2-112B | | AL2-343B |
| AL1-105 | AL1-105A | AL2-113 | AL2-113A | AL2-344 | AL2-344A |
| | AL1-105B | | AL2-113B | | AL2-344B |
| AL1-106 | AL1-106A | AL2-114 | AL2-114A | AL2-345 | AL2-345A |
| | AL1-106B | | AL2-114B | | AL2-345B |
| AL1-107 | AL1-107A | AL2-115 | AL2-115A | AL2-346 | AL2-346A |
| | AL1-107B | | AL2-115B | | AL2-346B |
| AL1-108 | AL1-108A | AL2-116 | AL2-116A | AL2-347 | AL2-347A |
| | AL1-108B | | AL2-116B | | AL2-347B |
| AL1-109 | AL1-109A | AL2-117 | AL2-117A | AL2-348 | AL2-348A |
| | AL1-109B | | AL2-117B | | AL2-348B |
| AL1-110 | AL1-110A | AL2-118 | AL2-118A | AL2-349 | AL2-349A |
| | AL1-110B | | AL2-118B | | AL2-349B |
| AL1-111 | AL1-111A | AL2-119 | AL2-119A | AL2-350 | AL2-350A |
| | AL1-111B | | AL2-119B | | AL2-350B |
| AL1-112 | AL1-112A | AL2-120 | AL2-120A | AL2-351 | AL2-351A |
| | AL1-112B | | AL2-120B | | AL2-351B |
| AL1-113 | AL1-113A | AL2-121 | AL2-121A | AL2-352 | AL2-352A |
| | AL1-113B | | AL2-121B | | AL2-352B |
| AL1-114 | AL1-114A | AL2-122 | AL2-122A | AL2-353 | AL2-353A |
| | AL1-114B | | AL2-122B | | AL2-353B |
| AL1-115 | AL1-115A | AL2-123 | AL2-123A | AL2-354 | AL2-354A |
| | AL1-115B | | AL2-123B | | AL2-354B |
| AL1-116 | AL1-116A | AL2-124 | AL2-124A | AL2-355 | AL2-355A |
| | AL1-116B | | AL2-124B | | AL2-355B |
| AL1-117 | AL1-117A | AL2-125 | AL2-125A | AL2-356 | AL2-356A |
| | AL1-117B | | AL2-125B | | AL2-356B |
| AL1-118 | AL1-118A | AL2-126 | AL2-126A | AL2-357 | AL2-357A |
| | AL1-118B | | AL2-126B | | AL2-357B |
| AL1-119 | AL1-119A | AL2-127 | AL2-127A | AL2-358 | AL2-358A |
| | AL1-119B | | AL2-127B | | AL2-358B |
| AL1-120 | AL1-120A | AL2-128 | AL2-128A | AL2-359 | AL2-359A |
| | AL1-120B | | AL2-128B | | AL2-359B |
| AL1-121 | AL1-121A | AL2-129 | AL2-129A | AL2-360 | AL2-360A |
| | AL1-121B | | AL2-129B | | AL2-360B |
| AL1-122 | AL1-122A | AL2-130 | AL2-130A | AL2-361 | AL2-361A |
| | AL1-122B | | AL2-130B | | AL2-361B |
| AL1-123 | AL1-123A | AL2-131 | AL2-131A | AL2-362 | AL2-362A |
| | AL1-123B | | AL2-131B | | AL2-362B |
| AL1-124 | AL1-124A | AL2-132 | AL2-132A | AL2-363 | AL2-363A |
| | AL1-124B | | AL2-132B | | AL2-363B |
| AL1-125 | AL1-125A | AL2-133 | AL2-133A | AL2-364 | AL2-364A |
| | AL1-125B | | AL2-133B | | AL2-364B |
| AL1-126 | AL1-126A | AL2-134 | AL2-134A | AL2-365 | AL2-365A |
| | AL1-126B | | AL2-134B | | AL2-365B |
| AL1-127 | AL1-127A | AL2-135 | AL2-135A | AL2-366 | AL2-366A |
| | AL1-127B | | AL2-135B | | AL2-366B |
| AL1-128 | AL1-128A | AL2-136 | AL2-136A | AL2-367 | AL2-367A |
| | AL1-128B | | AL2-136B | | AL2-367B |
| AL1-129 | AL1-129A | AL2-137 | AL2-137A | AL2-368 | AL2-368A |
| | AL1-129B | | AL2-137B | | AL2-368B |
| AL1-130 | AL1-130A | AL2-138 | AL2-138A | AL2-369 | AL2-369A |
| | AL1-130B | | AL2-138B | | AL2-369B |
| AL1-131 | AL1-131A | AL2-139 | AL2-139A | AL2-370 | AL2-370A |
| | AL1-131B | | AL2-139B | | AL2-370B |
| AL1-132 | AL1-132A | AL2-140 | AL2-140A | AL2-371 | AL2-371A |
| | AL1-132B | | AL2-140B | | AL2-371B |
| AL1-133 | AL1-133A | AL2-141 | AL2-141A | AL2-372 | AL2-372A |
| | AL1-133B | | AL2-141B | | AL2-372B |
| AL1-134 | AL1-134A | AL2-142 | AL2-142A | AL2-373 | AL2-373A |
| | AL1-134B | | AL2-142B | | AL2-373B |
| AL1-135 | AL1-135A | AL2-143 | AL2-143A | AL2-374 | AL2-374A |
| | AL1-135B | | AL2-143B | | AL2-374B |
| AL1-136 | AL1-136A | AL2-144 | AL2-144A | AL2-375 | AL2-375A |
| | AL1-136B | | AL2-144B | | AL2-375B |
| AL1-137 | AL1-137A | AL2-145 | AL2-145A | AL2-376 | AL2-376A |
| | AL1-137B | | AL2-145B | | AL2-376B |
| AL1-138 | AL1-138A | AL2-146 | AL2-146A | AL2-377 | AL2-377A |
| | AL1-138B | | AL2-146B | | AL2-377B |
| AL1-139 | AL1-139A | AL2-147 | AL2-147A | AL2-378 | AL2-378A |
| | AL1-139B | | AL2-147B | | AL2-378B |
| AL1-140 | AL1-140A | AL2-148 | AL2-148A | AL2-379 | AL2-379A |
| | AL1-140B | | AL2-148B | | AL2-379B |
| AL1-141 | AL1-141A | AL2-149 | AL2-149A | AL2-380 | AL2-380A |
| | AL1-141B | | AL2-149B | | AL2-380B |
| AL1-142 | AL1-142A | AL2-150 | AL2-150A | AL2-381 | AL2-381A |
| | AL1-142B | | AL2-150B | | AL2-381B |
| AL1-143 | AL1-143A | AL2-151 | AL2-151A | AL2-382 | AL2-382A |
| | AL1-143B | | AL2-151B | | AL2-382B |
| AL1-144 | AL1-144A | AL2-152 | AL2-152A | AL2-383 | AL2-383A |
| | AL1-144B | | AL2-152B | | AL2-383B |
| AL1-145 | AL1-145A | AL2-153 | AL2-153A | AL2-384 | AL2-384A |
| | AL1-145B | | AL2-153B | | AL2-384B |
| AL1-146 | AL1-146A | AL2-154 | AL2-154A | AL2-385 | AL2-385A |
| | AL1-146B | | AL2-154B | | AL2-385B |
| AL1-147 | AL1-147A | AL2-155 | AL2-155A | AL2-386 | AL2-386A |
| | AL1-147B | | AL2-155B | | AL2-386B |
| AL1-148 | AL1-148A | AL2-156 | AL2-156A | AL2-387 | AL2-387A |
| | AL1-148B | | AL2-156B | | AL2-387B |
| AL1-149 | AL1-149A | AL2-157 | AL2-157A | AL2-388 | AL2-388A |
| | AL1-149B | | AL2-157B | | AL2-388B |
| AL1-150 | AL1-150A | AL2-158 | AL2-158A | AL2-389 | AL2-389A |
| | AL1-150B | | AL2-158B | | AL2-389B |
| AL1-151 | AL1-151A | AL2-159 | AL2-159A | AL2-390 | AL2-390A |
| | AL1-151B | | AL2-159B | | AL2-390B |
| AL1-152 | AL1-152A | AL2-160 | AL2-160A | AL2-391 | AL2-391A |
| | AL1-152B | | AL2-160B | | AL2-391B |
| AL1-153 | AL1-153A | AL2-161 | AL2-161A | AL2-392 | AL2-392A |
| | AL1-153B | | AL2-161B | | AL2-392B |
| AL1-154 | AL1-154A | AL2-162 | AL2-162A | AL2-393 | AL2-393A |
| | AL1-154B | | AL2-162B | | AL2-393B |
| AL1-155 | AL1-155A | AL2-163 | AL2-163A | AL2-394 | AL2-394A |
| | AL1-155B | | AL2-163B | | AL2-394B |
| AL1-156 | AL1-156A | AL2-164 | AL2-164A | AL2-395 | AL2-395A |
| | AL1-156B | | AL2-164B | | AL2-395B |
| AL1-157 | AL1-157A | AL2-165 | AL2-165A | AL2-396 | AL2-396A |
| | AL1-157B | | AL2-165B | | AL2-396B |
| AL1-158 | AL1-158A | AL2-166 | AL2-166A | AL2-397 | AL2-397A |
| | AL1-158B | | AL2-166B | | AL2-397B |
| AL1-159 | AL1-159A | AL2-167 | AL2-167A | AL2-398 | AL2-398A |
| | AL1-159B | | AL2-167B | | AL2-398B |
| AL1-160 | AL1-160A | AL2-168 | AL2-168A | AL2-399 | AL2-399A |
| | AL1-160B | | AL2-168B | | AL2-399B |
| AL1-161 | AL1-161A | AL2-169 | AL2-169A | AL2-400 | AL2-400A |
| | AL1-161B | | AL2-169B | | AL2-400B |
| AL1-162 | AL1-162A | AL2-170 | AL2-170A | AL2-401 | AL2-401A |
| | AL1-162B | | AL2-170B | | AL2-401B |
| AL1-163 | AL1-163A | AL2-171 | AL2-171A | AL2-402 | AL2-402A |
| | AL1-163B | | AL2-171B | | AL2-402B |
| AL1-164 | AL1-164A | AL2-172 | AL2-172A | AL2-403 | AL2-403A |
| | AL1-164B | | AL2-172B | | AL2-403B |
| AL1-165 | AL1-165A | AL2-173 | AL2-173A | AL2-404 | AL2-404A |
| | AL1-165B | | AL2-173B | | AL2-404B |
| AL1-166 | AL1-166A | AL2-174 | AL2-174A | AL2-405 | AL2-405A |
| | AL1-166B | | AL2-174B | | AL2-405B |
| AL1-167 | AL1-167A | AL2-175 | AL2-175A | AL2-406 | AL2-406A |
| | AL1-167B | | AL2-175B | | AL2-406B |
| AL1-168 | AL1-168A | AL2-176 | AL2-176A | AL2-407 | AL2-407A |
| | AL1-168B | | AL2-176B | | AL2-407B |
| AL1-169 | AL1-169A | AL2-177 | AL2-177A | AL2-408 | AL2-408A |
| | AL1-169B | | AL2-177B | | AL2-408B |
| AL1-170 | AL1-170A | AL2-178 | AL2-178A | AL2-409 | AL2-409A |
| | AL1-170B | | AL2-178B | | AL2-409B |
| AL1-171 | AL1-171A | AL2-179 | AL2-179A | AL2-410 | AL2-410A |
| | AL1-171B | | AL2-179B | | AL2-410B |
| AL1-172 | AL1-172A | AL2-180 | AL2-180A | AL2-411 | AL2-411A |
| | AL1-172B | | AL2-180B | | AL2-411B |
| AL1-173 | AL1-173A | AL2-181 | AL2-181A | AL2-412 | AL2-412A |
| | AL1-173B | | AL2-181B | | AL2-412B |
| AL1-174 | AL1-174A | AL2-182 | AL2-182A | AL2-413 | AL2-413A |
| | AL1-174B | | AL2-182B | | AL2-413B |
| AL1-175 | AL1-175A | AL2-183 | AL2-183A | AL2-414 | AL2-414A |
| | AL1-175B | | AL2-183B | | AL2-414B |
| AL1-176 | AL1-176A | AL2-184 | AL2-184A | AL2-415 | AL2-415A |
| | AL1-176B | | AL2-184B | | AL2-415B |
| AL1-177 | AL1-177A | AL2-185 | AL2-185A | AL2-416 | AL2-416A |
| | AL1-177B | | AL2-185B | | AL2-416B |
| AL1-178 | AL1-178A | AL2-186 | AL2-186A | AL2-417 | AL2-417A |
| | AL1-178B | | AL2-186B | | AL2-417B |
| AL1-179 | AL1-179A | AL2-187 | AL2-187A | AL2-418 | AL2-418A |
| | AL1-179B | | AL2-187B | | AL2-418B |
| AL1-180 | AL1-180A | AL2-188 | AL2-188A | AL2-419 | AL2-419A |
| | AL1-180B | | AL2-188B | | AL2-419B |
| AL1-181 | AL1-181A | AL2-189 | AL2-189A | AL2-420 | AL2-420A |
| | AL1-181B | | AL2-189B | | AL2-420B |
| AL1-182 | AL1-182A | AL2-190 | AL2-190A | AL2-421 | AL2-421A |
| | AL1-182B | | AL2-190B | | AL2-421B |
| AL1-183 | AL1-183A | AL2-191 | AL2-191A | AL2-422 | AL2-422A |
| | AL1-183B | | AL2-191B | | AL2-422B |
| AL1-184 | AL1-184A | AL2-192 | AL2-192A | AL2-423 | AL2-423A |
| | AL1-184B | | AL2-192B | | AL2-423B |
| AL1-185 | AL1-185A | AL2-193 | AL2-193A | AL2-424 | AL2-424A |
| | AL1-185B | | AL2-193B | | AL2-424B |
| AL1-186 | AL1-186A | AL2-194 | AL2-194A | AL2-425 | AL2-425A |
| | AL1-186B | | AL2-194B | | AL2-425B |
| AL1-187 | AL1-187A | AL2-195 | AL2-195A | AL2-426 | AL2-426A |
| | AL1-187B | | AL2-195B | | AL2-426B |
| AL1-188 | AL1-188A | AL2-196 | AL2-196A | AL2-427 | AL2-427A |
| | AL1-188B | | AL2-196B | | AL2-427B |
| AL1-189 | AL1-189A | AL2-197 | AL2-197A | AL2-428 | AL2-428A |
| | AL1-189B | | AL2-197B | | AL2-428B |
| AL1-190 | AL1-190A | AL2-198 | AL2-198A | AL2-429 | AL2-429A |
| | AL1-190B | | AL2-198B | | AL2-429B |
| AL1-191 | AL1-191A | AL2-199 | AL2-199A | AL2-430 | AL2-430A |
| | AL1-191B | | AL2-199B | | AL2-430B |
| AL1-192 | AL1-192A | AL2-200 | AL2-200A | AL2-431 | AL2-431A |
| | AL1-192B | | AL2-200B | | AL2-431B |
| AL1-193 | AL1-193A | AL2-201 | AL2-201A | AL2-432 | AL2-432A |
| | AL1-193B | | AL2-201B | | AL2-432B |
| AL1-194 | AL1-194A | AL2-202 | AL2-202A | AL2-433 | AL2-433A |
| | AL1-194B | | AL2-202B | | AL2-433B |
| AL1-195 | AL1-195A | AL2-203 | AL2-203A | AL2-434 | AL2-434A |
| | AL1-195B | | AL2-203B | | AL2-434B |
| AL1-196 | AL1-196A | AL2-204 | AL2-204A | AL2-435 | AL2-435A |
| | AL1-196B | | AL2-204B | | AL2-435B |
| AL1-197 | AL1-197A | AL2-205 | AL2-205A | AL2-436 | AL2-436A |
| | AL1-197B | | AL2-205B | | AL2-436B |
| AL1-198 | AL1-198A | AL2-206 | AL2-206A | AL2-437 | AL2-437A |
| | AL1-198B | | AL2-206B | | AL2-437B |
| AL1-199 | AL1-199A | AL2-207 | AL2-207A | AL2-438 | AL2-438A |
| | AL1-199B | | AL2-207B | | AL2-438B |
| AL1-200 | AL1-200A | AL2-208 | AL2-208A | AL2-439 | AL2-439A |
| | AL1-200B | | AL2-208B | | AL2-439B |
| AL1-201 | AL1-201A | AL2-209 | AL2-209A | AL2-440 | AL2-440A |
| | AL1-201B | | AL2-209B | | AL2-440B |
| AL1-202 | AL1-202A | AL2-210 | AL2-210A | AL2-441 | AL2-441A |
| | AL1-202B | | AL2-210B | | AL2-441B |
| AL1-203 | AL1-203A | AL2-211 | AL2-211A | AL2-442 | AL2-442A |
| | AL1-203B | | AL2-211B | | AL2-442B |
| AL1-204 | AL1-204A | AL2-212 | AL2-212A | AL2-443 | AL2-443A |
| | AL1-204B | | AL2-212B | | AL2-443B |
| AL1-205 | AL1-205A | AL2-213 | AL2-213A | AL2-444 | AL2-444A |
| | AL1-205B | | AL2-213B | | AL2-444B |
| AL1-206 | AL1-206A | AL2-214 | AL2-214A | AL2-445 | AL2-445A |
| | AL1-206B | | AL2-214B | | AL2-445B |
| AL1-207 | AL1-207A | AL2-215 | AL2-215A | AL2-446 | AL2-446A |
| | AL1-207B | | AL2-215B | | AL2-446B |
| AL1-208 | AL1-208A | AL2-216 | AL2-216A | AL2-447 | AL2-447A |
| | AL1-208B | | AL2-216B | | AL2-447B |
| AL1-209 | AL1-209A | AL2-217 | AL2-217A | AL2-448 | AL2-448A |
| | AL1-209B | | AL2-217B | | AL2-448B |
| AL1-210 | AL1-210A | AL2-218 | AL2-218A | AL2-449 | AL2-449A |
| | AL1-210B | | AL2-218B | | AL2-449B |
| AL1-211 | AL1-211A | AL2-219 | AL2-219A | AL2-450 | AL2-450A |
| | AL1-211B | | AL2-219B | | AL2-450B |
| AL1-212 | AL1-212A | AL2-220 | AL2-220A | AL2-451 | AL2-451A |
| | AL1-212B | | AL2-220B | | AL2-451B |
| AL1-213 | AL1-213A | AL2-221 | AL2-221A | | |
| | AL1-213B | | AL2-221B | | |

### Summary Abbreviations Table

TABLE 3

| Abbreviation | Meaning |
|---|---|
| mA, mU, mC, | 2′-O-Methyl RNA nucleotides |
| mG | |
| 2′-OMe | 2′-O-Methyl modification |
| fA, fU, fC, fG | 2′ deoxy-2′-F RNA nucleotides |
| 2′-F | 2′-fluoro modification |
| (ps) | phosphorothioate |
| (ps2) | phosphorodithioate |
| (vp) | Vinyl-(E)-phosphonate |
| (vp)-mU | 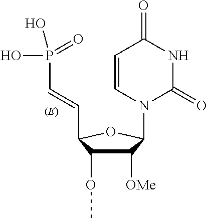 |
| (vp)-mU-phos | 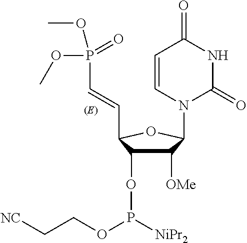 |
| ivA, ivC, ivU, | inverted RNA (3′-3′) nucleotides |
| ivG | |
| ST23 | 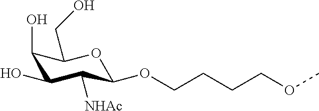 |
| ST23-phos | 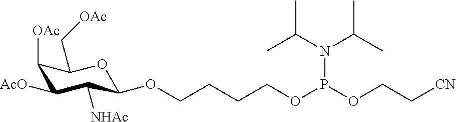 |
| ST43 (or C6XLT) | 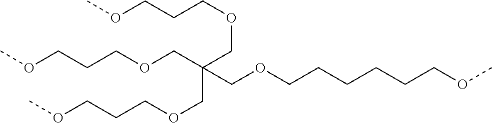 |
| ST43-phos (or C6XLT-phos) | 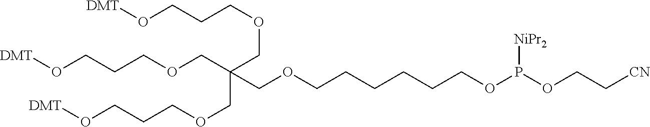 |
| Ser (GN) (when at the end of a chain, one of the O— is OH) | 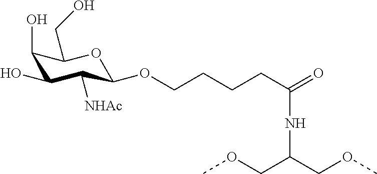 |
| [ST23 (ps)]3 ST43 (ps) | 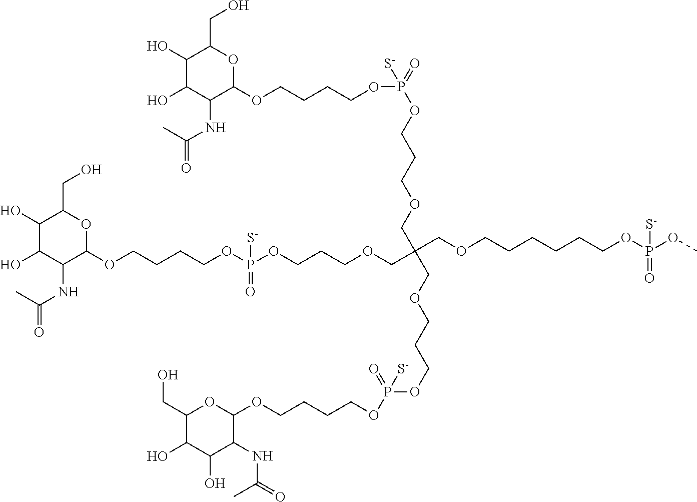 |
| [ST23]3 ST43 | 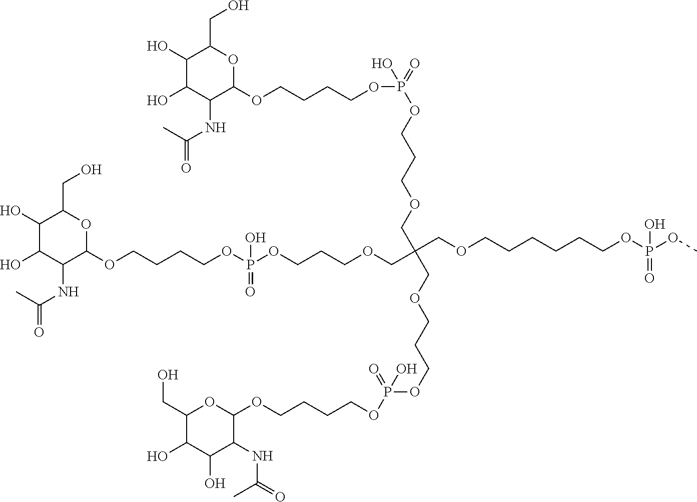 |
| [ST23(ps)]3 ST41(ps) | 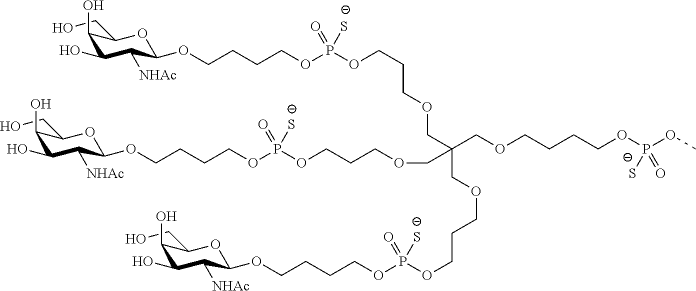 |
| [ST23]3 ST41 | 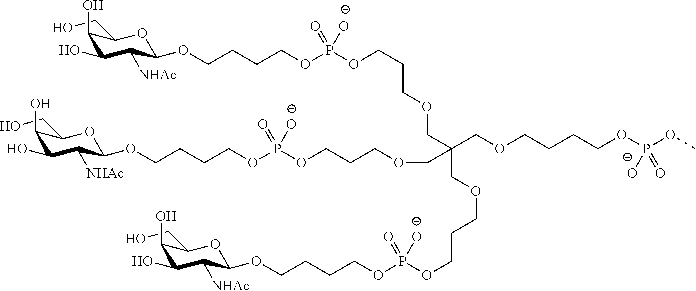 |

The abbreviations as shown in the above abbreviation table may be used herein. The list of abbreviations may not be exhaustive and further abbreviations and their meaning may be found throughout this document.

### Summary Sequence Table

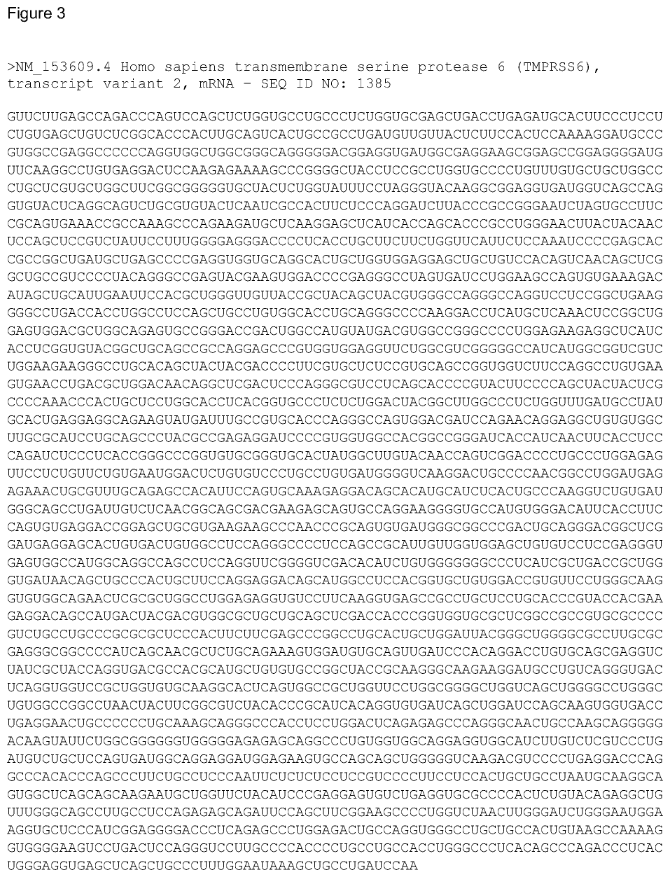

TABLE 4

| SEQ | Name | | |
|---|---|---|---|
| ID | (A = 1^{st} strand; | | Unmodified sequence 5′-3′ |
| NO: | B = 2^{nd} strand) | Sequence 5′-3′ | counterpart |
| 1 | EU400A | mU (ps) fC (ps) mG fA mA fG mU fA mU fU mC fC mG | UCGAAGUAUUCCGCGUACG |
| | | fC mG fU mA (ps) fC (ps) mG | |
| 2 | EU400B | [ST23 (ps)]3 ST43 (ps) mC mG mU mA mC mG FC fG fG | CGUACGCGGAAUACUUCGA |
| | | mA mA mU mA mC mU mU mC(ps)mG(ps)mA | |
| 3 | EU401A | mA (ps) fA (ps) mC fC mA fG mA fA mG fA mA fG mC | AACCAGAAGAAGCAGGUGA |
| | | fA mG fG mU (ps) fG (ps) mA | |
| 4 | EU401B | [ST23 (ps)]3 ST43 (ps) mU mC mA mC mC mU fG fC fU | UCACCUGCUUCUUCUGGUU |
| | | mU mC mU mU mC mU mG mG (ps) mU (ps) mU | |
| 5 | EU402B | [ST23 (ps)]3 ST41 (ps) fU mC fA mC fC mU fG mC fU | UCACCUGCUUCUUCUGGUU |
| | | mU fC mU fU mC fU mG fG (ps) mU (ps) fU | |
| 6 | EU401Aun | AACCAGAAGAAGCAGGUGA | AACCAGAAGAAGCAGGUGA |
| 7 | EU401Bun | UCACCUGCUUCUUCUGGUU | UCACCUGCUUCUUCUGGUU |
| 8 | TMPRSS6-hc-1A | AUGUCUUUCACACUGGCUU | AUGUCUUUCACACUGGCUU |
| 9 | TMPRSS6-hc-1B | AAGCCAGUGUGAAAGACAU | AAGCCAGUGUGAAAGACAU |
| 10 | TMPRSS6-h-2A | AUUGAGUACACGCAGACUG | AUUGAGUACACGCAGACUG |
| 11 | TMPRSS6-h-2B | CAGUCUGCGUGUACUCAAU | CAGUCUGCGUGUACUCAAU |
| 12 | TMPRSS6-h-3A | AAGUUGAUGGUGAUCCCGG | AAGUUGAUGGUGAUCCCGG |
| 13 | TMPRSS6-h-3B | CCGGGAUCACCAUCAACUU | CCGGGAUCACCAUCAACUU |
| 14 | TMPRSS6-hc-4A | UUCUGGAUCGUCCACUGGC | UUCUGGAUCGUCCACUGGC |
| 15 | TMPRSS6-hc-4B | GCCAGUGGACGAUCCAGAA | GCCAGUGGACGAUCCAGAA |
| 16 | TMPRSS6-h-5A | AUUCACAGAACAGAGGAAC | AUUCACAGAACAGAGGAAC |
| 17 | TMPRSS6-h-5B | GUUCCUCUGUUCUGUGAAU | GUUCCUCUGUUCUGUGAAU |
| 18 | TMPRSS6-h-6A | GUAGUCAUGGCUGUCCUCU | GUAGUCAUGGCUGUCCUCU |
| 19 | TMPRSS6-h-6B | AGAGGACAGCCAUGACUAC | AGAGGACAGCCAUGACUAC |
| 20 | TMPRSS6-h-7A | AGUUGUAGUAAGUUCCCAG | AGUUGUAGUAAGUUCCCAG |
| 21 | TMPRSS6-h-7B | CUGGGAACUUACUACAACU | CUGGGAACUUACUACAACU |
| 22 | TMPRSS6-hcmr-8A | UUGUACCCUAGGAAAUACC | UUGUACCCUAGGAAAUACC |
| 23 | TMPRSS6-hcmr-8B | GGUAUUUCCUAGGGUACAA | GGUAUUUCCUAGGGUACAA |
| 24 | TMPRSS6-hcm-9A | AACCAGAAGAAGCAGGUGA | AACCAGAAGAAGCAGGUGA |
| 25 | TMPRSS6-hcm-9B | UCACCUGCUUCUUCUGGUU | UCACCUGCUUCUUCUGGUU |
| 26 | TMPRSS6-hc-10A | UAACAACCCAGCGUGGAAU | UAACAACCCAGCGUGGAAU |
| 27 | TMPRSS6-hc-10B | AUUCCACGCUGGGUUGUUA | AUUCCACGCUGGGUUGUUA |
| 28 | TMPRSS6-hc-11A | GUUUCUCUCAUCCAGGCCG | GUUUCUCUCAUCCAGGCCG |
| 29 | TMPRSS6-hc-11B | CGGCCUGGAUGAGAGAAAC | CGGCCUGGAUGAGAGAAAC |
| 30 | TMPRSS6-hcm-12A | GCAUCUUCUGGGCUUUGGC | GCAUCUUCUGGGCUUUGGC |
| 31 | TMPRSS6-hcm-12B | GCCAAAGCCCAGAAGAUGC | GCCAAAGCCCAGAAGAUGC |
| 32 | TMPRSS6-hc-13A | UCACACUGGAAGGUGAAUG | UCACACUGGAAGGUGAAUG |
| 33 | TMPRSS6-hc-13B | CAUUCACCUUCCAGUGUGA | CAUUCACCUUCCAGUGUGA |
| 34 | TMPRSS6-hcmr-14A | CACAGAUGUGUCGACCCCG | CACAGAUGUGUCGACCCCG |
| 35 | TMPRSS6-hcmr-14B | CGGGGUCGACACAUCUGUG | CGGGGUCGACACAUCUGUG |
| 36 | TMPRSS6-hcmr-15A | UGUACCCUAGGAAAUACCA | UGUACCCUAGGAAAUACCA |
| 37 | TMPRSS6-hcmr-15B | UGGUAUUUCCUAGGGUACA | UGGUAUUUCCUAGGGUACA |
| 38 | AL1-1A | AGGUCAGCUCGCACCAGAG | AGGUCAGCUCGCACCAGAG |
| 39 | AL1-1B | CUCUGGUGCGAGCUGACCU | CUCUGGUGCGAGCUGACCU |
| 40 | AL1-2A | AGUGCAUCUCAGGUCAGCU | AGUGCAUCUCAGGUCAGCU |
| 41 | AL1-2B | AGCUGACCUGAGAUGCACU | AGCUGACCUGAGAUGCACU |
| 42 | AL1-3A | UGCCGAGACAGCUCACAGA | UGCCGAGACAGCUCACAGA |
| 43 | AL1-3B | UCUGUGAGCUGUCUCGGCA | UCUGUGAGCUGUCUCGGCA |
| 44 | AL1-4A | AGUGGGUGCCGAGACAGCU | AGUGGGUGCCGAGACAGCU |
| 45 | AL1-4B | AGCUGUCUCGGCACCCACU | AGCUGUCUCGGCACCCACU |
| 46 | AL1-5A | AAGUGGGUGCCGAGACAGC | AAGUGGGUGCCGAGACAGC |
| 47 | AL1-5B | GCUGUCUCGGCACCCACUU | GCUGUCUCGGCACCCACUU |
| 48 | AL1-6A | ACAUCAGGCGGCAGUGACU | ACAUCAGGCGGCAGUGACU |
| 49 | AL1-6B | AGUCACUGCCGCCUGAUGU | AGUCACUGCCGCCUGAUGU |
| 50 | AL1-7A | AACAACAUCAGGCGGCAGU | AACAACAUCAGGCGGCAGU |
| 51 | AL1-7B | ACUGCCGCCUGAUGUUGUU | ACUGCCGCCUGAUGUUGUU |
| 52 | AL1-8A | UAACAACAUCAGGCGGCAG | UAACAACAUCAGGCGGCAG |
| 53 | AL1-8B | CUGCCGCCUGAUGUUGUUA | CUGCCGCCUGAUGUUGUUA |
| 54 | AL1-9A | AGUAACAACAUCAGGCGGC | AGUAACAACAUCAGGCGGC |
| 55 | AL1-9B | GCCGCCUGAUGUUGUUACU | GCCGCCUGAUGUUGUUACU |
| 56 | AL1-10A | AAGAGUAACAACAUCAGGC | AAGAGUAACAACAUCAGGC |
| 57 | AL1-10B | GCCUGAUGUUGUUACUCUU | GCCUGAUGUUGUUACUCUU |
| 58 | AL1-11A | UCCUUUUGGAGUGGAAGAG | UCCUUUUGGAGUGGAAGAG |
| 59 | AL1-11B | CUCUUCCACUCCAAAAGGA | CUCUUCCACUCCAAAAGGA |
| 60 | AL1-12A | ACGGGCAUCCUUUUGGAGU | ACGGGCAUCCUUUUGGAGU |
| 61 | AL1-12B | ACUCCAAAAGGAUGCCCGU | ACUCCAAAAGGAUGCCCGU |
| 62 | AL1-13A | UUUCUCUUGGAGUCCUCAC | UUUCUCUUGGAGUCCUCAC |
| 63 | AL1-13B | GUGAGGACUCCAAGAGAAA | GUGAGGACUCCAAGAGAAA |
| 64 | AL1-14A | AGUAGCACCCCCGCCGAAG | AGUAGCACCCCCGCCGAAG |
| 65 | AL1-14B | CUUCGGCGGGGGUGCUACU | CUUCGGCGGGGGUGCUACU |
| 66 | AL1-15A | AGAGUAGCACCCCCGCCGA | AGAGUAGCACCCCCGCCGA |
| 67 | AL1-15B | UCGGCGGGGGUGCUACUCU | UCGGCGGGGGUGCUACUCU |
| 68 | AL1-16A | ACCAGAGUAGCACCCCCGC | ACCAGAGUAGCACCCCCGC |
| 69 | AL1-16B | GCGGGGGUGCUACUCUGGU | GCGGGGGUGCUACUCUGGU |
| 70 | AL1-17A | AUACCAGAGUAGCACCCCC | AUACCAGAGUAGCACCCCC |
| 71 | AL1-17B | GGGGGUGCUACUCUGGUAU | GGGGGUGCUACUCUGGUAU |
| 72 | AL1-18A | AAUACCAGAGUAGCACCCC | AAUACCAGAGUAGCACCCC |
| 73 | AL1-18B | GGGGUGCUACUCUGGUAUU | GGGGUGCUACUCUGGUAUU |
| 74 | AL1-19A | UACCCUAGGAAAUACCAGA | UACCCUAGGAAAUACCAGA |
| 75 | AL1-19B | UCUGGUAUUUCCUAGGGUA | UCUGGUAUUUCCUAGGGUA |
| 76 | AL1-20A | UGUACCCUAGGAAAUACCA | UGUACCCUAGGAAAUACCA |
| 77 | AL1-20B | UGGUAUUUCCUAGGGUACA | UGGUAUUUCCUAGGGUACA |
| 78 | AL1-21A | UUGUACCCUAGGAAAUACC | UUGUACCCUAGGAAAUACC |
| 79 | AL1-21B | GGUAUUUCCUAGGGUACAA | GGUAUUUCCUAGGGUACAA |
| 80 | AL1-22A | UGAGUACACCUGGCUGACC | UGAGUACACCUGGCUGACC |
| 81 | AL1-22B | GGUCAGCCAGGUGUACUCA | GGUCAGCCAGGUGUACUCA |
| 82 | AL1-23A | UGCCUGAGUACACCUGGCU | UGCCUGAGUACACCUGGCU |
| 83 | AL1-23B | AGCCAGGUGUACUCAGGCA | AGCCAGGUGUACUCAGGCA |
| 84 | AL1-24A | ACGCAGACUGCCUGAGUAC | ACGCAGACUGCCUGAGUAC |
| 85 | AL1-24B | GUACUCAGGCAGUCUGCGU | GUACUCAGGCAGUCUGCGU |
| 86 | AL1-25A | ACACGCAGACUGCCUGAGU | ACACGCAGACUGCCUGAGU |
| 87 | AL1-25B | ACUCAGGCAGUCUGCGUGU | ACUCAGGCAGUCUGCGUGU |
| 88 | AL1-26A | AGUACACGCAGACUGCCUG | AGUACACGCAGACUGCCUG |
| 89 | AL1-26B | CAGGCAGUCUGCGUGUACU | CAGGCAGUCUGCGUGUACU |
| 90 | AL1-27A | UGAGUACACGCAGACUGCC | UGAGUACACGCAGACUGCC |
| 91 | AL1-27B | GGCAGUCUGCGUGUACUCA | GGCAGUCUGCGUGUACUCA |
| 92 | AL1-28A | UUGAGUACACGCAGACUGC | UUGAGUACACGCAGACUGC |
| 93 | AL1-28B | GCAGUCUGCGUGUACUCAA | GCAGUCUGCGUGUACUCAA |
| 94 | AL1-29A | AUUGAGUACACGCAGACUG | AUUGAGUACACGCAGACUG |
| 95 | AL1-29B | CAGUCUGCGUGUACUCAAU | CAGUCUGCGUGUACUCAAU |
| 96 | AL1-30A | UGGCGAUUGAGUACACGCA | UGGCGAUUGAGUACACGCA |
| 97 | AL1-30B | UGCGUGUACUCAAUCGCCA | UGCGUGUACUCAAUCGCCA |
| 98 | AL1-31A | AGUGGCGAUUGAGUACACG | AGUGGCGAUUGAGUACACG |
| 99 | AL1-31B | CGUGUACUCAAUCGCCACU | CGUGUACUCAAUCGCCACU |
| 100 | AL1-32A | AAGUGGCGAUUGAGUACAC | AAGUGGCGAUUGAGUACAC |
| 101 | AL1-32B | GUGUACUCAAUCGCCACUU | GUGUACUCAAUCGCCACUU |
| 102 | AL1-33A | AGAAGUGGCGAUUGAGUAC | AGAAGUGGCGAUUGAGUAC |
| 103 | AL1-33B | GUACUCAAUCGCCACUUCU | GUACUCAAUCGCCACUUCU |
| 104 | AL1-34A | UAAGAUCCUGGGAGAAGUG | UAAGAUCCUGGGAGAAGUG |
| 105 | AL1-34B | CACUUCUCCCAGGAUCUUA | CACUUCUCCCAGGAUCUUA |
| 106 | AL1-35A | AUUCCCGGCGGGUAAGAUC | AUUCCCGGCGGGUAAGAUC |
| 107 | AL1-35B | GAUCUUACCCGCCGGGAAU | GAUCUUACCCGCCGGGAAU |
| 108 | AL1-36A | AGAUUCCCGGCGGGUAAGA | AGAUUCCCGGCGGGUAAGA |
| 109 | AL1-36B | UCUUACCCGCCGGGAAUCU | UCUUACCCGCCGGGAAUCU |
| 110 | AL1-37A | UAGAUUCCCGGCGGGUAAG | UAGAUUCCCGGCGGGUAAG |
| 111 | AL1-37B | CUUACCCGCCGGGAAUCUA | CUUACCCGCCGGGAAUCUA |
| 112 | AL1-38A | ACUAGAUUCCCGGCGGGUA | ACUAGAUUCCCGGCGGGUA |
| 113 | AL1-38B | UACCCGCCGGGAAUCUAGU | UACCCGCCGGGAAUCUAGU |
| 114 | AL1-39A | AGGCACUAGAUUCCCGGCG | AGGCACUAGAUUCCCGGCG |
| 115 | AL1-39B | CGCCGGGAAUCUAGUGCCU | CGCCGGGAAUCUAGUGCCU |
| 116 | AL1-40A | AAGGCACUAGAUUCCCGGC | AAGGCACUAGAUUCCCGGC |
| 117 | AL1-40B | GCCGGGAAUCUAGUGCCUU | GCCGGGAAUCUAGUGCCUU |
| 118 | AL1-41A | UUGGCGGUUUCACUGCGGA | UUGGCGGUUUCACUGCGGA |
| 119 | AL1-41B | UCCGCAGUGAAACCGCCAA | UCCGCAGUGAAACCGCCAA |
| 120 | AL1-42A | UUUGGCGGUUUCACUGCGG | UUUGGCGGUUUCACUGCGG |
| 121 | AL1-42B | CCGCAGUGAAACCGCCAAA | CCGCAGUGAAACCGCCAAA |
| 122 | AL1-43A | UGUAGUAAGUUCCCAGGCG | UGUAGUAAGUUCCCAGGCG |
| 123 | AL1-43B | CGCCUGGGAACUUACUACA | CGCCUGGGAACUUACUACA |
| 124 | AL1-44A | UUGUAGUAAGUUCCCAGGC | UUGUAGUAAGUUCCCAGGC |
| 125 | AL1-44B | GCCUGGGAACUUACUACAA | GCCUGGGAACUUACUACAA |
| 126 | AL1-45A | AGUUGUAGUAAGUUCCCAG | AGUUGUAGUAAGUUCCCAG |
| 127 | AL1-45B | CUGGGAACUUACUACAACU | CUGGGAACUUACUACAACU |
| 128 | AL1-46A | AGACGGAGCUGGAGUUGUA | AGACGGAGCUGGAGUUGUA |
| 129 | AL1-46B | UACAACUCCAGCUCCGUCU | UACAACUCCAGCUCCGUCU |
| 130 | AL1-47A | UAGACGGAGCUGGAGUUGU | UAGACGGAGCUGGAGUUGU |
| 131 | AL1-47B | ACAACUCCAGCUCCGUCUA | ACAACUCCAGCUCCGUCUA |
| 132 | AL1-48A | AAUAGACGGAGCUGGAGUU | AAUAGACGGAGCUGGAGUU |
| 133 | AL1-48B | AACUCCAGCUCCGUCUAUU | AACUCCAGCUCCGUCUAUU |
| 134 | AL1-49A | UGAGGGGUCCCUCCCCAAA | UGAGGGGUCCCUCCCCAAA |
| 135 | AL1-49B | UUUGGGGAGGGACCCCUCA | UUUGGGGAGGGACCCCUCA |
| 136 | AL1-50A | AGAAGCAGGUGAGGGGUCC | AGAAGCAGGUGAGGGGUCC |
| 137 | AL1-50B | GGACCCCUCACCUGCUUCU | GGACCCCUCACCUGCUUCU |
| 138 | AL1-51A | AGAAUGAACCAGAAGAAGC | AGAAUGAACCAGAAGAAGC |
| 139 | AL1-51B | GCUUCUUCUGGUUCAUUCU | GCUUCUUCUGGUUCAUUCU |
| 140 | AL1-52A | UGGAGAAUGAACCAGAAGA | UGGAGAAUGAACCAGAAGA |
| 141 | AL1-52B | UCUUCUGGUUCAUUCUCCA | UCUUCUGGUUCAUUCUCCA |
| 142 | AL1-53A | AGCAUCAGCCGGCGGUGCU | AGCAUCAGCCGGCGGUGCU |
| 143 | AL1-53B | AGCACCGCCGGCUGAUGCU | AGCACCGCCGGCUGAUGCU |
| 144 | AL1-54A | UACUCGGCCCUGUAGGGGA | UACUCGGCCCUGUAGGGGA |
| 145 | AL1-54B | UCCCCUACAGGGCCGAGUA | UCCCCUACAGGGCCGAGUA |
| 146 | AL1-55A | UCGUACUCGGCCCUGUAGG | UCGUACUCGGCCCUGUAGG |
| 147 | AL1-55B | CCUACAGGGCCGAGUACGA | CCUACAGGGCCGAGUACGA |
| 148 | AL1-56A | ACUUCGUACUCGGCCCUGU | ACUUCGUACUCGGCCCUGU |
| 149 | AL1-56B | ACAGGGCCGAGUACGAAGU | ACAGGGCCGAGUACGAAGU |
| 150 | AL1-57A | UCCACUUCGUACUCGGCCC | UCCACUUCGUACUCGGCCC |
| 151 | AL1-57B | GGGCCGAGUACGAAGUGGA | GGGCCGAGUACGAAGUGGA |
| 152 | AL1-58A | AGGAUCACUAGGCCCUCGG | AGGAUCACUAGGCCCUCGG |
| 153 | AL1-58B | CCGAGGGCCUAGUGAUCCU | CCGAGGGCCUAGUGAUCCU |
| 154 | AL1-59A | AGCUAUGUCUUUCACACUG | AGCUAUGUCUUUCACACUG |
| 155 | AL1-59B | CAGUGUGAAAGACAUAGCU | CAGUGUGAAAGACAUAGCU |
| 156 | AL1-60A | ACAACCCAGCGUGGAAUUC | ACAACCCAGCGUGGAAUUC |
| 157 | AL1-60B | GAAUUCCACGCUGGGUUGU | GAAUUCCACGCUGGGUUGU |
| 158 | AL1-61A | AACAACCCAGCGUGGAAUU | AACAACCCAGCGUGGAAUU |
| 159 | AL1-61B | AAUUCCACGCUGGGUUGUU | AAUUCCACGCUGGGUUGUU |
| 160 | AL1-62A | AGCGGUAACAACCCAGCGU | AGCGGUAACAACCCAGCGU |
| 161 | AL1-62B | ACGCUGGGUUGUUACCGCU | ACGCUGGGUUGUUACCGCU |
| 162 | AL1-63A | UAGCGGUAACAACCCAGCG | UAGCGGUAACAACCCAGCG |
| 163 | AL1-63B | CGCUGGGUUGUUACCGCUA | CGCUGGGUUGUUACCGCUA |
| 164 | AL1-64A | UGUAGCGGUAACAACCCAG | UGUAGCGGUAACAACCCAG |
| 165 | AL1-64B | CUGGGUUGUUACCGCUACA | CUGGGUUGUUACCGCUACA |
| 166 | AL1-65A | AGCUGUAGCGGUAACAACC | AGCUGUAGCGGUAACAACC |
| 167 | AL1-65B | GGUUGUUACCGCUACAGCU | GGUUGUUACCGCUACAGCU |
| 168 | AL1-66A | ACGUAGCUGUAGCGGUAAC | ACGUAGCUGUAGCGGUAAC |
| 169 | AL1-66B | GUUACCGCUACAGCUACGU | GUUACCGCUACAGCUACGU |
| 170 | AL1-67A | AGUUUGAGCAUGAGGUCCU | AGUUUGAGCAUGAGGUCCU |
| 171 | AL1-67B | AGGACCUCAUGCUCAAACU | AGGACCUCAUGCUCAAACU |
| 172 | AL1-68A | AGCCGGAGUUUGAGCAUGA | AGCCGGAGUUUGAGCAUGA |
| 173 | AL1-68B | UCAUGCUCAAACUCCGGCU | UCAUGCUCAAACUCCGGCU |
| 174 | AL1-69A | AGCCGUACACCGAGGUGAU | AGCCGUACACCGAGGUGAU |
| 175 | AL1-69B | AUCACCUCGGUGUACGGCU | AUCACCUCGGUGUACGGCU |
| 176 | AL1-70A | UGCAGCCGUACACCGAGGU | UGCAGCCGUACACCGAGGU |
| 177 | AL1-70B | ACCUCGGUGUACGGCUGCA | ACCUCGGUGUACGGCUGCA |
| 178 | AL1-71A | UCCAGACGACCGCCAUGAU | UCCAGACGACCGCCAUGAU |
| 179 | AL1-71B | AUCAUGGCGGUCGUCUGGA | AUCAUGGCGGUCGUCUGGA |
| 180 | AL1-72A | UUCCAGACGACCGCCAUGA | UUCCAGACGACCGCCAUGA |
| 181 | AL1-72B | UCAUGGCGGUCGUCUGGAA | UCAUGGCGGUCGUCUGGAA |
| 182 | AL1-73A | ACGAAGGGGUCGUAGUAGC | ACGAAGGGGUCGUAGUAGC |
| 183 | AL1-73B | GCUACUACGACCCCUUCGU | GCUACUACGACCCCUUCGU |
| 184 | AL1-74A | AAGACCACCGGCUGCACGG | AAGACCACCGGCUGCACGG |
| 185 | AL1-74B | CCGUGCAGCCGGUGGUCUU | CCGUGCAGCCGGUGGUCUU |
| 186 | AL1-75A | ACUUCACAGGCCUGGAAGA | ACUUCACAGGCCUGGAAGA |
| 187 | AL1-75B | UCUUCCAGGCCUGUGAAGU | UCUUCCAGGCCUGUGAAGU |
| 188 | AL1-76A | UCAGGUUCACUUCACAGGC | UCAGGUUCACUUCACAGGC |
| 189 | AL1-76B | GCCUGUGAAGUGAACCUGA | GCCUGUGAAGUGAACCUGA |
| 190 | AL1-77A | AGCGUCAGGUUCACUUCAC | AGCGUCAGGUUCACUUCAC |
| 191 | AL1-77B | GUGAAGUGAACCUGACGCU | GUGAAGUGAACCUGACGCU |
| 192 | AL1-78A | UCGAGCCUGUUGUCCAGCG | UCGAGCCUGUUGUCCAGCG |
| 193 | AL1-78B | CGCUGGACAACAGGCUCGA | CGCUGGACAACAGGCUCGA |
| 194 | AL1-79A | AGUCGAGCCUGUUGUCCAG | AGUCGAGCCUGUUGUCCAG |
| 195 | AL1-79B | CUGGACAACAGGCUCGACU | CUGGACAACAGGCUCGACU |
| 196 | AL1-80A | AGUACGGGGUGCUGAGGAC | AGUACGGGGUGCUGAGGAC |
| 197 | AL1-80B | GUCCUCAGCACCCCGUACU | GUCCUCAGCACCCCGUACU |
| 198 | AL1-81A | AAGUACGGGGUGCUGAGGA | AAGUACGGGGUGCUGAGGA |
| 199 | AL1-81B | UCCUCAGCACCCCGUACUU | UCCUCAGCACCCCGUACUU |
| 200 | AL1-82A | UGGGGAAGUACGGGGUGCU | UGGGGAAGUACGGGGUGCU |
| 201 | AL1-82B | AGCACCCCGUACUUCCCCA | AGCACCCCGUACUUCCCCA |
| 202 | AL1-83A | UUGGGGCGAGUAGUAGCUG | UUGGGGCGAGUAGUAGCUG |
| 203 | AL1-83B | CAGCUACUACUCGCCCCAA | CAGCUACUACUCGCCCCAA |
| 204 | AL1-84A | UUUGGGGCGAGUAGUAGCU | UUUGGGGCGAGUAGUAGCU |
| 205 | AL1-84B | AGCUACUACUCGCCCCAAA | AGCUACUACUCGCCCCAAA |
| 206 | AL1-85A | UGGGUUUGGGGCGAGUAGU | UGGGUUUGGGGCGAGUAGU |
| 207 | AL1-85B | ACUACUCGCCCCAAACCCA | ACUACUCGCCCCAAACCCA |
| 208 | AL1-86A | AGCAGUGGGUUUGGGGCGA | AGCAGUGGGUUUGGGGCGA |
| 209 | AL1-86B | UCGCCCCAAACCCACUGCU | UCGCCCCAAACCCACUGCU |
| 210 | AL1-87A | ACCGUGAGGUGCCAGGAGC | ACCGUGAGGUGCCAGGAGC |
| 211 | AL1-87B | GCUCCUGGCACCUCACGGU | GCUCCUGGCACCUCACGGU |
| 212 | AL1-88A | AGCCGUAGUCCAGAGAGGG | AGCCGUAGUCCAGAGAGGG |
| 213 | AL1-88B | CCCUCUCUGGACUACGGCU | CCCUCUCUGGACUACGGCU |
| 214 | AL1-89A | AACCAGAGGGCCAAGCCGU | AACCAGAGGGCCAAGCCGU |
| 215 | AL1-89B | ACGGCUUGGCCCUCUGGUU | ACGGCUUGGCCCUCUGGUU |
| 216 | AL1-90A | AAACCAGAGGGCCAAGCCG | AAACCAGAGGGCCAAGCCG |
| 217 | AL1-90B | CGGCUUGGCCCUCUGGUUU | CGGCUUGGCCCUCUGGUUU |
| 218 | AL1-91A | UCAAACCAGAGGGCCAAGC | UCAAACCAGAGGGCCAAGC |
| 219 | AL1-91B | GCUUGGCCCUCUGGUUUGA | GCUUGGCCCUCUGGUUUGA |
| 220 | AL1-92A | AUAGGCAUCAAACCAGAGG | AUAGGCAUCAAACCAGAGG |
| 221 | AL1-92B | CCUCUGGUUUGAUGCCUAU | CCUCUGGUUUGAUGCCUAU |
| 222 | AL1-93A | ACGGCAAAUCAUACUUCUG | ACGGCAAAUCAUACUUCUG |
| 223 | AL1-93B | CAGAAGUAUGAUUUGCCGU | CAGAAGUAUGAUUUGCCGU |
| 224 | AL1-94A | UGCACGGCAAAUCAUACUU | UGCACGGCAAAUCAUACUU |
| 225 | AL1-94B | AAGUAUGAUUUGCCGUGCA | AAGUAUGAUUUGCCGUGCA |
| 226 | AL1-95A | UGGGUGCACGGCAAAUCAU | UGGGUGCACGGCAAAUCAU |
| 227 | AL1-95B | AUGAUUUGCCGUGCACCCA | AUGAUUUGCCGUGCACCCA |
| 228 | AL1-96A | UCGUCCACUGGCCCUGGGU | UCGUCCACUGGCCCUGGGU |
| 229 | AL1-96B | ACCCAGGGCCAGUGGACGA | ACCCAGGGCCAGUGGACGA |
| 230 | AL1-97A | UGGAUCGUCCACUGGCCCU | UGGAUCGUCCACUGGCCCU |
| 231 | AL1-97B | AGGGCCAGUGGACGAUCCA | AGGGCCAGUGGACGAUCCA |
| 232 | AL1-98A | UCUGGAUCGUCCACUGGCC | UCUGGAUCGUCCACUGGCC |
| 233 | AL1-98B | GGCCAGUGGACGAUCCAGA | GGCCAGUGGACGAUCCAGA |
| 234 | AL1-99A | UUCUGGAUCGUCCACUGGC | UUCUGGAUCGUCCACUGGC |
| 235 | AL1-99B | GCCAGUGGACGAUCCAGAA | GCCAGUGGACGAUCCAGAA |
| 236 | AL1-100A | UCCUCUCGGCGUAGGGCUG | UCCUCUCGGCGUAGGGCUG |
| 237 | AL1-100B | CAGCCCUACGCCGAGAGGA | CAGCCCUACGCCGAGAGGA |
| 238 | AL1-101A | AUAGUGCACCCGCACACCG | AUAGUGCACCCGCACACCG |
| 239 | AL1-101B | CGGUGUGCGGGUGCACUAU | CGGUGUGCGGGUGCACUAU |
| 240 | AL1-102A | AGCCAUAGUGCACCCGCAC | AGCCAUAGUGCACCCGCAC |
| 241 | AL1-102B | GUGCGGGUGCACUAUGGCU | GUGCGGGUGCACUAUGGCU |
| 242 | AL1-103A | AAGCCAUAGUGCACCCGCA | AAGCCAUAGUGCACCCGCA |
| 243 | AL1-103B | UGCGGGUGCACUAUGGCUU | UGCGGGUGCACUAUGGCUU |
| 244 | AL1-104A | UACAAGCCAUAGUGCACCC | UACAAGCCAUAGUGCACCC |
| 245 | AL1-104B | GGGUGCACUAUGGCUUGUA | GGGUGCACUAUGGCUUGUA |
| 246 | AL1-105A | UUGUACAAGCCAUAGUGCA | UUGUACAAGCCAUAGUGCA |
| 247 | AL1-105B | UGCACUAUGGCUUGUACAA | UGCACUAUGGCUUGUACAA |
| 248 | AL1-106A | AGAGGAACUCUCCAGGGCA | AGAGGAACUCUCCAGGGCA |
| 249 | AL1-106B | UGCCCUGGAGAGUUCCUCU | UGCCCUGGAGAGUUCCUCU |
| 250 | AL1-107A | ACGCAGUUUCUCUCAUCCA | ACGCAGUUUCUCUCAUCCA |
| 251 | AL1-107B | UGGAUGAGAGAAACUGCGU | UGGAUGAGAGAAACUGCGU |
| 252 | AL1-108A | UGAGAUGCAUGUGCUGUCC | UGAGAUGCAUGUGCUGUCC |
| 253 | AL1-108B | GGACAGCACAUGCAUCUCA | GGACAGCACAUGCAUCUCA |
| 254 | AL1-109A | AGUGAGAUGCAUGUGCUGU | AGUGAGAUGCAUGUGCUGU |
| 255 | AL1-109B | ACAGCACAUGCAUCUCACU | ACAGCACAUGCAUCUCACU |
| 256 | AL1-110A | UGCCCAUCACAGACCUUGG | UGCCCAUCACAGACCUUGG |
| 257 | AL1-110B | CCAAGGUCUGUGAUGGGCA | CCAAGGUCUGUGAUGGGCA |
| 258 | AL1-111A | AGACAAUCAGGCUGCCCAU | AGACAAUCAGGCUGCCCAU |
| 259 | AL1-111B | AUGGGCAGCCUGAUUGUCU | AUGGGCAGCCUGAUUGUCU |
| 260 | AL1-112A | UUGAGACAAUCAGGCUGCC | UUGAGACAAUCAGGCUGCC |
| 261 | AL1-112B | GGCAGCCUGAUUGUCUCAA | GGCAGCCUGAUUGUCUCAA |
| 262 | AL1-113A | UGCCGUUGAGACAAUCAGG | UGCCGUUGAGACAAUCAGG |
| 263 | AL1-113B | CCUGAUUGUCUCAACGGCA | CCUGAUUGUCUCAACGGCA |
| 264 | AL1-114A | UUCGUCGCUGCCGUUGAGA | UUCGUCGCUGCCGUUGAGA |
| 265 | AL1-114B | UCUCAACGGCAGCGACGAA | UCUCAACGGCAGCGACGAA |
| 266 | AL1-115A | AUGGCACCCCUUCCUGGCA | AUGGCACCCCUUCCUGGCA |
| 267 | AL1-115B | UGCCAGGAAGGGGUGCCAU | UGCCAGGAAGGGGUGCCAU |
| 268 | AL1-116A | AUGUCCCACAUGGCACCCC | AUGUCCCACAUGGCACCCC |
| 269 | AL1-116B | GGGGUGCCAUGUGGGACAU | GGGGUGCCAUGUGGGACAU |
| 270 | AL1-117A | UGAAUGUCCCACAUGGCAC | UGAAUGUCCCACAUGGCAC |
| 271 | AL1-117B | GUGCCAUGUGGGACAUUCA | GUGCCAUGUGGGACAUUCA |
| 272 | AL1-118A | AAGGUGAAUGUCCCACAUG | AAGGUGAAUGUCCCACAUG |
| 273 | AL1-118B | CAUGUGGGACAUUCACCUU | CAUGUGGGACAUUCACCUU |
| 274 | AL1-119A | AGCUCCGGUCCUCACACUG | AGCUCCGGUCCUCACACUG |
| 275 | AL1-119B | CAGUGUGAGGACCGGAGCU | CAGUGUGAGGACCGGAGCU |
| 276 | AL1-120A | UGCGGGUUGGGCUUCUUCA | UGCGGGUUGGGCUUCUUCA |
| 277 | AL1-120B | UGAAGAAGCCCAACCCGCA | UGAAGAAGCCCAACCCGCA |
| 278 | AL1-121A | ACACUGCGGGUUGGGCUUC | ACACUGCGGGUUGGGCUUC |
| 279 | AL1-121B | GAAGCCCAACCCGCAGUGU | GAAGCCCAACCCGCAGUGU |
| 280 | AL1-122A | AACAAUGCGGCUGGAGGGG | AACAAUGCGGCUGGAGGGG |
| 281 | AL1-122B | CCCCUCCAGCCGCAUUGUU | CCCCUCCAGCCGCAUUGUU |
| 282 | AL1-123A | UGUGUCGACCCCGAACCUG | UGUGUCGACCCCGAACCUG |
| 283 | AL1-123B | CAGGUUCGGGGUCGACACA | CAGGUUCGGGGUCGACACA |
| 284 | AL1-124A | AUGUGUCGACCCCGAACCU | AUGUGUCGACCCCGAACCU |
| 285 | AL1-124B | AGGUUCGGGGUCGACACAU | AGGUUCGGGGUCGACACAU |
| 286 | AL1-125A | AGAUGUGUCGACCCCGAAC | AGAUGUGUCGACCCCGAAC |
| 287 | AL1-125B | GUUCGGGGUCGACACAUCU | GUUCGGGGUCGACACAUCU |
| 288 | AL1-126A | UAUCACCCAGCGGUCAGCG | UAUCACCCAGCGGUCAGCG |
| 289 | AL1-126B | CGCUGACCGCUGGGUGAUA | CGCUGACCGCUGGGUGAUA |
| 290 | AL1-127A | UUAUCACCCAGCGGUCAGC | UUAUCACCCAGCGGUCAGC |
| 291 | AL1-127B | GCUGACCGCUGGGUGAUAA | GCUGACCGCUGGGUGAUAA |
| 292 | AL1-128A | UGUUAUCACCCAGCGGUCA | UGUUAUCACCCAGCGGUCA |
| 293 | AL1-128B | UGACCGCUGGGUGAUAACA | UGACCGCUGGGUGAUAACA |
| 294 | AL1-129A | AGCUGUUAUCACCCAGCGG | AGCUGUUAUCACCCAGCGG |
| 295 | AL1-129B | CCGCUGGGUGAUAACAGCU | CCGCUGGGUGAUAACAGCU |
| 296 | AL1-130A | AGGAACACGGUCCACAGCA | AGGAACACGGUCCACAGCA |
| 297 | AL1-130B | UGCUGUGGACCGUGUUCCU | UGCUGUGGACCGUGUUCCU |
| 298 | AL1-131A | AGCGCGAGUUCUGCCACAC | AGCGCGAGUUCUGCCACAC |
| 299 | AL1-131B | GUGUGGCAGAACUCGCGCU | GUGUGGCAGAACUCGCGCU |
| 300 | AL1-132A | UCGUGGUACGGGUGCAGGA | UCGUGGUACGGGUGCAGGA |
| 301 | AL1-132B | UCCUGCACCCGUACCACGA | UCCUGCACCCGUACCACGA |
| 302 | AL1-133A | UUCGUGGUACGGGUGCAGG | UUCGUGGUACGGGUGCAGG |
| 303 | AL1-133B | CCUGCACCCGUACCACGAA | CCUGCACCCGUACCACGAA |
| 304 | AL1-134A | UCUUCGUGGUACGGGUGCA | UCUUCGUGGUACGGGUGCA |
| 305 | AL1-134B | UGCACCCGUACCACGAAGA | UGCACCCGUACCACGAAGA |
| 306 | AL1-135A | UCGAAGAAGUGGGAGCGCG | UCGAAGAAGUGGGAGCGCG |
| 307 | AL1-135B | CGCGCUCCCACUUCUUCGA | CGCGCUCCCACUUCUUCGA |
| 308 | AL1-136A | UAAUCCAGCAGUGCAGGCC | UAAUCCAGCAGUGCAGGCC |
| 309 | AL1-136B | GGCCUGCACUGCUGGAUUA | GGCCUGCACUGCUGGAUUA |
| 310 | AL1-137A | AGCCCGUAAUCCAGCAGUG | AGCCCGUAAUCCAGCAGUG |
| 311 | AL1-137B | CACUGCUGGAUUACGGGCU | CACUGCUGGAUUACGGGCU |
| 312 | AL1-138A | UGGGAUCAACUGCACAUCC | UGGGAUCAACUGCACAUCC |
| 313 | AL1-138B | GGAUGUGCAGUUGAUCCCA | GGAUGUGCAGUUGAUCCCA |
| 314 | AL1-139A | AGCGAUAGACCUCGCUGCA | AGCGAUAGACCUCGCUGCA |
| 315 | AL1-139B | UGCAGCGAGGUCUAUCGCU | UGCAGCGAGGUCUAUCGCU |
| 316 | AL1-140A | UAGCGAUAGACCUCGCUGC | UAGCGAUAGACCUCGCUGC |
| 317 | AL1-140B | GCAGCGAGGUCUAUCGCUA | GCAGCGAGGUCUAUCGCUA |
| 318 | AL1-141A | UGGUAGCGAUAGACCUCGC | UGGUAGCGAUAGACCUCGC |
| 319 | AL1-141B | GCGAGGUCUAUCGCUACCA | GCGAGGUCUAUCGCUACCA |
| 320 | AL1-142A | UCACCUGGUAGCGAUAGAC | UCACCUGGUAGCGAUAGAC |
| 321 | AL1-142B | GUCUAUCGCUACCAGGUGA | GUCUAUCGCUACCAGGUGA |
| 322 | AL1-143A | AGCAUGCGUGGCGUCACCU | AGCAUGCGUGGCGUCACCU |
| 323 | AL1-143B | AGGUGACGCCACGCAUGCU | AGGUGACGCCACGCAUGCU |
| 324 | AL1-144A | ACAGCAUGCGUGGCGUCAC | ACAGCAUGCGUGGCGUCAC |
| 325 | AL1-144B | GUGACGCCACGCAUGCUGU | GUGACGCCACGCAUGCUGU |
| 326 | AL1-145A | ACACAGCAUGCGUGGCGUC | ACACAGCAUGCGUGGCGUC |
| 327 | AL1-145B | GACGCCACGCAUGCUGUGU | GACGCCACGCAUGCUGUGU |
| 328 | AL1-146A | AGUUAGGCCGGCCACAGCC | AGUUAGGCCGGCCACAGCC |
| 329 | AL1-146B | GGCUGUGGCCGGCCUAACU | GGCUGUGGCCGGCCUAACU |
| 330 | AL1-147A | UAGUUAGGCCGGCCACAGC | UAGUUAGGCCGGCCACAGC |
| 331 | AL1-147B | GCUGUGGCCGGCCUAACUA | GCUGUGGCCGGCCUAACUA |
| 332 | AL1-148A | AGUAGUUAGGCCGGCCACA | AGUAGUUAGGCCGGCCACA |
| 333 | AL1-148B | UGUGGCCGGCCUAACUACU | UGUGGCCGGCCUAACUACU |
| 334 | AL1-149A | ACGCCGAAGUAGUUAGGCC | ACGCCGAAGUAGUUAGGCC |
| 335 | AL1-149B | GGCCUAACUACUUCGGCGU | GGCCUAACUACUUCGGCGU |
| 336 | AL1-150A | AGACGCCGAAGUAGUUAGG | AGACGCCGAAGUAGUUAGG |
| 337 | AL1-150B | CCUAACUACUUCGGCGUCU | CCUAACUACUUCGGCGUCU |
| 338 | AL1-151A | UAGACGCCGAAGUAGUUAG | UAGACGCCGAAGUAGUUAG |
| 339 | AL1-151B | CUAACUACUUCGGCGUCUA | CUAACUACUUCGGCGUCUA |
| 340 | AL1-152A | UGUAGACGCCGAAGUAGUU | UGUAGACGCCGAAGUAGUU |
| 341 | AL1-152B | AACUACUUCGGCGUCUACA | AACUACUUCGGCGUCUACA |
| 342 | AL1-153A | ACACCUGUGAUGCGGGUGU | ACACCUGUGAUGCGGGUGU |
| 343 | AL1-153B | ACACCCGCAUCACAGGUGU | ACACCCGCAUCACAGGUGU |
| 344 | AL1-154A | UGAUCACACCUGUGAUGCG | UGAUCACACCUGUGAUGCG |
| 345 | AL1-154B | CGCAUCACAGGUGUGAUCA | CGCAUCACAGGUGUGAUCA |
| 346 | AL1-155A | ACCACUUGCUGGAUCCAGC | ACCACUUGCUGGAUCCAGC |
| 347 | AL1-155B | GCUGGAUCCAGCAAGUGGU | GCUGGAUCCAGCAAGUGGU |
| 348 | AL1-156A | UUUGCAGGGGGGCAGUUCC | UUUGCAGGGGGGCAGUUCC |
| 349 | AL1-156B | GGAACUGCCCCCCUGCAAA | GGAACUGCCCCCCUGCAAA |
| 350 | AL1-157A | AGACAAGAUGCCACCUCCU | AGACAAGAUGCCACCUCCU |
| 351 | AL1-157B | AGGAGGUGGCAUCUUGUCU | AGGAGGUGGCAUCUUGUCU |
| 352 | AL1-158A | ACGAGACAAGAUGCCACCU | ACGAGACAAGAUGCCACCU |
| 353 | AL1-158B | AGGUGGCAUCUUGUCUCGU | AGGUGGCAUCUUGUCUCGU |
| 354 | AL1-159A | AGGGACGAGACAAGAUGCC | AGGGACGAGACAAGAUGCC |
| 355 | AL1-159B | GGCAUCUUGUCUCGUCCCU | GGCAUCUUGUCUCGUCCCU |
| 356 | AL1-160A | UCAGGGACGAGACAAGAUG | UCAGGGACGAGACAAGAUG |
| 357 | AL1-160B | CAUCUUGUCUCGUCCCUGA | CAUCUUGUCUCGUCCCUGA |
| 358 | AL1-161A | AUCAGGGACGAGACAAGAU | AUCAGGGACGAGACAAGAU |
| 359 | AL1-161B | AUCUUGUCUCGUCCCUGAU | AUCUUGUCUCGUCCCUGAU |
| 360 | AL1-162A | ACGUCUUGACCCCCAGCUG | ACGUCUUGACCCCCAGCUG |
| 361 | AL1-162B | CAGCUGGGGGUCAAGACGU | CAGCUGGGGGUCAAGACGU |
| 362 | AL1-163A | AGGGGACGUCUUGACCCCC | AGGGGACGUCUUGACCCCC |
| 363 | AL1-163B | GGGGGUCAAGACGUCCCCU | GGGGGUCAAGACGUCCCCU |
| 364 | AL1-164A | UCAGGGGACGUCUUGACCC | UCAGGGGACGUCUUGACCC |
| 365 | AL1-164B | GGGUCAAGACGUCCCCUGA | GGGUCAAGACGUCCCCUGA |
| 366 | AL1-165A | UUGCAUUAGGCAGCAGUGG | UUGCAUUAGGCAGCAGUGG |
| 367 | AL1-165B | CCACUGCUGCCUAAUGCAA | CCACUGCUGCCUAAUGCAA |
| 368 | AL1-166A | UGCCUUGCAUUAGGCAGCA | UGCCUUGCAUUAGGCAGCA |
| 369 | AL1-166B | UGCUGCCUAAUGCAAGGCA | UGCUGCCUAAUGCAAGGCA |
| 370 | AL1-167A | AGCCACUGCCUUGCAUUAG | AGCCACUGCCUUGCAUUAG |
| 371 | AL1-167B | CUAAUGCAAGGCAGUGGCU | CUAAUGCAAGGCAGUGGCU |
| 372 | AL1-168A | AGAACCAGCAUUCUUGCUG | AGAACCAGCAUUCUUGCUG |
| 373 | AL1-168B | CAGCAAGAAUGCUGGUUCU | CAGCAAGAAUGCUGGUUCU |
| 374 | AL1-169A | ACAGAGUGGGGCGCACCUC | ACAGAGUGGGGCGCACCUC |
| 375 | AL1-169B | GAGGUGCGCCCCACUCUGU | GAGGUGCGCCCCACUCUGU |
| 376 | AL1-170A | AGACCAGGGGCUUCCGAAG | AGACCAGGGGCUUCCGAAG |
| 377 | AL1-170B | CUUCGGAAGCCCCUGGUCU | CUUCGGAAGCCCCUGGUCU |
| 378 | AL1-171A | UUAGACCAGGGGCUUCCGA | UUAGACCAGGGGCUUCCGA |
| 379 | AL1-171B | UCGGAAGCCCCUGGUCUAA | UCGGAAGCCCCUGGUCUAA |
| 380 | AL1-172A | AGUUAGACCAGGGGCUUCC | AGUUAGACCAGGGGCUUCC |
| 381 | AL1-172B | GGAAGCCCCUGGUCUAACU | GGAAGCCCCUGGUCUAACU |
| 382 | AL1-173A | AAGUUAGACCAGGGGCUUC | AAGUUAGACCAGGGGCUUC |
| 383 | AL1-173B | GAAGCCCCUGGUCUAACUU | GAAGCCCCUGGUCUAACUU |
| 384 | AL1-174A | AUCCCAAGUUAGACCAGGG | AUCCCAAGUUAGACCAGGG |
| 385 | AL1-174B | CCCUGGUCUAACUUGGGAU | CCCUGGUCUAACUUGGGAU |
| 386 | AL1-175A | AGAUCCCAAGUUAGACCAG | AGAUCCCAAGUUAGACCAG |
| 387 | AL1-175B | CUGGUCUAACUUGGGAUCU | CUGGUCUAACUUGGGAUCU |
| 388 | AL1-176A | UUCCCAGAUCCCAAGUUAG | UUCCCAGAUCCCAAGUUAG |
| 389 | AL1-176B | CUAACUUGGGAUCUGGGAA | CUAACUUGGGAUCUGGGAA |
| 390 | AL1-177A | UGAGGGUCCCCUCCGAUGG | UGAGGGUCCCCUCCGAUGG |
| 391 | AL1-177B | CCAUCGGAGGGGACCCUCA | CCAUCGGAGGGGACCCUCA |
| 392 | AL1-178A | UACAGUGGCAGCAGGCCCA | UACAGUGGCAGCAGGCCCA |
| 393 | AL1-178B | UGGGCCUGCUGCCACUGUA | UGGGCCUGCUGCCACUGUA |
| 394 | AL1-179A | UUACAGUGGCAGCAGGCCC | UUACAGUGGCAGCAGGCCC |
| 395 | AL1-179B | GGGCCUGCUGCCACUGUAA | GGGCCUGCUGCCACUGUAA |
| 396 | AL1-180A | UUUGGCUUACAGUGGCAGC | UUUGGCUUACAGUGGCAGC |
| 397 | AL1-180B | GCUGCCACUGUAAGCCAAA | GCUGCCACUGUAAGCCAAA |
| 398 | AL1-181A | ACCUUUUGGCUUACAGUGG | ACCUUUUGGCUUACAGUGG |
| 399 | AL1-181B | CCACUGUAAGCCAAAAGGU | CCACUGUAAGCCAAAAGGU |
| 400 | AL1-182A | AGUCAGGACUUCCCCACCU | AGUCAGGACUUCCCCACCU |
| 401 | AL1-182B | AGGUGGGGAAGUCCUGACU | AGGUGGGGAAGUCCUGACU |
| 402 | AL1-183A | UGAUCAGGCAGCUUUAUUC | UGAUCAGGCAGCUUUAUUC |
| 403 | AL1-183B | GAAUAAAGCUGCCUGAUCA | GAAUAAAGCUGCCUGAUCA |
| 404 | AL1-184A | UUGAUCAGGCAGCUUUAUU | UUGAUCAGGCAGCUUUAUU |
| 405 | AL1-184B | AAUAAAGCUGCCUGAUCAA | AAUAAAGCUGCCUGAUCAA |
| 406 | AL1-185A | UUUUUUUGAUCAGGCAGCU | UUUUUUUGAUCAGGCAGCU |
| 407 | AL1-185B | AGCUGCCUGAUCAAAAAAA | AGCUGCCUGAUCAAAAAAA |
| 408 | AL1-186A | UUCGUACUCGGCCCUGUAG | UUCGUACUCGGCCCUGUAG |
| 409 | AL1-186B | CUACAGGGCCGAGUACGAA | CUACAGGGCCGAGUACGAA |
| 410 | AL1-187A | AGUCCUUGACCCCAUCACA | AGUCCUUGACCCCAUCACA |
| 411 | AL1-187B | UGUGAUGGGGUCAAGGACU | UGUGAUGGGGUCAAGGACU |
| 412 | AL1-188A | UUGAAGGACACCUCUCCAG | UUGAAGGACACCUCUCCAG |
| 413 | AL1-188B | CUGGAGAGGUGUCCUUCAA | CUGGAGAGGUGUCCUUCAA |
| 414 | AL1-189A | AUACAUGGCCAGUCGGUCC | AUACAUGGCCAGUCGGUCC |
| 415 | AL1-189B | GGACCGACUGGCCAUGUAU | GGACCGACUGGCCAUGUAU |
| 416 | AL1-190A | AGGUCCUGUGGGAUCAACU | AGGUCCUGUGGGAUCAACU |
| 417 | AL1-190B | AGUUGAUCCCACAGGACCU | AGUUGAUCCCACAGGACCU |
| 418 | AL1-191A | UUCUGGGCUUUGGCGGUUU | UUCUGGGCUUUGGCGGUUU |
| 419 | AL1-191B | AAACCGCCAAAGCCCAGAA | AAACCGCCAAAGCCCAGAA |
| 420 | AL1-192A | UUGCGGUAGCCGGCACACA | UUGCGGUAGCCGGCACACA |
| 421 | AL1-192B | UGUGUGCCGGCUACCGCAA | UGUGUGCCGGCUACCGCAA |
| 422 | AL1-193A | UAACAACCCAGCGUGGAAU | UAACAACCCAGCGUGGAAU |
| 423 | AL1-193B | AUUCCACGCUGGGUUGUUA | AUUCCACGCUGGGUUGUUA |
| 424 | AL1-194A | AUCACCCAGCGGUCAGCGA | AUCACCCAGCGGUCAGCGA |
| 425 | AL1-194B | UCGCUGACCGCUGGGUGAU | UCGCUGACCGCUGGGUGAU |
| 426 | AL1-195A | AUACUUGUCCCCCUGCUUG | AUACUUGUCCCCCUGCUUG |
| 427 | AL1-195B | CAAGCAGGGGGACAAGUAU | CAAGCAGGGGGACAAGUAU |
| 428 | AL1-196A | AUCACUGGAGCAGACAUCA | AUCACUGGAGCAGACAUCA |
| 429 | AL1-196B | UGAUGUCUGCUCCAGUGAU | UGAUGUCUGCUCCAGUGAU |
| 430 | AL1-197A | UCACCUUGAAGGACACCUC | UCACCUUGAAGGACACCUC |
| 431 | AL1-197B | GAGGUGUCCUUCAAGGUGA | GAGGUGUCCUUCAAGGUGA |
| 432 | AL1-198A | AAUACUUGUCCCCCUGCUU | AAUACUUGUCCCCCUGCUU |
| 433 | AL1-198B | AAGCAGGGGGACAAGUAUU | AAGCAGGGGGACAAGUAUU |
| 434 | AL1-199A | UCCAUUCCCAGAUCCCAAG | UCCAUUCCCAGAUCCCAAG |
| 435 | AL1-199B | CUUGGGAUCUGGGAAUGGA | CUUGGGAUCUGGGAAUGGA |
| 436 | AL1-200A | UUGUACCCUAGGAAAUACC | UUGUACCCUAGGAAAUACC |
| 437 | AL1-200B | GGUAUUUCCUAGGGUACAA | GGUAUUUCCUAGGGUACAA |
| 438 | AL1-201A | UCUUGCCCUUGCGGUAGCC | UCUUGCCCUUGCGGUAGCC |
| 439 | AL1-201B | GGCUACCGCAAGGGCAAGA | GGCUACCGCAAGGGCAAGA |
| 440 | AL1-202A | AGAAUACUUGUCCCCCUGC | AGAAUACUUGUCCCCCUGC |
| 441 | AL1-202B | GCAGGGGGACAAGUAUUCU | GCAGGGGGACAAGUAUUCU |
| 442 | AL1-203A | AGCAUUCUUGCUGCUGAGC | AGCAUUCUUGCUGCUGAGC |
| 443 | AL1-203B | GCUCAGCAGCAAGAAUGCU | GCUCAGCAGCAAGAAUGCU |
| 444 | AL1-204A | UUCCAUUCCCAGAUCCCAA | UUCCAUUCCCAGAUCCCAA |
| 445 | AL1-204B | UUGGGAUCUGGGAAUGGAA | UUGGGAUCUGGGAAUGGAA |
| 446 | AL1-205A | AGCAUCUUCUGGGCUUUGG | AGCAUCUUCUGGGCUUUGG |
| 447 | AL1-205B | CCAAAGCCCAGAAGAUGCU | CCAAAGCCCAGAAGAUGCU |
| 448 | AL1-206A | UUCUUGCCCUUGCGGUAGC | UUCUUGCCCUUGCGGUAGC |
| 449 | AL1-206B | GCUACCGCAAGGGCAAGAA | GCUACCGCAAGGGCAAGAA |
| 450 | AL1-207A | UUCCAAAGGGCAGCUGAGC | UUCCAAAGGGCAGCUGAGC |
| 451 | AL1-207B | GCUCAGCUGCCCUUUGGAA | GCUCAGCUGCCCUUUGGAA |
| 452 | AL1-208A | AUGCCACCUCCUGCCACCA | AUGCCACCUCCUGCCACCA |
| 453 | AL1-208B | UGGUGGCAGGAGGUGGCAU | UGGUGGCAGGAGGUGGCAU |
| 454 | AL1-209A | AGCCUCUGUACAGAGUGGG | AGCCUCUGUACAGAGUGGG |
| 455 | AL1-209B | CCCACUCUGUACAGAGGCU | CCCACUCUGUACAGAGGCU |
| 456 | AL1-210A | UGAGGGUCUGGGCUGUGAG | UGAGGGUCUGGGCUGUGAG |
| 457 | AL1-210B | CUCACAGCCCAGACCCUCA | CUCACAGCCCAGACCCUCA |
| 458 | AL1-211A | AAGCCGUAGUCCAGAGAGG | AAGCCGUAGUCCAGAGAGG |
| 459 | AL1-211B | CCUCUCUGGACUACGGCUU | CCUCUCUGGACUACGGCUU |
| 460 | AL1-212A | AGAUGCCACCUCCUGCCAC | AGAUGCCACCUCCUGCCAC |
| 461 | AL1-212B | GUGGCAGGAGGUGGCAUCU | GUGGCAGGAGGUGGCAUCU |
| 462 | AL1-213A | UAGACCAGGGGCUUCCGAA | UAGACCAGGGGCUUCCGAA |
| 463 | AL1-213B | UUCGGAAGCCCCUGGUCUA | UUCGGAAGCCCCUGGUCUA |
| 464 | AL1-214A | UCCAAAGGGCAGCUGAGCU | UCCAAAGGGCAGCUGAGCU |
| 465 | AL1-214B | AGCUCAGCUGCCCUUUGGA | AGCUCAGCUGCCCUUUGGA |
| 466 | AL1-215A | AGUUUCUCUCAUCCAGGCC | AGUUUCUCUCAUCCAGGCC |
| 467 | AL1-215B | GGCCUGGAUGAGAGAAACU | GGCCUGGAUGAGAGAAACU |
| 468 | AL1-216A | AAGAUGCCACCUCCUGCCA | AAGAUGCCACCUCCUGCCA |
| 469 | AL1-216B | UGGCAGGAGGUGGCAUCUU | UGGCAGGAGGUGGCAUCUU |
| 470 | AL1-217A | UGGAGGCCACAGUCACAGU | UGGAGGCCACAGUCACAGU |
| 471 | AL1-217B | ACUGUGACUGUGGCCUCCA | ACUGUGACUGUGGCCUCCA |
| 472 | AL1-218A | UCCCAAGUUAGACCAGGGG | UCCCAAGUUAGACCAGGGG |
| 473 | AL1-218B | CCCCUGGUCUAACUUGGGA | CCCCUGGUCUAACUUGGGA |
| 474 | AL1-219A | UAUUCCAAAGGGCAGCUGA | UAUUCCAAAGGGCAGCUGA |
| 475 | AL1-219B | UCAGCUGCCCUUUGGAAUA | UCAGCUGCCCUUUGGAAUA |
| 476 | AL1-220A | ACAGAUGUGUCGACCCCGA | ACAGAUGUGUCGACCCCGA |
| 477 | AL1-220B | UCGGGGUCGACACAUCUGU | UCGGGGUCGACACAUCUGU |
| 478 | AL1-221A | UGGAGCAGACAUCAGGGAC | UGGAGCAGACAUCAGGGAC |
| 479 | AL1-221B | GUCCCUGAUGUCUGCUCCA | GUCCCUGAUGUCUGCUCCA |
| 480 | AL1-222A | ACCCAGCGGUCAGCGAUGA | ACCCAGCGGUCAGCGAUGA |
| 481 | AL1-222B | UCAUCGCUGACCGCUGGGU | UCAUCGCUGACCGCUGGGU |
| 482 | AL1-223A | UUAUUCCAAAGGGCAGCUG | UUAUUCCAAAGGGCAGCUG |
| 483 | AL2-1A | UUGAAGGACACCUCUCCAGGCCA | UUGAAGGACACCUCUCCAGGCCA |
| 484 | AL2-1B | UGGCCUGGAGAGGUGUCCUUC | UGGCCUGGAGAGGUGUCCUUC |
| 485 | AL2-2A | AGGAAAUACCAGAGUAGCACCCC | AGGAAAUACCAGAGUAGCACCCC |
| 486 | AL2-2B | GGGGUGCUACUCUGGUAUUUC | GGGGUGCUACUCUGGUAUUUC |
| 487 | AL2-3A | AGUUUCUCUCAUCCAGGCCGUUG | AGUUUCUCUCAUCCAGGCCGUUG |
| 488 | AL2-3B | CAACGGCCUGGAUGAGAGAAA | CAACGGCCUGGAUGAGAGAAA |
| 489 | AL2-4A | AAGAUCCUGGGAGAAGUGGCGAU | AAGAUCCUGGGAGAAGUGGCGAU |
| 490 | AL2-4B | AUCGCCACUUCUCCCAGGAUC | AUCGCCACUUCUCCCAGGAUC |
| 491 | AL2-5A | ACAAGAUGCCACCUCCUGCCACC | ACAAGAUGCCACCUCCUGCCACC |
| 492 | AL2-5B | GGUGGCAGGAGGUGGCAUCUU | GGUGGCAGGAGGUGGCAUCUU |
| 493 | AL2-6A | ACGUCAUACAUGGCCAGUCGGUC | ACGUCAUACAUGGCCAGUCGGUC |
| 494 | AL2-6B | GACCGACUGGCCAUGUAUGAC | GACCGACUGGCCAUGUAUGAC |
| 495 | AL2-7A | AAGCCAUAGUGCACCCGCACACC | AAGCCAUAGUGCACCCGCACACC |
| 496 | AL2-7B | GGUGUGCGGGUGCACUAUGGC | GGUGUGCGGGUGCACUAUGGC |
| 497 | AL2-8A | ACGCAGUUUCUCUCAUCCAGGCC | ACGCAGUUUCUCUCAUCCAGGCC |
| 498 | AL2-8B | GGCCUGGAUGAGAGAAACUGC | GGCCUGGAUGAGAGAAACUGC |
| 499 | AL2-9A | UUGUACCCUAGGAAAUACCAGAG | UUGUACCCUAGGAAAUACCAGAG |
| 500 | AL2-9B | CUCUGGUAUUUCCUAGGGUAC | CUCUGGUAUUUCCUAGGGUAC |
| 501 | AL2-10A | AGAUCCCAAGUUAGACCAGGGGC | AGAUCCCAAGUUAGACCAGGGGC |
| 502 | AL2-10B | GCCCCUGGUCUAACUUGGGAU | GCCCCUGGUCUAACUUGGGAU |
| 503 | AL2-11A | ACGGCAAAUCAUACUUCUGCCUC | ACGGCAAAUCAUACUUCUGCCUC |
| 504 | AL2-11B | GAGGCAGAAGUAUGAUUUGCC | GAGGCAGAAGUAUGAUUUGCC |
| 505 | AL2-12A | AGCUAUGUCUUUCACACUGGCUU | AGCUAUGUCUUUCACACUGGCUU |
| 506 | AL2-12B | AAGCCAGUGUGAAAGACAUAG | AAGCCAGUGUGAAAGACAUAG |
| 507 | AL2-13A | AUACAUGGCCAGUCGGUCCCGGC | AUACAUGGCCAGUCGGUCCCGGC |
| 508 | AL2-13B | GCCGGGACCGACUGGCCAUGU | GCCGGGACCGACUGGCCAUGU |
| 509 | AL2-14A | AUGUGUCGACCCCGAACCUGGAG | AUGUGUCGACCCCGAACCUGGAG |
| 510 | AL2-14B | CUCCAGGUUCGGGGUCGACAC | CUCCAGGUUCGGGGUCGACAC |
| 511 | AL2-15A | GAUCCCAAGUUAGACCAGGGGCU | GAUCCCAAGUUAGACCAGGGGCU |
| 512 | AL2-15B | AGCCCCUGGUCUAACUUGGGA | AGCCCCUGGUCUAACUUGGGA |
| 513 | AL2-16A | UAAGAUCCUGGGAGAAGUGGCGA | UAAGAUCCUGGGAGAAGUGGCGA |
| 514 | AL2-16B | UCGCCACUUCUCCCAGGAUCU | UCGCCACUUCUCCCAGGAUCU |
| 515 | AL2-17A | UGUACCCUAGGAAAUACCAGAGU | UGUACCCUAGGAAAUACCAGAGU |
| 516 | AL2-17B | ACUCUGGUAUUUCCUAGGGUA | ACUCUGGUAUUUCCUAGGGUA |
| 517 | AL2-18A | UGUUAUCACCCAGCGGUCAGCGA | UGUUAUCACCCAGCGGUCAGCGA |
| 518 | AL2-18B | UCGCUGACCGCUGGGUGAUAA | UCGCUGACCGCUGGGUGAUAA |
| 519 | AL2-19A | UCUCUCAUCCAGGCCGUUGGGGC | UCUCUCAUCCAGGCCGUUGGGGC |
| 520 | AL2-19B | GCCCCAACGGCCUGGAUGAGA | GCCCCAACGGCCUGGAUGAGA |
| 521 | AL2-20A | GAAUACUUGUCCCCCUGCUUGGC | GAAUACUUGUCCCCCUGCUUGGC |
| 522 | AL2-20B | GCCAAGCAGGGGGACAAGUAU | GCCAAGCAGGGGGACAAGUAU |
| 523 | AL2-21A | UUCGUACUCGGCCCUGUAGGGGA | UUCGUACUCGGCCCUGUAGGGGA |
| 524 | AL2-21B | UCCCCUACAGGGCCGAGUACG | UCCCCUACAGGGCCGAGUACG |
| 525 | AL2-22A | UAGCUGUAGCGGUAACAACCCAG | UAGCUGUAGCGGUAACAACCCAG |
| 526 | AL2-22B | CUGGGUUGUUACCGCUACAGC | CUGGGUUGUUACCGCUACAGC |
| 527 | AL2-23A | UGAAGGACACCUCUCCAGGCCAG | UGAAGGACACCUCUCCAGGCCAG |
| 528 | AL2-23B | CUGGCCUGGAGAGGUGUCCUU | CUGGCCUGGAGAGGUGUCCUU |
| 529 | AL2-24A | UACAAGCCAUAGUGCACCCGCAC | UACAAGCCAUAGUGCACCCGCAC |
| 530 | AL2-24B | GUGCGGGUGCACUAUGGCUUG | GUGCGGGUGCACUAUGGCUUG |
| 531 | AL2-25A | AGACAAGAUGCCACCUCCUGCCA | AGACAAGAUGCCACCUCCUGCCA |
| 532 | AL2-25B | UGGCAGGAGGUGGCAUCUUGU | UGGCAGGAGGUGGCAUCUUGU |
| 533 | AL2-26A | ACUUCGUACUCGGCCCUGUAGGG | ACUUCGUACUCGGCCCUGUAGGG |
| 534 | AL2-26B | CCCUACAGGGCCGAGUACGAA | CCCUACAGGGCCGAGUACGAA |
| 535 | AL2-27A | AGAAUGAACCAGAAGAAGCAGGU | AGAAUGAACCAGAAGAAGCAGGU |
| 536 | AL2-27B | ACCUGCUUCUUCUGGUUCAUU | ACCUGCUUCUUCUGGUUCAUU |
| 537 | AL2-28A | AGUCCUUGACCCCAUCACAGGCA | AGUCCUUGACCCCAUCACAGGCA |
| 538 | AL2-28B | UGCCUGUGAUGGGGUCAAGGA | UGCCUGUGAUGGGGUCAAGGA |
| 539 | AL2-29A | UAGACCAGGGGCUUCCGAAGCUG | UAGACCAGGGGCUUCCGAAGCUG |
| 540 | AL2-29B | CAGCUUCGGAAGCCCCUGGUC | CAGCUUCGGAAGCCCCUGGUC |
| 541 | AL2-30A | CAGAUCCCAAGUUAGACCAGGGG | CAGAUCCCAAGUUAGACCAGGGG |
| 542 | AL2-30B | CCCCUGGUCUAACUUGGGAUC | CCCCUGGUCUAACUUGGGAUC |
| 543 | AL2-31A | UGGAGAAUGAACCAGAAGAAGCA | UGGAGAAUGAACCAGAAGAAGCA |
| 544 | AL2-31B | UGCUUCUUCUGGUUCAUUCUC | UGCUUCUUCUGGUUCAUUCUC |
| 545 | AL2-32A | UUUCUCUCAUCCAGGCCGUUGGG | UUUCUCUCAUCCAGGCCGUUGGG |
| 546 | AL2-32B | CCCAACGGCCUGGAUGAGAGA | CCCAACGGCCUGGAUGAGAGA |
| 547 | AL2-33A | UCGUAGUAGCUGUGCAGGCCCUU | UCGUAGUAGCUGUGCAGGCCCUU |
| 548 | AL2-33B | AAGGGCCUGCACAGCUACUAC | AAGGGCCUGCACAGCUACUAC |
| 549 | AL2-34A | CAUUCCCAGAUCCCAAGUUAGAC | CAUUCCCAGAUCCCAAGUUAGAC |
| 550 | AL2-34B | GUCUAACUUGGGAUCUGGGAA | GUCUAACUUGGGAUCUGGGAA |
| 551 | AL2-35A | UUAGACCAGGGGCUUCCGAAGCU | UUAGACCAGGGGCUUCCGAAGCU |
| 552 | AL2-35B | AGCUUCGGAAGCCCCUGGUCU | AGCUUCGGAAGCCCCUGGUCU |
| 553 | AL2-36A | UGCAGCUAUGUCUUUCACACUGG | UGCAGCUAUGUCUUUCACACUGG |
| 554 | AL2-36B | CCAGUGUGAAAGACAUAGCUG | CCAGUGUGAAAGACAUAGCUG |
| 555 | AL2-37A | AGAUGUGUCGACCCCGAACCUGG | AGAUGUGUCGACCCCGAACCUGG |
| 556 | AL2-37B | CCAGGUUCGGGGUCGACACAU | CCAGGUUCGGGGUCGACACAU |
| 557 | AL2-38A | UAGCGGUAACAACCCAGCGUGGA | UAGCGGUAACAACCCAGCGUGGA |
| 558 | AL2-38B | UCCACGCUGGGUUGUUACCGC | UCCACGCUGGGUUGUUACCGC |
| 559 | AL2-39A | AAUACUUGUCCCCCUGCUUGGCA | AAUACUUGUCCCCCUGCUUGGCA |
| 560 | AL2-39B | UGCCAAGCAGGGGGACAAGUA | UGCCAAGCAGGGGGACAAGUA |
| 561 | AL2-40A | CCACACAGCCUCCUGUUCUGGAU | CCACACAGCCUCCUGUUCUGGAU |
| 562 | AL2-40B | AUCCAGAACAGGAGGCUGUGU | AUCCAGAACAGGAGGCUGUGU |
| 563 | AL2-41A | GUGAGGGAGAUCUGGGAGGUGAA | GUGAGGGAGAUCUGGGAGGUGAA |
| 564 | AL2-41B | UUCACCUCCCAGAUCUCCCUC | UUCACCUCCCAGAUCUCCCUC |
| 565 | AL2-42A | CCAUGGCCACUCACCCUCGGAGG | CCAUGGCCACUCACCCUCGGAGG |
| 566 | AL2-42B | CCUCCGAGGGUGAGUGGCCAU | CCUCCGAGGGUGAGUGGCCAU |
| 567 | AL2-43A | GCCACACAGCCUCCUGUUCUGGA | GCCACACAGCCUCCUGUUCUGGA |
| 568 | AL2-43B | UCCAGAACAGGAGGCUGUGUG | UCCAGAACAGGAGGCUGUGUG |
| 569 | AL2-44A | GGCCACUCACCCUCGGAGGACAC | GGCCACUCACCCUCGGAGGACAC |
| 570 | AL2-44B | GUGUCCUCCGAGGGUGAGUGG | GUGUCCUCCGAGGGUGAGUGG |
| 571 | AL2-45A | CCCACAGAUGUGUCGACCCCGAA | CCCACAGAUGUGUCGACCCCGAA |
| 572 | AL2-45B | UUCGGGGUCGACACAUCUGUG | UUCGGGGUCGACACAUCUGUG |
| 573 | AL2-46A | CCCCACAGAUGUGUCGACCCCGA | CCCCACAGAUGUGUCGACCCCGA |
| 574 | AL2-46B | UCGGGGUCGACACAUCUGUGG | UCGGGGUCGACACAUCUGUGG |
| 575 | AL2-47A | GCCAUGCUGUCCUCCUGGAAGCA | GCCAUGCUGUCCUCCUGGAAGCA |
| 576 | AL2-47B | UGCUUCCAGGAGGACAGCAUG | UGCUUCCAGGAGGACAGCAUG |
| 577 | AL2-48A | UGUACCCUAGGAAAUACCAGAGU | UGUACCCUAGGAAAUACCAGAGU |
| 578 | AL2-48B | UCUGGUAUUUCCUAGGGUACA | UCUGGUAUUUCCUAGGGUACA |
| 579 | AL2-49A | AGGAAAUACCAGAGUAGCACCCC | AGGAAAUACCAGAGUAGCACCCC |
| 580 | AL2-49B | GGUGCUACUCUGGUAUUUCCU | GGUGCUACUCUGGUAUUUCCU |
| 581 | AL2-50A | UGUACCCUAGGAAAUACCAGAGU | UGUACCCUAGGAAAUACCAGAGU |
| 582 | AL2-50B | UCUGGUAUUUCCUAGGGUACA | UCUGGUAUUUCCUAGGGUACA |
| 583 | AL2-51A | UUGUACCCUAGGAAAUACCAGAG | UUGUACCCUAGGAAAUACCAGAG |
| 584 | AL2-51B | CUGGUAUUUCCUAGGGUACAA | CUGGUAUUUCCUAGGGUACAA |
| 585 | AL2-52A | UUUGUACCCUAGGAAAUACCAGA | UUUGUACCCUAGGAAAUACCAGA |
| 586 | AL2-52B | UGGUAUUUCCUAGGGUACAAA | UGGUAUUUCCUAGGGUACAAA |
| 587 | AL2-53A | UCUUGUACCCUAGGAAAUACCAG | UCUUGUACCCUAGGAAAUACCAG |
| 588 | AL2-53B | GGUAUUUCCUAGGGUACAAGA | GGUAUUUCCUAGGGUACAAGA |
| 589 | AL2-54A | UGUACCCUAGGAAAUACCA | UGUACCCUAGGAAAUACCA |
| 590 | AL2-54B | UGGUAUUUCCUAGGGUACA | UGGUAUUUCCUAGGGUACA |
| 591 | AL2-55A | UCCUUGUACCCUAGGAAAUACCA | UCCUUGUACCCUAGGAAAUACCA |
| 592 | AL2-55B | GUAUUUCCUAGGGUACAAGGA | GUAUUUCCUAGGGUACAAGGA |
| 593 | AL2-56A | UCGCCUUGUACCCUAGGAAAUAC | UCGCCUUGUACCCUAGGAAAUAC |
| 594 | AL2-56B | AUUUCCUAGGGUACAAGGCGA | AUUUCCUAGGGUACAAGGCGA |
| 595 | AL2-57A | UCCGCCUUGUACCCUAGGAAAUA | UCCGCCUUGUACCCUAGGAAAUA |
| 596 | AL2-57B | UUUCCUAGGGUACAAGGCGGA | UUUCCUAGGGUACAAGGCGGA |
| 597 | AL2-58A | AAGAUCCUGGGAGAAGUGGCGAU | AAGAUCCUGGGAGAAGUGGCGAU |
| 598 | AL2-58B | CGCCACUUCUCCCAGGAUCUU | CGCCACUUCUCCCAGGAUCUU |
| 599 | AL2-59A | UAAGAUCCUGGGAGAAGUGGCGA | UAAGAUCCUGGGAGAAGUGGCGA |
| 600 | AL2-59B | GCCACUUCUCCCAGGAUCUUA | GCCACUUCUCCCAGGAUCUUA |
| 601 | AL2-60A | AGAAUGAACCAGAAGAAGCAGGU | AGAAUGAACCAGAAGAAGCAGGU |
| 602 | AL2-60B | CUGCUUCUUCUGGUUCAUUCU | CUGCUUCUUCUGGUUCAUUCU |
| 603 | AL2-61A | UGGAGAAUGAACCAGAAGAAGCA | UGGAGAAUGAACCAGAAGAAGCA |
| 604 | AL2-61B | CUUCUUCUGGUUCAUUCUCCA | CUUCUUCUGGUUCAUUCUCCA |
| 605 | AL2-62A | UUCGUACUCGGCCCUGUAGGGGA | UUCGUACUCGGCCCUGUAGGGGA |
| 606 | AL2-62B | CCCUACAGGGCCGAGUACGAA | CCCUACAGGGCCGAGUACGAA |
| 607 | AL2-63A | ACUUCGUACUCGGCCCUGUAGGG | ACUUCGUACUCGGCCCUGUAGGG |
| 608 | AL2-63B | CUACAGGGCCGAGUACGAAGU | CUACAGGGCCGAGUACGAAGU |
| 609 | AL2-64A | AGCUAUGUCUUUCACACUGGCUU | AGCUAUGUCUUUCACACUGGCUU |
| 610 | AL2-64B | GCCAGUGUGAAAGACAUAGCU | GCCAGUGUGAAAGACAUAGCU |
| 611 | AL2-65A | UGCAGCUAUGUCUUUCACACUGG | UGCAGCUAUGUCUUUCACACUGG |
| 612 | AL2-65B | AGUGUGAAAGACAUAGCUGCA | AGUGUGAAAGACAUAGCUGCA |
| 613 | AL2-66A | UAGCGGUAACAACCCAGCGUGGA | UAGCGGUAACAACCCAGCGUGGA |
| 614 | AL2-66B | CACGCUGGGUUGUUACCGCUA | CACGCUGGGUUGUUACCGCUA |
| 615 | AL2-67A | UAGCUGUAGCGGUAACAACCCAG | UAGCUGUAGCGGUAACAACCCAG |
| 616 | AL2-67B | GGGUUGUUACCGCUACAGCUA | GGGUUGUUACCGCUACAGCUA |
| 617 | AL2-68A | AUACAUGGCCAGUCGGUCCCGGC | AUACAUGGCCAGUCGGUCCCGGC |
| 618 | AL2-68B | CGGGACCGACUGGCCAUGUAU | CGGGACCGACUGGCCAUGUAU |
| 619 | AL2-69A | ACGUCAUACAUGGCCAGUCGGUC | ACGUCAUACAUGGCCAGUCGGUC |
| 620 | AL2-69B | CCGACUGGCCAUGUAUGACGU | CCGACUGGCCAUGUAUGACGU |
| 621 | AL2-70A | UCGUAGUAGCUGUGCAGGCCCUU | UCGUAGUAGCUGUGCAGGCCCUU |
| 622 | AL2-70B | GGGCCUGCACAGCUACUACGA | GGGCCUGCACAGCUACUACGA |
| 623 | AL2-71A | ACGGCAAAUCAUACUUCUGCCUC | ACGGCAAAUCAUACUUCUGCCUC |
| 624 | AL2-71B | GGCAGAAGUAUGAUUUGCCGU | GGCAGAAGUAUGAUUUGCCGU |
| 625 | AL2-72A | CCACACAGCCUCCUGUUCUGGAU | CCACACAGCCUCCUGUUCUGGAU |
| 626 | AL2-72B | CCAGAACAGGAGGCUGUGUGG | CCAGAACAGGAGGCUGUGUGG |
| 627 | AL2-73A | GCCACACAGCCUCCUGUUCUGGA | GCCACACAGCCUCCUGUUCUGGA |
| 628 | AL2-73B | CAGAACAGGAGGCUGUGUGGC | CAGAACAGGAGGCUGUGUGGC |
| 629 | AL2-74A | GUGAGGGAGAUCUGGGAGGUGAA | GUGAGGGAGAUCUGGGAGGUGAA |
| 630 | AL2-74B | CACCUCCCAGAUCUCCCUCAC | CACCUCCCAGAUCUCCCUCAC |
| 631 | AL2-75A | UUGAGGGAGAUCUGGGAGGUGAA | UUGAGGGAGAUCUGGGAGGUGAA |
| 632 | AL2-75B | CACCUCCCAGAUCUCCCUCAA | CACCUCCCAGAUCUCCCUCAA |
| 633 | AL2-76A | AAGCCAUAGUGCACCCGCACACC | AAGCCAUAGUGCACCCGCACACC |
| 634 | AL2-76B | UGUGCGGGUGCACUAUGGCUU | UGUGCGGGUGCACUAUGGCUU |
| 635 | AL2-77A | UACAAGCCAUAGUGCACCCGCAC | UACAAGCCAUAGUGCACCCGCAC |
| 636 | AL2-77B | GCGGGUGCACUAUGGCUUGUA | GCGGGUGCACUAUGGCUUGUA |
| 637 | AL2-78A | AGGAACUCUCCAGGGCAGGGGUC | AGGAACUCUCCAGGGCAGGGGUC |
| 638 | AL2-78B | CCCCUGCCCUGGAGAGUUCCU | CCCCUGCCCUGGAGAGUUCCU |
| 639 | AL2-79A | UAGGAACUCUCCAGGGCAGGGGU | UAGGAACUCUCCAGGGCAGGGGU |
| 640 | AL2-79B | CCCUGCCCUGGAGAGUUCCUA | CCCUGCCCUGGAGAGUUCCUA |
| 641 | AL2-80A | AGAGGAACUCUCCAGGGCAGGGG | AGAGGAACUCUCCAGGGCAGGGG |
| 642 | AL2-80B | CCUGCCCUGGAGAGUUCCUCU | CCUGCCCUGGAGAGUUCCUCU |
| 643 | AL2-81A | UAGAGGAACUCUCCAGGGCAGGG | UAGAGGAACUCUCCAGGGCAGGG |
| 644 | AL2-81B | CUGCCCUGGAGAGUUCCUCUA | CUGCCCUGGAGAGUUCCUCUA |
| 645 | AL2-82A | AGUCCUUGACCCCAUCACAGGCA | AGUCCUUGACCCCAUCACAGGCA |
| 646 | AL2-82B | CCUGUGAUGGGGUCAAGGACU | CCUGUGAUGGGGUCAAGGACU |
| 647 | AL2-83A | UCCAGGCCGUUGGGGCAGUCCUU | UCCAGGCCGUUGGGGCAGUCCUU |
| 648 | AL2-83B | GGACUGCCCCAACGGCCUGGA | GGACUGCCCCAACGGCCUGGA |
| 649 | AL2-84A | UAUCCAGGCCGUUGGGGCAGUCC | UAUCCAGGCCGUUGGGGCAGUCC |
| 650 | AL2-84B | ACUGCCCCAACGGCCUGGAUA | ACUGCCCCAACGGCCUGGAUA |
| 651 | AL2-85A | UCAUCCAGGCCGUUGGGGCAGUC | UCAUCCAGGCCGUUGGGGCAGUC |
| 652 | AL2-85B | CUGCCCCAACGGCCUGGAUGA | CUGCCCCAACGGCCUGGAUGA |
| 653 | AL2-86A | UUCAUCCAGGCCGUUGGGGCAGU | UUCAUCCAGGCCGUUGGGGCAGU |
| 654 | AL2-86B | UGCCCCAACGGCCUGGAUGAA | UGCCCCAACGGCCUGGAUGAA |
| 655 | AL2-87A | UCUCAUCCAGGCCGUUGGGGCAG | UCUCAUCCAGGCCGUUGGGGCAG |
| 656 | AL2-87B | GCCCCAACGGCCUGGAUGAGA | GCCCCAACGGCCUGGAUGAGA |
| 657 | AL2-88A | UUCUCAUCCAGGCCGUUGGGGCA | UUCUCAUCCAGGCCGUUGGGGCA |
| 658 | AL2-88B | CCCCAACGGCCUGGAUGAGAA | CCCCAACGGCCUGGAUGAGAA |
| 659 | AL2-89A | UCUCUCAUCCAGGCCGUUGGGGC | UCUCUCAUCCAGGCCGUUGGGGC |
| 660 | AL2-89B | CCCAACGGCCUGGAUGAGAGA | CCCAACGGCCUGGAUGAGAGA |
| 661 | AL2-90A | UUUCUCUCAUCCAGGCCGUUGGG | UUUCUCUCAUCCAGGCCGUUGGG |
| 662 | AL2-90B | CAACGGCCUGGAUGAGAGAAA | CAACGGCCUGGAUGAGAGAAA |
| 663 | AL2-91A | AGUUUCUCUCAUCCAGGCCGUUG | AGUUUCUCUCAUCCAGGCCGUUG |
| 664 | AL2-91B | ACGGCCUGGAUGAGAGAAACU | ACGGCCUGGAUGAGAGAAACU |
| 665 | AL2-92A | ACGCAGUUUCUCUCAUCCAGGCC | ACGCAGUUUCUCUCAUCCAGGCC |
| 666 | AL2-92B | CCUGGAUGAGAGAAACUGCGU | CCUGGAUGAGAGAAACUGCGU |
| 667 | AL2-93A | UUGGAGGCCACAGUCACAGUGCU | UUGGAGGCCACAGUCACAGUGCU |
| 668 | AL2-93B | CACUGUGACUGUGGCCUCCAA | CACUGUGACUGUGGCCUCCAA |
| 669 | AL2-94A | GGCCACUCACCCUCGGAGGACAC | GGCCACUCACCCUCGGAGGACAC |
| 670 | AL2-94B | GUCCUCCGAGGGUGAGUGGCC | GUCCUCCGAGGGUGAGUGGCC |
| 671 | AL2-95A | UAUGGCCACUCACCCUCGGAGGA | UAUGGCCACUCACCCUCGGAGGA |
| 672 | AL2-95B | CUCCGAGGGUGAGUGGCCAUA | CUCCGAGGGUGAGUGGCCAUA |
| 673 | AL2-96A | CCAUGGCCACUCACCCUCGGAGG | CCAUGGCCACUCACCCUCGGAGG |
| 674 | AL2-96B | UCCGAGGGUGAGUGGCCAUGG | UCCGAGGGUGAGUGGCCAUGG |
| 675 | AL2-97A | AUGUGUCGACCCCGAACCUGGAG | AUGUGUCGACCCCGAACCUGGAG |
| 676 | AL2-97B | CCAGGUUCGGGGUCGACACAU | CCAGGUUCGGGGUCGACACAU |
| 677 | AL2-98A | AGAUGUGUCGACCCCGAACCUGG | AGAUGUGUCGACCCCGAACCUGG |
| 678 | AL2-98B | AGGUUCGGGGUCGACACAUCU | AGGUUCGGGGUCGACACAUCU |
| 679 | AL2-99A | CCCACAGAUGUGUCGACCCCGAA | CCCACAGAUGUGUCGACCCCGAA |
| 680 | AL2-99B | CGGGGUCGACACAUCUGUGGG | CGGGGUCGACACAUCUGUGGG |
| 681 | AL2-100A | UCCACAGAUGUGUCGACCCCGAA | UCCACAGAUGUGUCGACCCCGAA |
| 682 | AL2-100B | CGGGGUCGACACAUCUGUGGA | CGGGGUCGACACAUCUGUGGA |
| 683 | AL2-101A | CCCCACAGAUGUGUCGACCCCGA | CCCCACAGAUGUGUCGACCCCGA |
| 684 | AL2-101B | GGGGUCGACACAUCUGUGGGG | GGGGUCGACACAUCUGUGGGG |
| 685 | AL2-102A | UCCCACAGAUGUGUCGACCCCGA | UCCCACAGAUGUGUCGACCCCGA |
| 686 | AL2-102B | GGGGUCGACACAUCUGUGGGA | GGGGUCGACACAUCUGUGGGA |
| 687 | AL2-103A | UCCCCACAGAUGUGUCGACCCCG | UCCCCACAGAUGUGUCGACCCCG |
| 688 | AL2-103B | GGGUCGACACAUCUGUGGGGA | GGGUCGACACAUCUGUGGGGA |
| 689 | AL2-104A | UGUUAUCACCCAGCGGUCAGCGA | UGUUAUCACCCAGCGGUCAGCGA |
| 690 | AL2-104B | GCUGACCGCUGGGUGAUAACA | GCUGACCGCUGGGUGAUAACA |
| 691 | AL2-105A | GCCAUGCUGUCCUCCUGGAAGCA | GCCAUGCUGUCCUCCUGGAAGCA |
| 692 | AL2-105B | CUUCCAGGAGGACAGCAUGGC | CUUCCAGGAGGACAGCAUGGC |
| 693 | AL2-106A | UGAAGGACACCUCUCCAGGCCAG | UGAAGGACACCUCUCCAGGCCAG |
| 694 | AL2-106B | GGCCUGGAGAGGUGUCCUUCA | GGCCUGGAGAGGUGUCCUUCA |
| 695 | AL2-107A | UUGAAGGACACCUCUCCAGGCCA | UUGAAGGACACCUCUCCAGGCCA |
| 696 | AL2-107B | GCCUGGAGAGGUGUCCUUCAA | GCCUGGAGAGGUGUCCUUCAA |
| 697 | AL2-108A | AAUACUUGUCCCCCUGCUUGGCA | AAUACUUGUCCCCCUGCUUGGCA |
| 698 | AL2-108B | CCAAGCAGGGGGACAAGUAUU | CCAAGCAGGGGGACAAGUAUU |
| 699 | AL2-109A | GAAUACUUGUCCCCCUGCUUGGC | GAAUACUUGUCCCCCUGCUUGGC |
| 700 | AL2-109B | CAAGCAGGGGGACAAGUAUUC | CAAGCAGGGGGACAAGUAUUC |
| 701 | AL2-110A | ACAAGAUGCCACCUCCUGCCACC | ACAAGAUGCCACCUCCUGCCACC |
| 702 | AL2-110B | UGGCAGGAGGUGGCAUCUUGU | UGGCAGGAGGUGGCAUCUUGU |
| 703 | AL2-111A | AGACAAGAUGCCACCUCCUGCCA | AGACAAGAUGCCACCUCCUGCCA |
| 704 | AL2-111B | GCAGGAGGUGGCAUCUUGUCU | GCAGGAGGUGGCAUCUUGUCU |
| 705 | AL2-112A | UAGACCAGGGGCUUCCGAAGCUG | UAGACCAGGGGCUUCCGAAGCUG |
| 706 | AL2-112B | GCUUCGGAAGCCCCUGGUCUA | GCUUCGGAAGCCCCUGGUCUA |
| 707 | AL2-113A | UUAGACCAGGGGCUUCCGAAGCU | UUAGACCAGGGGCUUCCGAAGCU |
| 708 | AL2-113B | CUUCGGAAGCCCCUGGUCUAA | CUUCGGAAGCCCCUGGUCUAA |
| 709 | AL2-114A | GAUCCCAAGUUAGACCAGGGGCU | GAUCCCAAGUUAGACCAGGGGCU |
| 710 | AL2-114B | CCCCUGGUCUAACUUGGGAUC | CCCCUGGUCUAACUUGGGAUC |
| 711 | AL2-115A | AGAUCCCAAGUUAGACCAGGGGC | AGAUCCCAAGUUAGACCAGGGGC |
| 712 | AL2-115B | CCCUGGUCUAACUUGGGAUCU | CCCUGGUCUAACUUGGGAUCU |
| 713 | AL2-116A | CAGAUCCCAAGUUAGACCAGGGG | CAGAUCCCAAGUUAGACCAGGGG |
| 714 | AL2-116B | CCUGGUCUAACUUGGGAUCUG | CCUGGUCUAACUUGGGAUCUG |
| 715 | AL2-117A | CAUUCCCAGAUCCCAAGUUAGAC | CAUUCCCAGAUCCCAAGUUAGAC |
| 716 | AL2-117B | CUAACUUGGGAUCUGGGAAUG | CUAACUUGGGAUCUGGGAAUG |
| 717 | AL2-118A | CUGGACUGGGUCUGGCUCA | CUGGACUGGGUCUGGCUCA |
| 718 | AL2-118B | UGAGCCAGACCCAGUCCAG | UGAGCCAGACCCAGUCCAG |
| 719 | AL2-119A | ACCAGAGCUGGACUGGGUC | ACCAGAGCUGGACUGGGUC |
| 720 | AL2-119B | GACCCAGUCCAGCUCUGGU | GACCCAGUCCAGCUCUGGU |
| 721 | AL2-120A | CAGAGGGCAGGCACCAGAG | CAGAGGGCAGGCACCAGAG |
| 722 | AL2-120B | CUCUGGUGCCUGCCCUCUG | CUCUGGUGCCUGCCCUCUG |
| 723 | AL2-121A | UCAGCUCGCACCAGAGGGC | UCAGCUCGCACCAGAGGGC |
| 724 | AL2-121B | GCCCUCUGGUGCGAGCUGA | GCCCUCUGGUGCGAGCUGA |
| 725 | AL2-122A | UCUCAGGUCAGCUCGCACC | UCUCAGGUCAGCUCGCACC |
| 726 | AL2-122B | GGUGCGAGCUGACCUGAGA | GGUGCGAGCUGACCUGAGA |
| 727 | AL2-123A | GGAAGUGCAUCUCAGGUCA | GGAAGUGCAUCUCAGGUCA |
| 728 | AL2-123B | UGACCUGAGAUGCACUUCC | UGACCUGAGAUGCACUUCC |
| 729 | AL2-124A | CACAGAGGAGGGAAGUGCA | CACAGAGGAGGGAAGUGCA |
| 730 | AL2-124B | UGCACUUCCCUCCUCUGUG | UGCACUUCCCUCCUCUGUG |
| 731 | AL2-125A | GUGCCGAGACAGCUCACAG | GUGCCGAGACAGCUCACAG |
| 732 | AL2-125B | CUGUGAGCUGUCUCGGCAC | CUGUGAGCUGUCUCGGCAC |
| 733 | AL2-126A | UGCAAGUGGGUGCCGAGAC | UGCAAGUGGGUGCCGAGAC |
| 734 | AL2-126B | GUCUCGGCACCCACUUGCA | GUCUCGGCACCCACUUGCA |
| 735 | AL2-127A | CGGCAGUGACUGCAAGUGG | CGGCAGUGACUGCAAGUGG |
| 736 | AL2-127B | CCACUUGCAGUCACUGCCG | CCACUUGCAGUCACUGCCG |
| 737 | AL2-128A | AACAUCAGGCGGCAGUGAC | AACAUCAGGCGGCAGUGAC |
| 738 | AL2-128B | GUCACUGCCGCCUGAUGUU | GUCACUGCCGCCUGAUGUU |
| 739 | AL2-129A | AAGAGUAACAACAUCAGGC | AAGAGUAACAACAUCAGGC |
| 740 | AL2-129B | GCCUGAUGUUGUUACUCUU | GCCUGAUGUUGUUACUCUU |
| 741 | AL2-130A | UUUUGGAGUGGAAGAGUAA | UUUUGGAGUGGAAGAGUAA |
| 742 | AL2-130B | UUACUCUUCCACUCCAAAA | UUACUCUUCCACUCCAAAA |
| 743 | AL2-131A | ACGGGCAUCCUUUUGGAGU | ACGGGCAUCCUUUUGGAGU |
| 744 | AL2-131B | ACUCCAAAAGGAUGCCCGU | ACUCCAAAAGGAUGCCCGU |
| 745 | AL2-132A | GGGGCCUCGGCCACGGGCA | GGGGCCUCGGCCACGGGCA |
| 746 | AL2-132B | UGCCCGUGGCCGAGGCCCC | UGCCCGUGGCCGAGGCCCC |
| 747 | AL2-133A | ACCUGGGGGGCCUCGGCCA | ACCUGGGGGGCCUCGGCCA |
| 748 | AL2-133B | UGGCCGAGGCCCCCCAGGU | UGGCCGAGGCCCCCCAGGU |
| 749 | AL2-134A | CUGCCCGCCAGCCACCUGG | CUGCCCGCCAGCCACCUGG |
| 750 | AL2-134B | CCAGGUGGCUGGCGGGCAG | CCAGGUGGCUGGCGGGCAG |
| 751 | AL2-135A | CCUCCGUCCCCCUGCCCGC | CCUCCGUCCCCCUGCCCGC |
| 752 | AL2-135B | GCGGGCAGGGGGACGGAGG | GCGGGCAGGGGGACGGAGG |
| 753 | AL2-136A | CUCGCCAUCACCUCCGUCC | CUCGCCAUCACCUCCGUCC |
| 754 | AL2-136B | GGACGGAGGUGAUGGCGAG | GGACGGAGGUGAUGGCGAG |
| 755 | AL2-137A | UCCGCUUCCUCGCCAUCAC | UCCGCUUCCUCGCCAUCAC |
| 756 | AL2-137B | GUGAUGGCGAGGAAGCGGA | GUGAUGGCGAGGAAGCGGA |
| 757 | AL2-138A | UCCCCUCCGGCUCCGCUUC | UCCCCUCCGGCUCCGCUUC |
| 758 | AL2-138B | GAAGCGGAGCCGGAGGGGA | GAAGCGGAGCCGGAGGGGA |
| 759 | AL2-139A | CUUGAACAUCCCCUCCGGC | CUUGAACAUCCCCUCCGGC |
| 760 | AL2-139B | GCCGGAGGGGAUGUUCAAG | GCCGGAGGGGAUGUUCAAG |
| 761 | AL2-140A | UCCUCACAGGCCUUGAACA | UCCUCACAGGCCUUGAACA |
| 762 | AL2-140B | UGUUCAAGGCCUGUGAGGA | UGUUCAAGGCCUGUGAGGA |
| 763 | AL2-141A | UCUCUUGGAGUCCUCACAG | UCUCUUGGAGUCCUCACAG |
| 764 | AL2-141B | CUGUGAGGACUCCAAGAGA | CUGUGAGGACUCCAAGAGA |
| 765 | AL2-142A | CGGGCUUUUCUCUUGGAGU | CGGGCUUUUCUCUUGGAGU |
| 766 | AL2-142B | ACUCCAAGAGAAAAGCCCG | ACUCCAAGAGAAAAGCCCG |
| 767 | AL2-143A | GGCGGAGGUAGCCCCGGGC | GGCGGAGGUAGCCCCGGGC |
| 768 | AL2-143B | GCCCGGGGCUACCUCCGCC | GCCCGGGGCUACCUCCGCC |
| 769 | AL2-144A | AGGGGCACCAGGCGGAGGU | AGGGGCACCAGGCGGAGGU |
| 770 | AL2-144B | ACCUCCGCCUGGUGCCCCU | ACCUCCGCCUGGUGCCCCU |
| 771 | AL2-145A | ACAAACAGGGGCACCAGGC | ACAAACAGGGGCACCAGGC |
| 772 | AL2-145B | GCCUGGUGCCCCUGUUUGU | GCCUGGUGCCCCUGUUUGU |
| 773 | AL2-146A | AGGGCCAGCAGCACAAACA | AGGGCCAGCAGCACAAACA |
| 774 | AL2-146B | UGUUUGUGCUGCUGGCCCU | UGUUUGUGCUGCUGGCCCU |
| 775 | AL2-147A | AGCACGAGCAGGGCCAGCA | AGCACGAGCAGGGCCAGCA |
| 776 | AL2-147B | UGCUGGCCCUGCUCGUGCU | UGCUGGCCCUGCUCGUGCU |
| 777 | AL2-148A | CGCCGAAGCCAGCACGAGC | CGCCGAAGCCAGCACGAGC |
| 778 | AL2-148B | GCUCGUGCUGGCUUCGGCG | GCUCGUGCUGGCUUCGGCG |
| 779 | AL2-149A | AGAGUAGCACCCCCGCCGA | AGAGUAGCACCCCCGCCGA |
| 780 | AL2-149B | UCGGCGGGGGUGCUACUCU | UCGGCGGGGGUGCUACUCU |
| 781 | AL2-150A | CAGAGUAGCACCCCCGCCG | CAGAGUAGCACCCCCGCCG |
| 782 | AL2-150B | CGGCGGGGGUGCUACUCUG | CGGCGGGGGUGCUACUCUG |
| 783 | AL2-151A | CCAGAGUAGCACCCCCGCC | CCAGAGUAGCACCCCCGCC |
| 784 | AL2-151B | GGCGGGGGUGCUACUCUGG | GGCGGGGGUGCUACUCUGG |
| 785 | AL2-152A | ACCAGAGUAGCACCCCCGC | ACCAGAGUAGCACCCCCGC |
| 786 | AL2-152B | GCGGGGGUGCUACUCUGGU | GCGGGGGUGCUACUCUGGU |
| 787 | AL2-153A | UACCAGAGUAGCACCCCCG | UACCAGAGUAGCACCCCCG |
| 788 | AL2-153B | CGGGGGUGCUACUCUGGUA | CGGGGGUGCUACUCUGGUA |
| 789 | AL2-154A | AUACCAGAGUAGCACCCCC | AUACCAGAGUAGCACCCCC |
| 790 | AL2-154B | GGGGGUGCUACUCUGGUAU | GGGGGUGCUACUCUGGUAU |
| 791 | AL2-155A | AAAUACCAGAGUAGCACCC | AAAUACCAGAGUAGCACCC |
| 792 | AL2-155B | GGGUGCUACUCUGGUAUUU | GGGUGCUACUCUGGUAUUU |
| 793 | AL2-156A | GAAAUACCAGAGUAGCACC | GAAAUACCAGAGUAGCACC |
| 794 | AL2-156B | GGUGCUACUCUGGUAUUUC | GGUGCUACUCUGGUAUUUC |
| 795 | AL2-157A | GGAAAUACCAGAGUAGCAC | GGAAAUACCAGAGUAGCAC |
| 796 | AL2-157B | GUGCUACUCUGGUAUUUCC | GUGCUACUCUGGUAUUUCC |
| 797 | AL2-158A | UAGGAAAUACCAGAGUAGC | UAGGAAAUACCAGAGUAGC |
| 798 | AL2-158B | GCUACUCUGGUAUUUCCUA | GCUACUCUGGUAUUUCCUA |
| 799 | AL2-159A | CUAGGAAAUACCAGAGUAG | CUAGGAAAUACCAGAGUAG |
| 800 | AL2-159B | CUACUCUGGUAUUUCCUAG | CUACUCUGGUAUUUCCUAG |
| 801 | AL2-160A | CCUAGGAAAUACCAGAGUA | CCUAGGAAAUACCAGAGUA |
| 802 | AL2-160B | UACUCUGGUAUUUCCUAGG | UACUCUGGUAUUUCCUAGG |
| 803 | AL2-161A | CCCUAGGAAAUACCAGAGU | CCCUAGGAAAUACCAGAGU |
| 804 | AL2-161B | ACUCUGGUAUUUCCUAGGG | ACUCUGGUAUUUCCUAGGG |
| 805 | AL2-162A | ACCCUAGGAAAUACCAGAG | ACCCUAGGAAAUACCAGAG |
| 806 | AL2-162B | CUCUGGUAUUUCCUAGGGU | CUCUGGUAUUUCCUAGGGU |
| 807 | AL2-163A | GUACCCUAGGAAAUACCAG | GUACCCUAGGAAAUACCAG |
| 808 | AL2-163B | CUGGUAUUUCCUAGGGUAC | CUGGUAUUUCCUAGGGUAC |
| 809 | AL2-164A | CUUGUACCCUAGGAAAUAC | CUUGUACCCUAGGAAAUAC |
| 810 | AL2-164B | GUAUUUCCUAGGGUACAAG | GUAUUUCCUAGGGUACAAG |
| 811 | AL2-165A | CCUUGUACCCUAGGAAAUA | CCUUGUACCCUAGGAAAUA |
| 812 | AL2-165B | UAUUUCCUAGGGUACAAGG | UAUUUCCUAGGGUACAAGG |
| 813 | AL2-166A | GCCUUGUACCCUAGGAAAU | GCCUUGUACCCUAGGAAAU |
| 814 | AL2-166B | AUUUCCUAGGGUACAAGGC | AUUUCCUAGGGUACAAGGC |
| 815 | AL2-167A | CGCCUUGUACCCUAGGAAA | CGCCUUGUACCCUAGGAAA |
| 816 | AL2-167B | UUUCCUAGGGUACAAGGCG | UUUCCUAGGGUACAAGGCG |
| 817 | AL2-168A | CCGCCUUGUACCCUAGGAA | CCGCCUUGUACCCUAGGAA |
| 818 | AL2-168B | UUCCUAGGGUACAAGGCGG | UUCCUAGGGUACAAGGCGG |
| 819 | AL2-169A | CUCCGCCUUGUACCCUAGG | CUCCGCCUUGUACCCUAGG |
| 820 | AL2-169B | CCUAGGGUACAAGGCGGAG | CCUAGGGUACAAGGCGGAG |
| 821 | AL2-170A | CCUCCGCCUUGUACCCUAG | CCUCCGCCUUGUACCCUAG |
| 822 | AL2-170B | CUAGGGUACAAGGCGGAGG | CUAGGGUACAAGGCGGAGG |
| 823 | AL2-171A | ACCUCCGCCUUGUACCCUA | ACCUCCGCCUUGUACCCUA |
| 824 | AL2-171B | UAGGGUACAAGGCGGAGGU | UAGGGUACAAGGCGGAGGU |
| 825 | AL2-172A | CACCUCCGCCUUGUACCCU | CACCUCCGCCUUGUACCCU |
| 826 | AL2-172B | AGGGUACAAGGCGGAGGUG | AGGGUACAAGGCGGAGGUG |
| 827 | AL2-173A | UCACCUCCGCCUUGUACCC | UCACCUCCGCCUUGUACCC |
| 828 | AL2-173B | GGGUACAAGGCGGAGGUGA | GGGUACAAGGCGGAGGUGA |
| 829 | AL2-174A | AUCACCUCCGCCUUGUACC | AUCACCUCCGCCUUGUACC |
| 830 | AL2-174B | GGUACAAGGCGGAGGUGAU | GGUACAAGGCGGAGGUGAU |
| 831 | AL2-175A | CAUCACCUCCGCCUUGUAC | CAUCACCUCCGCCUUGUAC |
| 832 | AL2-175B | GUACAAGGCGGAGGUGAUG | GUACAAGGCGGAGGUGAUG |
| 833 | AL2-176A | CCAUCACCUCCGCCUUGUA | CCAUCACCUCCGCCUUGUA |
| 834 | AL2-176B | UACAAGGCGGAGGUGAUGG | UACAAGGCGGAGGUGAUGG |
| 835 | AL2-177A | ACCAUCACCUCCGCCUUGU | ACCAUCACCUCCGCCUUGU |
| 836 | AL2-177B | ACAAGGCGGAGGUGAUGGU | ACAAGGCGGAGGUGAUGGU |
| 837 | AL2-178A | GACCAUCACCUCCGCCUUG | GACCAUCACCUCCGCCUUG |
| 838 | AL2-178B | CAAGGCGGAGGUGAUGGUC | CAAGGCGGAGGUGAUGGUC |
| 839 | AL2-179A | UGACCAUCACCUCCGCCUU | UGACCAUCACCUCCGCCUU |
| 840 | AL2-179B | AAGGCGGAGGUGAUGGUCA | AAGGCGGAGGUGAUGGUCA |
| 841 | AL2-180A | CUGACCAUCACCUCCGCCU | CUGACCAUCACCUCCGCCU |
| 842 | AL2-180B | AGGCGGAGGUGAUGGUCAG | AGGCGGAGGUGAUGGUCAG |
| 843 | AL2-181A | UACACCUGGCUGACCAUCA | UACACCUGGCUGACCAUCA |
| 844 | AL2-181B | UGAUGGUCAGCCAGGUGUA | UGAUGGUCAGCCAGGUGUA |
| 845 | AL2-182A | ACUGCCUGAGUACACCUGG | ACUGCCUGAGUACACCUGG |
| 846 | AL2-182B | CCAGGUGUACUCAGGCAGU | CCAGGUGUACUCAGGCAGU |
| 847 | AL2-183A | UUGAGUACACGCAGACUGC | UUGAGUACACGCAGACUGC |
| 848 | AL2-183B | GCAGUCUGCGUGUACUCAA | GCAGUCUGCGUGUACUCAA |
| 849 | AL2-184A | GUGGCGAUUGAGUACACGC | GUGGCGAUUGAGUACACGC |
| 850 | AL2-184B | GCGUGUACUCAAUCGCCAC | GCGUGUACUCAAUCGCCAC |
| 851 | AL2-185A | AUCCUGGGAGAAGUGGCGA | AUCCUGGGAGAAGUGGCGA |
| 852 | AL2-185B | UCGCCACUUCUCCCAGGAU | UCGCCACUUCUCCCAGGAU |
| 853 | AL2-186A | GCGGGUAAGAUCCUGGGAG | GCGGGUAAGAUCCUGGGAG |
| 854 | AL2-186B | CUCCCAGGAUCUUACCCGC | CUCCCAGGAUCUUACCCGC |
| 855 | AL2-187A | ACUAGAUUCCCGGCGGGUA | ACUAGAUUCCCGGCGGGUA |
| 856 | AL2-187B | UACCCGCCGGGAAUCUAGU | UACCCGCCGGGAAUCUAGU |
| 857 | AL2-188A | GAAGGCACUAGAUUCCCGG | GAAGGCACUAGAUUCCCGG |
| 858 | AL2-188B | CCGGGAAUCUAGUGCCUUC | CCGGGAAUCUAGUGCCUUC |
| 859 | AL2-189A | UUUCACUGCGGAAGGCACU | UUUCACUGCGGAAGGCACU |
| 860 | AL2-189B | AGUGCCUUCCGCAGUGAAA | AGUGCCUUCCGCAGUGAAA |
| 861 | AL2-190A | UGGGCUUUGGCGGUUUCAC | UGGGCUUUGGCGGUUUCAC |
| 862 | AL2-190B | GUGAAACCGCCAAAGCCCA | GUGAAACCGCCAAAGCCCA |
| 863 | AL2-191A | CAUCUUCUGGGCUUUGGCG | CAUCUUCUGGGCUUUGGCG |
| 864 | AL2-191B | CGCCAAAGCCCAGAAGAUG | CGCCAAAGCCCAGAAGAUG |
| 865 | AL2-192A | GCUCCUUGAGCAUCUUCUG | GCUCCUUGAGCAUCUUCUG |
| 866 | AL2-192B | CAGAAGAUGCUCAAGGAGC | CAGAAGAUGCUCAAGGAGC |
| 867 | AL2-193A | CUGGUGAUGAGCUCCUUGA | CUGGUGAUGAGCUCCUUGA |
| 868 | AL2-193B | UCAAGGAGCUCAUCACCAG | UCAAGGAGCUCAUCACCAG |
| 869 | AL2-194A | UUCCCAGGCGGGUGCUGGU | UUCCCAGGCGGGUGCUGGU |
| 870 | AL2-194B | ACCAGCACCCGCCUGGGAA | ACCAGCACCCGCCUGGGAA |
| 871 | AL2-195A | UUGUAGUAAGUUCCCAGGC | UUGUAGUAAGUUCCCAGGC |
| 872 | AL2-195B | GCCUGGGAACUUACUACAA | GCCUGGGAACUUACUACAA |
| 873 | AL2-196A | CUGGAGUUGUAGUAAGUUC | CUGGAGUUGUAGUAAGUUC |
| 874 | AL2-196B | GAACUUACUACAACUCCAG | GAACUUACUACAACUCCAG |
| 875 | AL2-197A | AAUAGACGGAGCUGGAGUU | AAUAGACGGAGCUGGAGUU |
| 876 | AL2-197B | AACUCCAGCUCCGUCUAUU | AACUCCAGCUCCGUCUAUU |
| 877 | AL2-198A | UCCCCAAAGGAAUAGACGG | UCCCCAAAGGAAUAGACGG |
| 878 | AL2-198B | CCGUCUAUUCCUUUGGGGA | CCGUCUAUUCCUUUGGGGA |
| 879 | AL2-199A | GUGAGGGGUCCCUCCCCAA | GUGAGGGGUCCCUCCCCAA |
| 880 | AL2-199B | UUGGGGAGGGACCCCUCAC | UUGGGGAGGGACCCCUCAC |
| 881 | AL2-200A | AGAAGAAGCAGGUGAGGGG | AGAAGAAGCAGGUGAGGGG |
| 882 | AL2-200B | CCCCUCACCUGCUUCUUCU | CCCCUCACCUGCUUCUUCU |
| 883 | AL2-201A | AAUGAACCAGAAGAAGCAG | AAUGAACCAGAAGAAGCAG |
| 884 | AL2-201B | CUGCUUCUUCUGGUUCAUU | CUGCUUCUUCUGGUUCAUU |
| 885 | AL2-202A | GAUUUGGAGAAUGAACCAG | GAUUUGGAGAAUGAACCAG |
| 886 | AL2-202B | CUGGUUCAUUCUCCAAAUC | CUGGUUCAUUCUCCAAAUC |
| 887 | AL2-203A | GUGCUCGGGGAUUUGGAGA | GUGCUCGGGGAUUUGGAGA |
| 888 | AL2-203B | UCUCCAAAUCCCCGAGCAC | UCUCCAAAUCCCCGAGCAC |
| 889 | AL2-204A | AUCAGCCGGCGGUGCUCGG | AUCAGCCGGCGGUGCUCGG |
| 890 | AL2-204B | CCGAGCACCGCCGGCUGAU | CCGAGCACCGCCGGCUGAU |
| 891 | AL2-205A | UCGGGGCUCAGCAUCAGCC | UCGGGGCUCAGCAUCAGCC |
| 892 | AL2-205B | GGCUGAUGCUGAGCCCCGA | GGCUGAUGCUGAGCCCCGA |
| 893 | AL2-206A | UGCACCACCUCGGGGCUCA | UGCACCACCUCGGGGCUCA |
| 894 | AL2-206B | UGAGCCCCGAGGUGGUGCA | UGAGCCCCGAGGUGGUGCA |
| 895 | AL2-207A | ACCAGCAGUGCCUGCACCA | ACCAGCAGUGCCUGCACCA |
| 896 | AL2-207B | UGGUGCAGGCACUGCUGGU | UGGUGCAGGCACUGCUGGU |
| 897 | AL2-208A | UCCUCCACCAGCAGUGCCU | UCCUCCACCAGCAGUGCCU |
| 898 | AL2-208B | AGGCACUGCUGGUGGAGGA | AGGCACUGCUGGUGGAGGA |
| 899 | AL2-209A | UGGACAGCAGCUCCUCCAC | UGGACAGCAGCUCCUCCAC |
| 900 | AL2-209B | GUGGAGGAGCUGCUGUCCA | GUGGAGGAGCUGCUGUCCA |
| 901 | AL2-210A | GAGCUGUUGACUGUGGACA | GAGCUGUUGACUGUGGACA |
| 902 | AL2-210B | UGUCCACAGUCAACAGCUC | UGUCCACAGUCAACAGCUC |
| 903 | AL2-211A | ACGGCAGCCGAGCUGUUGA | ACGGCAGCCGAGCUGUUGA |
| 904 | AL2-211B | UCAACAGCUCGGCUGCCGU | UCAACAGCUCGGCUGCCGU |
| 905 | AL2-212A | UGUAGGGGACGGCAGCCGA | UGUAGGGGACGGCAGCCGA |
| 906 | AL2-212B | UCGGCUGCCGUCCCCUACA | UCGGCUGCCGUCCCCUACA |
| 907 | AL2-213A | UAGGCCCUCGGGGUCCACU | UAGGCCCUCGGGGUCCACU |
| 908 | AL2-213B | AGUGGACCCCGAGGGCCUA | AGUGGACCCCGAGGGCCUA |
| 909 | AL2-214A | UCCAGGAUCACUAGGCCCU | UCCAGGAUCACUAGGCCCU |
| 910 | AL2-214B | AGGGCCUAGUGAUCCUGGA | AGGGCCUAGUGAUCCUGGA |
| 911 | AL2-215A | CUGGCUUCCAGGAUCACUA | CUGGCUUCCAGGAUCACUA |
| 912 | AL2-215B | UAGUGAUCCUGGAAGCCAG | UAGUGAUCCUGGAAGCCAG |
| 913 | AL2-216A | AUGUCUUUCACACUGGCUU | AUGUCUUUCACACUGGCUU |
| 914 | AL2-216B | AAGCCAGUGUGAAAGACAU | AAGCCAGUGUGAAAGACAU |
| 915 | AL2-217A | AAUGCAGCUAUGUCUUUCA | AAUGCAGCUAUGUCUUUCA |
| 916 | AL2-217B | UGAAAGACAUAGCUGCAUU | UGAAAGACAUAGCUGCAUU |
| 917 | AL2-218A | CAGCGUGGAAUUCAAUGCA | CAGCGUGGAAUUCAAUGCA |
| 918 | AL2-218B | UGCAUUGAAUUCCACGCUG | UGCAUUGAAUUCCACGCUG |
| 919 | AL2-219A | CUGGCCCACGUAGCUGUAG | CUGGCCCACGUAGCUGUAG |
| 920 | AL2-219B | CUACAGCUACGUGGGCCAG | CUACAGCUACGUGGGCCAG |
| 921 | AL2-220A | CUGGCCCUGGCCCACGUAG | CUGGCCCUGGCCCACGUAG |
| 922 | AL2-220B | CUACGUGGGCCAGGGCCAG | CUACGUGGGCCAGGGCCAG |
| 923 | AL2-221A | AGCCGGAGGACCUGGCCCU | AGCCGGAGGACCUGGCCCU |
| 924 | AL2-221B | AGGGCCAGGUCCUCCGGCU | AGGGCCAGGUCCUCCGGCU |
| 925 | AL2-222A | GUCAGGCCCCUUCAGCCGG | GUCAGGCCCCUUCAGCCGG |
| 926 | AL2-222B | CCGGCUGAAGGGGCCUGAC | CCGGCUGAAGGGGCCUGAC |
| 927 | AL2-223A | AGGCCAGGUGGUCAGGCCC | AGGCCAGGUGGUCAGGCCC |
| 928 | AL2-223B | GGGCCUGACCACCUGGCCU | GGGCCUGACCACCUGGCCU |
| 929 | AL2-224A | GCAGCUGGAGGCCAGGUGG | GCAGCUGGAGGCCAGGUGG |
| 930 | AL2-224B | CCACCUGGCCUCCAGCUGC | CCACCUGGCCUCCAGCUGC |
| 931 | AL2-225A | AGGUGCCACAGGCAGCUGG | AGGUGCCACAGGCAGCUGG |
| 932 | AL2-225B | CCAGCUGCCUGUGGCACCU | CCAGCUGCCUGUGGCACCU |
| 933 | AL2-226A | GGCCCUGCAGGUGCCACAG | GGCCCUGCAGGUGCCACAG |
| 934 | AL2-226B | CUGUGGCACCUGCAGGGCC | CUGUGGCACCUGCAGGGCC |
| 935 | AL2-227A | GGUCCUUGGGGCCCUGCAG | GGUCCUUGGGGCCCUGCAG |
| 936 | AL2-227B | CUGCAGGGCCCCAAGGACC | CUGCAGGGCCCCAAGGACC |
| 937 | AL2-228A | UUGAGCAUGAGGUCCUUGG | UUGAGCAUGAGGUCCUUGG |
| 938 | AL2-228B | CCAAGGACCUCAUGCUCAA | CCAAGGACCUCAUGCUCAA |
| 939 | AL2-229A | UCCAGCCGGAGUUUGAGCA | UCCAGCCGGAGUUUGAGCA |
| 940 | AL2-229B | UGCUCAAACUCCGGCUGGA | UGCUCAAACUCCGGCUGGA |
| 941 | AL2-230A | CAGCGUCCACUCCAGCCGG | CAGCGUCCACUCCAGCCGG |
| 942 | AL2-230B | CCGGCUGGAGUGGACGCUG | CCGGCUGGAGUGGACGCUG |
| 943 | AL2-231A | CCGGCACUCUGCCAGCGUC | CCGGCACUCUGCCAGCGUC |
| 944 | AL2-231B | GACGCUGGCAGAGUGCCGG | GACGCUGGCAGAGUGCCGG |
| 945 | AL2-232A | UCGGUCCCGGCACUCUGCC | UCGGUCCCGGCACUCUGCC |
| 946 | AL2-232B | GGCAGAGUGCCGGGACCGA | GGCAGAGUGCCGGGACCGA |
| 947 | AL2-233A | UCAUACAUGGCCAGUCGGU | UCAUACAUGGCCAGUCGGU |
| 948 | AL2-233B | ACCGACUGGCCAUGUAUGA | ACCGACUGGCCAUGUAUGA |
| 949 | AL2-234A | CCGGCCACGUCAUACAUGG | CCGGCCACGUCAUACAUGG |
| 950 | AL2-234B | CCAUGUAUGACGUGGCCGG | CCAUGUAUGACGUGGCCGG |
| 951 | AL2-235A | UCUCCAGGGGCCCGGCCAC | UCUCCAGGGGCCCGGCCAC |
| 952 | AL2-235B | GUGGCCGGGCCCCUGGAGA | GUGGCCGGGCCCCUGGAGA |
| 953 | AL2-236A | AUGAGCCUCUUCUCCAGGG | AUGAGCCUCUUCUCCAGGG |
| 954 | AL2-236B | CCCUGGAGAAGAGGCUCAU | CCCUGGAGAAGAGGCUCAU |
| 955 | AL2-237A | GAGGUGAUGAGCCUCUUCU | GAGGUGAUGAGCCUCUUCU |
| 956 | AL2-237B | AGAAGAGGCUCAUCACCUC | AGAAGAGGCUCAUCACCUC |
| 957 | AL2-238A | UGCAGCCGUACACCGAGGU | UGCAGCCGUACACCGAGGU |
| 958 | AL2-238B | ACCUCGGUGUACGGCUGCA | ACCUCGGUGUACGGCUGCA |
| 959 | AL2-239A | UCCUGGCGGCUGCAGCCGU | UCCUGGCGGCUGCAGCCGU |
| 960 | AL2-239B | ACGGCUGCAGCCGCCAGGA | ACGGCUGCAGCCGCCAGGA |
| 961 | AL2-240A | ACCACGGGCUCCUGGCGGC | ACCACGGGCUCCUGGCGGC |
| 962 | AL2-240B | GCCGCCAGGAGCCCGUGGU | GCCGCCAGGAGCCCGUGGU |
| 963 | AL2-241A | AGAACCUCCACCACGGGCU | AGAACCUCCACCACGGGCU |
| 964 | AL2-241B | AGCCCGUGGUGGAGGUUCU | AGCCCGUGGUGGAGGUUCU |
| 965 | AL2-242A | CCGACGCCAGAACCUCCAC | CCGACGCCAGAACCUCCAC |
| 966 | AL2-242B | GUGGAGGUUCUGGCGUCGG | GUGGAGGUUCUGGCGUCGG |
| 967 | AL2-243A | AUGAUGGCCCCCGACGCCA | AUGAUGGCCCCCGACGCCA |
| 968 | AL2-243B | UGGCGUCGGGGGCCAUCAU | UGGCGUCGGGGGCCAUCAU |
| 969 | AL2-244A | CAGACGACCGCCAUGAUGG | CAGACGACCGCCAUGAUGG |
| 970 | AL2-244B | CCAUCAUGGCGGUCGUCUG | CCAUCAUGGCGGUCGUCUG |
| 971 | AL2-245A | CCUUCUUCCAGACGACCGC | CCUUCUUCCAGACGACCGC |
| 972 | AL2-245B | GCGGUCGUCUGGAAGAAGG | GCGGUCGUCUGGAAGAAGG |
| 973 | AL2-246A | CUGUGCAGGCCCUUCUUCC | CUGUGCAGGCCCUUCUUCC |
| 974 | AL2-246B | GGAAGAAGGGCCUGCACAG | GGAAGAAGGGCCUGCACAG |
| 975 | AL2-247A | GUCGUAGUAGCUGUGCAGG | GUCGUAGUAGCUGUGCAGG |
| 976 | AL2-247B | CCUGCACAGCUACUACGAC | CCUGCACAGCUACUACGAC |
| 977 | AL2-248A | AGCACGAAGGGGUCGUAGU | AGCACGAAGGGGUCGUAGU |
| 978 | AL2-248B | ACUACGACCCCUUCGUGCU | ACUACGACCCCUUCGUGCU |
| 979 | AL2-249A | UGCACGGAGAGCACGAAGG | UGCACGGAGAGCACGAAGG |
| 980 | AL2-249B | CCUUCGUGCUCUCCGUGCA | CCUUCGUGCUCUCCGUGCA |
| 981 | AL2-250A | AAGACCACCGGCUGCACGG | AAGACCACCGGCUGCACGG |
| 982 | AL2-250B | CCGUGCAGCCGGUGGUCUU | CCGUGCAGCCGGUGGUCUU |
| 983 | AL2-251A | CAGGCCUGGAAGACCACCG | CAGGCCUGGAAGACCACCG |
| 984 | AL2-251B | CGGUGGUCUUCCAGGCCUG | CGGUGGUCUUCCAGGCCUG |
| 985 | AL2-252A | AGGUUCACUUCACAGGCCU | AGGUUCACUUCACAGGCCU |
| 986 | AL2-252B | AGGCCUGUGAAGUGAACCU | AGGCCUGUGAAGUGAACCU |
| 987 | AL2-253A | UCCAGCGUCAGGUUCACUU | UCCAGCGUCAGGUUCACUU |
| 988 | AL2-253B | AAGUGAACCUGACGCUGGA | AAGUGAACCUGACGCUGGA |
| 989 | AL2-254A | GAGCCUGUUGUCCAGCGUC | GAGCCUGUUGUCCAGCGUC |
| 990 | AL2-254B | GACGCUGGACAACAGGCUC | GACGCUGGACAACAGGCUC |
| 991 | AL2-255A | UGGGAGUCGAGCCUGUUGU | UGGGAGUCGAGCCUGUUGU |
| 992 | AL2-255B | ACAACAGGCUCGACUCCCA | ACAACAGGCUCGACUCCCA |
| 993 | AL2-256A | CUGAGGACGCCCUGGGAGU | CUGAGGACGCCCUGGGAGU |
| 994 | AL2-256B | ACUCCCAGGGCGUCCUCAG | ACUCCCAGGGCGUCCUCAG |
| 995 | AL2-257A | UAGCUGGGGAAGUACGGGG | UAGCUGGGGAAGUACGGGG |
| 996 | AL2-257B | CCCCGUACUUCCCCAGCUA | CCCCGUACUUCCCCAGCUA |
| 997 | AL2-258A | GCGAGUAGUAGCUGGGGAA | GCGAGUAGUAGCUGGGGAA |
| 998 | AL2-258B | UUCCCCAGCUACUACUCGC | UUCCCCAGCUACUACUCGC |
| 999 | AL2-259A | UGGGUUUGGGGCGAGUAGU | UGGGUUUGGGGCGAGUAGU |
| 1000 | AL2-259B | ACUACUCGCCCCAAACCCA | ACUACUCGCCCCAAACCCA |
| 1001 | AL2-260A | CAGGAGCAGUGGGUUUGGG | CAGGAGCAGUGGGUUUGGG |
| 1002 | AL2-260B | CCCAAACCCACUGCUCCUG | CCCAAACCCACUGCUCCUG |
| 1003 | AL2-261A | ACCGUGAGGUGCCAGGAGC | ACCGUGAGGUGCCAGGAGC |
| 1004 | AL2-261B | GCUCCUGGCACCUCACGGU | GCUCCUGGCACCUCACGGU |
| 1005 | AL2-262A | AGAGAGGGCACCGUGAGGU | AGAGAGGGCACCGUGAGGU |
| 1006 | AL2-262B | ACCUCACGGUGCCCUCUCU | ACCUCACGGUGCCCUCUCU |
| 1007 | AL2-263A | CAAGCCGUAGUCCAGAGAG | CAAGCCGUAGUCCAGAGAG |
| 1008 | AL2-263B | CUCUCUGGACUACGGCUUG | CUCUCUGGACUACGGCUUG |
| 1009 | AL2-264A | AGAGGGCCAAGCCGUAGUC | AGAGGGCCAAGCCGUAGUC |
| 1010 | AL2-264B | GACUACGGCUUGGCCCUCU | GACUACGGCUUGGCCCUCU |
| 1011 | AL2-265A | UAGGCAUCAAACCAGAGGG | UAGGCAUCAAACCAGAGGG |
| 1012 | AL2-265B | CCCUCUGGUUUGAUGCCUA | CCCUCUGGUUUGAUGCCUA |
| 1013 | AL2-266A | CAGUGCAUAGGCAUCAAAC | CAGUGCAUAGGCAUCAAAC |
| 1014 | AL2-266B | GUUUGAUGCCUAUGCACUG | GUUUGAUGCCUAUGCACUG |
| 1015 | AL2-267A | ACUUCUGCCUCCUCAGUGC | ACUUCUGCCUCCUCAGUGC |
| 1016 | AL2-267B | GCACUGAGGAGGCAGAAGU | GCACUGAGGAGGCAGAAGU |
| 1017 | AL2-268A | AAAUCAUACUUCUGCCUCC | AAAUCAUACUUCUGCCUCC |
| 1018 | AL2-268B | GGAGGCAGAAGUAUGAUUU | GGAGGCAGAAGUAUGAUUU |
| 1019 | AL2-269A | UGGGUGCACGGCAAAUCAU | UGGGUGCACGGCAAAUCAU |
| 1020 | AL2-269B | AUGAUUUGCCGUGCACCCA | AUGAUUUGCCGUGCACCCA |
| 1021 | AL2-270A | UCCACUGGCCCUGGGUGCA | UCCACUGGCCCUGGGUGCA |
| 1022 | AL2-270B | UGCACCCAGGGCCAGUGGA | UGCACCCAGGGCCAGUGGA |
| 1023 | AL2-271A | UUCUGGAUCGUCCACUGGC | UUCUGGAUCGUCCACUGGC |
| 1024 | AL2-271B | GCCAGUGGACGAUCCAGAA | GCCAGUGGACGAUCCAGAA |
| 1025 | AL2-272A | CUCCUGUUCUGGAUCGUCC | CUCCUGUUCUGGAUCGUCC |
| 1026 | AL2-272B | GGACGAUCCAGAACAGGAG | GGACGAUCCAGAACAGGAG |
| 1027 | AL2-273A | AAGCCACACAGCCUCCUGU | AAGCCACACAGCCUCCUGU |
| 1028 | AL2-273B | ACAGGAGGCUGUGUGGCUU | ACAGGAGGCUGUGUGGCUU |
| 1029 | AL2-274A | GGAUGCGCAAGCCACACAG | GGAUGCGCAAGCCACACAG |
| 1030 | AL2-274B | CUGUGUGGCUUGCGCAUCC | CUGUGUGGCUUGCGCAUCC |
| 1031 | AL2-275A | UAGGGCUGCAGGAUGCGCA | UAGGGCUGCAGGAUGCGCA |
| 1032 | AL2-275B | UGCGCAUCCUGCAGCCCUA | UGCGCAUCCUGCAGCCCUA |
| 1033 | AL2-276A | AUCCUCUCGGCGUAGGGCU | AUCCUCUCGGCGUAGGGCU |
| 1034 | AL2-276B | AGCCCUACGCCGAGAGGAU | AGCCCUACGCCGAGAGGAU |
| 1035 | AL2-277A | ACCACGGGGAUCCUCUCGG | ACCACGGGGAUCCUCUCGG |
| 1036 | AL2-277B | CCGAGAGGAUCCCCGUGGU | CCGAGAGGAUCCCCGUGGU |
| 1037 | AL2-278A | CCGGCCGUGGCCACCACGG | CCGGCCGUGGCCACCACGG |
| 1038 | AL2-278B | CCGUGGUGGCCACGGCCGG | CCGUGGUGGCCACGGCCGG |
| 1039 | AL2-279A | AUGGUGAUCCCGGCCGUGG | AUGGUGAUCCCGGCCGUGG |
| 1040 | AL2-279B | CCACGGCCGGGAUCACCAU | CCACGGCCGGGAUCACCAU |
| 1041 | AL2-280A | GUGAAGUUGAUGGUGAUCC | GUGAAGUUGAUGGUGAUCC |
| 1042 | AL2-280B | GGAUCACCAUCAACUUCAC | GGAUCACCAUCAACUUCAC |
| 1043 | AL2-281A | AUCUGGGAGGUGAAGUUGA | AUCUGGGAGGUGAAGUUGA |
| 1044 | AL2-281B | UCAACUUCACCUCCCAGAU | UCAACUUCACCUCCCAGAU |
| 1045 | AL2-282A | CCGGUGAGGGAGAUCUGGG | CCGGUGAGGGAGAUCUGGG |
| 1046 | AL2-282B | CCCAGAUCUCCCUCACCGG | CCCAGAUCUCCCUCACCGG |
| 1047 | AL2-283A | ACACCGGGCCCGGUGAGGG | ACACCGGGCCCGGUGAGGG |
| 1048 | AL2-283B | CCCUCACCGGGCCCGGUGU | CCCUCACCGGGCCCGGUGU |
| 1049 | AL2-284A | AGUGCACCCGCACACCGGG | AGUGCACCCGCACACCGGG |
| 1050 | AL2-284B | CCCGGUGUGCGGGUGCACU | CCCGGUGUGCGGGUGCACU |
| 1051 | AL2-285A | UCCGACUGGUUGUACAAGC | UCCGACUGGUUGUACAAGC |
| 1052 | AL2-285B | GCUUGUACAACCAGUCGGA | GCUUGUACAACCAGUCGGA |
| 1053 | AL2-286A | CAGGGGUCCGACUGGUUGU | CAGGGGUCCGACUGGUUGU |
| 1054 | AL2-286B | ACAACCAGUCGGACCCCUG | ACAACCAGUCGGACCCCUG |
| 1055 | AL2-287A | AACUCUCCAGGGCAGGGGU | AACUCUCCAGGGCAGGGGU |
| 1056 | AL2-287B | ACCCCUGCCCUGGAGAGUU | ACCCCUGCCCUGGAGAGUU |
| 1057 | AL2-288A | AACAGAGGAACUCUCCAGG | AACAGAGGAACUCUCCAGG |
| 1058 | AL2-288B | CCUGGAGAGUUCCUCUGUU | CCUGGAGAGUUCCUCUGUU |
| 1059 | AL2-289A | AGUCCAUUCACAGAACAGA | AGUCCAUUCACAGAACAGA |
| 1060 | AL2-289B | UCUGUUCUGUGAAUGGACU | UCUGUUCUGUGAAUGGACU |
| 1061 | AL2-290A | AGGGACACAGAGUCCAUUC | AGGGACACAGAGUCCAUUC |
| 1062 | AL2-290B | GAAUGGACUCUGUGUCCCU | GAAUGGACUCUGUGUCCCU |
| 1063 | AL2-291A | AUCACAGGCAGGGACACAG | AUCACAGGCAGGGACACAG |
| 1064 | AL2-291B | CUGUGUCCCUGCCUGUGAU | CUGUGUCCCUGCCUGUGAU |
| 1065 | AL2-292A | UUGACCCCAUCACAGGCAG | UUGACCCCAUCACAGGCAG |
| 1066 | AL2-292B | CUGCCUGUGAUGGGGUCAA | CUGCCUGUGAUGGGGUCAA |
| 1067 | AL2-293A | GUUGGGGCAGUCCUUGACC | GUUGGGGCAGUCCUUGACC |
| 1068 | AL2-293B | GGUCAAGGACUGCCCCAAC | GGUCAAGGACUGCCCCAAC |
| 1069 | AL2-294A | CAUCCAGGCCGUUGGGGCA | CAUCCAGGCCGUUGGGGCA |
| 1070 | AL2-294B | UGCCCCAACGGCCUGGAUG | UGCCCCAACGGCCUGGAUG |
| 1071 | AL2-295A | GUUUCUCUCAUCCAGGCCG | GUUUCUCUCAUCCAGGCCG |
| 1072 | AL2-295B | CGGCCUGGAUGAGAGAAAC | CGGCCUGGAUGAGAGAAAC |
| 1073 | AL2-296A | UGCAAACGCAGUUUCUCUC | UGCAAACGCAGUUUCUCUC |
| 1074 | AL2-296B | GAGAGAAACUGCGUUUGCA | GAGAGAAACUGCGUUUGCA |
| 1075 | AL2-297A | UGGAAUGUGGCUCUGCAAA | UGGAAUGUGGCUCUGCAAA |
| 1076 | AL2-297B | UUUGCAGAGCCACAUUCCA | UUUGCAGAGCCACAUUCCA |
| 1077 | AL2-298A | CUUUGCACUGGAAUGUGGC | CUUUGCACUGGAAUGUGGC |
| 1078 | AL2-298B | GCCACAUUCCAGUGCAAAG | GCCACAUUCCAGUGCAAAG |
| 1079 | AL2-299A | UGUGCUGUCCUCUUUGCAC | UGUGCUGUCCUCUUUGCAC |
| 1080 | AL2-299B | GUGCAAAGAGGACAGCACA | GUGCAAAGAGGACAGCACA |
| 1081 | AL2-300A | AGAUGCAUGUGCUGUCCUC | AGAUGCAUGUGCUGUCCUC |
| 1082 | AL2-300B | GAGGACAGCACAUGCAUCU | GAGGACAGCACAUGCAUCU |
| 1083 | AL2-301A | ACCUUGGGCAGUGAGAUGC | ACCUUGGGCAGUGAGAUGC |
| 1084 | AL2-301B | GCAUCUCACUGCCCAAGGU | GCAUCUCACUGCCCAAGGU |
| 1085 | AL2-302A | CCCAUCACAGACCUUGGGC | CCCAUCACAGACCUUGGGC |
| 1086 | AL2-302B | GCCCAAGGUCUGUGAUGGG | GCCCAAGGUCUGUGAUGGG |
| 1087 | AL2-303A | AAUCAGGCUGCCCAUCACA | AAUCAGGCUGCCCAUCACA |
| 1088 | AL2-303B | UGUGAUGGGCAGCCUGAUU | UGUGAUGGGCAGCCUGAUU |
| 1089 | AL2-304A | GUUGAGACAAUCAGGCUGC | GUUGAGACAAUCAGGCUGC |
| 1090 | AL2-304B | GCAGCCUGAUUGUCUCAAC | GCAGCCUGAUUGUCUCAAC |
| 1091 | AL2-305A | UCGUCGCUGCCGUUGAGAC | UCGUCGCUGCCGUUGAGAC |
| 1092 | AL2-305B | GUCUCAACGGCAGCGACGA | GUCUCAACGGCAGCGACGA |
| 1093 | AL2-306A | UGGCACUGCUCUUCGUCGC | UGGCACUGCUCUUCGUCGC |
| 1094 | AL2-306B | GCGACGAAGAGCAGUGCCA | GCGACGAAGAGCAGUGCCA |
| 1095 | AL2-307A | ACCCCUUCCUGGCACUGCU | ACCCCUUCCUGGCACUGCU |
| 1096 | AL2-307B | AGCAGUGCCAGGAAGGGGU | AGCAGUGCCAGGAAGGGGU |
| 1097 | AL2-308A | UCCCACAUGGCACCCCUUC | UCCCACAUGGCACCCCUUC |
| 1098 | AL2-308B | GAAGGGGUGCCAUGUGGGA | GAAGGGGUGCCAUGUGGGA |
| 1099 | AL2-309A | AGGUGAAUGUCCCACAUGG | AGGUGAAUGUCCCACAUGG |
| 1100 | AL2-309B | CCAUGUGGGACAUUCACCU | CCAUGUGGGACAUUCACCU |
| 1101 | AL2-310A | UCACACUGGAAGGUGAAUG | UCACACUGGAAGGUGAAUG |
| 1102 | AL2-310B | CAUUCACCUUCCAGUGUGA | CAUUCACCUUCCAGUGUGA |
| 1103 | AL2-311A | AGCUCCGGUCCUCACACUG | AGCUCCGGUCCUCACACUG |
| 1104 | AL2-311B | CAGUGUGAGGACCGGAGCU | CAGUGUGAGGACCGGAGCU |
| 1105 | AL2-312A | UCUUCACGCAGCUCCGGUC | UCUUCACGCAGCUCCGGUC |
| 1106 | AL2-312B | GACCGGAGCUGCGUGAAGA | GACCGGAGCUGCGUGAAGA |
| 1107 | AL2-313A | GUUGGGCUUCUUCACGCAG | GUUGGGCUUCUUCACGCAG |
| 1108 | AL2-313B | CUGCGUGAAGAAGCCCAAC | CUGCGUGAAGAAGCCCAAC |
| 1109 | AL2-314A | UCACACUGCGGGUUGGGCU | UCACACUGCGGGUUGGGCU |
| 1110 | AL2-314B | AGCCCAACCCGCAGUGUGA | AGCCCAACCCGCAGUGUGA |
| 1111 | AL2-315A | CGGGCCGCCCAUCACACUG | CGGGCCGCCCAUCACACUG |
| 1112 | AL2-315B | CAGUGUGAUGGGCGGCCCG | CAGUGUGAUGGGCGGCCCG |
| 1113 | AL2-316A | GUCCCUGCAGUCGGGCCGC | GUCCCUGCAGUCGGGCCGC |
| 1114 | AL2-316B | GCGGCCCGACUGCAGGGAC | GCGGCCCGACUGCAGGGAC |
| 1115 | AL2-317A | AUCCGAGCCGUCCCUGCAG | AUCCGAGCCGUCCCUGCAG |
| 1116 | AL2-317B | CUGCAGGGACGGCUCGGAU | CUGCAGGGACGGCUCGGAU |
| 1117 | AL2-318A | UGCUCCUCAUCCGAGCCGU | UGCUCCUCAUCCGAGCCGU |
| 1118 | AL2-318B | ACGGCUCGGAUGAGGAGCA | ACGGCUCGGAUGAGGAGCA |
| 1119 | AL2-319A | ACAGUCACAGUGCUCCUCA | ACAGUCACAGUGCUCCUCA |
| 1120 | AL2-319B | UGAGGAGCACUGUGACUGU | UGAGGAGCACUGUGACUGU |
| 1121 | AL2-320A | CUGGAGGCCACAGUCACAG | CUGGAGGCCACAGUCACAG |
| 1122 | AL2-320B | CUGUGACUGUGGCCUCCAG | CUGUGACUGUGGCCUCCAG |
| 1123 | AL2-321A | CUGGAGGGGCCCUGGAGGC | CUGGAGGGGCCCUGGAGGC |
| 1124 | AL2-321B | GCCUCCAGGGCCCCUCCAG | GCCUCCAGGGCCCCUCCAG |
| 1125 | AL2-322A | AACAAUGCGGCUGGAGGGG | AACAAUGCGGCUGGAGGGG |
| 1126 | AL2-322B | CCCCUCCAGCCGCAUUGUU | CCCCUCCAGCCGCAUUGUU |
| 1127 | AL2-323A | AGCUCCACCAACAAUGCGG | AGCUCCACCAACAAUGCGG |
| 1128 | AL2-323B | CCGCAUUGUUGGUGGAGCU | CCGCAUUGUUGGUGGAGCU |
| 1129 | AL2-324A | UCGGAGGACACAGCUCCAC | UCGGAGGACACAGCUCCAC |
| 1130 | AL2-324B | GUGGAGCUGUGUCCUCCGA | GUGGAGCUGUGUCCUCCGA |
| 1131 | AL2-325A | UGGCCACUCACCCUCGGAG | UGGCCACUCACCCUCGGAG |
| 1132 | AL2-325B | CUCCGAGGGUGAGUGGCCA | CUCCGAGGGUGAGUGGCCA |
| 1133 | AL2-326A | CUGCCAUGGCCACUCACCC | CUGCCAUGGCCACUCACCC |
| 1134 | AL2-326B | GGGUGAGUGGCCAUGGCAG | GGGUGAGUGGCCAUGGCAG |
| 1135 | AL2-327A | CUGGAGGCUGGCCUGCCAU | CUGGAGGCUGGCCUGCCAU |
| 1136 | AL2-327B | AUGGCAGGCCAGCCUCCAG | AUGGCAGGCCAGCCUCCAG |
| 1137 | AL2-328A | UCGACCCCGAACCUGGAGG | UCGACCCCGAACCUGGAGG |
| 1138 | AL2-328B | CCUCCAGGUUCGGGGUCGA | CCUCCAGGUUCGGGGUCGA |
| 1139 | AL2-329A | GAUGUGUCGACCCCGAACC | GAUGUGUCGACCCCGAACC |
| 1140 | AL2-329B | GGUUCGGGGUCGACACAUC | GGUUCGGGGUCGACACAUC |
| 1141 | AL2-330A | AGGGCCCCCCCACAGAUGU | AGGGCCCCCCCACAGAUGU |
| 1142 | AL2-330B | ACAUCUGUGGGGGGGCCCU | ACAUCUGUGGGGGGGCCCU |
| 1143 | AL2-331A | GCGAUGAGGGCCCCCCCAC | GCGAUGAGGGCCCCCCCAC |
| 1144 | AL2-331B | GUGGGGGGGCCCUCAUCGC | GUGGGGGGGCCCUCAUCGC |
| 1145 | AL2-332A | UCACCCAGCGGUCAGCGAU | UCACCCAGCGGUCAGCGAU |
| 1146 | AL2-332B | AUCGCUGACCGCUGGGUGA | AUCGCUGACCGCUGGGUGA |
| 1147 | AL2-333A | GCUGUUAUCACCCAGCGGU | GCUGUUAUCACCCAGCGGU |
| 1148 | AL2-333B | ACCGCUGGGUGAUAACAGC | ACCGCUGGGUGAUAACAGC |
| 1149 | AL2-334A | CAGUGGGCAGCUGUUAUCA | CAGUGGGCAGCUGUUAUCA |
| 1150 | AL2-334B | UGAUAACAGCUGCCCACUG | UGAUAACAGCUGCCCACUG |
| 1151 | AL2-335A | UCCUCCUGGAAGCAGUGGG | UCCUCCUGGAAGCAGUGGG |
| 1152 | AL2-335B | CCCACUGCUUCCAGGAGGA | CCCACUGCUUCCAGGAGGA |
| 1153 | AL2-336A | GGCCAUGCUGUCCUCCUGG | GGCCAUGCUGUCCUCCUGG |
| 1154 | AL2-336B | CCAGGAGGACAGCAUGGCC | CCAGGAGGACAGCAUGGCC |
| 1155 | AL2-337A | ACCGUGGAGGCCAUGCUGU | ACCGUGGAGGCCAUGCUGU |
| 1156 | AL2-337B | ACAGCAUGGCCUCCACGGU | ACAGCAUGGCCUCCACGGU |
| 1157 | AL2-338A | ACGGUCCACAGCACCGUGG | ACGGUCCACAGCACCGUGG |
| 1158 | AL2-338B | CCACGGUGCUGUGGACCGU | CCACGGUGCUGUGGACCGU |
| 1159 | AL2-339A | UUGCCCAGGAACACGGUCC | UUGCCCAGGAACACGGUCC |
| 1160 | AL2-339B | GGACCGUGUUCCUGGGCAA | GGACCGUGUUCCUGGGCAA |
| 1161 | AL2-340A | UGCCACACCUUGCCCAGGA | UGCCACACCUUGCCCAGGA |
| 1162 | AL2-340B | UCCUGGGCAAGGUGUGGCA | UCCUGGGCAAGGUGUGGCA |
| 1163 | AL2-341A | AGCGCGAGUUCUGCCACAC | AGCGCGAGUUCUGCCACAC |
| 1164 | AL2-341B | GUGUGGCAGAACUCGCGCU | GUGUGGCAGAACUCGCGCU |
| 1165 | AL2-342A | UCCAGGCCAGCGCGAGUUC | UCCAGGCCAGCGCGAGUUC |
| 1166 | AL2-342B | GAACUCGCGCUGGCCUGGA | GAACUCGCGCUGGCCUGGA |
| 1167 | AL2-343A | AAGGACACCUCUCCAGGCC | AAGGACACCUCUCCAGGCC |
| 1168 | AL2-343B | GGCCUGGAGAGGUGUCCUU | GGCCUGGAGAGGUGUCCUU |
| 1169 | AL2-344A | CUCACCUUGAAGGACACCU | CUCACCUUGAAGGACACCU |
| 1170 | AL2-344B | AGGUGUCCUUCAAGGUGAG | AGGUGUCCUUCAAGGUGAG |
| 1171 | AL2-345A | GAGCAGGCGGCUCACCUUG | GAGCAGGCGGCUCACCUUG |
| 1172 | AL2-345B | CAAGGUGAGCCGCCUGCUC | CAAGGUGAGCCGCCUGCUC |
| 1173 | AL2-346A | UACGGGUGCAGGAGCAGGC | UACGGGUGCAGGAGCAGGC |
| 1174 | AL2-346B | GCCUGCUCCUGCACCCGUA | GCCUGCUCCUGCACCCGUA |
| 1175 | AL2-347A | CUCUUCGUGGUACGGGUGC | CUCUUCGUGGUACGGGUGC |
| 1176 | AL2-347B | GCACCCGUACCACGAAGAG | GCACCCGUACCACGAAGAG |
| 1177 | AL2-348A | AUGGCUGUCCUCUUCGUGG | AUGGCUGUCCUCUUCGUGG |
| 1178 | AL2-348B | CCACGAAGAGGACAGCCAU | CCACGAAGAGGACAGCCAU |
| 1179 | AL2-349A | UCGUAGUCAUGGCUGUCCU | UCGUAGUCAUGGCUGUCCU |
| 1180 | AL2-349B | AGGACAGCCAUGACUACGA | AGGACAGCCAUGACUACGA |
| 1181 | AL2-350A | AGCAGCGCCACGUCGUAGU | AGCAGCGCCACGUCGUAGU |
| 1182 | AL2-350B | ACUACGACGUGGCGCUGCU | ACUACGACGUGGCGCUGCU |
| 1183 | AL2-351A | UCGAGCUGCAGCAGCGCCA | UCGAGCUGCAGCAGCGCCA |
| 1184 | AL2-351B | UGGCGCUGCUGCAGCUCGA | UGGCGCUGCUGCAGCUCGA |
| 1185 | AL2-352A | ACCACCGGGUGGUCGAGCU | ACCACCGGGUGGUCGAGCU |
| 1186 | AL2-352B | AGCUCGACCACCCGGUGGU | AGCUCGACCACCCGGUGGU |
| 1187 | AL2-353A | CGGCCGAGCGCACCACCGG | CGGCCGAGCGCACCACCGG |
| 1188 | AL2-353B | CCGGUGGUGCGCUCGGCCG | CCGGUGGUGCGCUCGGCCG |
| 1189 | AL2-354A | CGCACGGCGGCCGAGCGCA | CGCACGGCGGCCGAGCGCA |
| 1190 | AL2-354B | UGCGCUCGGCCGCCGUGCG | UGCGCUCGGCCGCCGUGCG |
| 1191 | AL2-355A | AGGCAGACGGGGCGCACGG | AGGCAGACGGGGCGCACGG |
| 1192 | AL2-355B | CCGUGCGCCCCGUCUGCCU | CCGUGCGCCCCGUCUGCCU |
| 1193 | AL2-356A | CGCGCGGGCAGGCAGACGG | CGCGCGGGCAGGCAGACGG |
| 1194 | AL2-356B | CCGUCUGCCUGCCCGCGCG | CCGUCUGCCUGCCCGCGCG |
| 1195 | AL2-357A | AAGAAGUGGGAGCGCGCGG | AAGAAGUGGGAGCGCGCGG |
| 1196 | AL2-357B | CCGCGCGCUCCCACUUCUU | CCGCGCGCUCCCACUUCUU |
| 1197 | AL2-358A | CCGGGCUCGAAGAAGUGGG | CCGGGCUCGAAGAAGUGGG |
| 1198 | AL2-358B | CCCACUUCUUCGAGCCCGG | CCCACUUCUUCGAGCCCGG |
| 1199 | AL2-359A | AGCAGUGCAGGCCGGGCUC | AGCAGUGCAGGCCGGGCUC |
| 1200 | AL2-359B | GAGCCCGGCCUGCACUGCU | GAGCCCGGCCUGCACUGCU |
| 1201 | AL2-360A | UAAUCCAGCAGUGCAGGCC | UAAUCCAGCAGUGCAGGCC |
| 1202 | AL2-360B | GGCCUGCACUGCUGGAUUA | GGCCUGCACUGCUGGAUUA |
| 1203 | AL2-361A | CGCCCCAGCCCGUAAUCCA | CGCCCCAGCCCGUAAUCCA |
| 1204 | AL2-361B | UGGAUUACGGGCUGGGGCG | UGGAUUACGGGCUGGGGCG |
| 1205 | AL2-362A | UCGCGCAAGGCGCCCCAGC | UCGCGCAAGGCGCCCCAGC |
| 1206 | AL2-362B | GCUGGGGCGCCUUGCGCGA | GCUGGGGCGCCUUGCGCGA |
| 1207 | AL2-363A | AUGGGGCCGCCCUCGCGCA | AUGGGGCCGCCCUCGCGCA |
| 1208 | AL2-363B | UGCGCGAGGGCGGCCCCAU | UGCGCGAGGGCGGCCCCAU |
| 1209 | AL2-364A | UUGCUGAUGGGGCCGCCCU | UUGCUGAUGGGGCCGCCCU |
| 1210 | AL2-364B | AGGGCGGCCCCAUCAGCAA | AGGGCGGCCCCAUCAGCAA |
| 1211 | AL2-365A | UUCUGCAGAGCGUUGCUGA | UUCUGCAGAGCGUUGCUGA |
| 1212 | AL2-365B | UCAGCAACGCUCUGCAGAA | UCAGCAACGCUCUGCAGAA |
| 1213 | AL2-366A | UGCACAUCCACUUUCUGCA | UGCACAUCCACUUUCUGCA |
| 1214 | AL2-366B | UGCAGAAAGUGGAUGUGCA | UGCAGAAAGUGGAUGUGCA |
| 1215 | AL2-367A | AUCAACUGCACAUCCACUU | AUCAACUGCACAUCCACUU |
| 1216 | AL2-367B | AAGUGGAUGUGCAGUUGAU | AAGUGGAUGUGCAGUUGAU |
| 1217 | AL2-368A | GUCCUGUGGGAUCAACUGC | GUCCUGUGGGAUCAACUGC |
| 1218 | AL2-368B | GCAGUUGAUCCCACAGGAC | GCAGUUGAUCCCACAGGAC |
| 1219 | AL2-369A | UCGCUGCACAGGUCCUGUG | UCGCUGCACAGGUCCUGUG |
| 1220 | AL2-369B | CACAGGACCUGUGCAGCGA | CACAGGACCUGUGCAGCGA |
| 1221 | AL2-370A | UAGCGAUAGACCUCGCUGC | UAGCGAUAGACCUCGCUGC |
| 1222 | AL2-370B | GCAGCGAGGUCUAUCGCUA | GCAGCGAGGUCUAUCGCUA |
| 1223 | AL2-371A | UCACCUGGUAGCGAUAGAC | UCACCUGGUAGCGAUAGAC |
| 1224 | AL2-371B | GUCUAUCGCUACCAGGUGA | GUCUAUCGCUACCAGGUGA |
| 1225 | AL2-372A | CAUGCGUGGCGUCACCUGG | CAUGCGUGGCGUCACCUGG |
| 1226 | AL2-372B | CCAGGUGACGCCACGCAUG | CCAGGUGACGCCACGCAUG |
| 1227 | AL2-373A | CGGCACACAGCAUGCGUGG | CGGCACACAGCAUGCGUGG |
| 1228 | AL2-373B | CCACGCAUGCUGUGUGCCG | CCACGCAUGCUGUGUGCCG |
| 1229 | AL2-374A | UGCGGUAGCCGGCACACAG | UGCGGUAGCCGGCACACAG |
| 1230 | AL2-374B | CUGUGUGCCGGCUACCGCA | CUGUGUGCCGGCUACCGCA |
| 1231 | AL2-375A | UCCUUCUUGCCCUUGCGGU | UCCUUCUUGCCCUUGCGGU |
| 1232 | AL2-375B | ACCGCAAGGGCAAGAAGGA | ACCGCAAGGGCAAGAAGGA |
| 1233 | AL2-376A | UGACAGGCAUCCUUCUUGC | UGACAGGCAUCCUUCUUGC |
| 1234 | AL2-376B | GCAAGAAGGAUGCCUGUCA | GCAAGAAGGAUGCCUGUCA |
| 1235 | AL2-377A | CUGAGUCACCCUGACAGGC | CUGAGUCACCCUGACAGGC |
| 1236 | AL2-377B | GCCUGUCAGGGUGACUCAG | GCCUGUCAGGGUGACUCAG |
| 1237 | AL2-378A | AGCGGACCACCUGAGUCAC | AGCGGACCACCUGAGUCAC |
| 1238 | AL2-378B | GUGACUCAGGUGGUCCGCU | GUGACUCAGGUGGUCCGCU |
| 1239 | AL2-379A | UUGCACACCAGCGGACCAC | UUGCACACCAGCGGACCAC |
| 1240 | AL2-379B | GUGGUCCGCUGGUGUGCAA | GUGGUCCGCUGGUGUGCAA |
| 1241 | AL2-380A | CUGAGUGCCUUGCACACCA | CUGAGUGCCUUGCACACCA |
| 1242 | AL2-380B | UGGUGUGCAAGGCACUCAG | UGGUGUGCAAGGCACUCAG |
| 1243 | AL2-381A | ACCAGCGGCCACUGAGUGC | ACCAGCGGCCACUGAGUGC |
| 1244 | AL2-381B | GCACUCAGUGGCCGCUGGU | GCACUCAGUGGCCGCUGGU |
| 1245 | AL2-382A | CCCGCCAGGAACCAGCGGC | CCCGCCAGGAACCAGCGGC |
| 1246 | AL2-382B | GCCGCUGGUUCCUGGCGGG | GCCGCUGGUUCCUGGCGGG |
| 1247 | AL2-383A | CUGACCAGCCCCGCCAGGA | CUGACCAGCCCCGCCAGGA |
| 1248 | AL2-383B | UCCUGGCGGGGCUGGUCAG | UCCUGGCGGGGCUGGUCAG |
| 1249 | AL2-384A | CAGGCCCCAGCUGACCAGC | CAGGCCCCAGCUGACCAGC |
| 1250 | AL2-384B | GCUGGUCAGCUGGGGCCUG | GCUGGUCAGCUGGGGCCUG |
| 1251 | AL2-385A | CCGGCCACAGCCCAGGCCC | CCGGCCACAGCCCAGGCCC |
| 1252 | AL2-385B | GGGCCUGGGCUGUGGCCGG | GGGCCUGGGCUGUGGCCGG |
| 1253 | AL2-386A | AGUUAGGCCGGCCACAGCC | AGUUAGGCCGGCCACAGCC |
| 1254 | AL2-386B | GGCUGUGGCCGGCCUAACU | GGCUGUGGCCGGCCUAACU |
| 1255 | AL2-387A | UAGACGCCGAAGUAGUUAG | UAGACGCCGAAGUAGUUAG |
| 1256 | AL2-387B | CUAACUACUUCGGCGUCUA | CUAACUACUUCGGCGUCUA |
| 1257 | AL2-388A | GAUGCGGGUGUAGACGCCG | GAUGCGGGUGUAGACGCCG |
| 1258 | AL2-388B | CGGCGUCUACACCCGCAUC | CGGCGUCUACACCCGCAUC |
| 1259 | AL2-389A | ACACCUGUGAUGCGGGUGU | ACACCUGUGAUGCGGGUGU |
| 1260 | AL2-389B | ACACCCGCAUCACAGGUGU | ACACCCGCAUCACAGGUGU |
| 1261 | AL2-390A | UCCAGCUGAUCACACCUGU | UCCAGCUGAUCACACCUGU |
| 1262 | AL2-390B | ACAGGUGUGAUCAGCUGGA | ACAGGUGUGAUCAGCUGGA |
| 1263 | AL2-391A | ACUUGCUGGAUCCAGCUGA | ACUUGCUGGAUCCAGCUGA |
| 1264 | AL2-391B | UCAGCUGGAUCCAGCAAGU | UCAGCUGGAUCCAGCAAGU |
| 1265 | AL2-392A | CUCAGGUCACCACUUGCUG | CUCAGGUCACCACUUGCUG |
| 1266 | AL2-392B | CAGCAAGUGGUGACCUGAG | CAGCAAGUGGUGACCUGAG |
| 1267 | AL2-393A | GGGGCAGUUCCUCAGGUCA | GGGGCAGUUCCUCAGGUCA |
| 1268 | AL2-393B | UGACCUGAGGAACUGCCCC | UGACCUGAGGAACUGCCCC |
| 1269 | AL2-394A | UUUGCAGGGGGGCAGUUCC | UUUGCAGGGGGGCAGUUCC |
| 1270 | AL2-394B | GGAACUGCCCCCCUGCAAA | GGAACUGCCCCCCUGCAAA |
| 1271 | AL2-395A | GGUGGGCCCUGCUUUGCAG | GGUGGGCCCUGCUUUGCAG |
| 1272 | AL2-395B | CUGCAAAGCAGGGCCCACC | CUGCAAAGCAGGGCCCACC |
| 1273 | AL2-396A | UCCAGGAGGUGGGCCCUGC | UCCAGGAGGUGGGCCCUGC |
| 1274 | AL2-396B | GCAGGGCCCACCUCCUGGA | GCAGGGCCCACCUCCUGGA |
| 1275 | AL2-397A | GCUCUCUGAGUCCAGGAGG | GCUCUCUGAGUCCAGGAGG |
| 1276 | AL2-397B | CCUCCUGGACUCAGAGAGC | CCUCCUGGACUCAGAGAGC |
| 1277 | AL2-398A | UUGCCCUGGGCUCUCUGAG | UUGCCCUGGGCUCUCUGAG |
| 1278 | AL2-398B | CUCAGAGAGCCCAGGGCAA | CUCAGAGAGCCCAGGGCAA |
| 1279 | AL2-399A | UGCUUGGCAGUUGCCCUGG | UGCUUGGCAGUUGCCCUGG |
| 1280 | AL2-399B | CCAGGGCAACUGCCAAGCA | CCAGGGCAACUGCCAAGCA |
| 1281 | AL2-400A | CCCGCCAGAAUACUUGUCC | CCCGCCAGAAUACUUGUCC |
| 1282 | AL2-400B | GGACAAGUAUUCUGGCGGG | GGACAAGUAUUCUGGCGGG |
| 1283 | AL2-401A | CUCCCCCACCCCCCGCCAG | CUCCCCCACCCCCCGCCAG |
| 1284 | AL2-401B | CUGGCGGGGGGUGGGGGAG | CUGGCGGGGGGUGGGGGAG |
| 1285 | AL2-402A | CCUGCUCUCUCCCCCACCC | CCUGCUCUCUCCCCCACCC |
| 1286 | AL2-402B | GGGUGGGGGAGAGAGCAGG | GGGUGGGGGAGAGAGCAGG |
| 1287 | AL2-403A | ACCACAGGGCCUGCUCUCU | ACCACAGGGCCUGCUCUCU |
| 1288 | AL2-403B | AGAGAGCAGGCCCUGUGGU | AGAGAGCAGGCCCUGUGGU |
| 1289 | AL2-404A | ACCUCCUGCCACCACAGGG | ACCUCCUGCCACCACAGGG |
| 1290 | AL2-404B | CCCUGUGGUGGCAGGAGGU | CCCUGUGGUGGCAGGAGGU |
| 1291 | AL2-405A | GAGACAAGAUGCCACCUCC | GAGACAAGAUGCCACCUCC |
| 1292 | AL2-405B | GGAGGUGGCAUCUUGUCUC | GGAGGUGGCAUCUUGUCUC |
| 1293 | AL2-406A | UCAGGGACGAGACAAGAUG | UCAGGGACGAGACAAGAUG |
| 1294 | AL2-406B | CAUCUUGUCUCGUCCCUGA | CAUCUUGUCUCGUCCCUGA |
| 1295 | AL2-407A | ACUGGAGCAGACAUCAGGG | ACUGGAGCAGACAUCAGGG |
| 1296 | AL2-407B | CCCUGAUGUCUGCUCCAGU | CCCUGAUGUCUGCUCCAGU |
| 1297 | AL2-408A | CCUGCCAUCACUGGAGCAG | CCUGCCAUCACUGGAGCAG |
| 1298 | AL2-408B | CUGCUCCAGUGAUGGCAGG | CUGCUCCAGUGAUGGCAGG |
| 1299 | AL2-409A | UUCUCCAUCCUCCUGCCAU | UUCUCCAUCCUCCUGCCAU |
| 1300 | AL2-409B | AUGGCAGGAGGAUGGAGAA | AUGGCAGGAGGAUGGAGAA |
| 1301 | AL2-410A | UGCUGGCACUUCUCCAUCC | UGCUGGCACUUCUCCAUCC |
| 1302 | AL2-410B | GGAUGGAGAAGUGCCAGCA | GGAUGGAGAAGUGCCAGCA |
| 1303 | AL2-411A | UGACCCCCAGCUGCUGGCA | UGACCCCCAGCUGCUGGCA |
| 1304 | AL2-411B | UGCCAGCAGCUGGGGGUCA | UGCCAGCAGCUGGGGGUCA |
| 1305 | AL2-412A | GACGUCUUGACCCCCAGCU | GACGUCUUGACCCCCAGCU |
| 1306 | AL2-412B | AGCUGGGGGUCAAGACGUC | AGCUGGGGGUCAAGACGUC |
| 1307 | AL2-413A | UCCUCAGGGGACGUCUUGA | UCCUCAGGGGACGUCUUGA |
| 1308 | AL2-413B | UCAAGACGUCCCCUGAGGA | UCAAGACGUCCCCUGAGGA |
| 1309 | AL2-414A | UGGGCCUGGGUCCUCAGGG | UGGGCCUGGGUCCUCAGGG |
| 1310 | AL2-414B | CCCUGAGGACCCAGGCCCA | CCCUGAGGACCCAGGCCCA |
| 1311 | AL2-415A | AGAAGGGCUGGGUGUGGGC | AGAAGGGCUGGGUGUGGGC |
| 1312 | AL2-415B | GCCCACACCCAGCCCUUCU | GCCCACACCCAGCCCUUCU |
| 1313 | AL2-416A | AUUGGGAGGCAGAAGGGCU | AUUGGGAGGCAGAAGGGCU |
| 1314 | AL2-416B | AGCCCUUCUGCCUCCCAAU | AGCCCUUCUGCCUCCCAAU |
| 1315 | AL2-417A | AGGAGAGAGAAUUGGGAGG | AGGAGAGAGAAUUGGGAGG |
| 1316 | AL2-417B | CCUCCCAAUUCUCUCUCCU | CCUCCCAAUUCUCUCUCCU |
| 1317 | AL2-418A | AAGGGGACGGAGGAGAGAG | AAGGGGACGGAGGAGAGAG |
| 1318 | AL2-418B | CUCUCUCCUCCGUCCCCUU | CUCUCUCCUCCGUCCCCUU |
| 1319 | AL2-419A | AGUGGAGGAAGGGGACGGA | AGUGGAGGAAGGGGACGGA |
| 1320 | AL2-419B | UCCGUCCCCUUCCUCCACU | UCCGUCCCCUUCCUCCACU |
| 1321 | AL2-420A | UAGGCAGCAGUGGAGGAAG | UAGGCAGCAGUGGAGGAAG |
| 1322 | AL2-420B | CUUCCUCCACUGCUGCCUA | CUUCCUCCACUGCUGCCUA |
| 1323 | AL2-421A | ACUGCCUUGCAUUAGGCAG | ACUGCCUUGCAUUAGGCAG |
| 1324 | AL2-421B | CUGCCUAAUGCAAGGCAGU | CUGCCUAAUGCAAGGCAGU |
| 1325 | AL2-422A | UGCUGAGCCACUGCCUUGC | UGCUGAGCCACUGCCUUGC |
| 1326 | AL2-422B | GCAAGGCAGUGGCUCAGCA | GCAAGGCAGUGGCUCAGCA |
| 1327 | AL2-423A | CAUUCUUGCUGCUGAGCCA | CAUUCUUGCUGCUGAGCCA |
| 1328 | AL2-423B | UGGCUCAGCAGCAAGAAUG | UGGCUCAGCAGCAAGAAUG |
| 1329 | AL2-424A | UGUAGAACCAGCAUUCUUG | UGUAGAACCAGCAUUCUUG |
| 1330 | AL2-424B | CAAGAAUGCUGGUUCUACA | CAAGAAUGCUGGUUCUACA |
| 1331 | AL2-425A | UCCUCGGGAUGUAGAACCA | UCCUCGGGAUGUAGAACCA |
| 1332 | AL2-425B | UGGUUCUACAUCCCGAGGA | UGGUUCUACAUCCCGAGGA |
| 1333 | AL2-426A | ACCUCAGACACUCCUCGGG | ACCUCAGACACUCCUCGGG |
| 1334 | AL2-426B | CCCGAGGAGUGUCUGAGGU | CCCGAGGAGUGUCUGAGGU |
| 1335 | AL2-427A | AGUGGGGCGCACCUCAGAC | AGUGGGGCGCACCUCAGAC |
| 1336 | AL2-427B | GUCUGAGGUGCGCCCCACU | GUCUGAGGUGCGCCCCACU |
| 1337 | AL2-428A | CCUCUGUACAGAGUGGGGC | CCUCUGUACAGAGUGGGGC |
| 1338 | AL2-428B | GCCCCACUCUGUACAGAGG | GCCCCACUCUGUACAGAGG |
| 1339 | AL2-429A | CCAAACAGCCUCUGUACAG | CCAAACAGCCUCUGUACAG |
| 1340 | AL2-429B | CUGUACAGAGGCUGUUUGG | CUGUACAGAGGCUGUUUGG |
| 1341 | AL2-430A | GGCAAGGCUGCCCAAACAG | GGCAAGGCUGCCCAAACAG |
| 1342 | AL2-430B | CUGUUUGGGCAGCCUUGCC | CUGUUUGGGCAGCCUUGCC |
| 1343 | AL2-431A | UCUGCUCUCUGGAGGCAAG | UCUGCUCUCUGGAGGCAAG |
| 1344 | AL2-431B | CUUGCCUCCAGAGAGCAGA | CUUGCCUCCAGAGAGCAGA |
| 1345 | AL2-432A | CUGGAAUCUGCUCUCUGGA | CUGGAAUCUGCUCUCUGGA |
| 1346 | AL2-432B | UCCAGAGAGCAGAUUCCAG | UCCAGAGAGCAGAUUCCAG |
| 1347 | AL2-433A | GGCUUCCGAAGCUGGAAUC | GGCUUCCGAAGCUGGAAUC |
| 1348 | AL2-433B | GAUUCCAGCUUCGGAAGCC | GAUUCCAGCUUCGGAAGCC |
| 1349 | AL2-434A | AUGGGAGCACCUUCCAUUC | AUGGGAGCACCUUCCAUUC |
| 1350 | AL2-434B | GAAUGGAAGGUGCUCCCAU | GAAUGGAAGGUGCUCCCAU |
| 1351 | AL2-435A | UCCCCUCCGAUGGGAGCAC | UCCCCUCCGAUGGGAGCAC |
| 1352 | AL2-435B | GUGCUCCCAUCGGAGGGGA | GUGCUCCCAUCGGAGGGGA |
| 1353 | AL2-436A | CUCUGAGGGUCCCCUCCGA | CUCUGAGGGUCCCCUCCGA |
| 1354 | AL2-436B | UCGGAGGGGACCCUCAGAG | UCGGAGGGGACCCUCAGAG |
| 1355 | AL2-437A | GUCUCCAGGGCUCUGAGGG | GUCUCCAGGGCUCUGAGGG |
| 1356 | AL2-437B | CCCUCAGAGCCCUGGAGAC | CCCUCAGAGCCCUGGAGAC |
| 1357 | AL2-438A | AGGCCCACCUGGCAGUCUC | AGGCCCACCUGGCAGUCUC |
| 1358 | AL2-438B | GAGACUGCCAGGUGGGCCU | GAGACUGCCAGGUGGGCCU |
| 1359 | AL2-439A | AGUGGCAGCAGGCCCACCU | AGUGGCAGCAGGCCCACCU |
| 1360 | AL2-439B | AGGUGGGCCUGCUGCCACU | AGGUGGGCCUGCUGCCACU |
| 1361 | AL2-440A | UUUUGGCUUACAGUGGCAG | UUUUGGCUUACAGUGGCAG |
| 1362 | AL2-440B | CUGCCACUGUAAGCCAAAA | CUGCCACUGUAAGCCAAAA |
| 1363 | AL2-441A | CCCACCUUUUGGCUUACAG | CCCACCUUUUGGCUUACAG |
| 1364 | AL2-441B | CUGUAAGCCAAAAGGUGGG | CUGUAAGCCAAAAGGUGGG |
| 1365 | AL2-442A | GGAGUCAGGACUUCCCCAC | GGAGUCAGGACUUCCCCAC |
| 1366 | AL2-442B | GUGGGGAAGUCCUGACUCC | GUGGGGAAGUCCUGACUCC |
| 1367 | AL2-443A | CAAGGACCCUGGAGUCAGG | CAAGGACCCUGGAGUCAGG |
| 1368 | AL2-443B | CCUGACUCCAGGGUCCUUG | CCUGACUCCAGGGUCCUUG |
| 1369 | AL2-444A | AGGGGUGGGGCAAGGACCC | AGGGGUGGGGCAAGGACCC |
| 1370 | AL2-444B | GGGUCCUUGCCCCACCCCU | GGGUCCUUGCCCCACCCCU |
| 1371 | AL2-445A | UGGCAGGCAGGGGUGGGGC | UGGCAGGCAGGGGUGGGGC |
| 1372 | AL2-445B | GCCCCACCCCUGCCUGCCA | GCCCCACCCCUGCCUGCCA |
| 1373 | AL2-446A | UGAGGGCCCAGGUGGCAGG | UGAGGGCCCAGGUGGCAGG |
| 1374 | AL2-446B | CCUGCCACCUGGGCCCUCA | CCUGCCACCUGGGCCCUCA |
| 1375 | AL2-447A | CUGGGCUGUGAGGGCCCAG | CUGGGCUGUGAGGGCCCAG |
| 1376 | AL2-447B | CUGGGCCCUCACAGCCCAG | CUGGGCCCUCACAGCCCAG |
| 1377 | AL2-448A | GUGAGGGUCUGGGCUGUGA | GUGAGGGUCUGGGCUGUGA |
| 1378 | AL2-448B | UCACAGCCCAGACCCUCAC | UCACAGCCCAGACCCUCAC |
| 1379 | AL2-449A | GAGCUCACCUCCCAGUGAG | GAGCUCACCUCCCAGUGAG |
| 1380 | AL2-449B | CUCACUGGGAGGUGAGCUC | CUCACUGGGAGGUGAGCUC |
| 1381 | AL2-450A | AAGGGCAGCUGAGCUCACC | AAGGGCAGCUGAGCUCACC |
| 1382 | AL2-450B | GGUGAGCUCAGCUGCCCUU | GGUGAGCUCAGCUGCCCUU |
| 1383 | AL2-451A | AUCAGGCAGCUUUAUUCCA | AUCAGGCAGCUUUAUUCCA |
| 1384 | AL2-451B | UGGAAUAAAGCUGCCUGAU | UGGAAUAAAGCUGCCUGAU |
| 1385 | TMPRSS6 mRNA | See Fig. 3 | See Fig. 3 |
| 1386 | EU401B without | mU mC mA mC mC mU fG FC fU mU mC mU mU mC mU mG mG | UCACCUGCUUCUUCUGGUU |
| | linker | (ps) mU (ps) mU | |
| 1387 | EU402B without | fU mC fA mC fC mU fG mC fU mU fC mU fU mC fU mG fG | UCACCUGCUUCUUCUGGUU |
| | linker | (ps) mU (ps) fU | |

1. A double-stranded nucleic acid that is capable of inhibiting expression of TMPRSS6, for use in the prevention, decrease of the risk of suffering from or treatment of a myeloproliferative disorder, wherein the nucleic acid comprises a first strand and a second strand, wherein the first strand sequence comprises a sequence of at least 15 nucleotides differing by no more than 3 nucleotides from any one of the sequences selected from SEQ ID NO: 6, 8, 10, 12, 14, 16, 18, 20, 22, 24, 26, 28, 30, 32, 34 and 36.

2. The nucleic acid for use according to claim 1, wherein said first strand comprises a sequence of SEQ ID NO: 6.

3. The nucleic acid for use according to claim 1 or claim 2, wherein the second strand comprises a sequence of SEQ ID NO: 7.

4. The nucleic acid for use according to any of the preceding claims, wherein the first strand and the second strand form a duplex region of 17-25 nucleotides in length.

5. The nucleic acid for use according to any of the preceding claims, wherein at least one nucleotide of the first and/or second strand is a modified nucleotide.

6. The nucleic acid for use according to any of the preceding claims, wherein the nucleic acid comprises a phosphorothioate linkage between the terminal two or three 3′ nucleotides and/or 5′ nucleotides of the first and/or the second strand and optionally wherein the linkages between the remaining nucleotides are phosphodiester linkages.

7. The nucleic acid for use according to any of the preceding claims, wherein the first strand comprises a sequence of SEQ ID NO: 3.

8. The nucleic acid for use according to any of the preceding claims, wherein the second strand comprises a sequence of SEQ ID No: 1386 or of SEQ ID NO: 1387.

9. The nucleic acid for use according to any of the preceding claims, wherein the nucleic acid is conjugated to a ligand.

10. The nucleic acid for use according to claim 9, wherein the ligand comprises (i) one or more N-acetyl galactosamine (GalNAc) moieties or derivatives thereof, and (ii) a linker, wherein the linker conjugates the at least one GalNAc moiety or derivative thereof to the nucleic acid.

11. The nucleic acid for use according to claim 9 or claim 10, wherein the nucleic acid is conjugated to a ligand. of the following structure 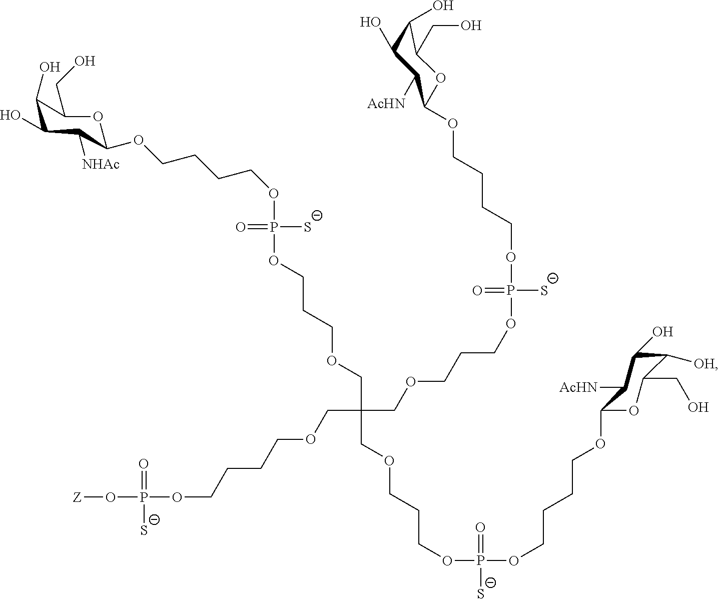
wherein the terminal phosphorothioate group of the ligand moiety is bonded to the 5′position of the 5′terminal nucleotide of the second strand of the nucleic acid (denoted by “Z”) or to the 3 position of the 3′terminal nucleotide of the second strand of the nucleic acid (denoted by “Z”).

12. A composition for use in the prevention, decrease of the risk of suffering from or treatment of a myeloproliferative disorder comprising a nucleic acid defined in any of claims 1-11 and a solvent and/or a delivery vehicle and/or a physiologically acceptable excipient and/or a carrier and/or a salt and/or a diluent and/or a buffer and/or a preservative and/or a further therapeutic agent selected from an oligonucleotide, a small molecule, a monoclonal antibody, a polyclonal antibody and a peptide.

13. A method of preventing, decreasing the risk of suffering from, or treating a myeloproliferative disorder comprising administering a pharmaceutically effective amount of a nucleic acid defined in any of claims 1-11, or of a composition defined in claim 12 to an individual in need of treatment.

14. The nucleic acid for use according to any of claims 1-9, or the composition for use according to claim 12, or the method according to claim 13, wherein the myeloproliferative disorder is:
a) a Philadelphia chromosome (BCR-ABL) negative myeloproliferative neoplasm;
b) one or several of polycythaemia vera (PV), essential thrombocythaemia (ET) and primary myelofibrosis (PMF); or
c) polycythemia vera (PV).

15. The nucleic acid for use according to any of claims 1-9, or the composition for use according to claim 12, or the method according to claim 13, wherein the myeloproliferative disorder is polycythaemia vera.

16. The nucleic acid for use according to any of claims 1-9, or the composition for use according to claim 12, or the method according to claim 13, wherein the myeloproliferative disorder is JAK2 positive polycythaemia vera.
